(19) United States
(12) Patent Application Publication
(10) Pub. No.: US 2025/0163410 A1
(43) Pub. Date: May 22, 2025

(54) CRISPR-TRANSPOSON SYSTEMS FOR DNA MODIFICATION

(71) Applicant: The Trustees of Columbia University in the City of New York, New York, NY (US)

(72) Inventors: Samuel Henry Sternberg, New York, NY (US); Sanne Eveline Klompe, New York, NY (US); Matthew Walker, New York, NY (US); Dennis James Zhang, New York, NY (US); George Davis Lampe, New York, NY (US)

(21) Appl. No.: 18/875,026

(22) PCT Filed: Jun. 13, 2023
(86) PCT No.: PCT/US23/68361

§ 371 Date: Dec. 13, 2024

Related U.S. Application Data
(60) Provisional application No. 63/351,753, filed on Jun. 13, 2022.
(60) Provisional application No. 63/380,330, filed on Oct. 20, 2022.
(60) Provisional application No. 63/479,481, filed on Jan. 11, 2023.

(51) Int. Cl. C12N 15/11; C12N 9/22; C12N 15/90

(52) U.S. Cl. CPC C12N 15/11; C12N 9/22; C12N 15/902; C12N 2310/20; C12N 2800/80

(57) ABSTRACT

This disclosure to the methods for nucleic acid modification, gene targeting, and gene tagging comprising an engineered Clustered Regularly Interspaced Short Palindromic Repeats (CRISPR)-associated transposon (CAST) system with a donor DNA comprising at least one engineered transposon end sequence and/or at least one integration co-factor protein. More particularly, the present disclosure provides systems comprising: an engineered CAST system or one or more nucleic acids encoding the engineered CAST system, wherein the CAST system comprises at least one or all of: i) at least one Cas protein, ii) one or more transposon-associated proteins, iii) at least one guide RNA (gRNA) complementary to at least a portion of a target nucleic acid sequence; and a donor nucleic acid comprising a cargo nucleic acid sequence flanked by at least one engineered transposon end sequence and/or at least one integration co-factor protein, or a nucleic acid encoding thereof.

### CROSS-REFERENCE TO RELATED APPLICATIONS

This application claims the benefit of U.S. Provisional Application Nos. 63/351,753, filed Jun. 13, 2022, 63/380,330, filed Oct. 20, 2022, and 63/479,481, filed Jan. 11, 2023, the contents of which are herein incorporated by reference in their entirety.

### STATEMENT REGARDING FEDERALLY SPONSORED RESEARCH OR DEVELOPMENT

This invention was made with government support under grant number HG011650 and AI168976 awarded by the National Institutes of Health. The government has certain rights in the invention.

### FIELD

The present invention relates to methods and systems for DNA modification, gene targeting, and gene tagging comprising an engineered Clustered Regularly Interspaced Short Palindromic Repeats (CRISPR)-associated transposon (CAST) system having a donor DNA comprising at least one engineered transposon end sequence and/or at least one integration co-factor protein.

### SEQUENCE LISTING STATEMENT

The contents of the electronic sequence listing titled COLUM_40991_601.xml (Size: 6,329,222 bytes; and Date of Creation: Jun. 13, 2023) is herein incorporated by reference in its entirety.

### BACKGROUND

CRISPR-Cas systems can be used for programmable DNA integration, in which the nuclease-deficient CRISPR-Cas machinery (either Cascade from Type I systems, or Cas12 from Type V systems) coordinates with Tn7 transposon-associated proteins to mediate RNA-guided DNA targeting and DNA integration, respectively. This activity may be leveraged in bacterial or eukaryotic cells for the targeted integration of user-defined genetic payloads at user-defined genomic loci, via a mechanism that obviates requirements for DNA double-strand breaks (DSBs) necessary for homology-directed repair.

### SUMMARY

Provided herein are systems for RNA-guided nucleic acid modification. The systems comprise a) an engineered Clustered Regularly Interspaced Short Palindromic Repeats (CRISPR)-associated transposon (CAST) system or one or more nucleic acids encoding the engineered CAST system, wherein the CAST system comprises at least one or all of: i) at least one Cas protein; ii) at least one transposon-associated protein; and iii) at least one guide RNA (gRNA) complementary to at least a portion of a target nucleic acid sequence; and b) a donor nucleic acid comprising a cargo nucleic acid sequence flanked by at least one or both of: an engineered transposon right end sequence or an engineered transposon left end sequence; and/or c) at least one integration co-factor protein, or a nucleic acid encoding thereof.

In some embodiments, the engineered transposon right end sequence and/or the engineered left end sequence encodes an amino acid linker sequence. In some embodiments, the engineered transposon right end sequence and/or the engineered left end sequence is fully or partially AT rich. In some embodiments, the engineered transposon right end sequence and/or the engineered left end sequence comprises a 5 to 8 bp terminal end sequence.

In some embodiments, the engineered transposon right end sequence and/or the engineered left end sequence comprises at least two TnsB binding sites (TBSs). In some embodiments, each TBS comprises a sequence individually selected from: SEQ ID NO: 11, or SEQ ID NO: 12, wherein each M is individually A or C; each W is independently A or T; each R is independently A or G; each D is independently A, G or T; each Y is independently T or C; each K is G or T; B is G, T, or C; and each H is independently A, C or T.

In some embodiments, the engineered transposon right end sequence is at least about 75 basepairs (bp). In some embodiments, the engineered transposon right end sequence comprises a sequence of: SEQ ID NO: 1, or a variant sequence having one or more additions, substitutions or deletions thereof; any of SEQ ID NOs: 2-8; any of SEQ ID NOs: 18-844; SEQ ID NOs: 9, or a variant sequence having one or more additions, substitutions or deletions thereof; any of SEQ ID NOs: 845-2690; any of SEQ ID NOs: 2691-2702; or any of SEQ ID NOs: 2703-3119.

In some embodiments, the engineered transposon left end sequence is at least about 115 basepairs (bp). In some embodiments, the engineered transposon left end sequence further comprises an Integration Host Factor (IHF) binding site (IBS), wherein the IBS comprises a sequence of WATCARNNNNTTR, wherein W is A or T, R is A or G, and N is any nucleotide. In some embodiments, the engineered transposon left end sequence comprises a sequence of: SEQ ID NO: 10, or a variant sequence having one or more substitutions thereof; any of SEQ ID NOs: 3120-4665; any of SEQ ID NOs: 4666-4673; or any of SEQ ID NOs: 4674-5135.

In some embodiments, the cargo nucleic acid sequence encodes a peptide tag or a polypeptide.

In some embodiments, the at least one integration co-factor protein comprises Integration Host Factor (IHF), Factor for Inversion Stimulation (Fis), or a combination thereof.

In some embodiments, the engineered transposon right end sequence and/or the engineered transposon left end sequence is derived from Vibrio cholerae Tn6677 or Pseudoalteromonas Tn7016.

Provided herein are systems for RNA-guided nucleic acid modification. The systems comprise a) an engineered Clustered Regularly Interspaced Short Palindromic Repeats (CRISPR)-associated transposon (CAST) system or one or more nucleic acids encoding the engineered CAST system, wherein the CAST system comprises at least one or all of: i) at least one Cas protein; ii) at least one transposon-associated protein; iii) at least one guide RNA (gRNA) complementary to at least a portion of a target nucleic acid sequence; and b) a donor nucleic acid comprising a cargo nucleic acid sequence flanked by at least one engineered transposon end sequence; and/or c) at least one integration co-factor protein, or a nucleic acid encoding thereof. In some embodiments, the at least one engineered transposon end sequence encodes an amino acid linker sequence.

In some embodiments, the systems comprise a) an engineered Clustered Regularly Interspaced Short Palindromic Repeats (CRISPR)-associated transposon (CAST) system or one or more nucleic acids encoding the engineered CAST system, wherein the CAST system comprises at least one or all of: i) at least one Cas protein; ii) at least one transposon-associated protein; iii) at least one guide RNA (gRNA) complementary to at least a portion of a target nucleic acid sequence; and b) a donor nucleic acid comprising a cargo nucleic acid sequence flanked by at least one engineered transposon end sequence. In some embodiments, the at least one engineered transposon end sequence encodes an amino acid linker sequence.

In some embodiments, the systems comprise a) an engineered Clustered Regularly Interspaced Short Palindromic Repeats (CRISPR)-associated transposon (CAST) system or one or more nucleic acids encoding the engineered CAST system, wherein the CAST system comprises at least one or all of: i) at least one Cas protein; ii) at least one transposon-associated protein; iii) at least one guide RNA (gRNA) complementary to at least a portion of a target nucleic acid sequence; and b) at least one integration co-factor protein, or a nucleic acid encoding thereof.

In some embodiments, the systems comprise a) an engineered Clustered Regularly Interspaced Short Palindromic Repeats (CRISPR)-associated transposon (CAST) system or one or more nucleic acids encoding the engineered CAST system, wherein the CAST system comprises at least one or all of: i) at least one Cas protein; ii) at least one transposon-associated protein; iii) at least one guide RNA (gRNA) complementary to at least a portion of a target nucleic acid sequence; b) a donor nucleic acid comprising a cargo nucleic acid sequence flanked by at least one engineered transposon end sequence; and c) at least one integration co-factor protein, or a nucleic acid encoding thereof. In some embodiments, the at least one engineered transposon end sequence encodes an amino acid linker sequence.

In some embodiments, the donor nucleic acid comprises a cargo nucleic acid sequence flanked by one native transposon end sequence and one engineered transposon end sequence.

In some embodiments, the at least one engineered transposon end sequence is fully or partially AT-rich.

In some embodiments, the at least one engineered transposon end sequence comprises at least two TnsB binding sites (TBSs). In some embodiments, each TBS comprises a sequence individually selected from: CAMCCATAWRDTGATAWYKH (SEQ ID NO: 11), or CMMCBRWAWNNTGAHWWYWN (SEQ ID NO: 12), wherein each M is individually A or C; each W is independently A or T; each R is independently A or G; each D is independently A, G or T; each Y is independently T or C; each K is G or T; B is G, T, or C; and each H is independently A, C or T.

In some embodiments, the at least one engineered transposon end sequence comprises a 5 to 8 bp terminal end sequence. In some embodiments, the terminal end sequence comprises a terminal TG dinucleotide. In some embodiments, the terminal end sequence is immediately adjacent to the distal end of the transposase binding site farthest from the cargo nucleic acid sequence. In some embodiments, the terminal end sequence is separated from the distal end of the transposase binding site farthest from the cargo nucleic acid sequence by 1 to 3 basepairs (bp).

In some embodiments, the at least one engineered transposon end sequence is a transposon right end sequence 3′ to the cargo nucleic acid sequence, relative to transcription direction. In some embodiments, the at least one engineered transposon end sequence is a transposon left end sequence 5′ to the cargo nucleic acid sequence, relative to transcription direction. In some embodiments, the donor nucleic acid comprises a cargo nucleic acid sequence flanked by two engineered transposon sequences: an engineered transposon right end sequence and an engineered transposon left end sequence.

In some embodiments, the engineered transposon right end sequence and/or the engineered transposon left end sequence is derived from a Vibrio cholerae Tn6677 native transposon end sequence. In some embodiments, the engineered transposon right end sequence and/or the engineered transposon left end sequence is derived from a Pseudoalteromonas Tn7016 native transposon end sequence.

In some embodiments, the engineered transposon right end sequence is at least about 50 basepairs (bp). In some embodiments, the engineered transposon right end sequence is at least about 75 basepairs (bp).

In some embodiments, the engineered transposon right end sequence comprises two TBSs.

In some embodiments, the engineered transposon right end sequence comprises a sequence of: TGTTGATACAACCATAAAATGATAATTACACCCATAAATTGATAATTATCACACCCA (SEQ ID NO: 1), or a variant sequence having one or more additions, deletions, or substitutions thereof.

In some embodiments, the engineered transposon right end sequence comprises a sequence of:

(SEQ ID NO: 2)
TGTgGATACAACCATAAAATGATAATTACACCCATAAATgGATcA
TTATCACcCCCA;
(SEQ ID NO: 3)
TGTgGATACAACCATAAAAcGATAATTACACCCATAAATgGATcA
TTATCACACCCA;
(SEQ ID NO: 4)
TGTgGATcCAACCATAAAATGATAATTACACCCATAAATgGATcA
TTATCACACCCA;
(SEQ ID NO: 5)
TGTTGATACAACCATAAAAgGATtATTACACCCATtAATTGATAA
TTATCACACCCA;
(SEQ ID NO: 6)
TGTTGATACAACCATcAAATGgTAATTACACCCATAAATTGATAA
TTATCACACCCA;
(SEQ ID NO: 7)
TGTTGATACAACCATtAAATGATAATTcCACCCATAAtTTGATAA
TTATCACACCCA;
or
(SEQ ID NO: 8)
TGTTGATACAACCATtAAATGgTAATTcCACCCAaAtATTGATAA
TTATCACACCCA.

In some embodiments, the engineered transposon right end sequence comprises a sequence of SEQ ID NOs: 18-844.

In some embodiments, the engineered transposon right end sequence comprises a sequence of: TGTTGATACAACCATAAAATGATAATTACACCCATAAATTGATAATTATCACACCCATAAA TTGATATTGCCTCT (SEQ ID NO: 9), or a variant sequence having one or more additions, deletions, or substitutions thereof.

In some embodiments, the engineered transposon right end sequence comprises a sequence of SEQ ID NOs: 845-2690.

In some embodiments, the engineered transposon right end sequence is hyperactive. In some embodiments, the engineered transposon right end sequence comprises a sequence of SEQ ID NOs: 2691-2702. In some embodiments, the engineered transposon right end sequence comprises a sequence of SEQ ID NOs: 2703-3119.

In some embodiments, the engineered transposon left end sequence is at least about 105 basepairs (bp). In some embodiments, the engineered transposon left end sequence is at least about 115 bp.

In some embodiments, the engineered transposon left end sequence comprises three transposase TBSs.

In some embodiments, the engineered transposon left end sequence comprises an Integration Host Factor (IHF) binding site (IBS). In some embodiments, the IBS comprises a sequence of WATCARNNNNTTR, wherein W is A or T, R is A or G, and N is any nucleotide. In some embodiments, the engineered transposon left end sequence does not include an Integration Host Factor (IHF) binding site (IBS).

In some embodiments, the engineered transposon left end sequence comprises a sequence of: TGTTGATGCAACCATAAAGTGATATTTAATAATITATTTATAATCAGCA ACTTAACCACAAA ACAACCATATATTGATATCTCACAAAACAACCATAAGTTGATATITITGTGAAT (SEQ ID NO: 10), or a variant sequence having one or more additions, deletions, or substitutions thereof.

In some embodiments, the engineered transposon left end sequence comprises a sequence of SEQ ID NOs: 3120-4665.

In some embodiments, the engineered transposon left end sequence is hyperactive. In some embodiments, the engineered transposon left end sequence comprises a sequence of SEQ ID NOs: 4666-4673. In some embodiments, the engineered transposon left end sequence comprises a sequence of SEQ ID NOs: 4674-5135.

In some embodiments, the cargo nucleic acid sequence encodes a peptide tag. In some embodiments, the cargo nucleic acid sequence encodes a polypeptide. In some embodiments, the polypeptide comprises a fluorescent protein.

In some embodiments, the at least one integration co-factor protein comprises Integration Host Factor (IHF), Factor for Inversion Stimulation (Fis), or a combination thereof. In some embodiments, the at least one integration co-factor protein is provided as a fusion protein with TnsA and TnsB, or a nucleic acid encoding thereof. In some embodiments, the at least one integration co-factor protein fused to a localization agent. In some embodiments, the at least one integration co-factor protein comprises an amino acid sequence of any of SEQ ID NOs: 5136-5152.

In some embodiments, the at least one Cas protein is derived from a Type-I CRISPR-Cas system. In some embodiments, the engineered CAST system is a Type I-F system.

In some embodiments, the at least one Cas protein comprises Cas5, Cas6, Cas7, and Cas8. In some embodiments, the at least one Cas protein comprises a Cas8-Cas5 fusion protein.

In some embodiments, the at least one transposon protein is derived from a Tn7 or Tn7-like transposon system. In some embodiments, the at least one transposon-associated protein comprises TnsA, TnsB, TnsC, or a combination thereof. In some embodiments, the at least one transposon protein comprises a TnsA-TnsB fusion protein. In some embodiments, the at least one transposon-associated protein comprises TnsD and/or TniQ.

In some embodiments, the engineered transposon system is derived from Vibrio cholerae Tn6677. In some embodiments, the engineered transposon system is derived from Pseudoalteromonas Tn7006.

In some embodiments, the at least one gRNA is a non-naturally occurring gRNA. In some embodiments, the at least one gRNA is encoded in a CRISPR RNA (crRNA) array.

In some embodiments, the systems further comprise a target nucleic acid. In some embodiments, the target nucleic acid sequence comprises a TSD region having a 5′-CWG-3′ sequence motif.

In some embodiments, the one or more nucleic acids encoding the engineered CAST system comprises one or more messenger RNAs, one or more vectors, or a combination thereof. In some embodiments, the at least one Cas protein, the at least one transposon-associated protein, and the at least one gRNA are encoded by different nucleic acids. In some embodiments, the one or more of the at least one Cas protein, the at least one transposon-associated protein, and the at least one gRNA are encoded by a single nucleic acid.

In some embodiments, the nucleic acid encoding the at least one integration co-factor protein comprises at least one messenger RNA, at least one vector, or a combination thereof. In some embodiments, the at least one integration co-factor protein is encoded on a nucleic acid encoding one or more of: the at least one Cas protein, the at least one transposon-associated protein, and the at least one gRNA.

Also provided are compositions and cells comprising the disclosed system. In some embodiments, the cell is a prokaryotic cell. In some embodiments, the cell is a eukaryotic cell. In some embodiments, the cell is a mammalian cell. In some embodiments, the cell is a human cell.

Further provided are methods for nucleic acid integration comprising contacting a target nucleic acid sequence with a disclosed system or composition. In some embodiments, the target nucleic acid sequence comprises a TSD region having a 5′-CWG-3′ sequence motif.

In some embodiments, the target nucleic acid encodes a polypeptide gene product or is adjacent to a sequence encoding a polypeptide gene product.

In some embodiments, the target nucleic acid sequence is in a cell. In some embodiments, the contacting a target nucleic acid sequence comprises introducing the system into the cell. In some embodiments, the cell is a prokaryotic cell. In some embodiments, the cell is a eukaryotic cell. In some embodiments, the cell is a mammalian cell. In some embodiments, the cell is a human cell.

In some embodiments, the introducing the system into the cell comprises administering the system to a subject. In some embodiments, introducing the system into the cell comprises administering the system to a subject. In some embodiments, the administering comprises in vivo administration. In some embodiments, the administering comprises transplantation of ex vivo treated cells comprising the system.

Other aspects and embodiments of the disclosure will be apparent in light of the following detailed description.

### BRIEF DESCRIPTION OF THE DRAWINGS

FIGS. 1A-1E show the pooled library approach to investigate transposon end mutability. FIG. 1A is a schematic of RNA-guided transposition with VchCAST. FIG. 1B is a graph of integration efficiency of the WT mini-transposon in both orientations when directed to a genomic lacZ target site, as measured by qPCR. FIG. 1C is a table of the number of transposon right and left end library variants tested in each category. FIG. 1D is a schematic of an exemplary pooled library transposition approach. Library members were synthesized as single-stranded oligos and cloned into a plasmid donor library (pDonor), with 8-bp barcodes (gray) located between the transposon end and cargo used to uniquely identify each variant. The donor library was used for transposition into the E. coli genome, and junction amplicons were generated to determine the representation of each library member within integrated products by NGS. FIG. 1E is a schematic of the native VchCAST system from Vibrio cholerae (top), and relative T-RL integration activity for library members in which the left and right ends were sequentially mutagenized beginning internally (bottom). Each point represents the average activity from two transposition experiments using the same pooled donor library. Left end sequence is SEQ ID NO: 5229; right end sequence is SEQ ID NO: 5230.

FIGS. 2A-2E show transposase binding site (TBS) characterization for VchCAST. FIG. 2A is a schematic representation of the VchCAST transposon end sequences. Bioinformatically predicted transposase binding site (TBS) sequences are indicated with blue boxes and labeled L1-L3 and R1-R3. The 8-bp terminal end sequences that dictate the transposon boundaries are marked with yellow boxes. Left end sequence is SEQ ID NO: 5231; right end sequence is SEQ ID NO: 5230. FIG. 2B is a WebLogo depicting the sequence conservation of the six bioinformatically predicted TBSs. FIG. 2C is a graph of the relative integration efficiencies (log 2-transformed) for mutagenized TBS sequences averaged over all six binding sites, shown as the mean for two biological replicates. FIG. 2D, top is Tn7002 transposon end sequences colored based VchCAST transposon end library data, where red indicates a relatively inefficient residue (L1-SEQ ID NO: 5232; L2-SEQ ID NO: 5233; L3-SEQ ID NO: 5234; R1-SEQ ID NO: 5235; R2-SEQ ID NO: 5236; R3-SEQ ID NO: 5237). FIG. 2D, bottom is relative integration efficiencies of VchCAST/Tn7002 chimeric ends verify critical compatibility sequence requirements of TBSs. Data are shown for two biological replicates. FIG. 2E is a graph of relative integration efficiencies for transposon variants containing altered distances between the indicated TBSs. Orange arrows highlight the 10-bp periodic pattern of activity. Data are shown for two biological replicates.

FIGS. 3A-3D shows transposase sequence preferences influence on integration site patterns. FIG. 3A shows VchCAST exhibits target-specific heterogeneity in the distance (d) between the target site and integration site, which could result from sequence preferences within the downstream region (top). Deep sequencing revealed biases in integration site preference, with integration patterns shown for four target sites (4-7) located in the lac operon of the E. coli BL21(DE3) genome (top row) or encoded on a separate target plasmid (second row). Chimeric target plasmids that either maintain the 32-bp target site (third row) or 60-bp downstream region (bottom row) of target 4 were also tested. These data reveal that sequence identity of the downstream region (including the integration site), but not the target site, governs the observed in integration distance distribution. FIG. 3B is a schematic of integration site library experiment, in which integration was directed into an 8-bp degenerate sequence encoded on a target plasmid (pTarget). FIG. 3C is a sequence logo of preferred integration site, generated by selecting nucleotides from the top 5000 enriched sequences across all integration positions in each library, with a minimum threshold of four-fold enrichment in the integrated products compared to the input. FIG. 3D shows the preferred 5′-CWG-3′ motif in the center of the TSD is predictive of integration site distribution, as the displacement of this motif within the degenerate sequence shifts the preferred integration site distance, indicated by the red number.

FIGS. 4A-4E show that engineered transposon right ends enable functional in-frame protein tagging. FIG. 4A is an illustration of a minimal transposon right end sequence (“WT-min.” SEQ ID NO:1) and the amino acids it encodes in three different reading frames. The 8-bp terminal end (yellow box) and TBSs (blue boxes) are shown. ORF-1 (SEQ ID NOs: 5238 and 5239); ORF-2 (SEQ ID NOs: 5240 and 5241) and ORF-3 (SEQ ID NOs: 5242 and 5243). FIG. 4B is a graph of integration efficiencies for individual pDonor variants in which stop codons and codons encoding bulky/charged amino acids were replaced, as determined by qPCR. “Vector only” refers to the negative control condition where pEffector was co-transformed with a vector that did not encode a transposon. FIG. 4C shows select right end linker variants cloned in between the 10^{th} and 11th β-strands of GFP, in order to identify stable polypeptide linkers that still allow for proper formation and fluorescence activity of GFP. Normalized fluorescence intensity (NFI) was calculated using the optical density of each culture and is plotted for each linker variant alongside wildtype GFP. A schematic of a proof-of-concept experiment in which the endogenous E. coli gene msrB is tagged by targeted, site-specific RNA-guided transposition (FIG. 4D, top). Fluorescence microscopy images reveal functional tagging of MsrB with the linker variant right end, but not the WT, stop codon-containing right end (FIG. 4D, bottom). Scale bar represents 10 μm. FIG. 4E is western blots with anti-GFP antibody (top) and anti-GAPDH antibody (bottom) as loading control. The four samples are unmodified BL21(DE3) cells (‘-’), cells that underwent transposition with a GFP-encoding donor plasmid using either the WT transposon end (‘WT’) or the modified ORF2a transposon end (‘Variant’), and cells expressing a plasmid encoding GFP driven by a T7 promoter (‘pGFP’). The expected size of GFP alone is 26.8 kDa, while the expected size of the MsrB-GFP fusion product is ˜42 kDa.

FIGS. 5A-5G show IHF involvement in RNA-guided transposition by VchCAST. FIG. 5A shows library mutagenesis data for the transposon left end (SEQ ID NO: 5244). Each point represents the effect of 4-bp mutations, averaged across 4 variants per base. FIG. 5B shows integration activity of VchCAST in WT, ΔihfA, and ΔihfB cells. Integration activity was rescued by a plasmid encoding both ihfA and ihfB (pRescue). Each point represents integration efficiency measured by qPCR for one independent biological replicate. FIG. 5C shows integration activity when the IHF binding site (IBS) is mutated (Mut), in which all consensus bases within the IBS were modified (from 5′-AATCAGCAAACTTA-3′ (SEQ ID NO: 13) to 5′-CCGACTCAACGGC-3′(SEQ ID NO: 14)). FIG. 5D shows conservation of the IBS in the transposon left end of twenty Type I-F CAST systems, described in Klompe et al., 2022 (Mol Cell, 82, 616-628.e5). IBS sequences are SEQ ID NOs: 5245-5264, top to bottom. FIG. 5E shows a sequence logo generated by aligning the left end sequence of all homologs around the conserved IHF binding site. FIG. 5F shows integration activity in WT and ΔIHF cells for five highly active Type I-F CAST systems. Asterisks indicate the degree of statistical significance:* p≤0.05, ** p≤0.01, ***p≤0.001. FIG. 5G shows an exemplary model: IHF binds the left end to resolve the spacing between the first two TBSs, bringing together TnsB protomers to form an active transpososome.

FIGS. 6A-6E show sequencing and characterization of pDonor right end and left end pooled libraries. FIG. 6A is a histogram showing read counts for each of the input libraries, as defined by barcode sequences. All library members are represented in both the transposon left end and right end libraries. FIG. 6B is a histogram showing the percentage of each library member's high-quality reads in which the correct barcode is coupled to the correct transposon end sequence. Library members are identified by their barcodes. FIG. 6C is a histogram showing the highest percentage of each library member's uncoupled reads mapping to a single incorrect sequence. In other words, for a given library member, the incorrect (uncoupled) sequence with the highest read count was selected and expressed as the percent of total reads for that library member. These analyses demonstrate that only a small minority of all barcode reads for a given library member are associated with an incorrect (uncoupled) transposon end sequence. FIG. 6D shows all enrichment scores for library members in either integration orientation, for both the left end and right end libraries. Enrichment scores were calculated by dividing the abundance of each member in the output library by its abundance in the input library, and then taking the log 2 transformation of that value. Library member dropouts were arbitrarily assigned a score of −15, which fell below the minimum enrichment score across all samples, in order to be plotted on the same graphs. FIG. 6E shows the correlation between two independent biological replicates for the transposon left and right end library transposition experiments. For each graph, the upper R² value (black) includes enrichment scores for all transposon end variants, where dropouts were arbitrarily set to −15. The lower R² value (colored) includes only the enrichment scores for transposon end variants that were detected in both output libraries.

FIGS. 7A-7D show the sequence and spatial characterization of VchCAST TBSs. FIG. 7A shows sequence conservation among the six bioinformatically predicted TBS sequences, with nucleotides conserved among all six sites highlighted in gray. L1 is SEQ ID NO: 5265; L2 is SEQ ID NO: 5266; L3 is SEQ ID NO: 5267; R1 is SEQ ID NO: 5268; R2 is SEQ ID NO: 5269; R #is SEQ ID NO: 5270. FIG. 7B is integration activity for mutagenized TBS sequences at individual binding sites, shown as the mean of two biological replicates. Integration activity is represented as the library variant enrichment score normalized to WT. A schematic representation of the transposon end architecture is shown in FIG. 7C, top. Enrichment of individual transposon end variants for which the TBS were shuffled are shown as a heatmap (FIG. 7C, bottom left). The overall effect of each TBS is represented in a boxplot for the individual sites within both the left and right transposon ends, including their numerical mean (FIG. 7C, bottom right). A schematic representation of the spacing in between the TBS sequences of the transposon left and right ends is shown in FIG. 7D, top left. Integration efficiencies, calculated from enrichments within the larger transposon end library dataset, are shown for alternative spacing between the TBS sequences of the left and right end sequences.

FIGS. 8A-8E show transposase sequence preferences at the site of DNA integration. FIG. 8A is a schematic of target A integration products, with corresponding sequence logos of enriched sequences at each integration position. Sequence logos were generated by selecting all sequences with 4-fold enrichment in the integrated products compared to the input libraries. The y-axis of each sequence logo was set to a maximum of 1 bit. FIG. 8B shows integration site distance distribution for degenerate sequences containing multiple preferred CWG motifs, with preferred distances indicated in red. FIG. 8C shows integration site distance distributions of previously tested genomic target sites, as determined through deep sequencing. The TSD sequence+/−3-bp is shown for distances of 48, 49, and 50 bp. Integration occurs primarily 49-bp downstream of the target site but can be biased to occur 48- and/or 50-bp downstream due to sequence preferences at the site of integration. The TSD is bold, and favored (green) or disfavored (orange and red) nucleotides according to the preference sequence logo are indicated. SEQ ID NOs: 5282-5284 in the upper left panel; SEQ ID NOs: 5285-5287 in the upper middle panel: SEQ ID NOs: 5288-5290 in the upper right panel: SEQ ID NOs: 5291-5293 in the lower left panel; SEQ ID NOs: 5294-5296 in the lower middle panel; SEQ ID NOs: 5297-5299 in the lower right panel. FIG. 8D shows integration site distance distribution for two targets, A and B, with preferred distances indicated in red. FIG. 8E shows nucleotide preferences surrounding the degenerate sequence may be responsible for differences in the overall integration site distance distribution.

FIGS. 9A-9F show the effect of target-transposon boundary sequences and internal sequences on DNA integration. A schematic representation of DNA cleavage by TnsA and TnsB, leading to full excision of the transposon from the donor site is shown in FIG. 9A, top. Different transposon-flanking sequences were tested on both the left and right transposon boundaries, and integration efficiencies were determined by calculating the enrichment of each library member from within the larger transposon end pool (FIG. 9A, bottom). An illustration of the imperfect 8-bp terminal end sequences for VchCAST is shown in FIG. 9B, top. Calculated integration efficiencies are plotted for transposon end variants in which either the left or right terminal end sequence was mutated (FIG. 9B, bottom). An illustration of the transposon end sequences including the target site duplication (TSD), 8-bp terminal end, and first transposase binding site (TBS1) is shown in FIG. 9C, top. The specific sequence shown (SEQ ID NO: 5302) is derived from the VchCAST left end. Integration efficiencies relative to WT are shown for transposon end variants in which the distance between the 8-bp terminal end and TBS1 was altered for either the transposon left or right end (FIG. 9C, middle). Analysis of deep sequencing data revealed TnsB cleavage sites for the right end and left end variants that were functional for transposition; cleavage sites are indicated with red arrows (FIG. 9C, bottom). TBS1 sequence is SEQ ID NO: 5304. Right end sequences are SEQ ID NOs: 5303, 5305 and 5306 for WT, +1 and +3, respectively. Left end sequences are SEQ ID NOs: 5307-5311 for −3, −2, WT, +1 and +3, respectively. FIG. 9D is an illustration of WT and modified transposon right end sequences. The 8-bp terminal end (yellow boxes), transposase binding sites (blue boxes), and palindromic sequences (blue and pink lines), are indicated. The native sequence (SEQ ID NO: 5312) encompasses 130 bp from V. cholerae Tn6677, whereas only 75 bp were used in the “WT” sequence (SEQ ID NO: 5313) used in library experiments. FIG. 9E is a graph of the integration activity of right end library variants, in which the palindromic sequence was altered. Integration activity is represented as the library variant enrichment score normalized to WT. Each variant included a distinct combination of palindromic sequences P_{B} and P_{A}, with the ordering as shown. Blue text (“native”) indicates the native palindromic sequence. Orange text (“G-T”) refers to variants in which palindrome nucleotides were mutated from G to T and A to C. Green text (“G-C”) refers to variants in which palindrome nucleotides were mutated from G to C and A to T. FIG. 9F is a graph of the integration efficiencies of right end variants in which different internal promoter sequences point inwards of the transposon (In) or outwards across the transposon end (Out). Promoter strengths are indicated pJ23114 (+), pJ23111 (++), pJ23119 (+++).

FIGS. 10A-10D show engineering of the VchCAST right end. FIG. 10A is integration data for transposon right end variants that were modified to encode functional protein linker sequences in each of three open reading frames (ORF1-3). Integration efficiencies were calculated based on enrichment values within the library dataset. A schematic representation of the linker functionality assay in which GFP includes a linker sequence encoded by a mutated right end is shown in FIG. 10B, top. The fluorescence of E. coli cells expressing each of the indicated GFP constructs was visualized upon excitation with blue light (FIG. 10B, bottom). FIG. 10C shows fluorescence microscopy images of negative control samples for the C-terminal GFP-tagging experiment, showing a brightfield image (left), fluorescence image (center), and composite merge (right). Controls included experiments testing a non-targeting pEffector alone (top) or in combination with either a transposon encoding a functional linker variant (middle) or a wildtype transposon (bottom). Scale bar represents 10 μm. FIG. 10D is a schematic of transposon right end linker variants. Shading indicates amino acids that differ from the WT ORF. WT-min is SEQ ID NO: 1. WT ORF-1 is SEQ ID NOs: 5238 and 5239; WT is ORF-2 SEQ ID NOs: 5240 and 5241 and WT ORF-3 is SEQ ID NOs: 5242 and 5243. Variant ORF1a DNA sequence is SEQ ID NO: 2 and amino acid sequence is SEQ ID NO: 5354. Variant ORF1b DNA sequence is SEQ ID NO: 3 and amino acid sequence is SEQ ID NO: 5355. Variant ORF1v DNA sequence is SEQ ID NO: 4 and amino acid sequence is SEQ ID NO: 5356. Variant ORF2a DNA sequence is SEQ ID NO: 5 and amino acid sequence is SEQ ID NO: 5357. Variant ORF3a DNA sequence is SEQ ID NO: 6 and amino acid sequence is SEQ ID NO: 5358. Variant ORF3b DNA sequence is SEQ ID NO: 7 and amino acid sequence is SEQ ID NO: 5359. Variant ORF3c DNA sequence is SEQ ID NO: 8 and amino acid sequence is SEQ ID NO: 5360.

FIGS. 11A-11F show transposition efficiency of VchCAST and other Type I-F CAST systems in WT and NAP-knockout cells. FIG. 11A is the integration efficiency under different expression systems and induction conditions for VchCAST in WT and ΔihfA cells. pSPIN is a single plasmid that encodes both the donor molecule and transposition machinery, as described in Vo, et al (2021) Nat Biotechnol, 39, 480-489. pEffector+pDonor refers to separate plasmids that encode the transposition machinery and donor DNA, respectively. The indicated promoters were also tested, with J23119 and J23101 being constitutively active whereas the T7 promoter is induced by growing cells on IPTG. FIG. 1B is an alignment of the sequence between the first two TnsB binding sites (L1 and L2) in the left end, generated by Clustal Omega and colored in Jalview to highlight conserved residues. The consensus IHF binding site (IBS) is shown below the alignment. Sequences listed are from top to bottom SEQ ID NOs: 5314-5332, respectively, except for SEQ ID NO: 5321 for both Tn6677 and Tn7000. FIG. 11C shows integration orientation preference in WT and ΔihfA cells for VchCAST and Tn7000. For Tn7000, T-RL integration products were not detected (N.D.) after 35 cycles of qPCR, indicating an integration efficiency less than 0.01%. Integration orientation (FIG. 11D) and efficiency (FIG. 11E) of transposons with symmetric end sequences in WT and ΔihfA cells. R-L refers to a WT-like sequence in which the transposon end identity has not been changed, whereas R-R or L-L refer to transposons in which the left or right end sequence have been mutated to the opposite end sequence, resulting in a transposon with symmetric ends. FIG. 11F shows the effect of nucleoid associated protein knockouts for VchCAST. Transposition was measured by qPCR after expressing pSPIN in each of the indicated E. coli knockout strains.

FIGS. 12A-12C show the effect of NAP knockouts on Tn7 transposition efficiency and fidelity. FIG. 12A is a schematic of an NGS-based Tn7 transposition assay. The transposon cargo encodes genomic primer binding sites (“P1”) adjacent to the right and left ends, such that the NGS amplicon length (“C”) is the same for unintegrated products and for integrated products in both orientations. Using this strategy, a single NGS library reports both the integrated and unintegrated products, while avoiding PCR bias that might arise from amplifying products of different lengths or primer binding sites. FIG. 12B shows the Tn7 integration efficiencies in the indicated NAP knockout strains are shown, quantified using both qPCR and NGS. The dotted line shows the WT integration value as measured by NGS. ΔihfA or ΔihfB have no effect on integration activity, whereas Δfis increases integration activity ˜4-fold. FIG. 12C shows the integration distance and orientation distribution downstream of the glmS locus for Tn7 in WT and Δfis cells. The x-axis refers to the distance in bp between the stop codon of glmS and the integration site. For WT and knockout cells, the dominant distance is the canonical 25 bp downstream of glmS. The y-axes are shown as linear scale (top) and as log 10 scale (bottom), in order to highlight low frequency integration events at non-canonical distances and orientations.

FIG. 13, similar to FIG. 4A, shows the sequence of the native transposon right end derived from Vibrio cholerae Tn6677 (SEQ ID NO: 5333) and the amino acids it encodes Frame 1 (SEQ ID NOs: 5238 and 5239); Frame 2 (SEQ ID NOs: 5240 and 5241); Frame 3 (SEQ ID NOs: 5242 and 5243); Frame 4 (SEQ ID NO: 5334); Frame 5 (SEQ ID NO: 5335); and Frame 6 (SEQ ID NO: 5336-5337). Shown in the middle is the DNA sequence of the transposon right end, orientated such that the end of the transposon, including the 8-bp terminal repeat colored in yellow, is at the far left, whereby the genomic flanking sequence would be to the left of the right end, and the internal cargo encoded within the mini-transposon would be to the right of the right end sequence shown. TnsB binding sites are colored in light blue. Were this sequence to be transcribed and translated into protein, it would yield the six potential coding sequences shown about and below the DNA sequence, according to the direction of translation and the specific open reading frame (ORF) selected during the integration event.

FIGS. 14A and 14B are schematics of the advantages of CAST-based protein tagging. Multi-spacer CRISPR arrays allow multiplexing, meaning CASTs can be harnessed for tagging multiple target genes in parallel through a single plasmid construct (FIG. 14A). The ability of CASTs to efficiently integrate large cargos (e.g., ˜10 kb) suggests lengthier tags and, for example, low tandem FP arrays are well-suited for CAST-based insertion, enabling signaling amplification (FIG. 14B).

FIG. 15 shows the result of the mutational panel revealing high sequence plasticity for certain positions within the TnsB binding sites and critical sequence constraints in others. These data support a consensus sequence of: CMMCBRWAWNNTGAHWWYWN (SEQ ID NO: 12).

FIG. 16 shows the preferential transposase binding site spacing. Manipulating the spacing between the first and the distal two TnsB binding sites on the right or left transposon end revealed a ˜10-bp periodic preference for integration. The distance of this preference corresponds to a single turn of the DNA double helix, which suggests that TnsB protomers are able to form an active paired-end complex if they are positioned on a consistent side of donor DNA.

FIG. 17 is a graph showing that mutating the putative IBS decreases integration efficiency in WT but not ihfA knockout cells. The first mutant, “AT< >CG” (SEQ ID NO: 5339), has all adenines and thymines substituted with cytosines and guanines, respectively, which disrupts all non-N bases in the E. coli IBS consensus (5′-WATCARNNNNTTR). The second mutant (SEQ ID NO: 5340) has the IBS inverted to the reverse complement, which would cause IHF to bind on the reverse strand in the opposite direction. WT sequence is SEQ ID NO: 5338.

FIG. 18 shows a proposed model of IHF binding to the transposon end and bending the left transposon end between two TnsB binding sites, facilitating formation of the strand transfer complex.

FIG. 19A is a schematic of exemplary TnsA-IHF-B fusion constructs. The single chain IHF sequence was encoded internally between TnsA-NLS and TnsB. Different linkers were screened between scIHF and the surrounding subunits to ensure proper flexibility and spatial requirements were met to maintain functional TnsA and TnsB subunits. FIG. 19B is a graph of E. coli transposition assays to measure the efficiency of various TnsA-IHF-TnsB variants. All variants showed robust transposition activity. ΔIHF represents a construct in which no IHF or linker sequences were present between TnsA-NLS and TnsB. GSGSGG is SEQ ID NO: 5341 and (GGS)₆ is SEQ ID NO: 5342.

FIG. 20 is a schematic of exemplary transposon end sequences (SEQ ID NOs: 3120-4665 for left end transposon sequences and SEQ ID NOs: 845-2690 for right end transposon sequences). Transposon end library sequences were designed to include the minimally necessary transposon end sequence—115-bp for the Tn6677 transposon left end (SEQ ID NO: 5345), and 75-bp for the Tn6677 transposon right end (SEQ ID NO: 5346)—together with a ‘stuffer’ sequence that was designed in order to facilitate oligoarray synthesis of the library members with a constant oligonucleotide length across all library members and added protein binding sites or modified AT content. Additionally, ‘stuffer’ sequences enabled consistency when designing transposon end variants in which the spacing between TnsB binding sites was increased by N nucleotides, which necessitated eliminating a corresponding number of N nucleotides from the ‘stuffer’ sequence to maintain a constant total length of transposon end variant. The starting point ‘stuffer’ sequence used for transposon left end variants was 32-bp in length, and contained the sequence 5′-CGAGTATTTCAGCAAAACTACTGCAGTAAGAA-3′ (SEQ ID NO: 5343). The starting point ‘stuffer’ sequence used for transposon right end variants was 47-bp in length, and contained the sequence 5′-GATCATAGTCAGACCAACATTGCTACGACCCGTATTCGCACCGACAC-3′ (SEQ ID NO: 5344).

FIGS. 21A-21C show identification of hyperactive transposon end variants. A hypoactive background was established in order to facilitate identification of modified transposon end sequences that increase activity relative to the WT, native transposon end sequence. To reduce overall integration activity, cells were plated on solid LB-agar media lacking any inducer (IPTG). When compared to plating cells on ˜0.1 mM IPTG (+ column), the integration efficiency without IPTG (− column) decreased approximately 3-fold, from ˜80% to ˜25% (FIG. 21A). Transposon library experiments were performed within this hypoactive background to identify hyperactive transposon end variants that were improved relative to WT (FIG. 21B). The four barcoded WT transposon end library members are indicated by dashed horizontal lines, and the left and right graphs show transposon right end and left end variants, respectively, as described at the top of the graph. Each transposon end variant is identified with a description of the sequence, or with an identifier; in both cases, the sequences of the modified transposon ends can be found in Table 5 (SEQ ID NOs: 291-2702) or Table 6 (SEQ ID NOs:4666-4673). “rc” denotes the reverse complement of a binding site sequence. Integration data are reported as a fold-change, normalized to WT, based on the number of sequencing reads in the integration product library divided by the starting abundance in the input library, relative to the four barcoded WT library members. FIG. 21C shows the validation of hyperactive variants by cloning select right end variants into a pDonor substrate and measuring integration efficiency via qPCR. Sequences of the variant transposon ends are illustrated, along with their corresponding integration efficiencies. A WT pDonor substrate with native transposon left and right ends is shown for comparison. WT is SEQ ID NO: 5347; IHF is SEQ ID NO: 5348; IHF(rc) is SEQ ID NO: 5349; H-NC is SEQ ID NO: 5350; and H-NS(rc) is SEQ ID NO: 5351.

### DETAILED DESCRIPTION

The disclosed systems, kits, and methods provide systems and methods for nucleic acid integration utilizing engineered CRISPR-associated transposon systems. The disclosed systems, kits, and methods provide systems and methods for RNA-guided DNA integration utilizing engineered CRISPR-associated transposon systems.

What distinguishes mobile DNA from other non-mobile DNA are the transposon end sequences. These transposon ends contain repetitive sequence elements to which the transposase binds, thereby identifying the mobilized genetic payload. Although CRISPR-associated transposons hold great potential for many different types of genome engineering purposes, the integration events are not scarless, as the desired payload must be flanked by the transposon end sequences recognized by the transposases, thus leaving scars behind at these regions within the integrated site in the genome. Because the transposon ends are essential for DNA mobilization, the scars cannot be outright eliminated, however their sequences can be modified through both rational engineering or directed evolution.

Herein, pooled library screening and high-throughput sequencing reveal sequence preferences during transposition by the Type I-F Vibrio cholerae CAST system. On the donor DNA, large mutagenic libraries identified core binding sites recognized by the TnsB transposase, as well as an additional conserved region that encoded a consensus binding site for integration host factor (IHF). Remarkably, VchCAST utilized IHF for efficient transposition, thus revealing a cellular factor involved in CRISPR-associated transpososome assembly. In fact, two host factors can aid in RNA-guided DNA integration. The first factor is IHF, which in Escherichia coli is encoded by two genes, ihfA and ihfB. The second factor is factor for inversion stimulation (Fis), encoded by one gene, fis. Loss of either component decreased integration activity. On the target DNA, preferred sequence motifs were uncovered at the integration site that explained previously observed heterogeneity with single-base pair resolution. Finally, the library data was utilized to design modified transposon variants to enable in-frame protein tagging.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of cell and tissue culture, molecular biology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, “nucleic acid” or “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see. e.g., Braasch and Corey, Biochemistry. 41(14): 4503-4510 (2002)) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

Nucleic acid or amino acid sequence “identity,” as described herein, can be determined by comparing a nucleic acid or amino acid sequence of interest to a reference nucleic acid or amino acid sequence. The percent identity is the number of nucleotides or amino acid residues that are the same (e.g., that are identical) as between the sequence of interest and the reference sequence divided by the length of the longest sequence (e.g., the length of either the sequence of interest or the reference sequence, whichever is longer). A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAST™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

The term “homology” and “homologous” refers to a degree of identity. There may be partial homology or complete homology. A partially homologous sequence is one that is less than 100% identical to another sequence.

As used herein, the term “hybridization” is used in reference to the pairing of complementary nucleic acids. Hybridization and the strength of hybridization (e.g., the strength of the association between the nucleic acids) is influenced by such factors as the degree of complementary between the nucleic acids, stringency of the conditions involved, and the Tm of the formed hybrid. Hybridization methods involve the annealing of one nucleic acid to another, complementary nucleic acid, e.g., a nucleic acid having a complementary nucleotide sequence. The ability of two polymers of nucleic acid containing complementary sequences to find each other and “anneal” or “hybridize” through base pairing interaction is a well-recognized phenomenon. The initial observations of the “hybridization” process by Marmur and Lane, Proc. Nal. Acad. Sci. USA, 46: 453 (1960) and Doty et al., Proc. Nal. Acad. Sci. USA, 46: 461 (1960), have been followed by the refinement of this process into an essential tool of modem biology. For example, hybridization and washing conditions are now well known and exemplified in Sambrook et al., supra. The conditions of temperature and ionic strength determine the “stringency” of the hybridization.

As used herein, a “double-stranded nucleic acid” may be a portion of a nucleic acid, a region of a longer nucleic acid, or an entire nucleic acid. A “double-stranded nucleic acid” may be, e.g., without limitation, a double-stranded DNA, a double-stranded RNA, a double-stranded DNA/RNA hybrid, etc. A single-stranded nucleic acid having secondary structure (e.g., base-paired secondary structure) and/or higher order structure (e.g., a stem-loop structure) may also be considered a “double-stranded nucleic acid.” For example, triplex structures are considered to be “double-stranded.” In some embodiments, any base-paired nucleic acid is a “double-stranded nucleic acid.”

The term “gene” refers to a DNA sequence that comprises control and coding sequences necessary for the production of an RNA having a non-coding function (e.g., a ribosomal or transfer RNA), a polypeptide, or a precursor of any of the foregoing. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this disclosure, it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

A “vector” or “expression vector” is a replicon, such as plasmid, phage, virus, or cosmid, to which another DNA segment, e.g., an “insert,” may be attached or incorporated so as to bring about the replication of the attached segment in a cell.

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. For example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

The term “contacting” as used herein refers to bring or put in contact, to be in or come into contact. The term “contact” as used herein refers to a state or condition of touching or of immediate or local proximity. Contacting a composition to a target destination, such as, but not limited to, an organ, tissue, cell, or tumor, may occur by any means of administration known to the skilled artisan.

As used herein, the terms “providing,” “administering,” and “introducing,” are used interchangeably herein and refer to the placement of the systems of the disclosure into a cell, organism, or subject by a method or route which results in at least partial localization of the system to a desired site. The systems can be administered by any appropriate route which results in delivery to a desired location in the cell, organism, or subject.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Systems

In bacteria and archaea, CRISPR/Cas systems provide immunity by incorporating fragments of invading phage, virus, and plasmid DNA into CRISPR loci and using corresponding CRISPR RNAs (“crRNAs”) to guide the degradation of homologous sequences. Transcription of a CRISPR locus produces a “pre-crRNA,” which is processed to yield crRNAs containing spacer-repeat fragments that guide effector nuclease complexes to cleave dsDNA sequences complementary to the spacer. Several different types of CRISPR systems are known, (e.g., type I, type II, or type III), and classified based on the Cas protein type and the use of a proto-spacer-adjacent motif (PAM) for selection of proto-spacers in invading DNA.

Although RNA-guided targeting typically leads to endonucleolytic cleavage of the bound substrate, recent studies have uncovered a range of noncanonical pathways in which CRISPR protein-RNA effector complexes have been naturally repurposed for alternative functions. For example, some Type I (Cascade) and Type II (Cas9) systems leverage truncated guide RNAs to achieve potent transcriptional repression without cleavage and other Type I (Cascade) and Type V (Cas12) systems lie inside unusual bacterial Tn7-like transposons and lack nuclease components altogether.

Disclosed herein are systems or kits for nucleic acid modification comprising: a) an engineered Clustered Regularly Interspaced Short Palindromic Repeats (CRISPR)-associated transposon (CAST) system or one or more nucleic acids encoding the engineered CAST system, wherein the CAST system comprises at least one or all of: i) at least one Cas protein; ii) at least one transposon-associated protein; iii) at least one guide RNA (gRNA) complementary to at least a portion of a target nucleic acid sequence; and b) a donor nucleic acid comprising a cargo nucleic acid sequence flanked by at least one engineered transposon end sequence; and/or c) at least one integration co-factor protein, or a nucleic acid encoding thereof.

In some embodiments, the systems comprise a) an engineered Clustered Regularly Interspaced Short Palindromic Repeats (CRISPR)-associated transposon (CAST) system or one or more nucleic acids encoding the engineered CAST system, wherein the CAST system comprises at least one or all of: i) at least one Cas protein; ii) at least one transposon-associated protein; iii) at least one guide RNA (gRNA) complementary to at least a portion of a target nucleic acid sequence; and b) a donor nucleic acid comprising a cargo nucleic acid sequence flanked by at least one engineered transposon end sequence.

In some embodiments, the systems comprise a) an engineered Clustered Regularly Interspaced Short Palindromic Repeats (CRISPR)-associated transposon (CAST) system or one or more nucleic acids encoding the engineered CAST system, wherein the CAST system comprises at least one or all of: i) at least one Cas protein; ii) at least one transposon-associated protein; iii) at least one guide RNA (gRNA) complementary to at least a portion of a target nucleic acid sequence; and b) at least one integration co-factor protein, or a nucleic acid encoding thereof.

In some embodiments, the systems comprise a) an engineered Clustered Regularly Interspaced Short Palindromic Repeats (CRISPR)-associated transposon (CAST) system or one or more nucleic acids encoding the engineered CAST system, wherein the CAST system comprises at least one or all of: i) at least one Cas protein; ii) at least one transposon-associated protein; iii) at least one guide RNA (gRNA) complementary to at least a portion of a target nucleic acid sequence; b) a donor nucleic acid comprising a cargo nucleic acid sequence flanked by at least one engineered transposon end sequence; and c) at least one integration co-factor protein, or a nucleic acid encoding thereof.

In some embodiments, one or more of the at least one Cas protein are part of a ribonucleoprotein complex with the gRNA.

In some embodiments, the engineered CRISPR-Tn system is derived from Vibrio parahaemolyticus, Aliibrio sp., Pseudoalteromonas sp., Endozoicomonas ascidiicola, Vibrio cholerae, Photobacterium iliopiscarium, Vibrio parahaemolyticus, Pseudoalteromonas sp., Pseudoalteromonas ruthenica, Photobacterium ganghwense, Shewanella sp., Vibrio diazotrophicus, Vibrio sp. 16, Vibrio sp. F12, Vibrio splendidus, Aliivibrio wodanis, and Aliivibrio sp. Pseudoalteromonas sp. includes, but is not limited to, Pseudoalteromonas sp. SG43-3, Pseudoalteromonas sp. P1-13-1a, Pseudoalteromonas arabiensis, Pseudoalteromonas sp. Strain P1-25, Pseudoalteromonas sp. strain S983.

In some embodiments, the engineered transposon system is from a bacteria selected from the group consisting of: Vibrio cholerae strain 4874, Photobacterium iliopiscarium strain NCIMB, Pseudoalteromonas sp. P1-25, Pseudoalteromonas ruthenica strain S3245, Photobacterium ganghwense strain JCM, Shewanella sp. UCD-KL21, Vibrio cholerae strain OYP7G04, Vibrio cholerae strain M1517, Vibrio diazotrophicus strain 60.6F, Vibrio sp. 16, Vibrio sp. F12, Vibrio splendidus strain UCD-SED10, Aliivibrio wodanis 06/09/160, and Parashewanella spongiae strain HJ039. In an exemplary embodiment, the engineered transposon system is derived from Vibrio cholerae Tn6677. In an exemplary embodiment, the engineered transposon system is derived from Pseudoalteromonas Tn7016.

In some embodiments, the system comprises two or more engineered CAST systems. Pairing of orthogonal systems with their orthogonal donor substrates enables tandem insertion of multiple distinct payloads directly adjacent to each other without any risk of repressive effects from target immunity. For example, one, two, three, four, five, or more orthogonal CAST systems may be used to integrate large tandem arrays of payload DNA.

The system may be a cell free system. Also disclosed is a cell comprising the system described herein. In some embodiments, the cell is a prokaryotic cell. In some embodiments, the cell is a eukaryotic cell. In some embodiments, the cell is a mammalian cell (e.g., a cell of a non-human primate or a human cell). Thus, in some embodiments, disclosed herein are systems or kits for nucleic acid integration into a target nucleic acid sequence in a eukaryotic cell (e.g., a mammalian cell, a human cell).

a. Donor Nucleic Acid and Engineered Transposon Sequences

The system may further include a donor nucleic acid to be integrated. The donor nucleic acid may be a part of a bacterial plasmid, bacteriophage, a virus, autonomously replicating extra chromosomal DNA element, linear plasmid, linear DNA, linear covalently closed DNA, mitochondrial or other organellar DNA, chromosomal DNA, and the like.

In some embodiments, the donor nucleic acid comprises a cargo nucleic acid sequence. In some embodiments, the donor nucleic acid comprises a cargo nucleic acid sequence flanked by at least one engineered transposon end sequence. In some embodiments, the donor nucleic acid is flanked on the 5′ and the 3′ end with a transposon end sequence. In some embodiments, the donor nucleic acid comprises a cargo nucleic acid sequence flanked by one native transposon end sequence and one engineered transposon end sequence. In some embodiments, the donor nucleic acid comprises a cargo nucleic acid sequence flanked by two engineered transposon end sequences, a left end sequence 5′ to the cargo nucleic acid sequence, relative to transcription direction, and a right end sequence 3′ to the cargo nucleic acid sequence, relative to transcription direction.

The term “transposon end sequence” refers to any nucleic acid comprising a sequence capable of forming a complex with the transposase enzymes thus designating the nucleic acid between the two ends for rearrangement. Usually, native CRISPR-transposon end sequences contain inverted repeats and may be about 10-150 base pairs long. The engineered transposon end sequences, comprise sequences which have one or more basepair or nucleotide additions, deletions, or substitutions as compared to a native transposon end sequence. The engineered transposon ends sequences may or may not include additional sequences that promote or augment transposition, enhance binding to other protein factors, or allow the sequence to adopt an energetically favorable conformation state for binding.

In some embodiments, the engineered transposon end sequence comprises a sequence having one or more substitutions (e.g., 1, 2, 3, 4, 5, 6, 7, 8, 9, 10, 11, 12, 13, 14, 15, 16, 17, 18, 19, 20, 21, 22, 23, 24, 25, or more) as compared to a native transposon end sequence. In some embodiments, the engineered transposon end sequence comprises a sequence having one or more additions (e.g., 1, 2, 3, 4, 5, 6, 7, 8, 9, 10, 11, 12, 13, 14, 15, 16, 17, 18, 19, 20, 21, 22, 23, 24, 25, or more) as compared to a native transposon end sequence. In some embodiments, the engineered transposon end sequence comprises a sequence having one or more deletions (e.g., 1, 2, 3, 4, 5, 6, 7, 8, 9, 10, 11, 12, 13, 14, 15, 16, 17, 18, 19, 20, 21, 22, 23, 24, 25, or more) as compared to a native transposon end sequence. The engineered transposon end sequence may comprise a truncation of the native transposon end sequences. For example, in some embodiments, the transposon end sequence may have an approximate 10, 20, 30, 40, 50, 60, or more base pair (bp) deletion relative to the native CRISPR-transposon end sequence. The deletion may be in the form of a truncation at the distal (in relation to the cargo) end of the transposon end sequences. The deletion may be in the form of a truncation at the proximal (in relation to the cargo) end of the transposon end sequences.

In some embodiments, the at least one engineered transposon end sequence encodes an amino acid linker sequence. The engineered transposon end sequence may comprise a sequence related to the native transposon end sequence but lacking any stop codons. For example, the engineered transposon end sequence may comprise one or more point mutations which alter the encoded amino acids.

In some embodiments, the engineered transposon right end sequence and/or the engineered transposon left end sequence is derived from a Vibrio cholerae Tn6677 native transposon end sequence. In some embodiments, the engineered transposon right end sequence and/or the engineered transposon left end sequence is derived from a Pseudoalteromonas Tn7016 native transposon end sequence.

In some embodiments, the at least one engineered transposon end sequence is fully or partially AT rich. In some embodiments, the entirety of the transposon end sequence is AT rich. In some embodiments, a region of the transposon end sequence distal to the cargo nucleic acid is AT rich. For example, the distal 10 bp, 20 bp, 30 bp, 40 bp, 50 bp, or 60 bp may be AT rich. In some embodiments, a region of the transposon end sequence proximal to the cargo nucleic acid is AT rich. For example, the proximal 10 bp, 20 bp, 30 bp, 40 bp, 50 bp, or 60 bp may be AT rich. In some embodiments, regions outside of specific protein binding sites (e.g., TnsB binding sites) are AT rich.

Nucleic acid sequences containing a high level of A or T bases compared to the level of G or C bases are referred as AT rich or having high AT content. Accordingly, AT rich sequences can have relatively high levels of A bases, T bases or both A and T bases. Nucleic acid sequences having greater than about 52% AT content are AT rich sequences. In some embodiments, a portion of, as described above, or the entire transposon end sequence is greater than 55%, greater than 60%, greater than 65%, greater than 70%, greater than 75%, greater than 80%, greater than 85%, greater than 90%, greater than 95% or greater than 99% AT content.

In a CAST system, TnsB confers sequence specificity for the transposon ends through recognition of repetitive sequence elements known as TnsB binding sites (TBSs). The at least one engineered transposon end sequence(s) may comprise at least one (e.g., 1, 2, 3, 4, 5, or more) TBSs. In some embodiments, the at least one engineered transposon end sequence comprises two TBSs. In some embodiments, the at least one engineered transposon end sequence comprises three TBSs.

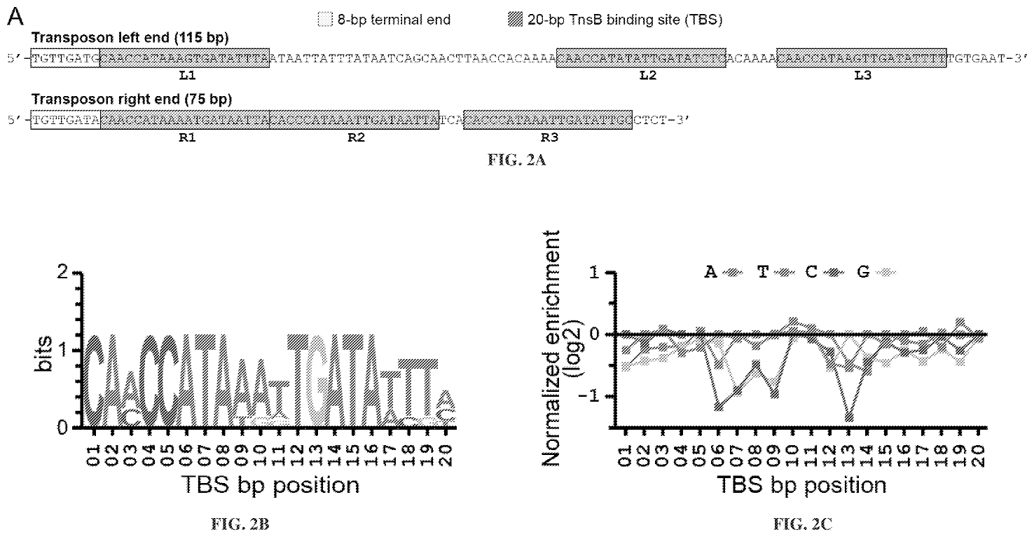

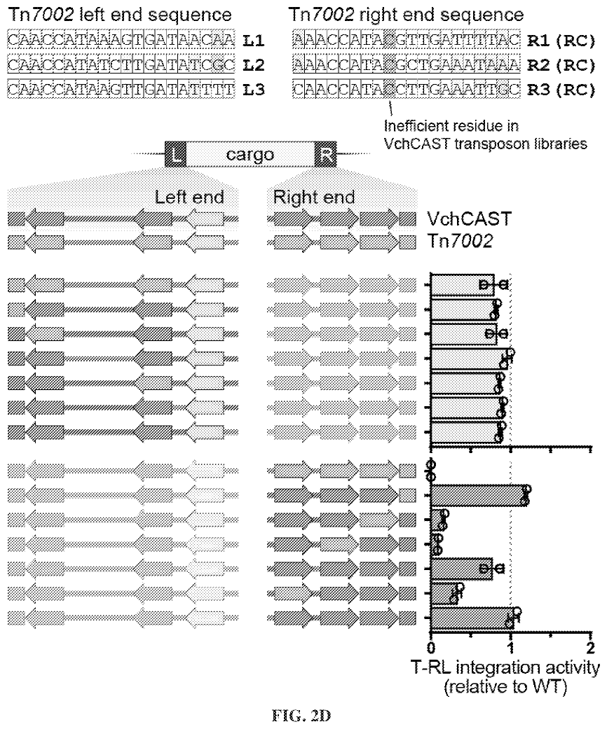

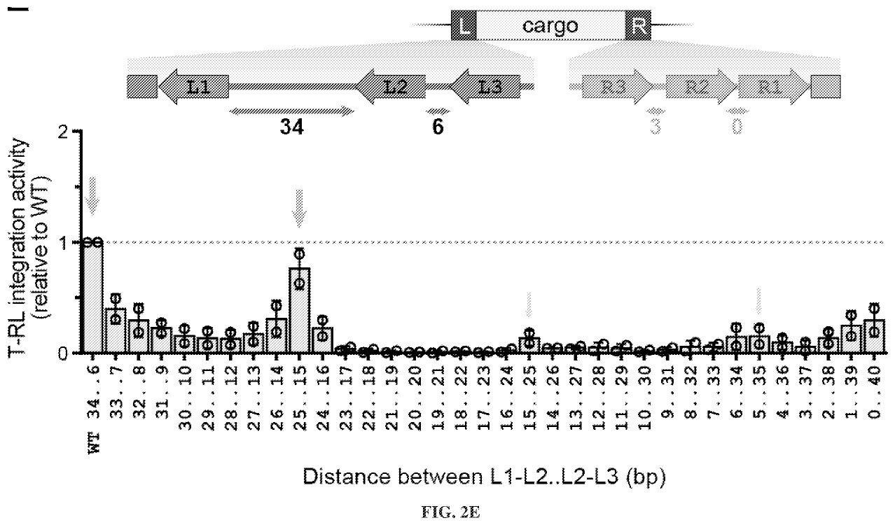

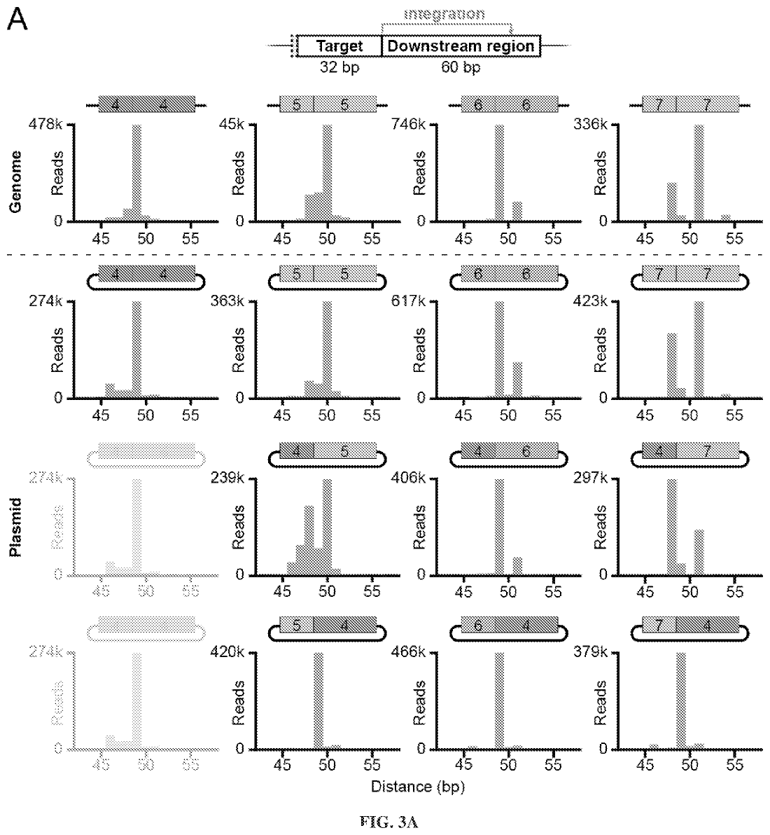

The engineered transposon sequence may comprise native transposase binding sites and/or engineered transposase binding sites which facilitate TnsB binding as the native site. The TBS may comprise any native or engineered sequence that facilitates recognitions by TnsB. In some embodiments, each TBS comprises a sequence individually selected from: CAMCCATAWRDTGATAWYKH (SEQ ID NO: 11), or CMMCBRWAWNNTGAHWWYWN (SEQ ID NO: 12), wherein each M is individually A or C: each W is independently A or T; each R is independently A or G; each D is independently A, G or T; each Y is independently T or C; each K is G or T; B is G, T, or C; and each H is independently A, C or T. In some embodiments, the TBS sequences are selected from those shown in FIGS. 2 & 7.

Each individual TBS may be separated from another TBS by one or more basepairs (bp). For example, any one TBS may be separated from the adjacent TBS by 0, 1, 2, 3, 4, 5, 6, 7, 8, 9, 10, or more bp. In some embodiments, the transposon end sequence comprises two immediately adjacent TBSs. In some embodiments, the transposon end sequence comprises two TBS separated by one to ten bp. In some embodiments, the transposon end sequence comprises two TBS separated by 30-40 bp.

In some embodiments, the at least one engineered transposon end sequence further comprises a 5 to 8 bp terminal end sequence. A terminal end sequence is any sequence that dictates the transposon boundary. In some embodiments, the terminal end sequence comprises a terminal TG dinucleotide. In some embodiments, the terminal end sequence is immediately adjacent to the distal end of TBS farthest from the cargo nucleic acid sequence. In some embodiments, the terminal end sequence is separated from the distal end of the transposase binding site farthest from the cargo nucleic acid sequence by 1, 2 or 3 basepairs (bp).

In some embodiments, the at least one engineered transposon end sequence is a transposon right end sequence 3 to the cargo nucleic acid sequence, relative to transcription direction. The engineered transposon right end sequence is at least about 50 basepairs (bp). In some embodiments, the engineered transposon right end sequence is at least about 55 bp, 60 bp, 70 bp, 75 bp, or more. In some embodiments, engineered transposon right end sequence is about 50 bp, about 55 bp, about 60 bp, about 65 bp, about 70 bp, about 75 bp, about 80 bp, about 85 bp, about 90 bp, about 95 bp, about 100 bp, about 105 bp, about 110 bp, about 115 bp, about 120 bp, about 125 bp, or more.

In some embodiments, the engineered transposon right end sequence comprises two TBSs. In some embodiments, the engineered transposon right end sequence comprises three TBSs. In some embodiments, the TBSs in the engineered transposon right end sequence are each less than 10 bp from the adjacent TBS. In select embodiments, the TBSs in the engineered transposon right end sequence are immediately adjacent or separated by 1 to 5 bp.

In some embodiments, the engineered transposon right end sequence comprises a sequence of: TGTTGATACAACCATAAAATGATAATTACACCCATAAATTGATAATTATCACACCCA (SEQ ID NO: 1), or a variant sequence having one or more substitutions thereof. In some embodiments, the engineered transposon right end sequence comprises a sequence of:

(SEQ ID NO: 2)
TGTgGATACAACCATAAAATGATAATTACACCCATAAATgGATcA
TTATCACcCCCA;
(SEQ ID NO: 3)
TGTgGATACAACCATAAAAcGATAATTACACCCATAAATgGATcA
TTATCACACCCA;
(SEQ ID NO: 4)
TGTgGATcCAACCATAAAATGATAATTACACCCATAAATgGATcA
TTATCACACCCA;
(SEQ ID NO: 5)
TGTTGATACAACCATAAAAgGATtATTACACCCATtAATTGATAA
TTATCACACCCA;
(SEQ ID NO: 6)
TGTTGATACAACCATcAAATGgTAATTACACCCATAAATTGATAA
TTATCACACCCA;
(SEQ ID NO: 7)
TGTTGATACAACCATtAAATGATAATTcCACCCATAAtTTGATAA
TTATCACACCCA;
or
(SEQ ID NO: 8)
TGTTGATACAACCATtAAATGgTAATTcCACCCAaAtATTGATAA
TTATCACACCCA.

In some embodiments, the engineered transposon right end sequence comprises a sequence of SEQ ID NOs: 18-844.

In some embodiments, the engineered transposon right end sequence comprises a sequence of: TGTTGATACAACCATAAAATGATAATTACACCCATAAATTGATAATATCACACCCATAAA TTGATATTGCCTCT (SEQ ID NO: 9), or a variant sequence having one or more substitutions thereof. In some embodiments, the engineered transposon right end sequence comprises a sequence of SEQ ID NOs: 845-2690.

In some embodiments, the engineered transposon right end sequence is hyperactive. Hyperactive transposon end sequences are those sequences which result in improved integration activity compared to wildtype, For example, hyperactive transposon end sequences may increase integration activity about 1.1 fold, about 1.2 fold, about 1.3 fold, about 1.4 fold, about 1.5 fold, about 1.6 fold, about 1.7 fold, about 1.8 fold, about 1.9 fold, about 2.0 fold, about 2.1 fold, about 2.2 fold, about 2.3 fold, about 2.5 fold, about 2.6 fold, about 2.7 fold, about 2.8 fold, about 2.9 fold, about 3.0 fold, or more. In some embodiments, the engineered transposon right end sequence comprises a sequence of SEQ ID NOs: 2691-2702. In some embodiments, the engineered transposon right end sequence comprises a sequence of SEQ ID NOs: 2703-3119.

In some embodiments, the at least one engineered transposon end sequence is a transposon left end sequence 5′ to the cargo nucleic acid sequence, relative to transcription direction. In some embodiments, the engineered transposon left end sequence is at least about 105 basepairs (bp). In some embodiments, the engineered transposon left end sequence is at least about 115 basepairs (bp). The engineered transposon left end sequence may be about 105 bp, about 110 bp, about 115 bp, about 120 bp, about 125 bp, about 130 bp, about 135 bp, about 140 bp, about 145 bp, about 150 bp, about 155 bp, about 160 bp, about 165 bp, about 170 bp, about 175 bp, about 180 bp, about 185 bp, about 190 bp, about 195 bp, about 200 bp, or more.

In some embodiments, the engineered transposon left end sequence comprises three transposase TBSs. The distal TBS, in reference to the cargo sequence may be separated from the next closest TBS by at least 10 bp. In some embodiments, the distal TBS is separated from the next closest TBS by about 20 bp to about 40 bp. In select embodiments, the distal TBS is separated from the next closest TBS by about 23-26 bp or about 30-35 bp. In some embodiments, the two proximal TBSs are separated from each other by less than 10 bp. In some embodiments the two proximal TBSs are separated from each other by 5-7 bp.

In some embodiments, the engineered transposon left end sequence further comprises an Integration Host Factor (IHF) binding site (IBS), as described above. In some embodiments, the engineered transposon left end sequence does not include an Integration Host Factor (IHF) binding site (IBS).

In some embodiments, the engineered transposon left end sequence comprises a sequence of: TGTTGATGCAACCATAAAGTGATATTTAATAATTATTTATAATCAGCAACTTAACCACAAA ACAACCATATATTGATATCTCACAAAACAACCATAAGTTGATATTITGTGAAT (SEQ ID NO: 10), or a variant sequence having one or more substitutions thereof. In some embodiments, the engineered transposon left end sequence comprises a sequence of SEQ ID NOs: 3120-4665.

In some embodiments, the engineered transposon left end sequence is hyperactive. In some embodiments, the engineered transposon left end sequence comprises a sequence of SEQ ID NOs: 4666-4673. In some embodiments, the engineered transposon left end sequence comprises a sequence of SEQ ID NOs: 4674-5135.

In some embodiments, the donor nucleic acid comprises a cargo nucleic acid sequence flanked by two engineered transposon end sequences; an engineered transposon right end sequence, as described above, and an engineered transposon left end sequence, as described above.

The cargo nucleic acid comprises a sequence encoding the desired nucleic acid to be inserted into the target nucleic acid.

The cargo nucleic acid may encode any peptide or polypeptide which is desired to be inserted into the target nucleic acid and is not limited by the type or identity of the peptide or polypeptide. For example, if the target site encodes an endogenous protein, the peptide or polypeptide may be so configured to form a fusion protein with the endogenous protein and the amino acid linker encoded by the transposon end sequence.

In some embodiments, the cargo nucleic acid sequence includes a peptide tag. The invention is not limited by the choice of peptide tag. Usually, a peptide tag is an amino acid sequence which facilitates the identification, detection, measurement, purification and/or isolation of the protein to which it is linked or fused. Peptide tags are usually relatively short compared to the protein fused to the peptide tag. As an example, peptide tags, in some embodiments, have amino acids of 4 or more lengths, such as 5, 6, 7, 8, 9, 10, 15, 20, or 25. Peptide tabs include, but are not limited to: HA (blood cell agglutinin), c-myc, simple herpesvirus glycoprotein D (gD), T7, GST, MBP, Strep tags, His tags, Myc tags, TAP tags, and FLAG tags. For example, if the target site encodes an endogenous protein, the cargo and peptide tag may be so configured to tag or label an endogenous protein and the amino acid linker encoded by the transposon end sequence.

In some embodiments, the cargo nucleic acid encodes a polypeptide. The invention is not limited by the choice of polypeptide. In select embodiments, the polypeptide comprises a fluorescent protein. “Fluorescent protein” refers to any protein capable of fluorescence when excited with appropriate electromagnetic radiation. This includes fluorescent proteins whose amino acid sequences are either natural or engineered.

The donor nucleic acid, and by extension the cargo nucleic acid, may of any suitable length, including, for example, about 50-100 bp (base pairs), about 100-1000 bp, at least or about 10 bp, at least or about 20 bp, at least or about 25 bp, at least or about 30 bp, at least or about 35 bp, at least or about 40 bp, at least or about 45 bp, at least or about 50 bp, at least or about 55 bp, at least or about 60 bp, at least or about 65 bp, at least or about 70 bp, at least or about 75 bp, at least or about 80 bp, at least or about 85 bp, at least or about 90 bp, at least or about 95 bp, at least or about 100 bp, at least or about 200 bp, at least or about 300 bp, at least or about 400 bp, at least or about 500 bp, at least or about 600 bp, at least or about 700 bp, at least or about 800 bp, at least or about 900 bp, at least or about 1 kb (kilobase pair), at least or about 2 kb, at least or about 3 kb, at least or about 4 kb, at least or about 5 kb, at least or about 6 kb, at least or about 7 kb, at least or about 8 kb, at least or about 9 kb, at least or about 10 kb, or greater.

b. Integration Co-Factor Protein

The present systems may further include at least one integration co-factor protein. The at least one integration co-factor protein may comprise Integration Host Factor (IHF), Factor for Inversion Stimulation (Fis), variants or derivatives thereof, or a combination thereof.

In some embodiments, the at least one integration co-factor protein comprises Integration Host Factor (IHF). In one embodiment, IHFα (also referred to as IHFα) and IHFβ (also referred to as IHFb) are provided as separate polypeptides. Alternatively, the IHFα and IHFβ subunits can be fused together to be expressed as a single polypeptide (See, Corona et al., Nucleic Acids Research 31, 5140-5148 (2003)). In certain embodiments, the single chain IHF (scIHF) is appended with various short sequences, such as NLS tags, on either the N-terminus or the C-terminus, or both termini, or encoded internally.

The at least one integration co-factor protein is not limited from which organism it is derived. In some embodiments, the IHF sequence is derived from the E. coli genome. In other embodiments, the IHF sequence is derived from the cognate strain from which the CRISPR-associated sequence is derived. For example, the IHFα and IHFβ sequences from Vibrio cholerae HE-45 can be used alongside RNA-guided DNA integration machinery derived from Tn6677, while IHFα and IHFβ sequences from Psuedoalteromonas sp. 5983 can be used alongside RNA-guided DNA integration machinery derived from Tn7016. In some embodiments, the at least one integration co-factor protein comprises an amino acid sequence of any of SEQ ID NOs: 5136-5152, See Table 3.

In some embodiments, the at least one integration factor protein sequences are fused to a localization agent (e.g., proteins or domains thereof to promote localization to the transposon ends). In one such embodiment, the at least one integration co-factor protein sequence is fused to a nuclease deficient Cas9 (dCas9). Then, using a sgRNA for Cas9 that targets nearby the at least one integration co-factor protein binding sequence within the transposon end, the local concentration of the at least one integration co-factor protein is increased to promote correct binding and bending of the transposon end. In other embodiments, other DNA-binding proteins are used to promote the localization of the at least one integration co-factor protein to the transposon, such as, but not limited to, TALE proteins and zinc-finger domain proteins.

The integration co-factor protein may be fused to protein components of Type I-F CRISPR-associated transposon systems to tether its location proximally to integration co-factor protein binding sites in the transposon ends. In some embodiments, the at least one integration co-factor protein is fused internally to a fusion construct of transposase proteins TnsA and TnsB, as described elsewhere herein. In some embodiments, the at least one integration co-factor protein is fused within the linker of the TnsA-TnsB fusion protein.

In some embodiments, the at least one integration co-factor protein is purified and pre-complexed with the donor DNA to ensure proper protein-DNA interactions. In such embodiments, the pre-formed complexes may be electroporated into cells or delivered via other means.

c. CAST System

CRISPR-Cas systems are currently grouped into two classes (1-2), six types (I-VI) and dozens of subtypes, depending on the signature and accessory genes that accompany the CRISPR array. The engineered CAST system herein may be derived from a Class 1 CRISPR-Cas system or a Class 2 CRISPR-Cas system.

Type I CRISPR-Cas systems encode a multi-subunit protein-RNA complex called Cascade, which utilizes a crRNA (or guide RNA) to target double-stranded DNA during an immune response. Cascade itself has no nuclease activity, and degradation of targeted DNA is instead mediated by a trans-acting nuclease known as Cas3.

The CAST system may be derived from a Type I CRISPR-Cas system (such as subtypes I-B and I-F, including I-F variants). In some embodiments, the engineered CAST is a Type I-F system. In some embodiments, the engineered CAST system is a Type I-F3 system.

In some embodiments, the engineered CAST system comprises Cas5, Cas6, Cas7, Cas8, or any combination thereof. In some embodiments, the engineered CAST system comprises Cas8-Cas5 fusion protein.

A CAST system of the present invention may comprise one or more transposon-associated proteins (e.g., transposases or other components of a transposon). The transposon-associated proteins may facilitate recognition or cleavage of the target nucleic acid and subsequent insertion of the donor nucleic acid into the target nucleic acid.

In some embodiments, the transposon-associated proteins are derived from a Tn7 or Tn7-like transposon. Tn7 and Tn7-like transposons may be categorized based on the presence of the hallmark DDE-like transposase gene, tnsB (also referred to as tniA), the presence of a gene encoding a protein within the AAA+ATPase family, tnsC (also referred to as tniB), one or more targeting factors that define integration sites (which may include a protein within the tniQ family, also referred to as tnsD, but sometimes includes other distinct targeting factors), and inverted repeat transposon ends that typically comprise multiple binding sites thought to be specifically recognized by the TnsB transposase protein. In Tn7, the targeting factors, or “target selectors,” comprise the genes tnsD and insE. Based on biochemical and genetics studies, it is known that TnsD binds a conserved attachment site in the 3′ end of the glmS gene, directing downstream integration, whereas TnsE binds the lagging strand replication fork and directs sequence-non-specific integration primarily into replicating/mobile plasmids.

The most well-studied member of this family of transposons is Tn7, hence why the broader family of transposons may be referred to as Tn7-like. “Tn7-like” term does not imply any particular evolutionary relationship between Tn7 and related transposons; in some cases, a Tn7-like transposon will be even more basal in the phylogenetic tree and thus Tn7 can be considered as having evolved from, or derived from, this related Tn7-like transposon.

Whereas Tn7 comprises tnsD and tnsE target selectors, related transposons comprise other genes for targeting. For example, Tn5090/Tn5053 encode a member of the tniQ family (a homolog of E. coli tnsD) as well as a resolvase gene tniR; Tn6230 encodes the protein TnsF; and Tn6022 encodes two uncharacterized open reading frames orf2 and orf3; Tn6677 and related transposons encode variant Type I-F and Type I-B CRISPR-Cas systems that work together with TniQ for RNA-guided mobilization; and other transposons encode Type V-U5 CRISPR-Cas systems that work together with TniQ for random and RNA-guided mobilization. Any of the above transposon systems are compatible with the systems and methods described herein.

In some embodiments, the one or more transposon-associated proteins comprise TnsA, TnsB, TnsC, or a combination thereof. In some embodiments, the one or more transposon-associated proteins comprise TnsB and TnsC. In some embodiments, the one or more transposon-associated proteins comprise TnsA, TnsB, and TnsC.

In some embodiments, the at least one transposon protein comprises a TnsA-TnsB fusion protein. TnsA and TnsB can be fused in any orientation: N-terminus to C-terminus: C-terminus to N-terminus; N-terminus to N-terminus; or C-terminus to C-terminus, respectively. Preferably the C-terminus of TnsA is fused to the N-terminus of TnsB.

In some embodiments, the TnsA-TnsB fusion may be fused using an amino acid linker peptide of various lengths to provide greater physical separation and allow more spatial mobility between the fused portions. The linker may comprise any amino acids and may be of any length. In some embodiments, the linker may be less than about 50 (e.g., 40, 30, 20, 10, or 5) amino acid residues.

In some embodiments, the linker is a flexible linker, such that TnsA and TnsB can have orientation freedom in relationship to each other. For example, a flexible linker may include amino acids having relatively small side chains, and which may be hydrophilic. Without limitation, the flexible linker may contain a stretch of glycine and/or serine residues. In some embodiments, the linker comprises at least one glycine-rich region. For example, the glycine-rich region may comprise a sequence comprising [GS]n, wherein n is an integer between 1 and 10.

In some embodiments, the linker further comprises a nuclear localization sequence (NLS). The NLS may be embedded within a linker sequence, such that it is flanked by additional amino acids. In some embodiments, the NLS is flanked on each end by at least a portion of a flexible linker. In some embodiments, the NLS is flanked on each end by a glycine rich region of the linker. Suitable nuclear localization sequences for use with the disclosed system are described further below and are applicable to use with the TnsA-TnsB fusion protein.

In some embodiments, the CAST system comprises TnsA, TnsB, TnsC, TnsD and TniQ. In some embodiments, the CAST system comprises Cas5, Cas6, Cas7, Cas8, TnsA, TnsB, TnsC, and at least one or both of TnsD or TniQ. In certain embodiments, the CAST system comprises TnsD. In certain embodiments, the CAST system comprises TniQ. In certain embodiments, the CAST system comprises TnsD and TniQ.

In some embodiments, any combination of the at least one Cas protein and the at least one transposon associated protein may be expressed as a single fusion protein.

Sequences of exemplary Cas proteins and transposon-associated proteins can also be found in International Patent Applications WO2020181264 and PCT/US22/32541, incorporated herein by reference. However, the invention is not limited to the disclosed or referenced exemplary sequences. Indeed, genetic sequences can vary between different strains, and this natural scope of allelic variation is included within the scope of the invention.

In other embodiments, any of the proteins described or referenced herein may comprise a sequence corresponding to, or substantially corresponding to, the wild-type version of the protein. For example, the sequence may substantially correspond to the wild-type protein sequence except for changes made for facile cloning or removal of known restriction sites. Thus, protein products from potential alternative start codons compared to the predicted nucleic acid sequences in this document are therefore not excluded.

Any of the proteins described or referenced herein may comprise one or more amino acid substitutions as compared to the recited sequences. An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or He), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gin), lysine (K or Lys), and arginine (R or Arg).

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra). Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH₂ can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

In some embodiments, the engineered CAST systems further comprise a gRNA complementary to at least a portion of the target nucleic acid sequence, or a nucleic acid encoding the at least one gRNA.

The gRNA may be a crRNA, crRNA/tracrRNA (or single guide RNA, sgRNA). The terms “gRNA,” “guide RNA,” “crRNA,” and “CRISPR guide sequence” may be used interchangeably throughout and refer to a nucleic acid comprising a sequence that determines the binding specificity of the CAST system. A gRNA hybridizes to (complementary to, partially or completely) a target nucleic acid sequence (e.g., the genome in a host cell). In some embodiments, the at least one gRNA is encoded in a CRISPR RNA (crRNA) array.

The system may further comprise a target nucleic acid. In some embodiments, target nucleic acid sequence comprises a human sequence.

gRNAs or sgRNA(s) used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer). In some embodiments, the gRNA sequence that hybridizes to the target nucleic acid is 15, 16, 17, 18, 19, 20, 21, 22, 23, 24, 25, 26, 27, 28, 29, 30, 31, 32, 33, 34, 35, 36, 37, 38, 39, or 40 nucleotides in length.

To facilitate gRNA design, many computational tools have been developed (See Prykhozhij et al. (PLoS ONE, 10(3): (2015)); Zhu et al. (PLoS ONE, 9(9) (2014)); Xiao et al. (Bioinformatics. January 21 (2014)); Heigwer et al. (Nat Methods, 11(2): 122-123 (2014)). Methods and tools for guide RNA design are discussed by Zhu (Frontiers in Biology, 10 (4) pp 289-296 (2015)), which is incorporated by reference herein. Additionally, there are many publicly available software tools that can be used to facilitate the design of sgRNA(s); including but not limited to, Genscript Interactive CRISPR gRNA Design Tool, W U-CRISPR, and Broad Institute GPP sgRNA Designer. There are also publicly available pre-designed gRNA sequences to target many genes and locations within the genomes of many species (human, mouse, rat, zebrafish, C. elegans), including but not limited to, IDT DNA Predesigned Alt-R CRISPR-Cas9 guide RNAs, Addgene Validated gRNA Target Sequences, and GenScript Genome-wide gRNA databases.

In addition to a sequence that binds to a target nucleic acid, in some embodiments, the gRNA may also comprise a scaffold sequence (e.g., tracrRNA). In some embodiments, such a chimeric gRNA may be referred to as a single guide RNA (sgRNA). Exemplary scaffold sequences will be evident to one of skill in the art and can be found, for example, in Jinek, et al. Science (2012) 337(6096):816-821, and Ran, et al. Nature Protocols (2013) 8:2281-2308, incorporated herein by reference in their entireties.

In some embodiments, the gRNA sequence does not comprise a scaffold sequence and a scaffold sequence is expressed as a separate transcript. In such embodiments, the gRNA sequence further comprises an additional sequence that is complementary to a portion of the scaffold sequence and functions to bind (hybridize) the scaffold sequence.

As described elsewhere herein the protein and gRNA components of the system may be expressed and transcribed from the nucleic acids using any promoter or regulatory sequences known in the art. In some embodiments, the gRNA is transcribed under control of an RNA Polymerase II promoter. In some embodiments, the gRNA is transcribed under control of an RNA Polymerase III promoter.

In some embodiments, the gRNA sequence is at least 50%, 55%, 60%, 65%, 70%, 75%, 80%, 85%, 90%, 95%, 96%, 97%, 98%, 99%, or at least 100% complementary to a target nucleic acid. In some embodiments, the gRNA sequence is at least 50%, 55%, 60%, 65%, 70%, 75%, 80%, 85%, 90%, 95%, 96%, 97%, 98%, 99%, or at least 100% complementary to the 3′ end of the target nucleic acid (e.g., the last 5, 6, 7, 8, 9, or 10 nucleotides of the 3′ end of the target nucleic acid).

The gRNA may be a non-naturally occurring gRNA.

The system may further comprise a target nucleic acid having a target nucleic acid sequence. The target nucleic acid sequence may be any sequence of interest which facilitates modification. In some embodiments, the target nucleic acid sequence may comprise regions and sequence motifs which promote, influence, or facilitate TnsB strand transfer for integration of the donor nucleic acid.

The target nucleic acid sequence comprises both the site of gRNA binding and recognition but also the site of integration. Accordingly, the target nucleic acid sequence comprises the target-site duplication (TSD) region which upon insertion generates identical sequences on both sides of the insert. The TSD regions can be of variable length, usually between about 3 bp and about 8 bp, but sometimes longer. In some embodiments, the TSD region is 5 bp. In some embodiments, the TSD region comprises a YWR motif within the central three nucleotides of the target-site duplication (TSD). In some embodiments, the TSD region comprises a 5′-CWG-3′ motif.

The site of integration may be influenced by TSD motif as well as sequences upstream and/or downstream of the TSD region. In some embodiments, the nucleotide 3-bp upstream of the TSD is A, G, or T. In some embodiments, the nucleotide 3 bp downstream of the TSD is T, A, or C. Overall, C and G are less preferred for nucleotides 3 bp upstream and 3 bp downstream from the TSD.

In some embodiments, gRNAs may be selected for integration at defined and desired distances, ranging from ˜47-52 bp, or integration properties (e.g., homogenous vs. heterogeneous integration site) based on the target nucleic acid sequence, specifically the TSD region and the nucleotides 3 bp upstream and 3 bp downstream from the TSD. For example, the 3 end of the gRNA may be ˜47-52-bp upstream from the desired site of integration.

The target nucleic acid may be flanked by a protospacer adjacent motif (PAM). A PAM site is a nucleotide sequence in proximity to a target sequence. For example, PAM may be a DNA sequence immediately following the DNA sequence targeted by the CRISPR-Tn system.

The target sequence may or may not be flanked by a protospacer adjacent motif (PAM) sequence. In certain embodiments, a nucleic acid-guided nuclease can only cleave a target sequence if an appropriate PAM is present, see, for example Doudna et al., Science, 2014, 346(6213): 1258096, incorporated herein by reference. A PAM can be 5′ or 3′ of a target sequence. A PAM can be upstream or downstream of a target sequence. In one embodiment, the target sequence is immediately flanked on the 3′ end by a PAM sequence. A PAM can be 1, 2, 3, 4, 5, 6, 7, 8, 9, 10 or more nucleotides in length. In certain embodiments, a PAM is between 2-6 nucleotides in length. The target sequence may or may not be located adjacent to a PAM sequence (e.g., PAM sequence located immediately 3′ of the target sequence) (e.g., for Type I CRISPR/Cas systems). In some embodiments, e.g., Type I systems, the PAM is on the alternate side of the protospacer (the 5′ end). Makarova et al. describes the nomenclature for all the classes, types, and subtypes of CRISPR systems (Nature Reviews Microbiology 13:722-736 (2015)). Guide structures and PAMs are described in by R. Barrangou (Genome Biol. 16:247 (2015)).

Non-limiting examples of the PAM sequences include: CC, CA, AG, GT, TA, AC, CA, GC, CG, GG, CT, TG, GA, AGG, TGG, T-rich PAMs (such as TTT, TTG, TITC, etc.), NGG, NGA, NAG, NGGNG and NNAGAAW, NNNNGATT, NAAR (R=A or G), NNGRR (R=A or G), NNAGAA, and NAAAAC, where N is any nucleotide. In some embodiments, the PAM may comprise a sequence of CN, in which N is any nucleotide. In select embodiments, the PAM may comprise a sequence of CC.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization. There may be mismatches distal from the PAM.

In some embodiments, when the system comprises TnsA, TnsB, TnsC, TnsD and TniQ binding to the target nucleic acid may be mediated through a TnsD binding site within the target nucleic acid sequence. Thus, the recognition of the target nucleic acid utilizing the systems described herein may proceed in a gRNA-dependent and/or -independent manner.

d. Nuclear Localization Sequence

In the systems disclosed herein, one or more of the at least one Cas protein, the at least one transposon-associated protein, or the integration co-factor protein may comprise a nuclear localization signal (NLS). The nuclear localization sequence may be appended to the one or more of the at least one Cas protein, the at least one transposon-associated protein and the integration co-factor protein at a N-terminus, a C-terminus, embedded in the protein (e.g., inserted internally within the open reading frame (ORF)), or a combination thereof.

In some embodiments, one or more of the at least one Cas protein, the at least one transposon-associated protein, and integration co-factor protein comprises two or more NLSs. The two or more NLSs may be in tandem, separated by a linker, at either end terminus of the protein, or embedded in the protein (e.g., inserted internally within the ORF instead).

The nuclear localization sequence may comprise any amino acid sequence known in the art to functionally tag or direct a protein for import into a cell's nucleus (e.g., for nuclear transport). Usually, a nuclear localization sequence comprises one or more positively charged amino acids, such as lysine and arginine.

In some embodiments, the NLS is a monopartite sequence. A monopartite NLS comprises a single cluster of positively charged or basic amino acids. In some embodiments, the monopartite NLS comprises a sequence of K-K/R-X-K/R, wherein X can be any amino acid. Exemplary monopartite NLS sequences include those from the SV40 large T-antigen, c-Myc, and TUS-proteins.

In some embodiments, the NLS is a bipartite sequence. Bipartite NLSs comprise two clusters of basic amino acids, separated by a spacer of about 9-12 amino acids. Exemplary bipartite NLSs include the NLS of nucleoplasmin, KR[PAATKKAGQA]KKKK (SEQ ID NO: 15), and the NLS of EGL-13, MSRRRKANPTKLSENAKKLAKEVEN (SEQ ID NO: 16). In some embodiments, the NLS comprises a bipartite SV40 NLS. In certain embodiments, the NLS comprises an amino acid sequence having at least 70% similarity to KRTADGSEFESPKKKRKV (SEQ ID NO: 17). In select embodiments, the NLS consists of an amino acid sequence of KRTADGSEFESPKKKRKV (SEQ ID NO: 17).

The protein components of the disclosed system (e.g., the Cas proteins, the transposon-associated proteins, or the integration co-factor protein) may further comprise an epitope tag (e.g., 3×FLAG tag, an HA tag, a Myc tag, and the like). In some embodiments, the epitope tag may be adjacent, either upstream or downstream, to a nuclear localization sequence. The epitope tags may be at the N-terminus, a C-terminus, or a combination thereof of the corresponding protein.

e. Nucleic Acids

The one or more nucleic acids encoding the engineered CAST system or the nucleic acid encoding the integration co-factor protein may be any nucleic acid including DNA, RNA, or combinations thereof. In some embodiments, nucleic acids comprise one or more messenger RNAs, one or more vectors, or any combination thereof.

The at least one Cas protein, the at least one transposon-associated protein, the at least one integration co-factor protein, the at least one gRNA, and the donor nucleic acid may be on the same or different nucleic acids (e.g., vector(s)). In some embodiments, the at least one Cas protein, the at least one transposon associated protein, and the at least one integration co-factor protein are encoded by different nucleic acids. In some embodiments, the at least one Cas protein and the at least one transposon associated protein encoded by a single nucleic acid. In some embodiments, the at least one Cas protein, the at least one transposon associated protein, and the at least one integration co-factor protein are encoded by a single nucleic acid. In some embodiments, the at least one gRNA is encoded by a nucleic acid different from the nucleic acid(s) encoding the at least one Cas protein, the at least one transposon associated protein, and the at least one integration co-factor protein. In some embodiments, the at least one gRNA is encoded by a nucleic acid also encoding the at least one Cas protein, the at least one transposon associated protein, the at least one integration co-factor protein, or a combination thereof. In some embodiments, the nucleic acid encoding the at least one Cas protein, at least one transposon associated protein, the at least one integration co-factor protein, the at least one gRNA, or any combination thereof further comprises the donor nucleic acid.

In select embodiments, a single nucleic acid encodes the gRNA and at least one Cas protein. The gRNA may be encoded anywhere in the nucleic acid encoding the at least one Cas protein. In some embodiments, the gRNA is encoded in the 3′ UTR of the Cas protein-coding gene.

The one or more nucleic acids encoding the protein components may further comprise, in the case of RNA, or encode, as in the case of DNA, a sequence capable of forming a triple helix adjacent to the sequence encoding the protein component. In some embodiments, the sequence capable of forming a triple helix is downstream of the protein coding sequence. In some embodiments, the sequence capable of forming a triple helix is in a 3′ untranslated region of the protein coding sequence.

A triple helix is formed after the binding of a third strand to the major groove of a duplex nucleic acid through Hoogsteen base pairing (e.g., hydrogen bonds) while maintaining the duplex structure of two strands making the major groove. Pyrimidine-rich and purine-rich sequences (e.g., two pyrimidine tracts and one purine tract or vice versa) can form stable triplex structures as a consequence of the formation of triplets (e.g., A-U-A and C-G-C).

In some embodiments, the triple helix forming sequence comprises two uracil-rich tracts and an adenosine-rich tract, each separated by linker or loop regions. As used herein, the term “A-rich tract” refers to a strand of consecutive nucleosides in which at least 80% of the consecutive nucleosides are adenosine. Similarly, the term “U-rich motif” refers to a strand of consecutive nucleosides in which at least 80% of the consecutive nucleosides are uridine.

In some embodiments, the triple helix sequence is derived from the 3′ terminal triple helix sequences of triple helix terminators from a long non-coding RNAs (lncRNAs), e.g., metastasis-associated lung adenocarcinoma transcript 1 (MALAT1).

One or more of the protein components of the system (e.g., the at least one Cas protein, the at least one transposon associated protein, the at least one integration co-factor protein) may comprise a sequence of an internal ribosome entry site (IRES) or a ribosome skipping peptide. This is particularly advantageous when a single nucleic acid or vector is used to express multiple components of the system.

The ribosome skipping peptide may comprise a 2A family peptide. 2A peptides are short (˜18-25 aa) peptides derived from viruses. There are four commonly used 2A peptides, P2A, T2A, E2A and F2A, that are derived from four different viruses. Any known 2A peptide sequence is suitable for use in the disclosed system.

In certain embodiments, engineering the system for use in eukaryotic cells may involve codon-optimization. It will be appreciated that changing native codons to those most frequently used in mammals allows for maximum expression of the system proteins in mammalian cells (e.g., human cells). Such modified nucleic acid sequences are commonly described in the art as “codon-optimized,” or as utilizing “mammalian-preferred” or “human-preferred” codons. In some embodiments, the nucleic acid sequence is considered codon-optimized if at least about 60% (e.g., 65%, 70%, 75%, 80%, 85%, 90%, 95%, or 98%) of the codons encoded therein are mammalian preferred codons. Furthermore, in some embodiments, engineering the CRISPR-Cas system involves incorporating elements of the native CRISPR array into the disclosed system.

The present disclosure also provides for DNA segments encoding the proteins and nucleic acids disclosed herein, vectors containing these segments and cells containing the vectors. The vectors may be used to propagate the segment in an appropriate cell and/or to allow expression from the segment (e.g., an expression vector). The person of ordinary skill in the art would be aware of the various vectors available for propagation and expression of a nucleic acid sequence.

The present disclosure further provides engineered, non-naturally occurring vectors and vector systems, which can encode one or more or all of the components of the present system. The vector(s) can be introduced into a cell that is capable of expressing the polypeptide encoded thereby, including any suitable prokaryotic or eukaryotic cell.

The vectors of the present disclosure may be delivered to a eukaryotic cell in a subject. Modification of the eukaryotic cells via the present system can take place in a cell culture, where the method comprises isolating the eukaryotic cell from a subject prior to the modification. In some embodiments, the method further comprises returning said eukaryotic cell and/or cells derived therefrom to the subject.

Viral and non-viral based gene transfer methods can be used to introduce nucleic acids encoding components of the present system into cells, tissues, or a subject. Such methods can be used to administer nucleic acids encoding components of the present system to cells in culture, or in a host organism. Non-viral vector delivery systems include DNA plasmids, cosmids, RNA (e.g., a transcript of a vector described herein), a nucleic acid, and a nucleic acid complexed with a delivery vehicle. Viral vector delivery systems include DNA and RNA viruses, which have either episomal or integrated genomes after delivery to the cell. Viral vectors include, for example, retroviral, lentiviral, adenoviral, adeno-associated and herpes simplex viral vectors.

In certain embodiments, plasmids that are non-replicative, or plasmids that can be cured by high temperature may be used, such that any or all of the necessary components of the system may be removed from the cells under certain conditions. For example, this may allow for DNA integration by transforming bacteria of interest, but then being left with engineered strains that have no memory of the plasmids or vectors used for the integration.

Drug selection strategies may be adopted for positively selecting for cells that underwent DNA integration. A donor nucleic acid may contain one or more drug-selectable markers within the cargo. Then presuming that the original donor plasmid is removed, drug selection may be used to enrich for integrated clones. Colony screenings may be used to isolate clonal events.

A variety of viral constructs may be used to deliver the present system (such as one or more Cas proteins, Tns proteins, integration co-factor protein(s), gRNA(s), donor DNA, etc.) to the targeted cells and/or a subject. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1):33-40; and Walther W. and Stein U., 2000 Drugs, 60(2): 249-71, incorporated herein by reference.

In one embodiment, a DNA segment encoding the present protein(s) is contained in a plasmid vector that allows expression of the protein(s) and subsequent isolation and purification of the protein produced by the recombinant vector. Accordingly, the proteins disclosed herein can be purified following expression, obtained by chemical synthesis, or obtained by recombinant methods.

To construct cells that express the present system, expression vectors for stable or transient expression of the present system may be constructed via conventional methods as described herein and introduced into host cells. For example, nucleic acids encoding the components of the present system may be cloned into a suitable expression vector, such as a plasmid or a viral vector in operable linkage to a suitable promoter. The selection of expression vectors/plasmids/viral vectors should be suitable for integration and replication in eukaryotic cells.

In certain embodiments, vectors of the present disclosure can drive the expression of one or more sequences in prokaryotic cells. Promoters that may be used include T7 RNA polymerase promoters, constitutive E. coli promoters, and promoters that could be broadly recognized by transcriptional machinery in a wide range of bacterial organisms. The system may be used with various bacterial hosts.

In certain embodiments, vectors of the present disclosure can drive the expression of one or more sequences in mammalian cells using a mammalian expression vector. Examples of mammalian expression vectors include pCDM8 (Seed. Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible and tissue specific expression can be accomplished by placing the nucleic acid encoding such a molecule under the control of an inducible or tissue specific promoter/regulatory sequence. Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. Various commercially available ubiquitous as well as tissue-specific promoters and tumor-specific are available, for example from InvivoGen. In addition, promoters which are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

The vectors of the present disclosure may direct expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′- and 3′-untranslated regions for mRNA stability and translation efficiency from highly-expressed genes like α-globin or β-globin; SV40 polyoma origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric receptor. Suitable vectors and methods for producing vectors containing transgenes are well known and available in the art. Selectable markers also include chloramphenicol resistance, tetracycline resistance, spectinomycin resistance, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

When introduced into the cell, the vectors may be maintained as an autonomously replicating sequence or extrachromosomal element or may be integrated into host DNA.

In one embodiment, the donor DNA may be delivered using the same gene transfer system as used to deliver the Cas protein, and/or transposon associated proteins (included on the same vector) or may be delivered using a different delivery system. In another embodiment, the donor DNA may be delivered using the same transfer system as used to deliver gRNA(s).

In one embodiment, the present disclosure comprises integration of exogenous DNA into the endogenous gene. Alternatively, an exogenous DNA is not integrated into the endogenous gene. The DNA may be packaged into an extrachromosomal or episomal vector (such as AAV vector), which persists in the nucleus in an extrachromosomal state, and offers donor-template delivery and expression without integration into the host genome. Use of extrachromosomal gene vector technologies has been discussed in detail by Wade-Martins R (Methods Mol Biol. 2011; 738:1-17, incorporated herein by reference).

The present system (e.g., proteins, polynucleotides encoding these proteins, donor polynucleotides and compositions comprising the proteins and/or polynucleotides described herein) may be delivered by any suitable means. In certain embodiments, the system is delivered in vivo. In other embodiments, the system is delivered to isolated/cultured cells (e.g., autologous iPS cells) in vitro to provide modified cells useful for in vivo delivery to patients afflicted with a disease or condition.

Vectors according to the present disclosure can be transformed, transfected, or otherwise introduced into a wide variety of cells. Transfection refers to the taking up of a vector by a cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Any of the vectors comprising a nucleic acid sequence that encodes the components of the present system is also within the scope of the present disclosure. Such a vector may be delivered into host cells by a suitable method. Methods of delivering vectors to cells are well known in the art and may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110(6): 2082-2087, incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more transgenes can be delivered by any method appropriate for introducing nucleic acids into a cell. In some embodiments, the construct or the nucleic acid encoding the components of the present system is a DNA molecule. In some embodiments, the nucleic acid encoding the components of the present system is a DNA vector and may be electroporated to cells. In some embodiments, the nucleic acid encoding the components of the present system is an RNA molecule, which may be electroporated to cells.

Additionally, delivery vehicles such as nanoparticle- and lipid-based mRNA or protein delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1: 27) and Ibraheem et al. (Int J Pharm. 2014 Jan. 1; 459(1-2):70-83), incorporated herein by reference.

### Methods

Also disclosed herein are methods for nucleic acid modification (e.g., insertion or deletion) utilizing the disclosed systems or kits. The methods may comprise contacting a target nucleic acid sequence with a system disclosed herein or a composition comprising the system. The descriptions and embodiments provided above for the engineered CAST system, the at least one integration co-factor protein, the gRN A, and the donor nucleic acid are applicable to the methods described herein.

The target nucleic acid sequence may be in a cell. In some embodiments, contacting a target nucleic acid sequence comprises introducing the system into the cell. As described above the system may be introduced into eukaryotic or prokaryotic cells by methods known in the art. In some embodiments, the cell is a mammalian cell. In some embodiments, the cell is a human cell.

In some embodiments, the target nucleic acid is a nucleic acid endogenous to a target cell. In some embodiments, the target nucleic acid is a genomic DNA sequence. The term “genomic,” as used herein, refers to a nucleic acid sequence (e.g., a gene or locus) that is located on a chromosome in a cell.

In some embodiments, the target nucleic acid encodes a gene or gene product. The term “gene product,” as used herein, refers to any biochemical product resulting from expression of a gene. Gene products may be RNA or protein. RNA gene products include non-coding RNA, such as tRNA, rRNA, micro RNA (miRNA), and small interfering RNA (siRNA), and coding RNA, such as messenger RNA (mRNA). In some embodiments, the target nucleic acid sequence encodes a protein or polypeptide.

The methods may be used for a variety of purposes. For example, the methods may include, but are not limited to, inactivation of a microbial gene, RNA-guided DNA integration in a plant or animal cell, methods of treating a subject suffering from a disease or disorder (e.g., cancer, Duchenne muscular dystrophy (DMD), sickle cell disease (SCD), fp-thalassemia, and hereditary tyrosinemia type I (HTI)), and methods of treating a diseased cell (e.g., a cell deficient in a gene which causes cancer).

The disclosed methods may be used to fuse or link an endogenous protein with the protein cargo encoded in the donor nucleic acid. In some embodiments, when the target nucleic acid sequence encodes a protein or polypeptide or is adjacent to a sequence encoding a protein or polypeptide, the donor nucleic acid having the engineered transposon end sequence encoding an amino acid linker and a peptide or polypeptide cargo fuses or links the endogenous protein with the peptide or polypeptide cargo upon successful insertion. Thus, the disclosure also provides methods of tagging a protein, e.g., an endogenous protein in a cell.

Polynucleotides containing the target nucleic acid sequence may include, but is not limited to, purified chromosomal DNA, total cDNA, cDNA fractionated according to tissue or expression state (e.g., after heat shock or after cytokine treatment other treatment) or expression time (after any such treatment) or developmental stage, plasmid, cosmid, BAC, YAC, phage library, etc. Polynucleotides containing the target site may include DNA from organisms such as Homo sapiens, Mus domesticus, Mus spretus, Canis domesticus, Bos, Caenorhabditis elegans, Plasmodium falciparum, Plasmodiwn vivax, Onchocerca volvulus, Brugia malayi, Dirofilaria immitis, Leishmania, Zea maize. Arabidopsis thaliana, Glycine max, Drosophila melanogaster, Saccharomnyces cerevisiae, Schizosaccharomyces pombe, Neurospora, Escherichia coli, Salmonella typhimuriwn, Bacillus subtilis, Neisseria gonorrhoeae, Staphylococcus aureus, Streptococcus pneumonia, Mycobacterium tuberculosis, Aquifex, Thermus aquaticus, Pyrococcus furiosus, Thermus littoralis, Methanobacterium thermoautotrophicum, Sulfolobus caldoaceticus, and others.

The methods may comprise administering to the subject, in vivo, or by transplantation of ex vivo treated cells, an effective amount of the described system. In some embodiments, the vector(s) is delivered to the tissue of interest by, for example, an intramuscular, intravenous, transdermal, intranasal, oral, mucosal, or other delivery methods.

The components of the present system or ex vivo treated cells may be administered with a pharmaceutically acceptable carrier or excipient as a pharmaceutical composition. In some embodiments, the components of the present system may be mixed, individually or in any combination, with a pharmaceutically acceptable carrier to form pharmaceutical compositions, which are also within the scope of the present disclosure.

In some embodiments, an effective amount of the components of the present system or compositions as described herein can be administered. As used herein the term “effective amount” may be used interchangeably with the term “therapeutically effective amount” and refers to that quantity that is sufficient to result in a desired activity upon administration to a subject in need thereof. Within the context of the present disclosure, the term “effective amount” refers to that quantity of the components of the system such that successful DNA integration is achieved.

When utilized as a method of treatment, the effective amount may depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

In the context of the present disclosure insofar as it relates to any of the disease conditions recited herein, the terms “treat,” “treatment,” and the like mean to relieve or alleviate at least one symptom associated with such condition, or to slow or reverse the progression of such condition. Within the meaning of the present disclosure, the term “treat” also denotes to arrest, delay the onset (e.g., the period prior to clinical manifestation of a disease) and/or reduce the risk of developing or worsening a disease. For example, in connection with cancer the term “treat” may mean eliminate or reduce a patient's tumor burden, or prevent, delay, or inhibit metastasis, etc.

The phrase “pharmaceutically acceptable,” as used in connection with compositions and/or cells of the present disclosure, refers to molecular entities and other ingredients of such compositions that are physiologically tolerable and do not typically produce untoward reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers: monosaccharides; disaccharides; and other carbohydrates; metal complexes; and/or non-ionic surfactants. See, e.g., Remington: The Science and Practice of Pharmacy 20th Ed. (2000) Lippincott Williams and Wilkins, Ed. K. E. Hoover.

### Kits

Also within the scope of the present disclosure are kits that include the components of the present system.

The kit may include instructions for use in any of the methods described herein. The instructions can comprise a description of administration of the present system or composition to a subject to achieve the intended effect. The instructions generally include information as to dosage, dosing schedule, and route of administration for the intended treatment. The kit may further comprise a description of selecting a subject suitable for treatment based on identifying whether the subject is in need of the treatment.

The kits provided herein are in suitable packaging. Suitable packaging includes, but is not limited to, vials, bottles, jars, flexible packaging, and the like.

The packaging may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

The kit may further comprise a device for holding or administering the present system or composition. The device may include an infusion device, an intravenous solution bag, a hypodermic needle, a vial, and/or a syringe.

The present disclosure also provides for kits for performing DNA integration in vitro. The kit may include the components of the present system. Optional components of the kit include one or more of the following: buffer constituents, control plasmid, sequencing primers, cells, and the like.

### EXAMPLES

The following are examples of the present invention and are not to be construed as limiting.

### Materials and Methods

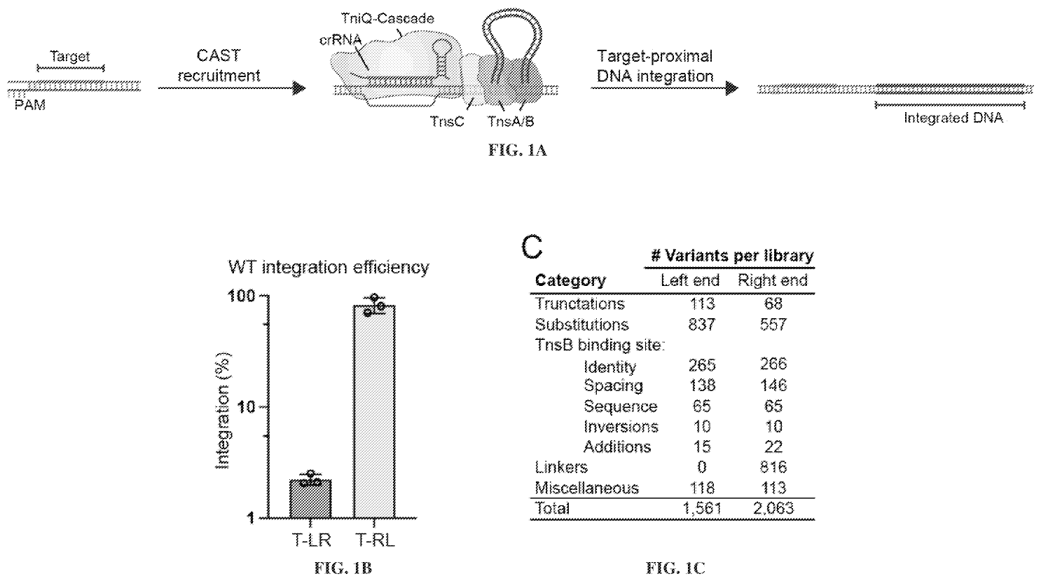

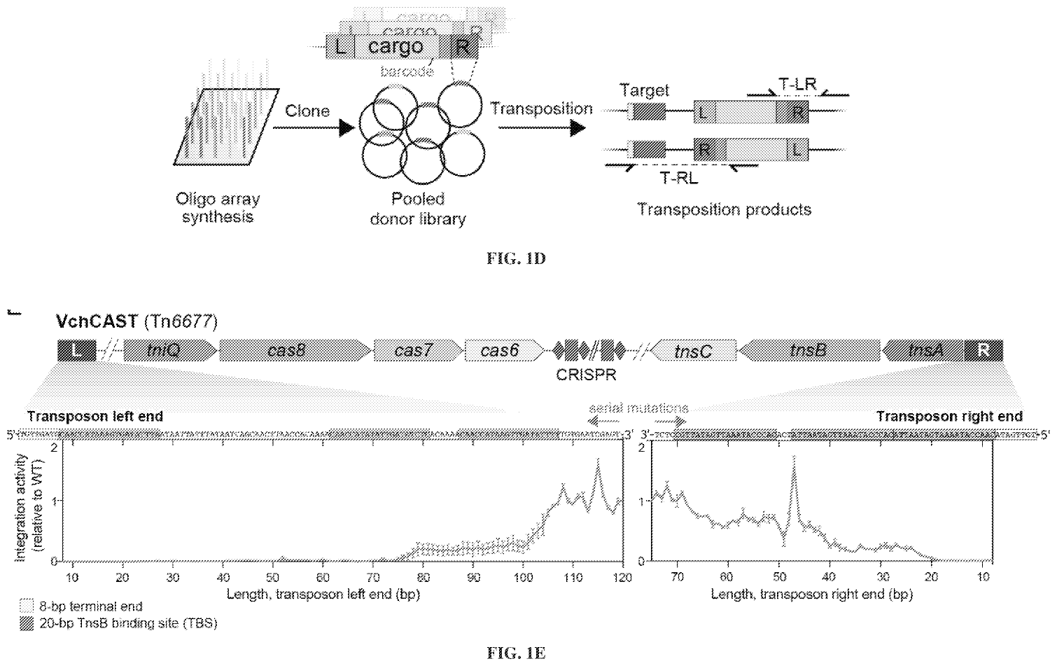

Cloning, testing, and analysis of pooled pDonor libraries. Donor plasmid (pDonor) libraries were generated by cloning transposon left or end variants into a donor plasmid, which was co-transformed with an effector plasmid (pEffector) that directed transposition into the E. coli genome (schematized in FIG. 1D). Each transposon end variant was associated with a unique 10-bp barcode that was used to uniquely identify variants in the sequencing approach, which relied on sequencing the starting plasmid libraries (input) and integrated products from genomic DNA (output) by NGS to determine the representation of each library member before and after transposition. To sequence the output, integration events in the T-RL and T-LR orientations were independently amplified using a cargo-specific primer flanking the transposon end and a genomic primer either upstream or downstream of the integration site. Custom python scripts compared each library member's representation in the output to its representation in the input, allowing calculation of the relative transposition efficiency of the custom transposon end variants.

To clone the transposon donor libraries, library variants were first generated as 200-nt single stranded pooled oligos (Twist Bioscience). 1 ng of oligoarray library DNA was PCR amplified for 12 cycles in 40 μL reactions using Q5 High-Fidelity DNA Polymerase (NEB) and primers specific to the right or left end library, in order to add restriction enzyme digestion sites. Amplicons were cleaned up and eluted in 45 μL mQ H₂O (QIAquick PCR Purification Kit). As the backbone vector, a plasmid encoding a 775-bp mini-transposon, delineated by 147-bp of the native transposon left end and 75-bp of the native transposon right end, on a pUC57 backbone was used. The backbone vector and library insert amplicons were digested (AscI and SapI for the right end library, and NcoI and NotI for the left end library) at 37° C. for 1 h, gel purified, and ligated in 20 μL reactions with T4 DNA Ligase (NEB) at 25° C. for 30 min. Ligation reactions were cleaned up and eluted in 10 μL mQ H₂O (MinElute PCR Purification Kit), and then used to transform electrocompetent NEB 10-beta cells in five individual electroporation reactions according to the manufacturer's protocol. After recovery (37° C. for 1 h), transformed cells were plated on large 245 mm×245 mm bioassay plates containing LB-agar with 100 μg/mL carbenicillin. Plates were scraped to collect cells, and plasmid DNA was isolated using the QIAGEN Plasmid Midi Kit.

Transposition experiments were performed in E. coli BL2I(DE3) cells, pEffector encoded a CRISPR array (repeat-spacer-repeat), a native tniQ-cas8-cas7-cas6 operon, and a native tnsA-tnsB-tnsC operon, all under the control of a single T7 promoter on a pCDFDuet-1 backbone. 2 μL of DNA solution containing 200 ng of pDonor and pEffector in equal molar amount was used to co-transform electrocompetent cells according to the manufacturer's protocol (Sigma-Aldrich). Four transformations were performed for each sample, and following recovery at 37° C. for 1 h, each transformation was plated on a large bioassay plate containing LB-agar with 100 μg/mL spectinomycin, 100 μg/mL carbenicillin, and 0.1 mM IPTG. Cells were grown at 37° C. for 18 h. Thousands of colonies were scraped from each plate, and genomic DNA was extracted using the Wizard Genomic DNA Purification Kit (Promega).

Next-generation sequencing (NGS) amplicons were prepared by PCR amplification using Q5 High-Fidelity DNA Polymerase (NEB). 250 ng of template DNA was amplified in 15 cycles during the PCR1 step. PCR1 samples were diluted 20-fold and amplified in 10 cycles during the PCR2 step. PCR1 primer pairs contained one pDonor backbone-specific primer and one transposon-specific primer (input library), or one genomic target-specific primer and one transposon-specific primer (output library). PCR amplicons were resolved by 2% agarose gel electrophoresis and gel-purified (QIAGEN Gel Extraction Kit). Libraries were quantified by qPCR using the NEBNext Library Quant Kit (NEB). Sequencing for both input and output libraries were performed using a NextSeq Mid or High Output Kit with 150-cycles (Illumina). Additionally, the input libraries were also sequenced using a MiSeq with 300-cycles (Illumina).

NGS data analysis was performed using custom Python scripts. Demultiplexed reads were filtered to remove reads that did not contain a perfect match to the 19-bp primer binding sequence at the 3′-terminus of the transposon end. Then, the 10-bp sequence directly downstream of the primer binding sequence was extracted, which encodes a barcode that uniquely identifies each transposon end variant. The number of reads containing each library member barcode was counted. If a read did not contain a barcode that matched a library member barcode, it was discarded. The barcode counts were summed across two NGS runs using the same PCR2 samples for the input libraries. Two biologically independent replicates were performed for the output libraries. The relative abundance of each library member was then determined by dividing the barcode count of each library member by the total number of barcode counts. The fold-change between the output and input libraries was calculated by dividing the relative abundance of each library member in the output library by its relative abundance in the input library. This fold-change was then normalized by dividing the fold-change of each library member by the average fold-change of four wildtype library members that contained identical transposon ends but unique barcodes.

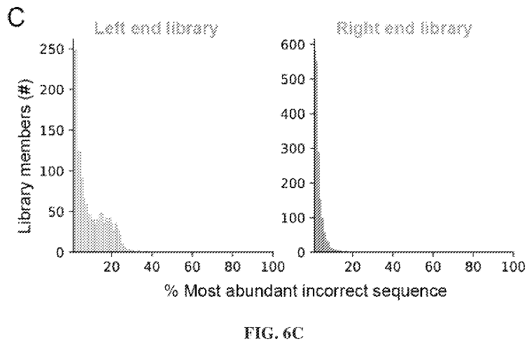

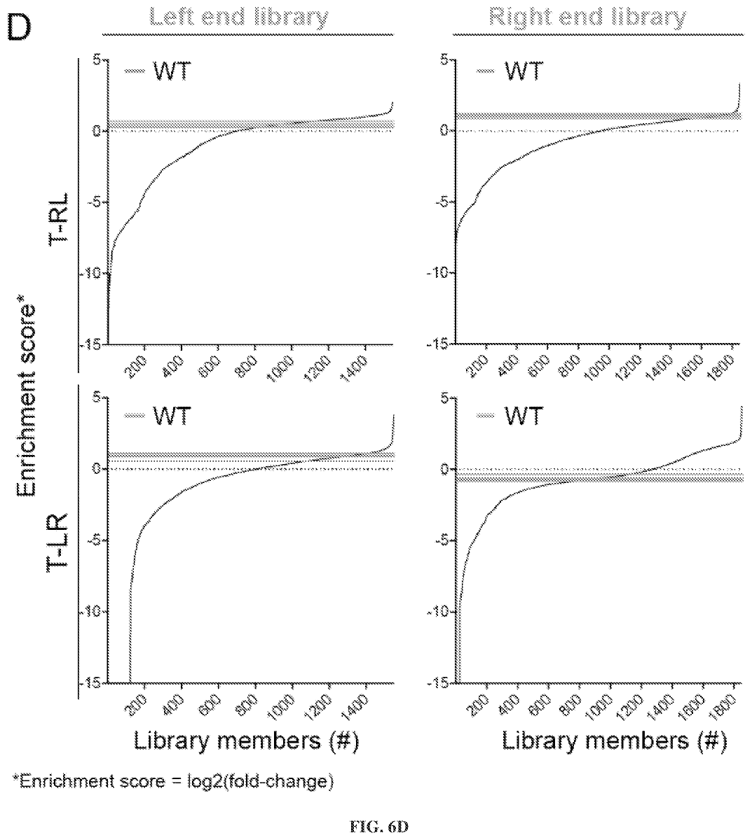

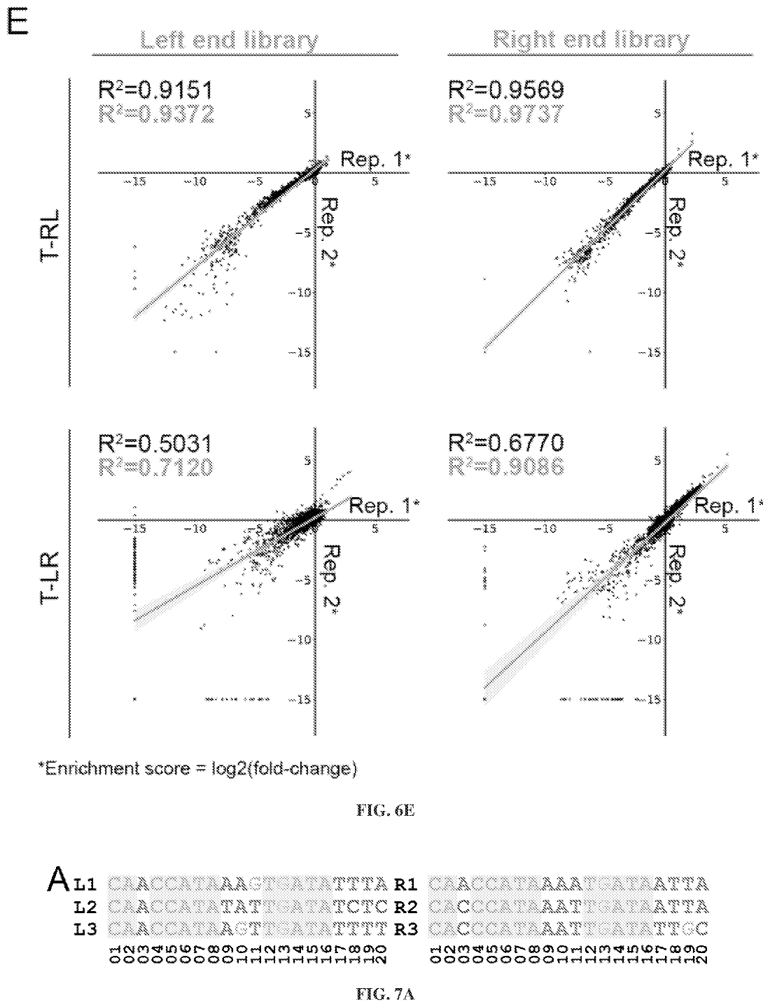

One source of experimental noise in the approach came from PCR recombination, in which barcodes became uncoupled from their associated transposon end variants during PCR amplification. The frequency of uncoupling was quantified by performing long-read Illumina sequencing (MiSeq, 250 cycles) to sequence both the barcode and full-length transposon end, and found that not all barcodes were coupled to their correct transposon end sequence (FIG. 6B). However, uncoupled reads mapped to a diverse pool of sequences, with the most abundant incorrect sequence for each library member representing only a low percentage of total reads (FIG. 6C). These data therefore indicate that uncoupling events did not largely affect the ability to calculate relative integration efficiencies for each library member.

Sequence logos were generated with WebLogo 3.7.4, and the VchCAST sequence logo in FIG. 2B was generated from the six predicted TnsB binding sites. Consensus sequences were generated from the logo where bases with a bitscore >1 are represented as capital letters and bases with a bit score >1 are represented as small letters.

One limitation of the experimental setup is the inability to directly compare relative integration orientation within the same NGS libraries since integration events were amplified independently in the T-RL and T-LR orientations. Instead, approximate integration efficiencies were inferred by comparing the enrichment scores of transposon end variants to those of wildtype variants within the same library. All transposition assays with pDonor libraries were performed heterologously in E. coli under overexpression conditions, and thus subtleties of transposon end recognition and binding that depend on regulated TnsB expression levels may be obscured.

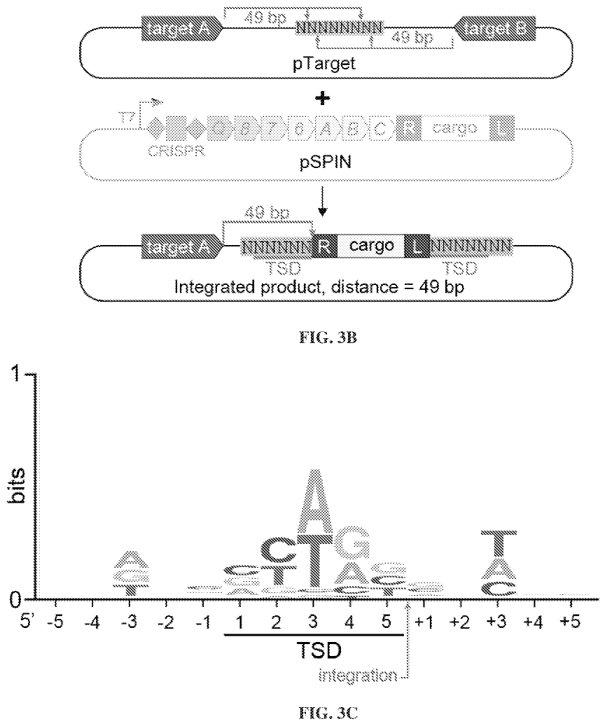

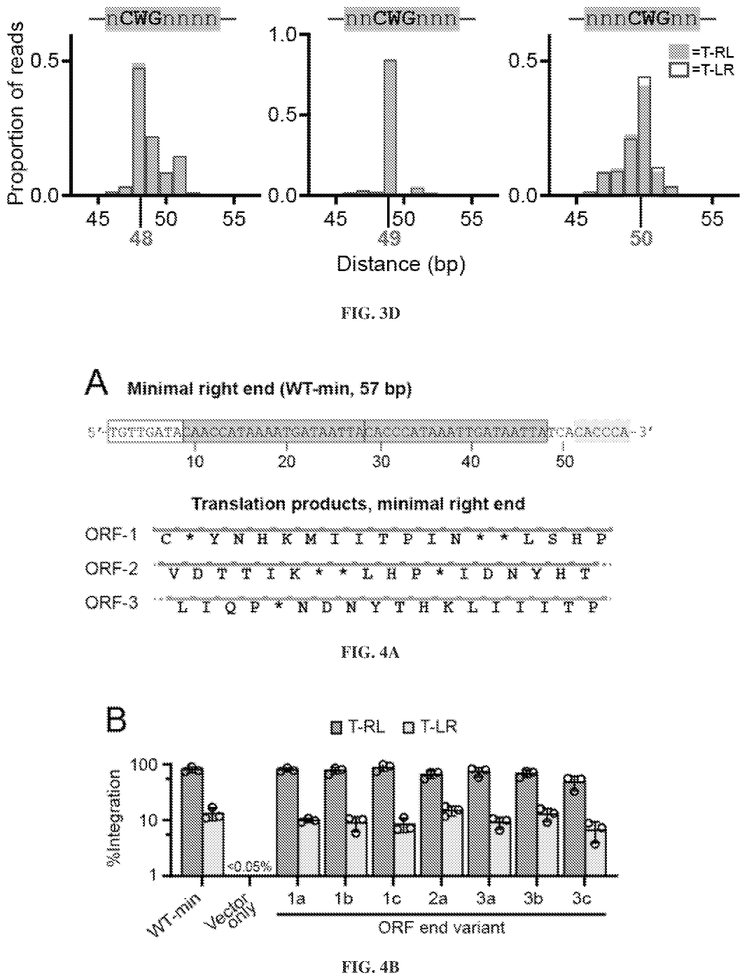

Cloning, testing, and analysis of pooled pTarget libraries. pTarget libraries were designed to include an 8-bp degenerate sequence positioned 42 bp downstream of one of two potential target sites, as schematized in FIG. 3B. Integration was directed to one of the two target sites flanking the degenerate sequence by a single plasmid (pSPIN) encoding both the donor molecule and transposition machinery under the control of a T7 promoter, on a pCDF backbone. To generate insert DNA for cloning the pTarget libraries, two partially overlapping oligos were annealed by heating to 95° C. for 2 min and then cooling to room temperature. Annealed DNA was treated with DNA Polymerase I, Large (Klenow) Fragment (NEB) in 40 μL reactions and incubated at 37° C. for 30 min, then gel-purified (QIAGEN Gel Extraction Kit). Double-stranded insert DNA and vector backbone was digested with BamHI and AvrII (37° C., I h); the digested insert was cleaned-up (MinElute PCR Purification Kit) and the digested backbone was gel-purified. Backbone and insert were ligated with T4 DNA Ligase (NEB), and ligation reactions were used to transform electrocompetent NEB 10-beta cells in four individual electroporation reactions according to the manufacturer's protocol. After recovery (37° C. for 1 h), cells were plated on large bioassay plates containing LB-agar with 50 μg/mL kanamycin. Thousands of colonies were scraped from each plate, and plasmid DNA was isolated using the QIAGEN Plasmid Midi Kit. Plasmid DNA was further purified by mixing with Mag-Bind TotalPure NGS Beads (Omega) at a vol:vol ratio of 0.60× and extracting the supernatant to remove contaminating fragments smaller than ˜450 bp.

2 μL of DNA solution containing 200 ng of pTarget and pSPIN at equal mass amounts were used to co-transform electrocompetent E. coli BL21(DE3) cells according to the manufacturer's protocol (Sigma-Aldrich). Three transformations were performed and plated on large bioassay plates containing LB-agar with 100 μg/mL spectinomycin and 50 μg/mL kanamycin. Thousands of colonies were scraped from each plate, and plasmid DNA was isolated using the QIAGEN Plasmid Midi Kit.

Integration into pTarget yielded a larger plasmid than the starting input plasmid. To isolate the larger plasmid, a digestion step was performed that facilitated resolution of the integrated and unintegrated bands on an agarose gel, for extraction of the larger integrated plasmid. This digestion step was performed on both input and output libraries, digesting with NcoI-HF (37° C. for 1 h) and running them on a 0.7% agarose gel. The products were gel-purified (QIAGEN Gel Extraction Kit) and eluted in 15 μL EB in a MinElute Column (QIAGEN). 6.5 μL of cleaned-up DNA was used in each PCR1 amplification with Q5 High-Fidelity DNA Polymerase (NEB) for 15 cycles. PCR1 samples were diluted 20-fold and amplified in 10 cycles for PCR2. PCR1 primer pairs contained pTarget backbone-specific primers flanking a 45-bp region encompassing the degenerate sequence. Sequencing was performed with a paired-end run using a NextSeq High Output Kit with 150-cycles (Illumina).

NGS data analysis was performed using a custom Python script. Demultiplexed reads were filtered to remove reads that did not contain a perfect match to the 34- to 35-bp sequence upstream of the degenerate sequence for any i5-reads, or to the 45- to 46-bp sequence for any i7-reads. 35-bp and 46-bp was used for reads that were amplified from primers containing an additional nucleotide, which were used in PCR I to generate cluster diversity during sequencing. For all reads that passed filtering, the 8-bp degenerate sequence was extracted and counted. The integration distance was determined in the output libraries by examining the i5 read sequence at an integration distance of 43-bp to 56-bp downstream of each target for the presence of the transposon right or left end sequence (20-nt of each end). The degenerate sequence was then extracted from either or both of the i5 and i7 reads, depending on the integration position. The degenerate sequence counts were summed across the two primer pairs. The relative abundance was determined by dividing the degenerate sequence count by the total number of degenerate sequence counts. Finally, the fold-change between the output and input libraries was calculated by dividing the relative abundance of each degenerate sequence at each integration position in the output library by its relative abundance in the input library, and then log 2-transformed.

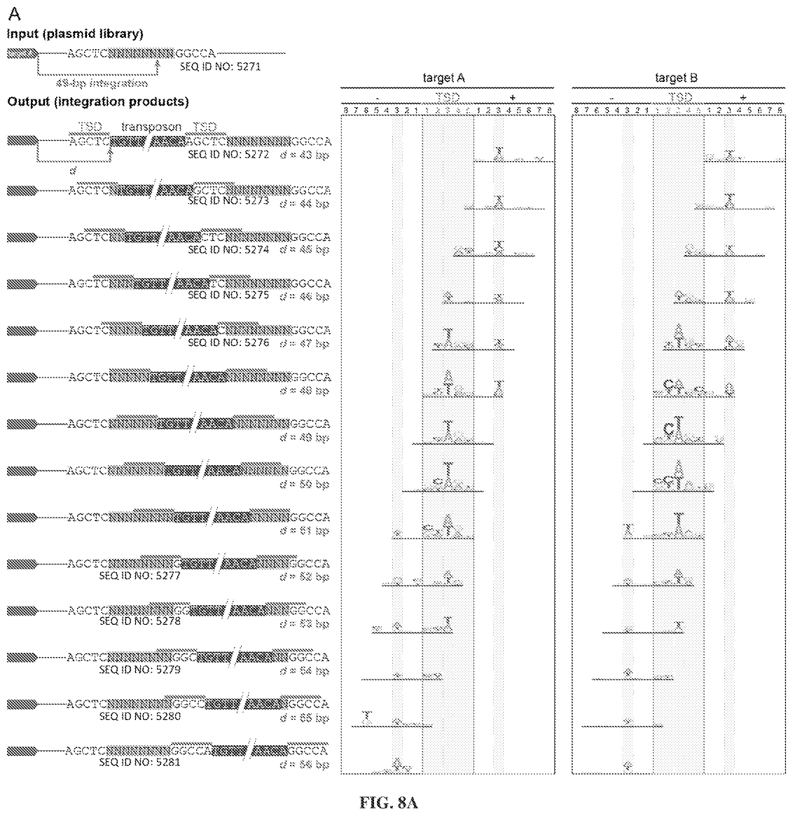

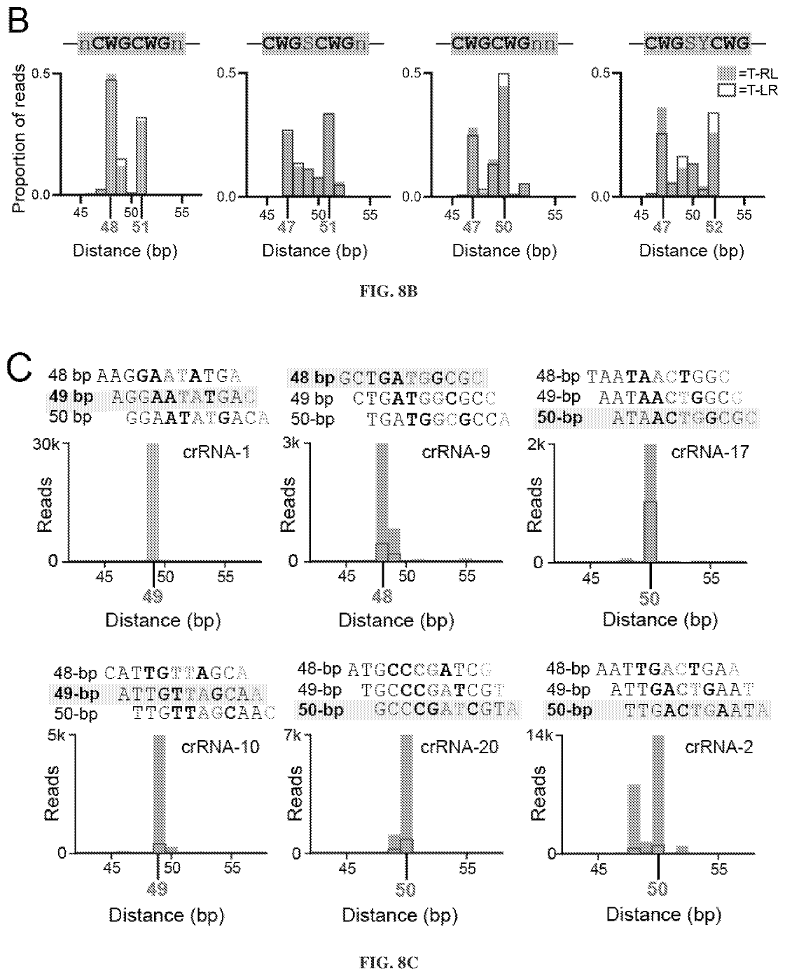

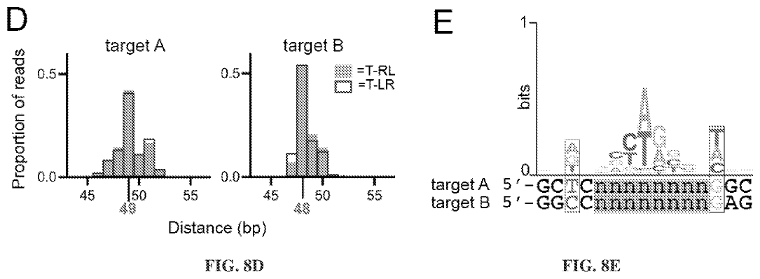

Sequence logos were generated with WebLogo 3.7.4. The preferred integration site logos in FIG. 8A were generated from all degenerate sequences that were enriched four-fold in the integrated products compared to the input. The overall preferred integration site logos in FIGS. 3C and 8D were generated by first applying the minimum threshold of four-fold enrichment in the integrated products compared to the input, and then selecting nucleotides from the top 5,000 enriched sequences across all integration positions. Nucleotides were selected from the top 5,000 sequences from each library, yielding a total of 10,000 nucleotides at each position.

Endogenous gene tagging experiments. All VchCAST constructs were subcloned from pEffector and pDonor as described previously, using a combination of inverse (around-the-horn) PCR, Gibson assembly, restriction digestion-ligation, and ligation of hybridized oligonucleotides. pEffector encodes a CRISPR array (repeat-spacer-repeat), a native tniQ-cas8-cas7-cas6 operon, and a native tnsA-tnsB-tnsC operon, all under the control of a single T7 promoter on a pCDFDuet-1 backbone. Donor plasmids (pDonor) were designed to encode a mini-transposon (mini-Tn) with a wild-type 147-bp transposon left end and 57-bp linker-coding right end variant, on a pUC19 backbone. For endogenous gene tagging experiments, superfolder GFP (sfGFP) lacking a ribosome binding site (rbs) and start codon was cloned into the mini-Tn cargo region, and the mini-Tn was further cloned into a temperature-sensitive pSIM6 backbone.

Linker functionality constructs were designed to encode sfGFP with an extended 32-amino acid (aa) loop region between the 10th and 11th β-strands, under the control of a single T7 promoter, as described by Feng and colleagues. Linker variants encoding 18-19 aa were subcloned into the 32-aa loop region as follows. An entry vector was generated on a pCOLADuet-I (pCOLA) vector harboring sfGFP, such that the 11th β-strand (GFP11) was replaced by the aforementioned extended 32-aa loop. Fragments encoding transposon right end linker variants and GFP11 were then amplified by conventional PCR and inserted into the extended loop region of the entry vector downstream of β-strands 1-10 (GFP1-10), such that total length of the loop remained constant at 32 aa.

To perform linker functionality assays, chemically competent E. coli BL21(DE3) cells were co-transformed with T7-controlled sfGFP linker functionality constructs (pCOLA) and an equal mass amount of empty pUC19 vector. Negative control transformants harbored either unfused sfGFP1-10 and sfGFP11 fragments on separate pCOLA and pUC19 backbones, respectively, or isolated sfGFP fragments. Transformed cells were plated on LB-agar plates with antibiotic selection (100 μg/mL carbenicillin, 50 μg/mL kanamycin), and single colonies were used to inoculate 200 μL of LB medium (100 μg/mL carbenicillin, 50 μg/mL kanamycin, 0.1 mM IPTG) in a 96-well optical-bottom plate. The optical density at 600 nm (OD600) was measured every 10 min, in parallel with the fluorescence signal for sfGFP, using a Synergy Neo2 microplate reader (Biotek) while shaking at 37° C. for 15 h. To derive normalized fluorescence intensities (NFI), all measured fluorescence intensities were divided by their corresponding OD600 values across all time points. A single representative NFI value was calculated per well by averaging all NFI values per well corresponding to OD600 values between 0.20 and 0.30, inclusive.

Transposition experiments were performed by transforming chemically competent E. coli BL21(DE3) cells harboring pEffector plasmids with pDonor plasmids by heat shock at 42° C. for 30 sec, followed by recovery in fresh LB medium. Recovery was performed at 30° C. for 1.5 h for temperature-sensitive pDonor plasmids, and 37° C. for 1 h for all other pDonor plasmids. Transformants were isolated on LB-agar plates containing the proper antibiotics and inducer (100 μg/mL carbenicillin, 50 μg/mL spectinomycin, 0.1 mM IPTG). After 43 h growth at 30° C. for temperature-sensitive pDonor plasmids, and 18 h growth at 37° C. for all other pDonor plasmids, samples were prepared for downstream qPCR analysis of integration efficiency or colony PCR identification of integration events.

For qPCR quantification, colonies were scraped from plates and resuspended in LB medium, and cell lysates were prepared for qPCR as described in Klompe, et al., (2019) Nature, 571, 219-225. Pairs of transposon- and target DNA-specific primers were designed to amplify fragments from integrated transposition products at the expected loci in either of two possible orientations. In parallel, a separate pair of genome-specific primers was designed to amplify an E. coli reference gene (rssA) for normalization purposes. qPCR reactions (10 μL) contained 5 μL of SsoAdvanced Universal SYBR Green Supermix (BioRad), 1 μL H2O, 2 μL of 2.5 μM primers, and 2 μL of hundredfold-diluted cell lysate and were prepared following transposition experiments as described above. Reactions were prepared in 384-well clear/white PCR plates (BioRad), and measurements were obtained in a CFX384 Real-Time PCR Detection System (BioRad). The following thermal cycling parameters were used: polymerase activation and DNA denaturation (98° C. for 3 min), and 35 cycles of amplification (98° C. for 10 s, 60° C. for 30 s). Each biological sample was analyzed in three parallel reactions: one reaction contained a primer pair for the E. coli reference gene, a second reaction contained a primer pair for one integration orientation, and a third reaction contained a primer pair for the other integration orientation. Transposition efficiency was calculated for each orientation as 2ΔCq, in which ΔCq is the Cq difference between the experimental and control reactions. Total transposition efficiency for a given experiment was calculated by summing transposition efficiencies across both orientations. All measurements presented were determined from three independent biological replicates.

For colony PCR identification of integration events, colonies were scraped from plates after transposition assays, resuspended in fresh LB medium, and re-streaked on LB-agar plates with the appropriate antibiotics and without IPTG inducer. To generate lysates, individual colonies were each transferred to 10 μL of H2O, followed by incubation at 95° C. for 2 min and centrifugation at 4,000 g for 5 min to pellet cell debris. Pairs of transposon- and target DNA-specific primers were designed to amplify fragments from integrated transposition products in the expected locus and orientation. In parallel, a separate pair of genome-specific primers was designed to amplify an E. coli reference gene (rssA) and determine whether the crude lysates were sufficiently dilute to allow successful amplification of the integrated transposition product. Transposition-less negative control samples were always analyzed in parallel with experimental samples to identify mispriming products that could result from the pDonor-containing crude lysates. PCR reactions (15 μL) contained 7.5 μL of 2× OneTaq 2× Master Mix with Standard Buffer (NEB), 5.9 μL H₂O, 0.6 μL of 10 μM primers, and 1 μL of undiluted cell lysate as described above. PCR amplicons were resolved by 1% agarose gel electrophoresis and visualized by staining with SYBR Safe (Thermo Scientific). To verify in-frame integration events, amplicons of the expected length were excised after gel electrophoresis, isolated by the Gel Extraction Kit (Qiagen), and sent for Sanger sequencing (GENEWIZ).

Fluorescence microscopy experiments were performed as follows. A pEffector plasmid was designed to C-terminally tag the native E. coli msrB gene by integrating a mini-Tn encoding a linker variant (ORF2a) and sfGFP cargo in-frame with the coding sequence, thereby interrupting the endogenous stop codon. Transposition experiments were performed as described above by transforming chemically competent E. coli BL21(DE3) cells harboring pEffector plasmids with temperature-sensitive pDonor plasmids. Colonies were then scraped and resuspended in fresh LB medium. Resuspensions were diluted and re-streaked on double antibiotic LB-agar plates lacking IPTG (100 μg/mL carbenicillin, 50 μg/mL spectinomycin). After overnight growth on solid medium at 37° C., individual colonies were used to inoculate liquid cultures (50 μg/mL spectinomycin) for overnight heat-curing at 37° C., followed by replica plating on single and double antibiotic plates to isolate heat-cured samples. In tandem, colony PCR and Sanger sequencing (GENEWIZ) were performed to identify colonies with in-frame transposition products as described above. In preparation for fluorescence microscopy, Sanger-verified samples were inoculated in overnight 37° C. liquid cultures. On the day of imaging, 500 μL of saturated overnight cultures were transferred to 5 ml of fresh LB medium with the appropriate antibiotics. Aliquots of the newly inoculated cultures were removed around the stationary or mid-log phases and immobilized in glass slides coated with partially dehydrated aqueous I % agarose-TAE pads. Immediately after immobilization, fluorescent microscopy was performed with a Nikon ECLIPSE 80i microscope using an oil immersion ×100 objective lens, which was equipped with a Spot CCD camera and SpotAdvance software. All images were processed in ImageJ by normalizing background fluorescence.

Generating and testing E. coli knockout mutants. E. coli genomic knockouts of ihfA, ihfB, ycbG, hupA, hupB, hns, and fis were generated using Lambda Red recombineering, as previously described (Sharan, S. K., et al., (2009) Nat Protoc, 4, 206-223). Knockouts were designed to replace of each gene with a kanamycin resistance cassette, which was PCR amplified with Q5 High-Fidelity DNA Polymerase (NEB) using primers that contained 50-nt homology arms to knockout gene locus. PCR amplicons were resolved on a 1% agarose gel and gel-purified, eluting with 40 μL MQ (QIAGEN Gel Extraction Kit). Electrocompetent E. coli BL21(DE3) cells were prepared containing a temperature-sensitive plasmid that encodes the Lambda Red machinery under the control of a temperature-sensitive promoter (pSIM6). Protein expression from the temperature-sensitive promoter was induced by incubating cells at 42° C. for 25 min immediately prior to electrocompetent cell preparation. 300-600 ng of each insert was used to transform cells via electroporation (2 kV, 200 Ω, 25 μF), and cells were recovered overnight at 30° C. by shaking in 3 mL of SOC media. After recovery, 250 μL of culture was spread on 100 mm standard plates (LB-agar with 50 μg/mL kanamycin) and grown overnight at 30° C. Kanamycin-resistant colonies were picked, and the genomic knock-in was confirmed by PCR amplification and Sanger sequencing using primer pairs flanking the knock-in locus.

VchCAST transposition experiments in E. coli knockout strains were performed by first preparing chemically competent WT and mutant cells and then transforming these strains with a single plasmid (pSPIN), which encodes the donor molecule and the native transposition machinery under the control of a T7 promoter and a crRNA targeting the lacZ genomic locus, on a pCDF backbone. After transformation by heat shock, cells were plated onto LB-agar with 100 μg/mL spectinomycin and 0.1 mM IPTG to induce protein expression, and incubated at 37° C. for 18 h. Hundreds of colonies were scraped from each plate, and integration efficiencies were quantified by the same qPCR assay described for the endogenous gene tagging experiments. Transposition experiments for other Type I-F homologs were performed as in the VchCAST experiments, except that the concentration of IPTG was reduced to 0.01 mM to mitigate toxicity.

Experiments that tested protein expression conditions in WT and ΔIHF cells were performed as described in the VchCAST transposition experiments. Promoters were varied from constitutive promoters (J23119, J23101) to inducible promoters (T7), for which different concentrations of IPTG were also tested.

For the complementation experiments, cells were co-transformed with pSPIN and a rescue plasmid (pRescue) that encoded both E. coli ihfA and ihfB under the control of separate T7 promoters on a pACYC backbone, and plated onto LB-agar with 100 μg/mL spectinomycin, 25 μg/mL chloramphenicol, and 0.1 mM IPTG to induce protein expression. Cells were incubated at 37° C. for 18 h, before colonies were scraped from each plate and integration efficiencies in both orientations were measured by qPCR.

To test DNA donor molecules with symmetric transposon ends, mutant pDonor encoding two right or two left transposon ends was cloned, and integration efficiency was measured by co-transforming pDonor with pEffector under the control of a T7 promoter on a pCDF backbone. Cells were plated onto LB-agar with 100 μg/mL spectinomycin, 100 μg/mL carbenicillin, and 0.1 mM IPTG and incubated at 37° C. for 18 h, before colonies were scraped from each plate and integration efficiencies in both orientations were measured by qPCR.

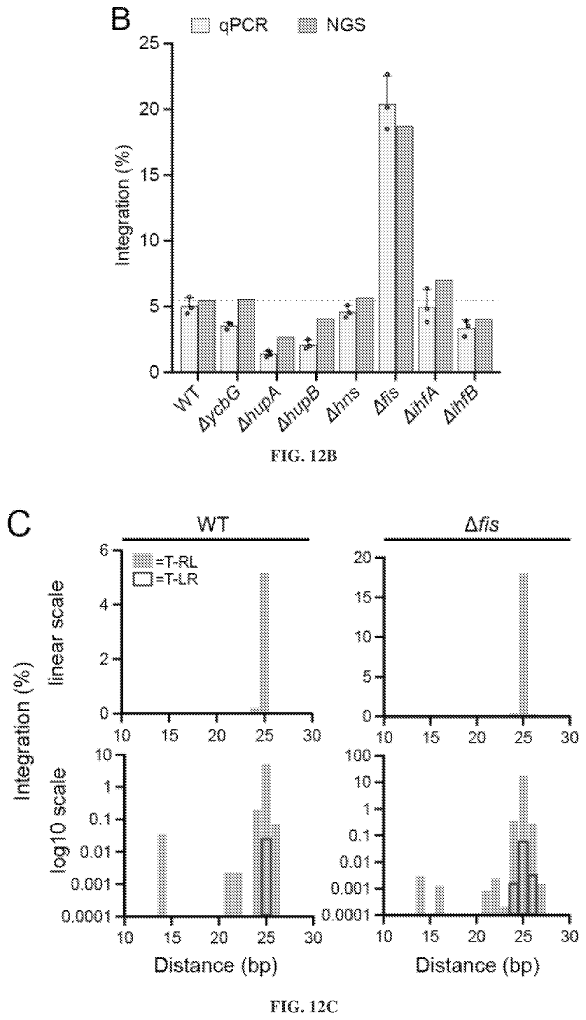

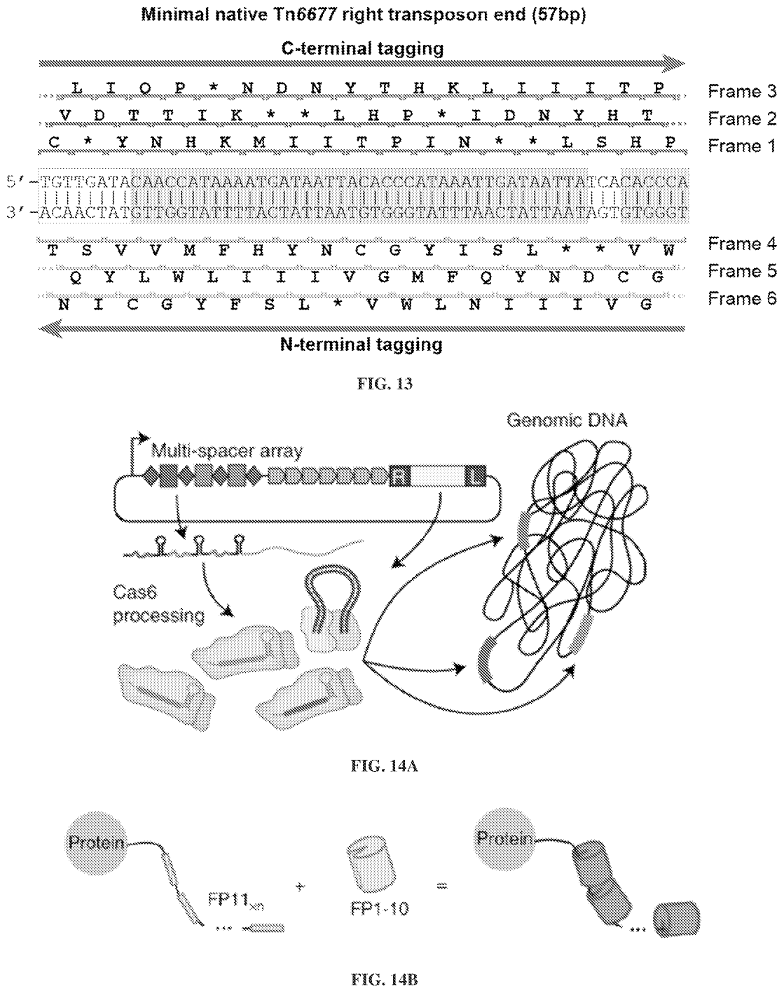

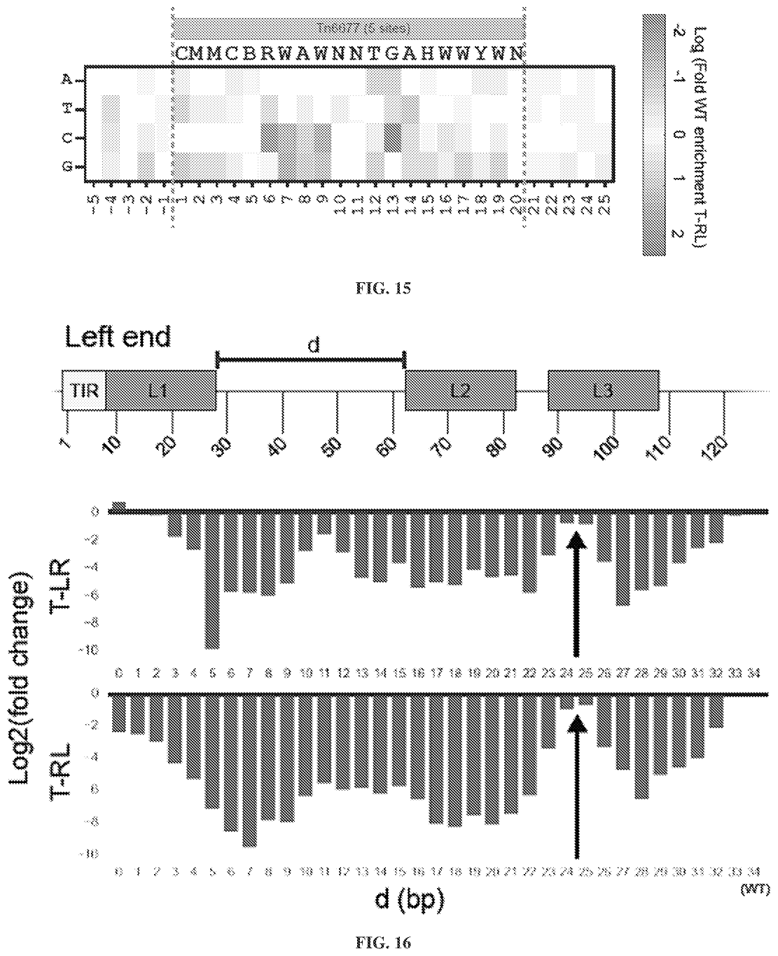

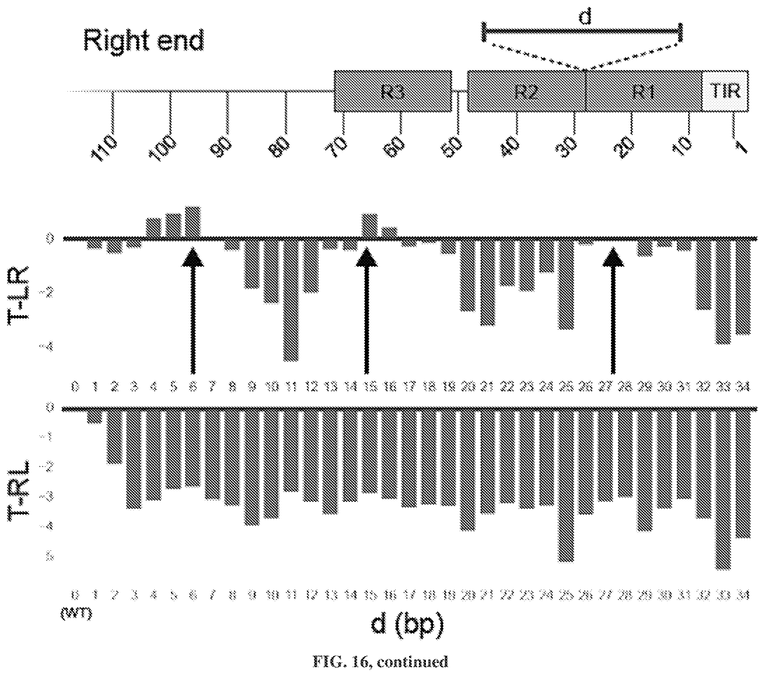

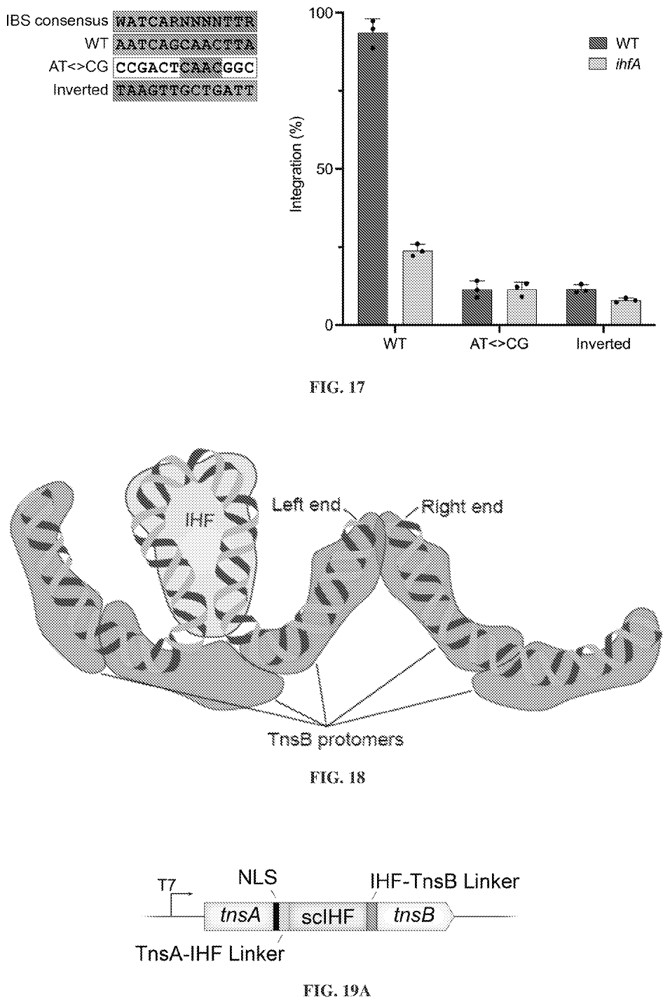

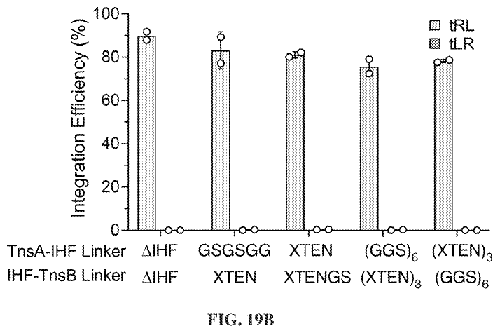

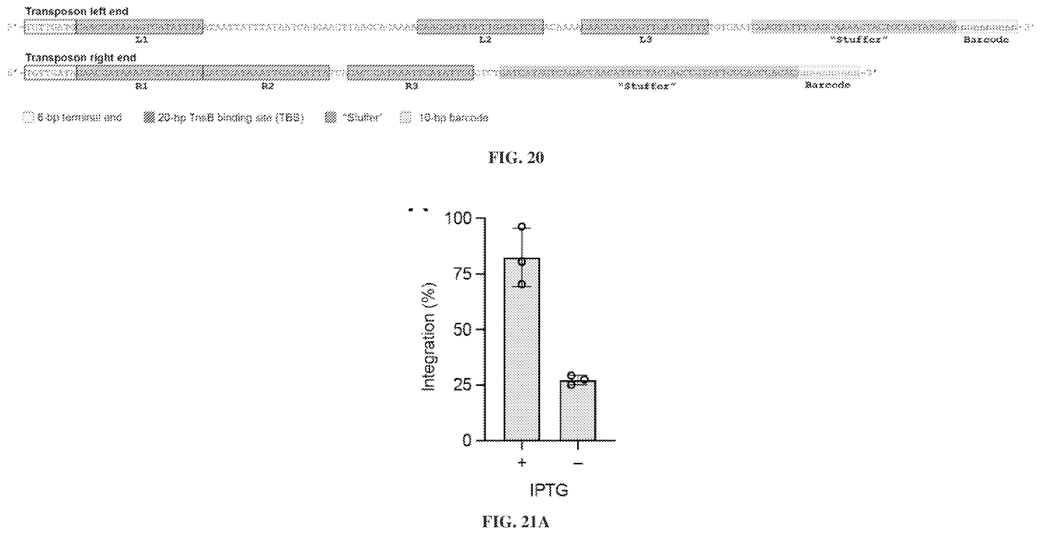

EcoTn7 transposition experiments and NGS analysis. To measure the integration efficiencies and distance distributions of EcoTn7 in WT and E. coli mutant cells, genomic primer binding sites were cloned into the mini-Tn cargo of a single plasmid for Tn7 transposition, which encoded a native tnsA-tnsB-tnsC-tnsD operon under the control of a constitutive pJ23119 promoter, on a pCDF backbone. The genomic primer binding sites were cloned adjacent to the transposon left and right ends such that the NGS amplicon length would be the same for unintegrated products and integrated products in either orientation (schematized in FIG. 12A). To quantify integration efficiencies using qPCR, primer pairs designed to amplify integrated products in both orientations, with one primer adjacent to the right transposon end a second primer either upstream or downstream of the integration site were used.

To quantify integration efficiencies by NGS, genomic DNA was amplified using a single primer pair with one primer complementary to the genomic primer binding site and the second primer complementary to the 3′-end of the glmS locus. Genomic DNA was extracted using the Wizard Genomic DNA Purification Kit (Promega). 250 ng of genomic was used in each PCR1 amplification with Q5 High-Fidelity DNA Polymerase (NEB) for 15 cycles. PCR1 samples were diluted 20-fold and amplified in 10 cycles for PCR2. Sequencing was performed with a paired-end run using a NextSeq High Output Kit with 150-cycles (Illumina).

NGS data analysis was performed using a custom Python script. Demultiplexed reads were filtered to remove reads that did not contain a perfect match to the first 65-bp of expected sequence resulting from either non-integrated genomic products or from integration events spanning 0-bp to 30-bp downstream of the glmS locus, and then counted the number of reads matching each of these possible products.

A table of plasmids used is provided in Table 9.

### Example 1

### Pooled Library to Characterize Transposon End Sequences

To systematically mutagenize the transposon left and right end sequences of V. cholerae Tn6677 large pooled oligoarray libraries, built off the previous study of the VchCAST system (Klompe, et al. (2019) Nature, 571, 219-225), were used. Starting with a minimal pDonor design that directed efficient genomic integration in both of two possible orientations (FIG. 1B), thousands of variants of the left (L) and right (R) end sequences, including truncations, base-pair substitutions, and transposase binding site modifications (FIGS. 1C, SEQ ID NOs: 845-2690 (right end) and SEQ ID NOs: 3120-4665 (left end)) were designed. Each variant was assigned a unique 8-bp barcode located between the mutagenized transposon end and the cargo, obviating the requirement to sequence across the entire transposon end to identify each variant. Each library also included four wildtype (WT) variants associated with unique barcodes, which were used to approximate the relative integration efficiency of each mutagenized library member. Libraries were then synthesized as single-stranded oligos, cloned into a mini-transposon donor (pDonor), and carefully characterized using next-generation sequencing (NGS), which demonstrated that all members were represented in the input sample for both transposon left and right end libraries (FIGS. 6A-D).

Transposition experiments were performed by transforming E. coli BL21(DE3) cells expressing the transposition machinery with pDonor encoding either the left end or right end library, amplifying successful genomic integration products in both orientations via junction PCR (FIG. 1D), and subjecting PCR products to NGS analysis. An enrichment score was then calculated for each variant, revealing a wide range of integration efficiencies, with most library members exhibiting diminished integration relative to the four WT samples (FIG. 6D). Finally, enrichment scores of the WT library members were used for normalization, yielding a score for each variant that represented its relative activity. To validate the approach, two biological replicates for each library transposition experiment were performed and strong concordance between both datasets was found, especially in the dominant T-RL integration orientation (FIG. 6E). Importantly, given the high degree of sequence similarity between library members, the background level of library member-barcode uncoupling was also rigorously determined, which established contributors of experimental noise in our datasets (FIGS. 6B-C and Methods).

The strength of the pooled-library approach was apparent by examining the effect of one category of variations, in which the transposon end sequences were sequentially mutated starting 120-nt into the transposon end, effectively creating end truncations, albeit without a change in overall mini-transposon size (FIG. 1E). These results revealed the minimal transposon end sequence length: in the left end, ˜105 bp were required for efficient integration, corresponding to all three predicted transposase (TnsB) binding sites, whereas in the right end, only ˜50 bp were required, corresponding to the first two transposase binding sites. These findings add single-bp resolution to the minimal transposon end sequences needed for efficient integration.

### Example 2

### Transposase Activity and Transposon End Sequences

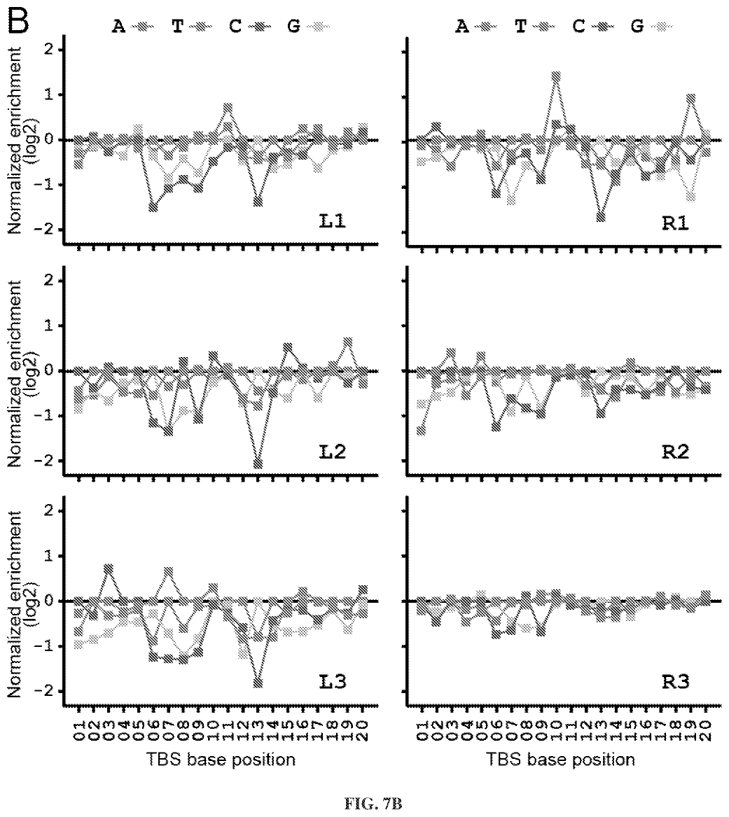

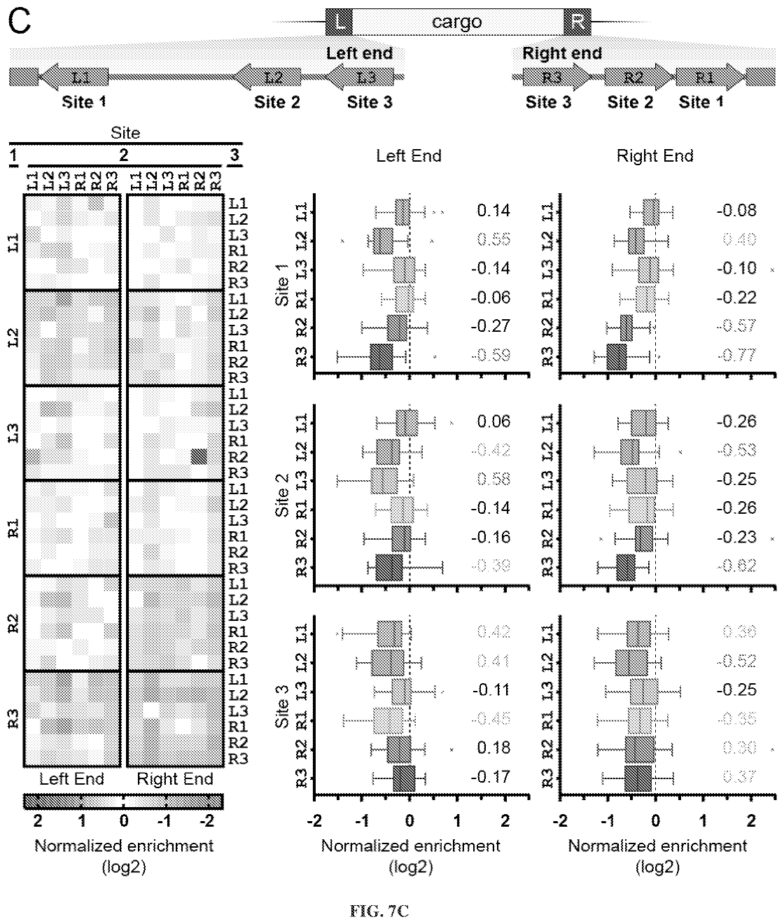

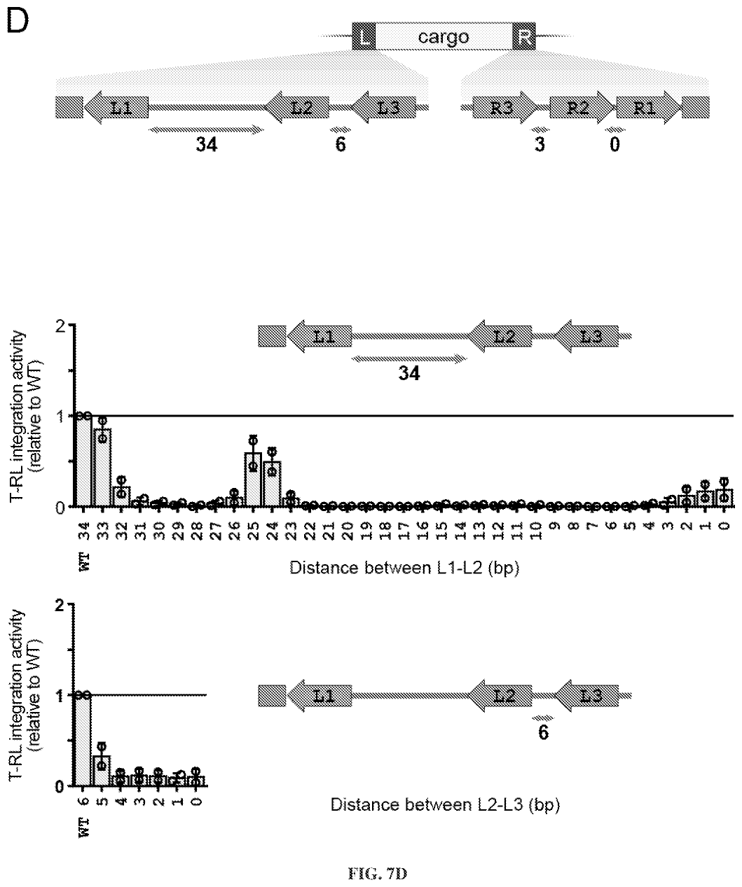

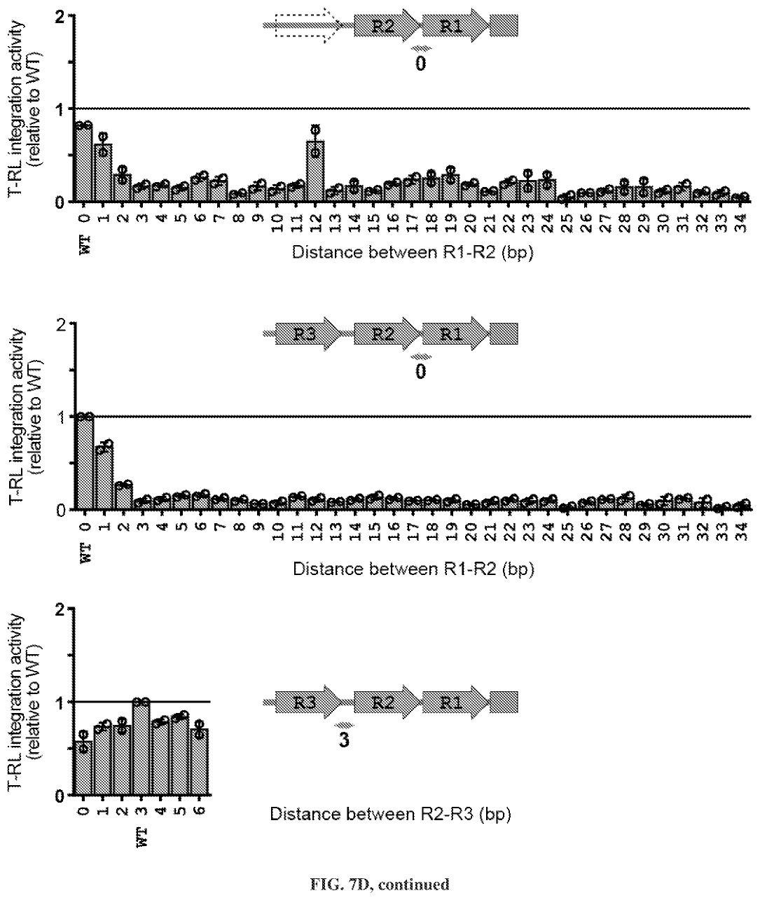

TnsB is integral to the mobilization of Tn7-like transposons, in that it catalyzes the excision and integration chemistry while also conferring sequence specificity for the transposon ends through recognition of repetitive sequence elements known as TnsB binding sites (TBSs). Sequence analysis of the native VchCAST ends revealed three conserved TBSs in both the left and right ends (FIGS. 2A, 2B and 7A), and these sequences were verified by examining a mutational panel at single-bp resolution (FIGS. 2C and 7B). This dataset revealed that individual TBS point mutations can affect efficiency, particularly for positions 1, 6-9, and 12-14, but are not critical for integration. This more lenient sequence requirement is in line with recently published cryo-EM structures of DNA-bound TnsB from Tn7 and Type V-K CAST systems, which revealed that many protein-DNA interactions occur with the phosphodiester backbone rather than specific nucleobases.

Experiments with E. coli Tn7 showed that the internal TBSs are occupied before the more terminal sites. To test this if the few bases which account for the difference in the six TBSs of VchCAST, all possible combinations of TBSs for the left and right ends were tested, which are defined herein as L1-L3 and R1-R3 (FIG. 7C). For both VchCAST ends, site 1 displayed the greatest TBS preference and preferred the L1/L3/R1 sequence, whereas site 2 preferred L1/R1/R2 and site 3 exhibited the least TBS preference but favored L3. A preference for R1 was observed in the first position on the left end, and a preference for L I was observed in the first position on the right end, suggesting that transposition might be favored when the terminal end sequences are identical (whether based on equal affinity or otherwise).

Apart from regulating transposition frequency, TBS sequence identity could also explain the propensity of a given CAST system to cross-react with related transposon substrates. Previously VchCAST was shown to efficiently mobilize mini-transposon substrates from three homologous CAST systems, but not Tn7002. To determine which Tn7002 sequences were incompatible with mobilization by VchCAST machinery, chimeric transposon ends that contain parts of both the VchCAST and Tn7002 transposon ends were designed (FIG. 2D). The data revealed that chimeric left ends allowed for near WT integration efficiencies whereas chimeric right ends drastically decreased integration efficiency, likely due to the deleterious presence of a cytidine at position 9 of R1-R3 (FIG. 2D). Thus, TBS sequence identity imparts at least some constraints on the substrate recognition of a transposase for its cognate transposon DNA.

After testing a mutagenic panel in which the length between TBSs was systematically varied (FIGS. 2E and 7D), it was found that even single-bp perturbations caused drastic changes in integration efficiency. Additionally, an intriguing pattern of increasing and decreasing integration efficiencies were detected at roughly 10-bp intervals, suggesting that the three-dimensional positioning of transposase proteins on helical DNA is important for transposition.

### Example 3

### Transposase Sequence Preferences Influence Integration Site Patterns

VchCAST integration patterns differed in subtle but reproducible ways between distinct genomic target sites. Integration site patterns were compared for four endogenous E. coli target sequences, designated 4-7, either at their native genomic location or on an ectopic target plasmid by deep sequencing (FIG. 3A). Integration site patterns were notably distinct between the four targets but were highly consistent between genomic and plasmid contexts, suggesting that these patterns are dependent on local sequence alone and independent of other factors such as DNA replication or local transcription. Next, to disentangle contributions of the 32-bp target sequence (complementary to crRNA guide) from the downstream region including the integration site, target plasmids that contained chimeras of the four target regions were tested (FIG. 3A). Remarkably, integration patterns for these chimeric substrates closely mirrored the patterns observed for the non-chimeric substrates when the ‘downstream region’ was kept constant, indicating that the 32-bp target sequence does not modulate selection of the integration site.

To test if TnsB might exhibit local sequence preferences immediately at the site of DNA insertion, and explain the observed heterogeneity in integration site patterns, a target plasmid (pTarget) library encoding two target sequences flanking an 8-bp degenerate sequence was generated, such that integration events directed by a crRNA matching either target would lead to insertion directly into the degenerate 8-mer sequence (FIG. 3B). The target plasmids were sequenced before and after transposition and the representation of integration site sequences were compared to determine which sequences were enriched after transposition. These analyses revealed striking nucleotide preferences at conserved positions relative to the integration site (FIGS. 3C and 8A). Specifically, there were clear biases for a YWR motif within the central three nucleotides of the target-site duplication (TSD), as well as a preference for D (A, T, or G) and H (A, T, or C) at the −3 and +3 positions relative to the TSD, respectively.

To further explore the deterministic role of the preferred motif within the TSD, the distribution of reads containing a central 5′-CWG-3′ motif at different positions within the degenerate sequence was plotted. This motif was a focus because it favored a more unimodal distribution for the integration site by avoiding a centrally-preferred A or T nucleotide flanking the W. This motif was predictive of the preferred integration site distance that was sampled by VchCAST (FIG. 3D). By plotting the distribution of reads containing multiple 5′-CWG-3′ motifs within the integration site, it was found that two copies of this preferred motif within the integration site conferred a bimodal distribution, wherein there were not one but two preferred integration sites within the degenerate sequence (FIG. 8B). Finally, the library data was leveraged to predict the integration site distribution of previously targeted locations and could explain their differences at single-bp resolution (FIG. 8E).

Both of the two distinct crRNAs and corresponding target sites on pTarget yielded consistent sequence preferences for both the TSD and +/−3-bp positions (FIG. 8A), but it was surprising to find that the preferred integration distance was shifted by 1-bp when comparing the two (FIG. 8C). This difference could have been due to sequence preferences at the +/−3-bp position that fell outside the degenerate sequence, and indeed, when the sequences flanking the 8-mer library were examined, it was found that the downstream target (target B) contained a disfavored nucleotide in the −3-bp position for insertions that would occur with the 49-bp distance (FIG. 8D).

### Example 4

### Role of Boundary Sequences and Right End Internal Features on DNA Integration

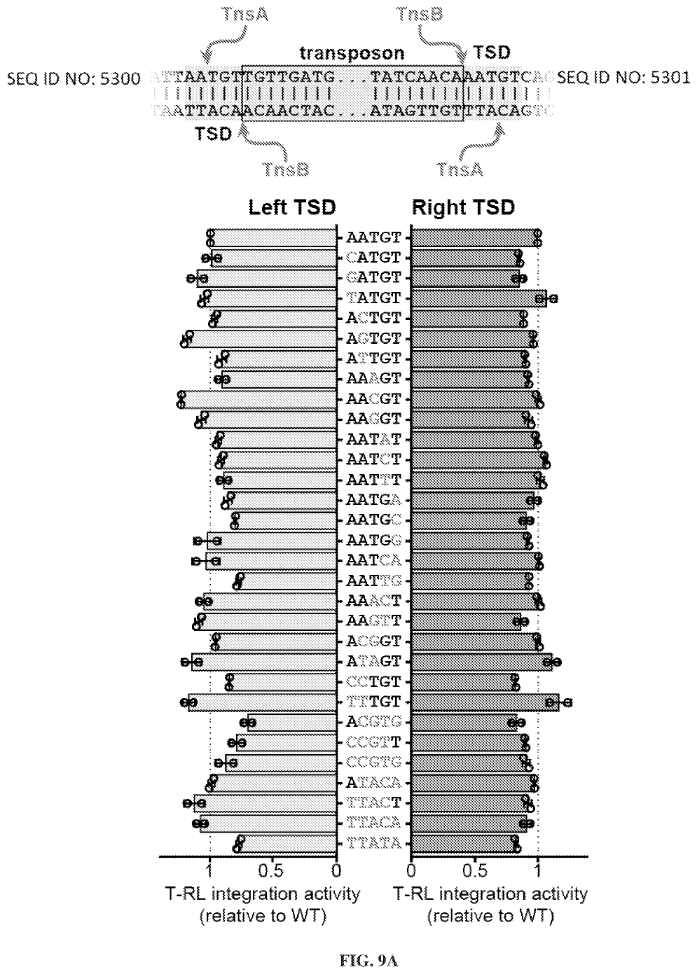

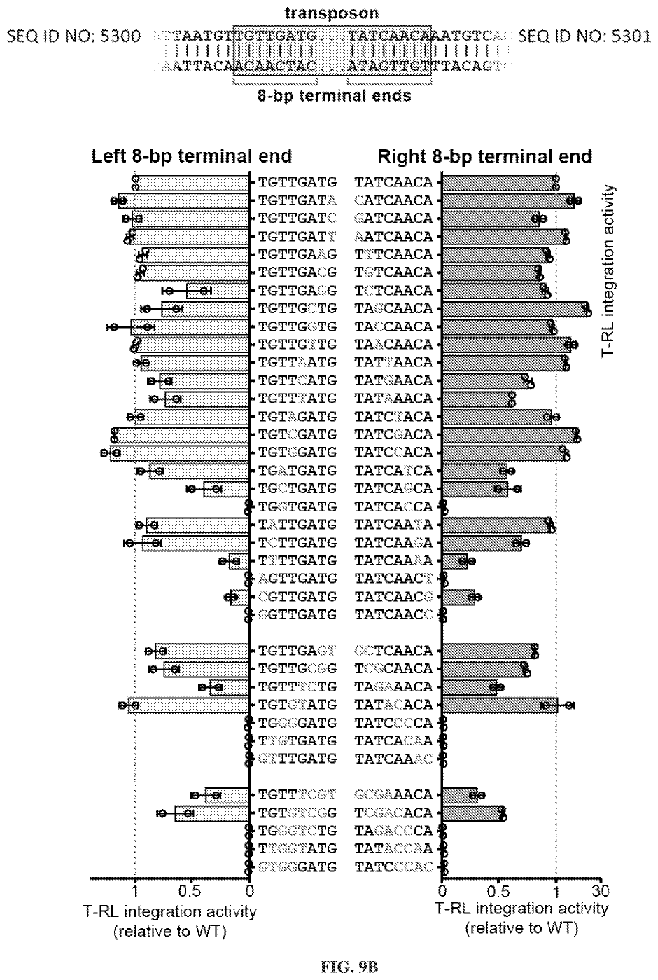

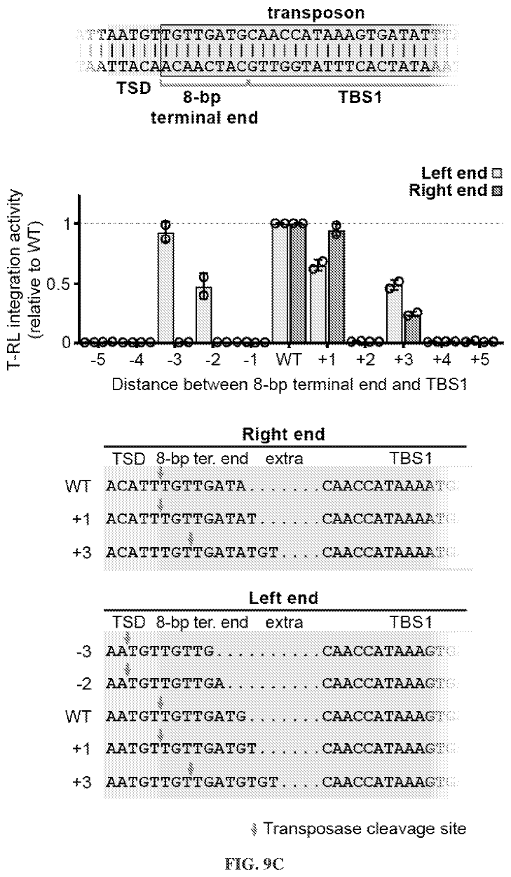

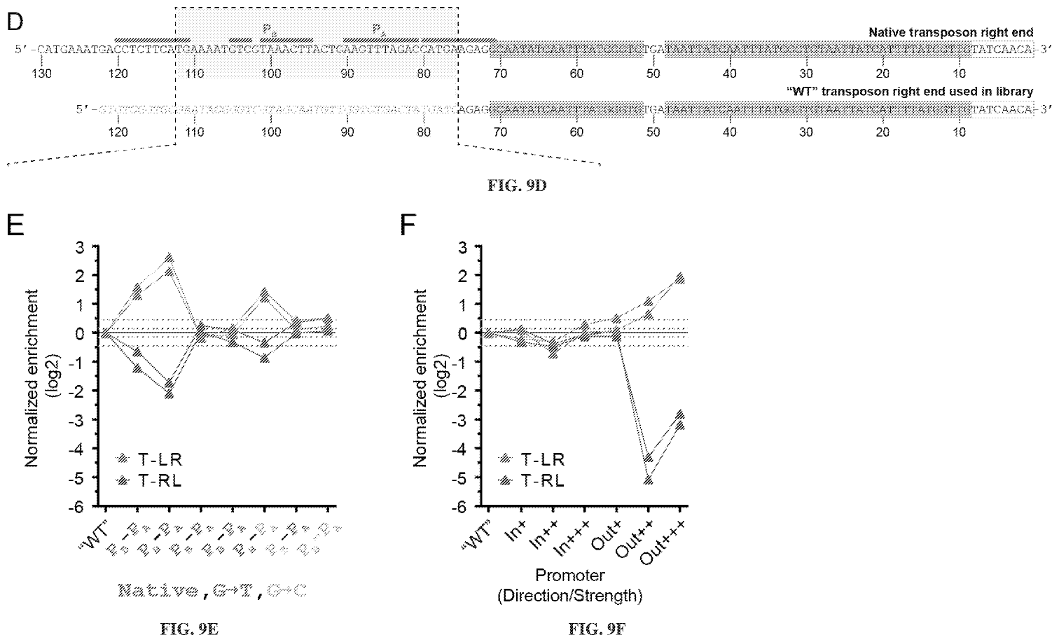

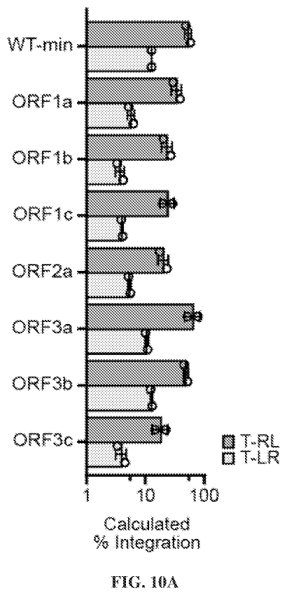

VchCAST and many other Tn7-like transposons encode an 8-bp terminal end immediately adjacent to the first transposase binding site, with the terminal TG dinucleotide highly conserved among a broad spectrum of transposons including IS3, Tn7, Mu and even retrotransposons. Integration data with library variants that featured mutations within these terminal residues revealed that positions 1-3, but not 4-8, were critical for efficient transposition (FIG. 9B). This result is consistent with the DNA-bound cryo-EM structure of TnsB from a Type V-K CAST system. However, library variants with mutations in the 5-bp sequence flanking the mini-transposon were integrated with equivalent efficiencies (FIG. 9A), indicating that transposition machinery does not exhibit sequence specificity within this region.

To investigate whether the spacing between the terminal TG dinucleotide and the first TBS mattered, variants that modulated the distance between the 8-bp terminal end and TBS1 were tested (FIG. 9C). Adding a single base pair in either the left or right end still allowed for efficient transposition, whereas transposition was completely ablated with the removal of 1 bp or addition of 2 bp, indicating tight control over this spacing. Interestingly, larger bp additions or deletions between the TG dinucleotide and first TBS were in some cases also permitted, but always with a concomitant shift in the transposon boundary that was actually mobilized and integrated at the target site (FIG. 9C); in all cases, transposition still required a terminal TG. These data therefore suggest that a controlling feature within the terminal end sequence is the TG dinucleotide, and that the ˜8-bp spacing between this dinucleotide and the first TBS is a constraint for efficient transposition.

Previous work suggested that the palindromic sequence found 97-107 bp from the transposon right end boundary might affect integration orientation, possibly by promoting transcription of the tnsABC operon, which would be consistent with empirical expression data and the AT-richness of the transposon end. To test this possibility, the palindromic sequence was mutated and variants with this sequence shifted the orientation preference towards T-LR, with just one arm of the palindrome (Pa) being sufficient to shift the orientation bias (FIGS. 9D-E). Constitutive promoters were included in place of the palindromic sequence and it was found that promoters directing transcription inwards (towards the cargo) did not impact integration orientation, whereas promoters directed outwards (across the right end) shifted the orientation preference towards T-LR, perhaps by antagonizing stable assembly of TnsB selectively at the right end (FIG. 9F).

### Example 5

Endogenous Protein Tagging with Rationally Engineered Right Ends

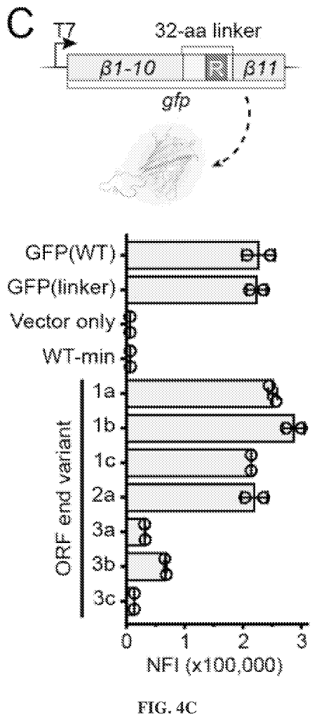

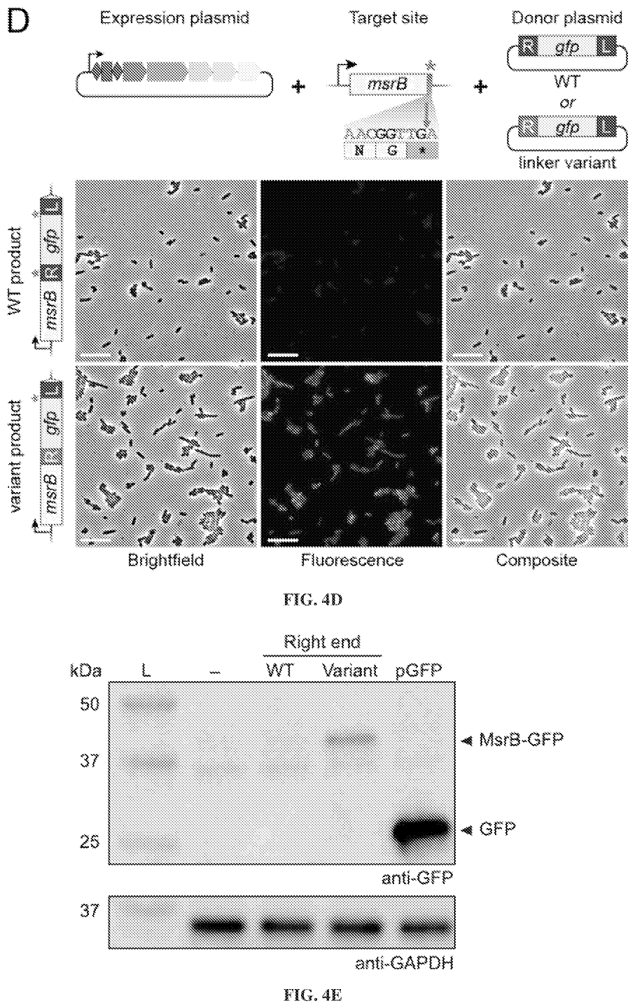

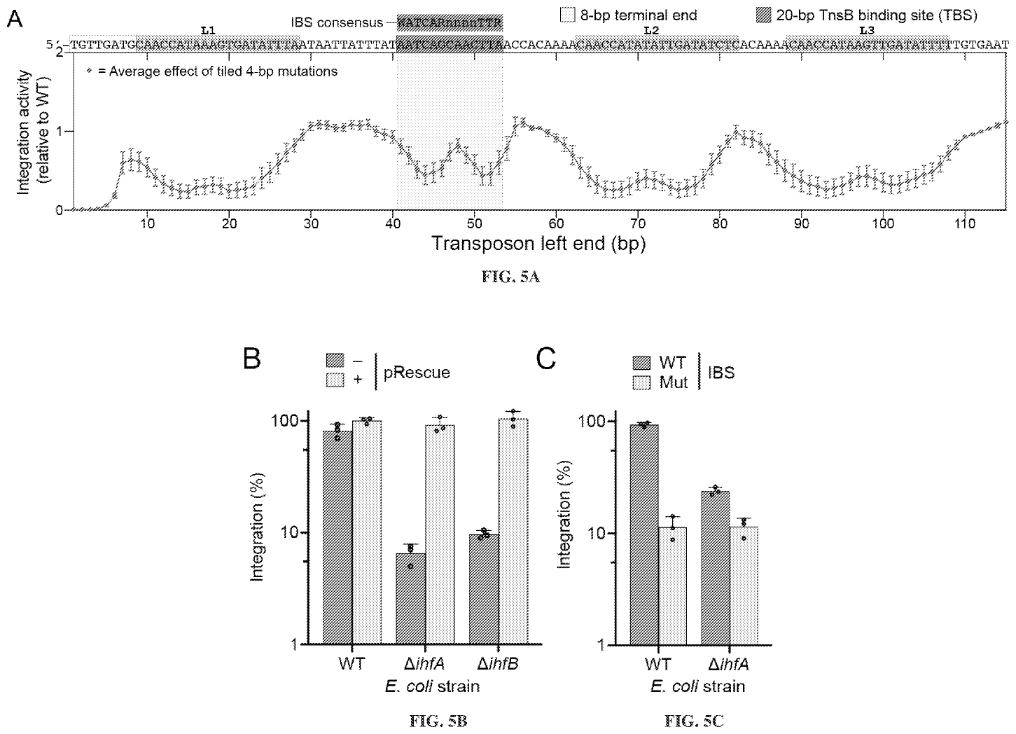

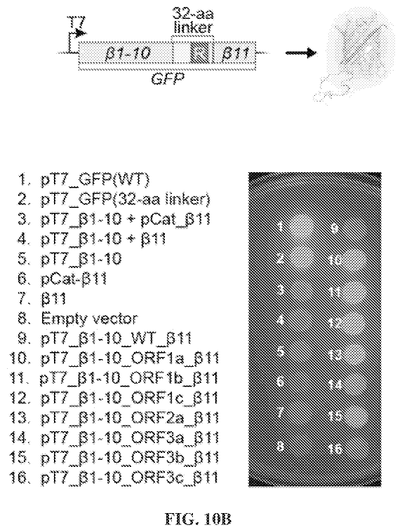

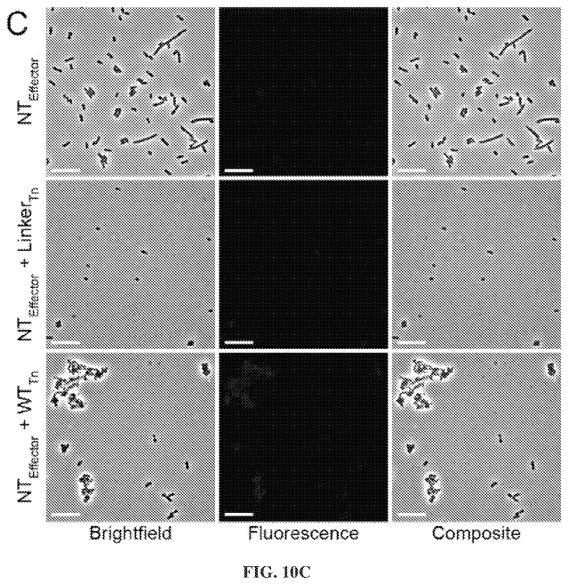

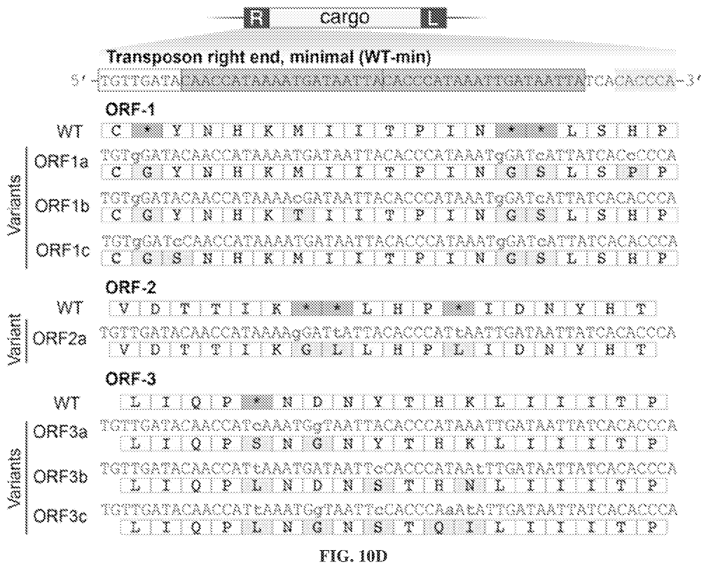

The left and right end sequences facilitate transposon DNA recognition and excision/integration, and transposition products therefore include these sequences as ‘scars’ at the site of insertion. To convert these scars into functional sequences that encode amino acid linkers for downstream protein tagging applications, the shorter right end, starting with a minimal 57-bp sequence, was found to have stop codons in all three possible open reading frames (ORF) for the WT sequence (FIG. 4A). When a library of rationally designed right end variants (SEQ ID NOs: 18-844, Tables 1 &2) that replaced stop codons and codons encoding bulky and/or charged amino acids was tested (FIG. 10D), numerous candidates for each possible ORF that maintained near-wild-type integration efficiency were identified (FIG. 10A; SEQ ID NOs: 1-8; Tables 2 and 4). After validating library data by testing individual linker variants for genomic integration in E. coli (FIG. 4B), a fluorescence-based assay was designed to test for functionality of the encoded amino acid linkers.

GFP naturally consists of eleven β-strands that are connected by small loop regions, and a prior study demonstrated that the loop region between the 10^{th} and 11^{th} β-strand can be extended with novel linker sequences while still allowing for proper folding and fluorescence of the variant GFP protein. Selected transposon right end variants were cloned into the loop region between J3-strand 10 and 11 and GFP fluorescence intensity was measured after expression of each construct, revealing a subset of variants that were fully functional (FIGS. 4C and 10B). Next, the endogenous E. coli gene nsrB was selected for C-terminal tagging in a proof-of-concept experiment (FIG. 4D). After generating a pDonor construct that encodes a right end linker variant with an adjacent, in-frame GFP gene lacking a promoter or start codon, transposition experiments followed by Sanger sequencing were used to verify that integration interrupted the endogenous stop codon while placing the linker and GFP sequence directly in-frame. Finally, proper expression of MsrB-GFP fusion proteins was analyzed by analyzing cells via fluorescence microscopy that received either the WT transposon right end or the linker variant, demonstrating that only the modified right end variant elicited the expected cellular fluorescence (FIGS. 4D and IOC). To confirm that GFP was translationally fused to MsrB, we performed an anti-GFP western blot and found that GFP was not detected in the WT transposon end fusion but was detected at the expected size in the modified linker variant (FIG. 4E). Together, these data provide the basis for new genome engineering tools that allow for facile, endogenous gene tagging with single-bp control.

### Example 6

### Integration Host Factor (IHF) Binds the Left Transposon End to Stimulate Transposition

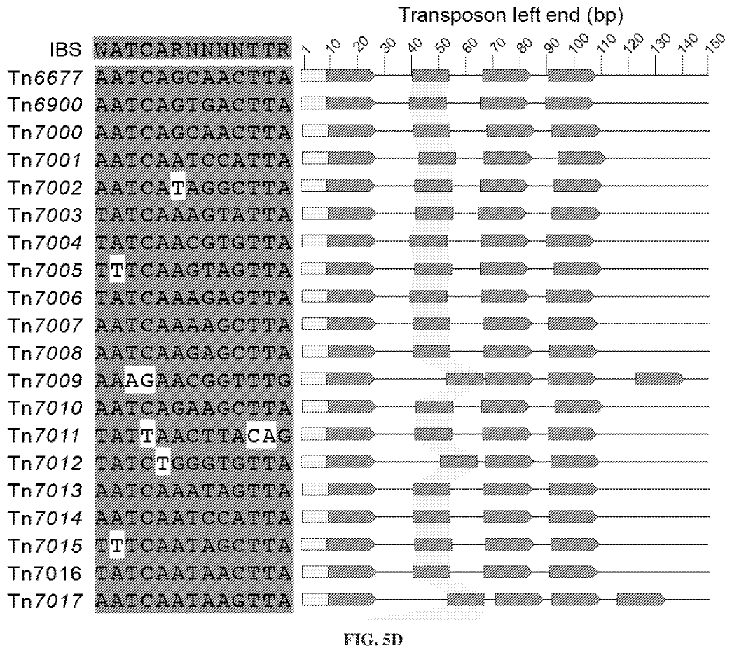

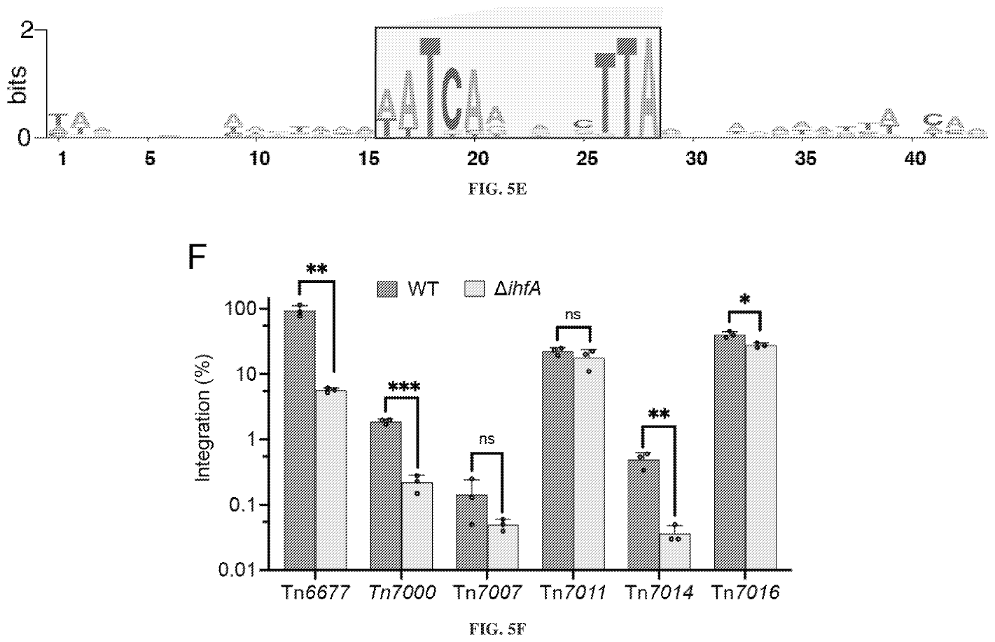

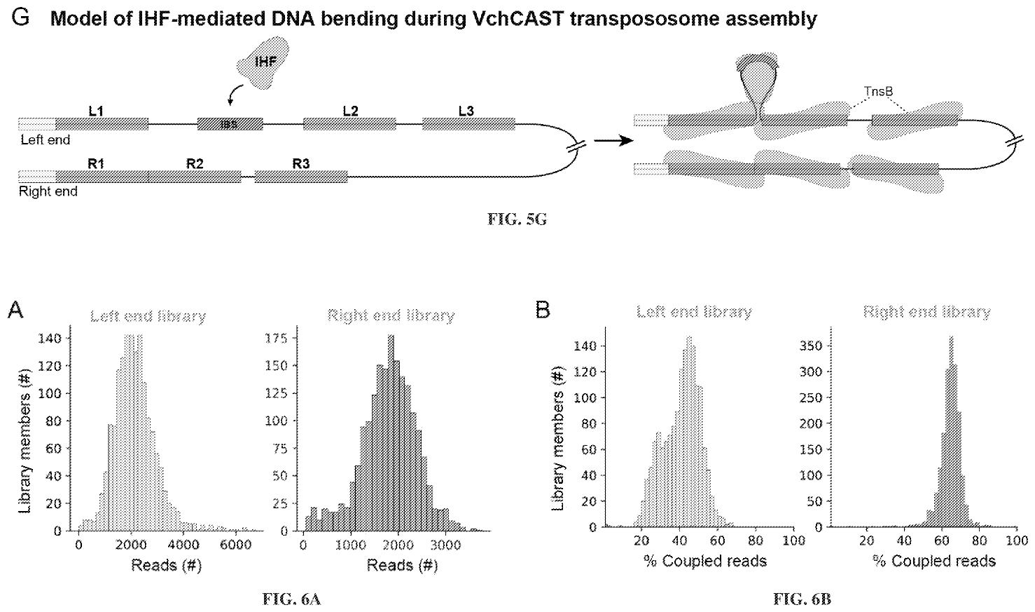

Closer inspection of the transposon left end mutational data revealed a sequence between the two terminal TnsB binding sites (TBSs) that, when mutated, led to reproducible transposition defects (FIG. 5A). The corresponding DNA sequence perfectly matched a consensus binding sequence for Integration Host Factor (IHF), a heterodimeric nucleoid-associated protein (NAP) that binds to the consensus sequence 5′-WATCARNNNNTTR-3′ and induces a DNA bend of more than 160°. First identified as a host factor for bacteriophage λ integration, IHF is also involved in diverse cellular activities including chromosome replication initiation, transcriptional regulation, and various site-specific recombination pathways.

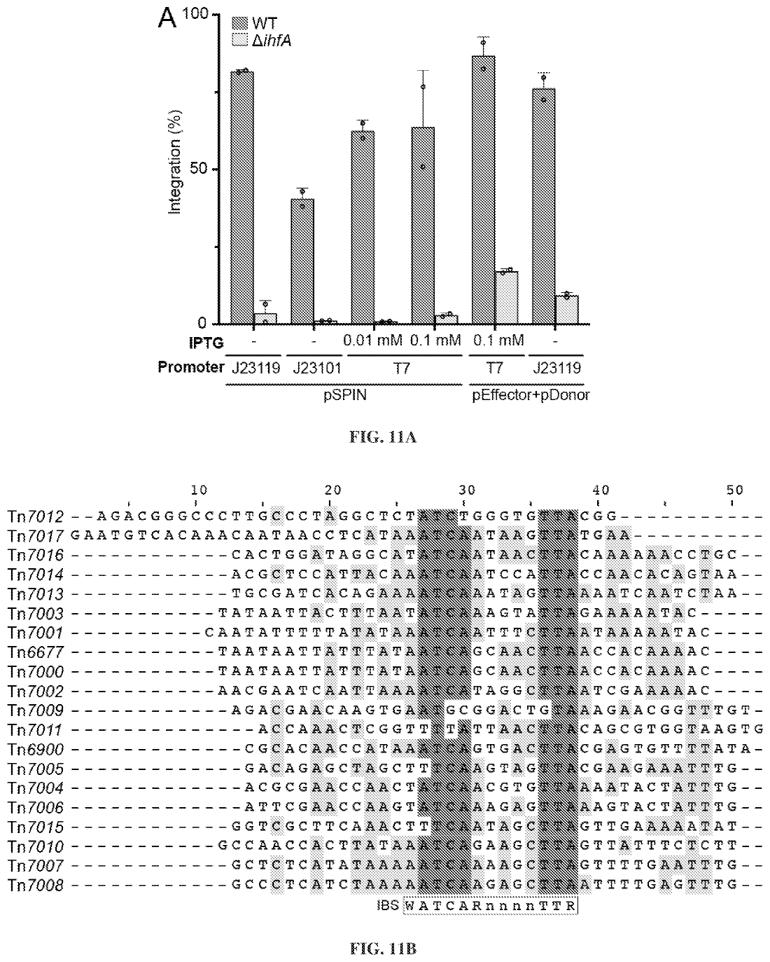

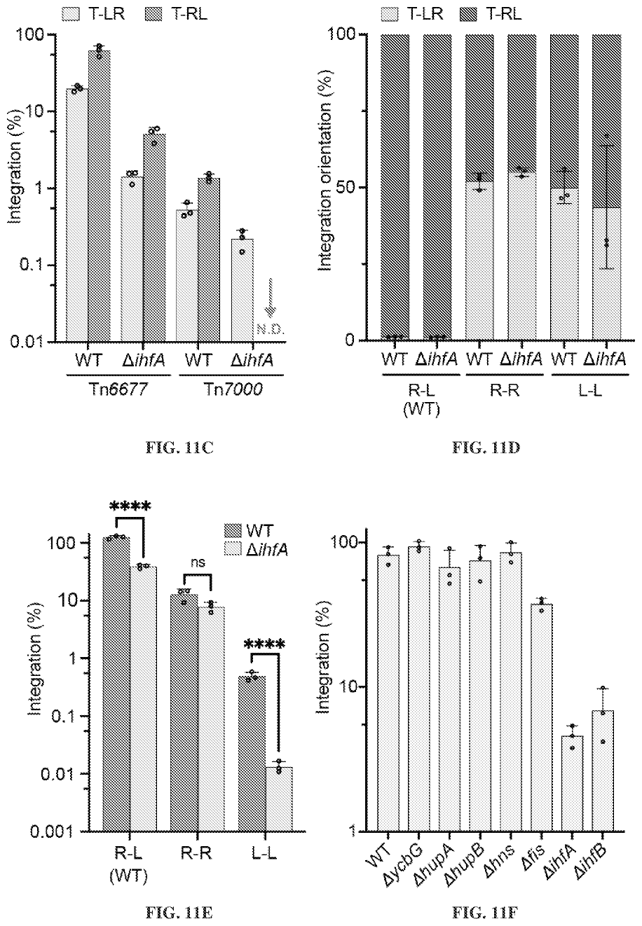

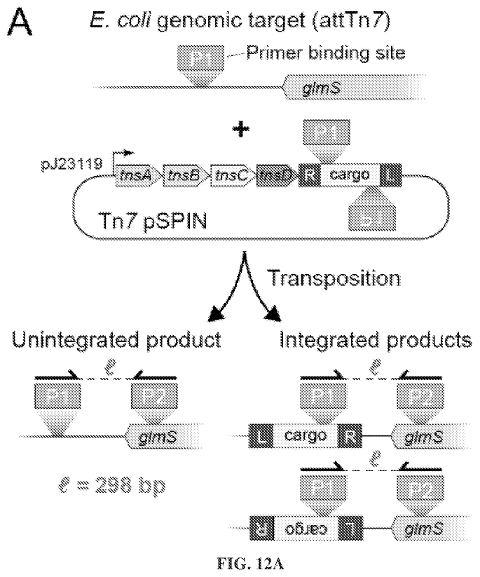

Visual examination of the transposon left ends of twenty homologous systems revealed a highly conserved IBS across all homologs (FIGS. 5D and 5E), and aligning the sequence between the first two TBSs using Clustal Omega also revealed the IBS consensus as a conserved feature (FIG. 11B). To test whether IHF stimulated transposition for these systems, experiments were performed in WT and ΔIHF cells for five other systems and only two (Tn7000 and Tn7014) showed a strong IHF dependence (FIG. 5F).

Given the involvement of IHF and, more generally, the importance of donor/target DNA supercoiling and topology for other mobile elements, we decided to broadly investigate whether other E. coli NAPs might play a role in transposition. After generating individual knockouts of 5 additional nucleoid-associated proteins (NAP) genes (ycbG, hupA, hupB, hns, and fis) and measuring integration efficiency within these mutant backgrounds, only the loss of fis decreased integration efficiency, by 2-fold (FIG. 11F). When the same cohort of NAP knockouts were tested for transposition with the prototypic Tn7 system, IHF had no effect whereas Fis (factor for inversion stimulation) again influenced integration efficiency, though with a ˜4-fold increase in the knockout strain (FIG. 12B).

Interestingly, the amplicon-sequencing detection approach for E. coli Tn7 transposition also yielded new information about the nature of DNA integration products for the well-studied TnsABCD pathway. Whereas prior studies concluded that TnsD binding defines a single integration site downstream of the essential glmS gene, surprisingly heterogeneous insertion patterns were observed that sampled a wider sequence space, including rare but reproducible transposition products in the less-common T-LR orientation (FIG. 12C). These findings highlight the value of deep sequencing to thoroughly and unbiasedly query the range of potential integration products for a given transposable element.

After testing bidirectional transposition for two CAST systems in both a WT and ΔIHF strain of E. coli, it was found that although the loss of IHF did not affect orientation preference for VchCAST, its loss reversed the dominant orientation for Tn7000 from T-RL to T-LR (FIG. 12C). This result raised the intriguing possibility that IHF may be involved in establishing a transpososome architecture that controls the directionality of DNA insertions, at least for some systems. Previous work with the prototypic Tn7 system found that transposon substrates with two right ends were competent for integration whereas two left ends were not. The loss of IHF had no impact on transposition with a substrate containing two transposon right ends, which was integrated without orientation bias, while a substrate containing two left ends exhibited severely reduced integration efficiency that retained a dependence on IHF (FIGS. 12D-E). Overall, the data support a model (FIG. 5G) in which IHF binds the region between TBSs L1 and L2 to bend the transposon left end and drive DNA integration, akin to the proposed role of HU in Mu transposition. Exemplary sequences of IHF constructs are shown in Table 3.

### Example 7

### Hyperactive Tn6677 Transposon End Variants

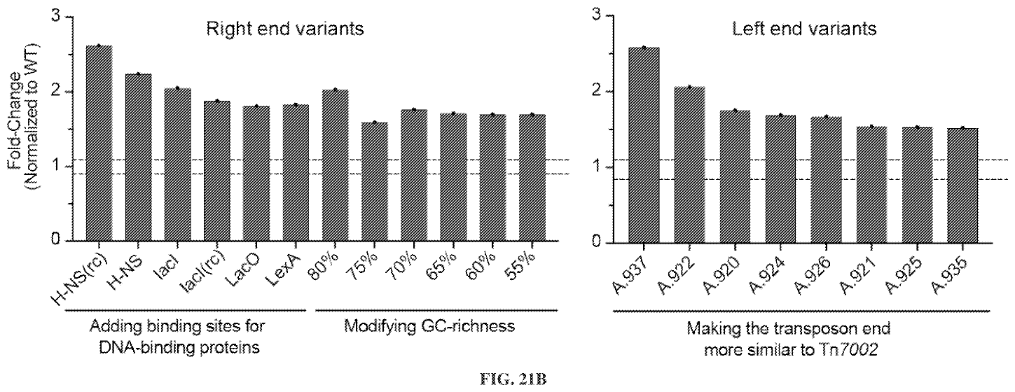

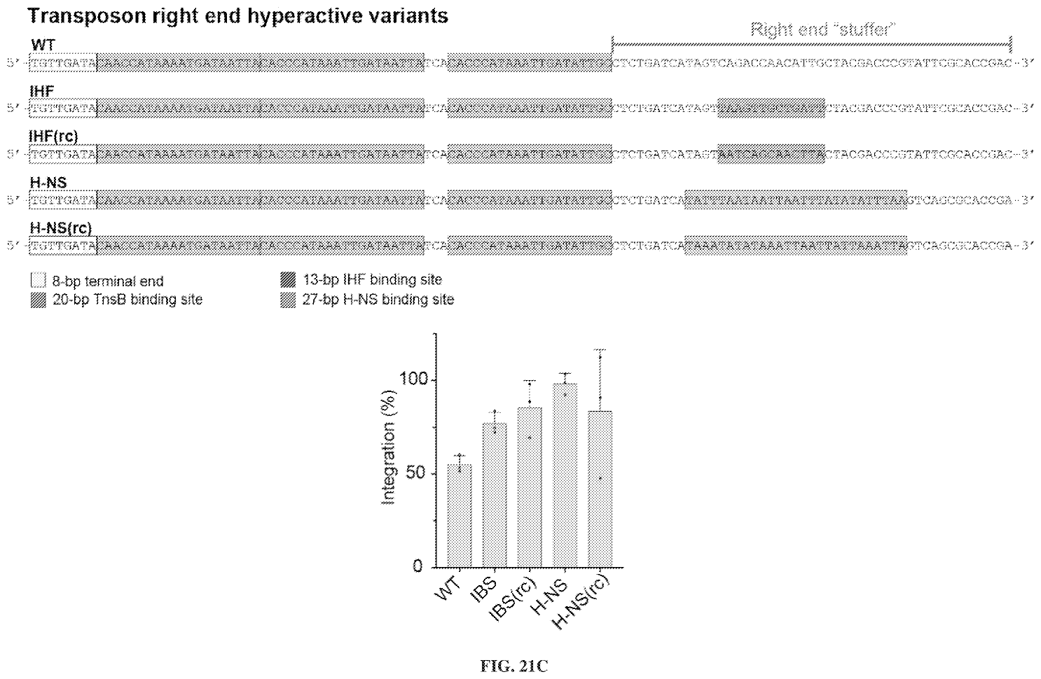

A pooled library-based cellular transposition assay was developed in order to test a large panel of modified transposon end variants. In initial transposon end library experiments, the efficiency of the wild-type (unmodified) transposon substrate, with native end sequences, was high (˜80% efficiency), which limited the ability to confidently identify variants with improved integration activity compared to wildtype. In order to identify hyperactive variants, a modified experimental approach was established in which the overall system on WT transposon end substrates was less active. Cells were plated on media lacking inducer (IPTG), which reduced integration efficiency in the dominant T-RL orientation by approximately 3-fold (FIG. 21A). Then, the transposon end library experiment were repeated using this hypoactive condition, allowing detection of transposon end variants that exhibited hyperactive activity relative to WT. These variants increased transposition efficiency by between 1.5-2.5-fold (FIG. 21B, Tables 5 and 6).

In the transposon right end, hyperactive variants contained mutations in the sequence adjacent to the TnsB binding sites (the right end “stuffer” sequence, illustrated in FIG. 21C). The strongest hyperactive variant contained a binding site for the factor H-NS in this region, while other hyperactive variants contained mutations in this region, either through the addition of binding sites for other DNA-binding proteins, or through mutations that randomly varied the GC-richness of this region. In the left transposon end, hyperactive variants contained mutations in the transposon ends that converted the sequence to be more similar to the transposon end sequence of a related Type I-F CAST homolog, known as Tn7002.

To confirm that mutating the right end “stuffer” sequence was able to increase transposition efficiency, several transposon end variants with mutations in this sequence were cloned and the integration efficiency of these variants was directly measured individually, in a non-library format. Mutations that introduced binding sites for two DNA-binding and bending proteins, IHF or H-NS, both increased transposition efficiency relative to WT (FIG. 21C). Although these variants increased integration in a E. coli bacterial cell context in which these factors are naturally expressed, the improved integration efficiencies may be generalizable across any cell type of interest for these engineered transposon end sequences, whether or not the DNA binding/bending protein factors are present.

### Example 8

### Hyperactive Tn7016 Transposon End Variants

Using the above, a panel of putative hyperactive transposon end sequences were designed for a related CAST system, Tn7016, which shows significantly higher RNA-guided DNA integration activity in mammalian cells. The design of these variants, listed as SEQ ID NOs: 2703-3119 for right end variants and SEQ ID NOs: 4674-5135 for left end variants, was directly informed by mutations that increased the activity of RNA-guided DNA integration for Tn6677. The tested variants include rationally engineered modifications with added binding sites for DNA-binding and bending proteins; modifications that convert the transposon ends to be more similar to the transposon end sequences from homologous CAST systems; modifications that mutate the transposon right end such that the modified sequence encodes functional protein linkers without any in-frame stop codons; and modifications that systematically vary the GC-richness of the sequence adjacent to the TnsB binding sites within either transposon end. Mutations to either the left or right transposon end sequence, or to both transposon end sequences concurrently, in order to incorporate these aforementioned sequence features, result in increased DNA integration activity of the Tn7016 CAST system. These mutations also modify the orientation preference between T-RL and T-LR of a CAST system of interest. These variants are currently designed with modifications to either the transposon right end or the transposon left end, however hyperactive transposon left and right end variants are combined to further increase DNA integration activity.

This transposon end library is cloned into a pDonor substrate which is used in various cell types that may include bacterial cells, plant cells, animal cells, or human cells. For example, the pDonor library is used to transfect mammalian cells together with the necessary CAST protein and RNA machinery, and targeted sequencing of the integration product is performed, in order to uncover transposon end modifications with hyperactivity. Library members with enriched sequence abundances after integration are further investigated as highly active transposon end variants in human cells.

Library members may include variants in which the transposon end does not contain stop codons in any reading frame. These modifications enable mini-transposon genetic payloads to be integrated directly into or downstream of a gene body, such that read-through translation across the transposon end enables seamless fusions, at the protein level, with custom polypeptides encoded within the genetic payload of the transposon. These transposon end variants are used to enable protein tagging, in which targeted integration occurs immediately downstream of the start codon, or immediately upstream of the stop codon, of a gene of interest. Therefore, translation will read through the transposon, appending a sequence of interest to a target protein encoded within the genome.

TABLE 1
Library of transposon right end variant sequences tested for transposition

| | | |
|---|---|---|
| SEQ | | |
| ID | DNA sequence 5′ → 3′ (57 bp: | |
| NO | TIR-R1-R2-Space-[R3) | Amino Acid sequence |

Frame 1

| | | |
|---|---|---|
| 18 | TGTTGATACAACCATAAAATGATAATTACACCCAT | YNHKMIITPIN (SEQ ID NO: 5352) * *LSHP (SEQ ID |
| | AAATTGATAATTATCACACCCA | NO: 5353) |
| 22 | TGTCGATACAACCATAAAATGATAATTACACCCAT | CRYNHKMIITPIN (SEQ ID NO: 5361)* *LSHP (SEQ |
| | AAATTGATAATTATCACACCCA | DD NO: 5353) |
| 23 | TGTGGATACAACCATAAAATGATAATTACACCCAT | CGYNHKMIITPIN(SEQ ID NO: 5362)* *LSHP (SEQ |
| | AAATTGATAATTATCACACCCA | ID NO: 5353) |
| 24 | TGTTCATACAACCATAAAATGATAATTACACCCAT | CSYNHKMIITPIN(SEQ ID NO: 5363)* *LSHP (SEQ |
| | AAATTGATAATTATCACACCCA | ID NO: 5353) |
| 25 | TGTTGATACAACCATAAAATGATAATTACACCCAT | C*YNHKMIITPING(SEQ ID NO: 5364)* LSHP (SEQ |
| | AAATGGATAATTATCACACCCA | ID NO: 5353) |
| 26 | TGTTGATACAACCATAAAATGATAATTACACCCAT | C*YNHKMIITPINS(SEQ ID NO: 5365)* LSHP (SEQ |
| | AAATTCATAATTATCACACCCA | ID NO: 5353) |
| 27 | TGTCGATACAACCATAAAATGATAATTACACCCAT | CRYNHKMIITPING(SEQ ID NO: 5366)* LSHP (SEQ |
| | AAATGGATAATTATCACACCCA | ID NO: 5353) |
| 28 | TGTCGATACAACCATAAAATGATAATTACACCCAT | CRYNHKMIITPINS(SEQ ID NO: 5367)* LSHP (SEQ |
| | AAATTCATAATTATCACACCCA | ID NO: 5353) |
| 29 | TGTGGATACAACCATAAAATGATAATTACACCCAT | CGYNHKMIITPING(SEQ ID NO: 5368) * LSHP |
| | AAATGGATAATTATCACACCCA | (SEQ ID NO: 5353) |
| 30 | TGTGGATACAACCATAAAATGATAATTACACCCAT | CGYNHKMIITPINS(SEQ ID NO: 5369) * LSHP (SEQ |
| | AAATTCATAATTATCACACCCA | ID NO: 5353) |
| 31 | TGTTCATACAACCATAAAATGATAATTACACCCAT | CSYNHKMIITPING(SEQ ID NO: 5370) * LSHP (SEQ |
| | AAATGGATAATTATCACACCCA | ID NO: 5353) |
| 32 | TGTTCATACAACCATAAAATGATAATTACACCCAT | CSYNHKMIITPINS(SEQ ID NO: 5371) * LSHP (SEQ |
| | AAATTCATAATTATCACACCCA | DD NO: 5353) |
| 33 | TGTTGATACAACCATAAAATGATAATTACACCCAT | C*YNHKMIITPIN (SEQ ID NO: 5352) *SLSHP (SEQ |
| | AAATTGATCATTATCACACCCA | ID NO: 5372) |
| 34 | TGTCGATACAACCATAAAATGATAATTACACCCAT | CRYNHKMIITPIN(SEQ ID NO: 5361) * SLSHP (SEQ |
| | AAATTGATCATTATCACACCCA | ID NO: 5372) |
| 35 | TGTGGATACAACCATAAAATGATAATTACACCCAT | CGYNHKMIITPIN(SEQ ID NO: 5362) * SLSHP (SEQ |
| | AAATTGATCATTATCACACCCA | ID NO: 5372) |
| 36 | TGTTCATACAACCATAAAATGATAATTACACCCAT | CSYNHKMIITPIN(SEQ ID NO: 5363) * SLSHP (SEQ |
| | AAATTGATCATTATCACACCCA | ID NO: 5372) |
| 37 | TGTTGATACAACCATAAAATGATAATTACACCCAT | C*YNHKMIITPINGSLSHP (SEQ ID NO: 5373) |
| | AAATGGATCATTATCACACCCA | |
| 38 | TGTTGATACAACCATAAAATGATAATTACACCCAT | C*YNHKMITPINSSLSHP (SEQ ID NO: 5374) |
| | AAATTCATCATTATCACACCCA | |
| 39 | TGTCGATACAACCATAAAATGATAATTACACCCAT | CRYNHKMITPINGSLSHP (SEQ ID NO: 5375) |
| | AAATGGATCATTATCACACCCA | |
| 40 | TGTCGATACAACCATAAAATGATAATTACACCCAT | CRYNHKMIITPINSSLSHP (SEQ ID NO: 5376) |
| | AAATTCATCATTATCACACCCA | |
| 41 | TGTGGATACAACCATAAAATGATAATTACACCCAT | CGYNHKMIITPINGSLSHP (SEQ ID NO: 5377) |
| | AAATGGATCATTATCACACCCA | |
| 42 | TGTGGATACAACCATAAAATGATAATTACACCCAT | CGYNHKMIITPINSSLSHP (SEQ ID NO: 5378) |
| | AAATTCATCATTATCACACCCA | |
| 43 | TGTTCATACAACCATAAAATGATAATTACACCCAT | CSYNHKMITPINGSLSHP (SEQ ID NO: 5379) |
| | AAATGGATCATTATCACACCCA | |
| 44 | TGTTCATACAACCATAAAATGATAATTACACCCAT | CSYNHKMIITPINSSLSHP (SEQ ID NO: 5380) |
| | AAATTCATCATTATCACACCCA | |
| 45 | GGTTGATACAACCATAAAATGATAATTACACCCAT | G*YNHKMIITPIN (SEQ ID NO: 5352)**LSHP (SEQ |
| | AAATTGATAATTATCACACCCA | ID NO: 5353) |
| 46 | GGTCGATACAACCATAAAATGATAATTACACCCAT | GRYNHKMIITPIN (SEQ ID NO: 5381) * *LSHP |
| | AAATTGATAATTATCACACCCA | (SEQ ID NO: 5353) |
| 47 | GGTGGATACAACCATAAAATGATAATTACACCCAT | GGYNHKMIITPIN (SEQ ID NO: 5382) * *LSHP |
| | AAATTGATAATTATCACACCCA | (SEQ ID NO: 5353) |
| 48 | GGTTCATACAACCATAAAATGATAATTACACCCAT | GSYNHKMIITPIN (SEQ ID NO: 5383) **LSHP (SEQ |
| | AAATTGATAATTATCACACCCA | ID NO: 5353) |
| 49 | GGTTGATACAACCATAAAATGATAATTACACCCAT | G*YNHKMIITPING(SEQ ID NO: 5364) * LSHP (SEQ |
| | AAATGGATAATTATCACACCCA | ID NO: 5353) |
| 50 | GGTTGATACAACCATAAAATGATAATTACACCCAT | G*YNHKMIITPINS(SEQ ID NO: 5365) * LSHP (SEQ |
| | AAATTCATAATTATCACACCCA | ID NO: 5353) |
| 51 | GGTCGATACAACCATAAAATGATAATTACACCCAT | GRYNHKMIITPING (SEQ ID NO: 5385) *LSHP |
| | AAATGGATAATTATCACACCCA | (SEQ ID NO: 5353) |
| 52 | GGTCGATACAACCATAAAATGATAATTACACCCAT | GRYNHKMIITPINS (SEQ ID NO: 5384) *LSHP (SEQ |
| | AAATTCATAATTATCACACCCA | ID NO: 5353) |
| 53 | GGTGGATACAACCATAAAATGATAATTACACCCAT | GGYNHKMIITPING (SEQ ID NO: 5387) *LSHP |
| | AAATGGATAATTATCACACCCA | (SEQ ID NO: 5353) |
| 54 | GGTGGATACAACCATAAAATGATAATTACACCCAT | GGYNHKMIITPINS(SEQ ID NO: 5388) *LSHP (SEQ |
| | AAATTCATAATTATCACACCCA | ID NO: 5353) |
| 55 | GGTTCATACAACCATAAAATGATAATTACACCCAT | GSYNHKMIITPING (SEQ ID NO: 5389) *LSHP (SEQ |
| | AAATGGATAATTATCACACCCA | ID NO: 5353) |
| 56 | GGTTCATACAACCATAAAATGATAATTACACCCAT | GSYNHKMIITPINS(SEQ ID NO: 5390) *LSHP (SEQ |
| | AAATTCATAATTATCACACCCA | ID NO: 5353) |
| 57 | GGTTGATACAACCATAAAATGATAATTACACCCAT | G*YNHKMIITPIN (SEQ ID NO: 5352)*SLSHP (SEQ |
| | AAATTGATCATTATCACACCCA | ID NO: 5372) |
| 58 | GGTCGATACAACCATAAAATGATAATTACACCCAT | GRYNHKMIITPIN (SEQ ID NO: 5381) * SLSHP |
| | AAATTGATCATTATCACACCCA | (SEQ ID NO: 5372) |
| 59 | GGTGGATACAACCATAAAATGATAATTACACCCAT | GGYNHKMIITPIN (SEQ ID NO: 5382) * SLSHP |
| | AAATTGATCATTATCACACCCA | (SEQ ID NO: 5372) |
| 60 | GGTTCATACAACCATAAAATGATAATTACACCCAT | GSYNHKMIITPIN (SEQ ID NO: 5383) *SLSHP (SEQ |
| | AAATTGATCATTATCACACCCA | ID NO: 5372) |
| 61 | GGTTGATACAACCATAAAATGATAATTACACCCAT | G*YNHKMIITPINGSLSHP (SEQ ID NO: 5373) |
| | AAATGGATCATTATCACACCCA | |
| 62 | GGTTGATACAACCATAAAATGATAATTACACCCAT | G*YNHKMIITPINSSLSHP (SBQ ID NO: 5374) |
| | AAATTCATCATTATCACACCCA | |
| 63 | GGTCGATACAACCATAAAATGATAATTACACCCAT | GRYNHKMIITPINGSLSHP (SEQ ID NO: 5391) |
| | AAATGGATCATTATCACACCCA | |
| 64 | GGTCGATACAACCATAAAATGATAATTACACCCAT | GRYNHKMIITPINSSLSHP (SEQ ID NO: 5392) |
| | AAATTCATCATTATCACACCCA | |
| 65 | GGTGGATACAACCATAAAATGATAATTACACCCAT | GGYNHKMIITPINGSLSHP (SEQ ID NO: 5393) |
| | AAATGGATCATTATCACACCCA | |
| 66 | GGTGGATACAACCATAAAATGATAATTACACCCAT | GGYNHKMIITPINSSLSHP (SEQ ID NO: 5394) |
| | AAATTCATCATTATCACACCCA | |
| 67 | GGTTCATACAACCATAAAATGATAATTACACCCAT | GSYNHKMIITPINGSLSHP (SEQ ID NO: 5395) |
| | AAATGGATCATTATCACACCCA | |
| 68 | GGTTCATACAACCATAAAATGATAATTACACCCAT | GSYNHKMIITPINSSLSHP (SEQ ID NO: 5396) |
| | AAATTCATCATTATCACACCCA | |
| 69 | TCTTGATACAACCATAAAATGATAATTACACCCAT | S*YNHKMIITPIN (SEQ ID NO: 5352)**LSHP (SEQ |
| | AAATTGATAATTATCACACCCA | ID NO: 5353) |
| 70 | TCTCGATACAACCATAAAATGATAATTACACCCAT | SRYNHKMIITPIN (SEQ ID NO: 5397) **LSHP (SEQ |
| | AAATTGATAATTATCACACCCA | ID NO: 5353) |
| 71 | TCTGGATACAACCATAAAATGATAATTACACCCAT | SGYNHKMIITPIN (SEQ ID NO: 5398) **LSHP (SEQ |
| | AAATTGATAATTATCACACCCA | ID NO: 5353) |
| 72 | TCTTCATACAACCATAAAATGATAATTACACCCAT | SSYNHKMIITPIN (SEQ ID NO: 5399) **LSHP (SEQ |
| | AAATTGATAATTATCACACCCA | ID NO: 5353) |
| 73 | TCTTGATACAACCATAAAATGATAATTACACCCAT | S*YNHKMIITPING(SEQ ID NO: 5364) * LSHP (SEQ |
| | AAATGGATAATTATCACACCCA | ID NO: 5353) |
| 74 | TCTTGATACAACCATAAAATGATAATTACACCCAT | S*YNHKMIITPINS(SEQ ID NO: 5365) ** LSHP |
| | AAATTCATAATTATCACACCCA | (SEQ ID NO: 5353) |
| 75 | TCTCGATACAACCATAAAATGATAATTACACCCAT | SRYNHKMITPING (SEQ ID NO: 5400) *LSHP (SEQ |
| | AAATGGATAATTATCACACCCA | ID NO: 5353) |
| 76 | TCTCGATACAACCATAAAATGATAATTACACCCAT | SRYNHKMIITPINS (SEQ ID NO: 5401) *LSHP (SEQ |
| | AAATTCATAATTATCACACCCA | ID NO: 5353) |
| 77 | TCTGGATACAACCATAAAATGATAATTACACCCAT | SGYNHKMITPING (SEQ ID NO: 5402) *LSHP (SEQ |
| | AAATGGATAATTATCACACCCA | ID NO: 5353) |
| 78 | TCTGGATACAACCATAAAATGATAATTACACCCAT | SGYNHKMIITPINS (SEQ ID NO: 5403) *LSHP (SEQ |
| | AAATTCATAATTATCACACCCA | ID NO: 5353) |
| 79 | TCTTCATACAACCATAAAATGATAATTACACCCAT | SSYNHKMIITPING (SEQ ID NO: 5404) *LSHP (SEQ |
| | AAATGGATAATTATCACACCCA | ID NO: 5353) |
| 80 | TCTTCATACAACCATAAAATGATAATTACACCCAT | SSYNHKMIITPINS (SEQ ID NO: 5405) *LSHP (SEQ |
| | AAATTCATAATTATCACACCCA | ID NO: 5353) |
| 81 | TCTTGATACAACCATAAAATGATAATTACACCCAT | S*YNHKMIITPIN (SEQ ID NO: 5352)*SLSHP (SEQ |
| | AAATTGATCATTATCACACCCA | ID NO: 5372) |
| 82 | TCTCGATACAACCATAAAATGATAATTACACCCAT | SRYNHKMIITPIN (SEQ ID NO: 5397) *SLSHP (SEQ |
| | AAATTGATCATTATCACACCCA | ID NO: 5372) |
| 83 | TCTGGATACAACCATAAAATGATAATTACACCCAT | SGYNHKMIITPIN (SEQ ID NO: 5398) *SLSHP (SEQ |
| | AAATTGATCATTATCACACCCA | ID NO: 5372) |
| 84 | TCTTCATACAACCATAAAATGATAATTACACCCAT | SSYNHKMIITPIN (SEQ ID NO: 5399) *SLSHP (SEQ |
| | AAATTGATCATTATCACACCCA | ID NO: 5372) |
| 85 | TCTTGATACAACCATAAAATGATAATTACACCCAT | S*YNHKMIITPINGSLSHP (SEQ ID NO: 5373) |
| | AAATGGATCATTATCACACCCA | |
| 86 | TCTTGATACAACCATAAAATGATAATTACACCCAT | S*YNHKMIITPINSSLSHP (SEQ ID NO: 5374) |
| | AAATTCATCATTATCACACCCA | |
| 87 | TCTCGATACAACCATAAAATGATAATTACACCCAT | SRYNHKMIITPINGSLSHP (SEQ ID NO: 5406) |
| | AAATGGATCATTATCACACCCA | |
| 88 | TCTCGATACAACCATAAAATGATAATTACACCCAT | SRYNHKMITPINSSLSHP (SEQ ID NO: 5407) |
| | AAATTCATCATTATCACACCCA | |
| 89 | TCTGGATACAACCATAAAATGATAATTACACCCAT | SGYNHKMIITPINGSLSHP (SEQ ID NO: 5408) |
| | AAATGGATCATTATCACACCCA | |
| 90 | TCTGGATACAACCATAAAATGATAATTACACCCAT | SGYNHKMIITPINSSLSHP (SEQ ID NO: 5409) |
| | AAATTCATCATTATCACACCCA | |
| 91 | TCTTCATACAACCATAAAATGATAATTACACCCAT | SSYNHKMIITPINGSLSHP (SEQ ID NO: 5410) |
| | AAATGGATCATTATCACACCCA | |
| 92 | TCTTCATACAACCATAAAATGATAATTACACCCAT | SSYNHKMIITPINSSLSHP (SEQ ID NO: 5411) |
| | AAATTCATCATTATCACACCCA | |
| 93 | AGTTGATACAACCATAAAATGATAATTACACCCAT | S*YNHKMIITPIN (SEQ ID NO: 5352) ** LSHP (SEQ |
| | AAATTGATAATTATCACACCCA | ID NO: 5353) |
| 94 | AGTCGATACAACCATAAAATGATAATTACACCCAT | SRYNHKMIITPIN (SEQ ID NO: 5397) ** LSHP (SEQ |
| | AAATTGATAATTATCACACCCA | ID NO: 5353) |
| 95 | AGTGGATACAACCATAAAATGATAATTACACCCAT | SGYNHKMIITPIN (SEQ ID NO: 5398) ** LSHP (SEQ |
| | AAATTGATAATTATCACACCCA | ID NO: 5353) |
| 96 | AGTTCATACAACCATAAAATGATAATTACACCCAT | SSYNHKMIITPIN (SEQ ID NO: 5399) **LSHP (SEQ |
| | AAATTGATAATTATCACACCCA | ID NO: 5353) |
| 97 | AGTTGATACAACCATAAAATGATAATTACACCCAT | S*YNHKMIITPING(SEQ ID NO: 5364) * LSHP (SEQ |
| | AAATGGATAATTATCACACCCA | ID NO: 5353) |
| 98 | AGTTGATACAACCATAAAATGATAATTACACCCAT | S*YNHKMIITPINS(SEQ ID NO: 5365) * LSHP (SEQ |
| | AAATTCATAATTATCACACCCA | ID NO: 5353) |
| 99 | AGTCGATACAACCATAAAATGATAATTACACCCAT | SRYNHKMIITPING (SEQ ID NO: 5400) *LSHP (SEQ |
| | AAATGGATAATTATCACACCCA | ID NO: 5353) |
| 100 | AGTCGATACAACCATAAAATGATAATTACACCCAT | SRYNHKMIITPINS (SEQ ID NO: 5401) *LSHP (SEQ |
| | AAATTCATAATTATCACACCCA | ID NO: 5353) |
| 101 | AGTGGATACAACCATAAAATGATAATTACACCCAT | SGYNHKMIITPING (SEQ ID NO: 5402) *LSHP (SEQ |
| | AAATGGATAATTATCACACCCA | ID NO: 5353) |
| 102 | AGTGGATACAACCATAAAATGATAATTACACCCAT | SGYNHKMIITPINS (SEQ ID NO: 5403) *LSHP (SEQ |
| | AAATTCATAATTATCACACCCA | ID NO: 5353) |
| 103 | AGTTCATACAACCATAAAATGATAATTACACCCAT | SSYNHKMIITPING (SEQ ID NO: 5404) *LSHP (SEQ |
| | AAATGGATAATTATCACACCCA | ID NO: 5353) |
| 104 | AGTTCATACAACCATAAAATGATAATTACACCCAT | SSYNHKMIITPINS (SEQ ID NO: 5405) *LSHP (SEQ |
| | AAATTCATAATTATCACACCCA | ID NO: 5353) |
| 105 | AGTTGATACAACCATAAAATGATAATTACACCCAT | S*YNHKMIITPIN (SEQ ID NO: 5352)*SLSHP (SEQ |
| | AAATTGATCATTATCACACCCA | ID NO: 5372) |
| 106 | AGTCGATACAACCATAAAATGATAATTACACCCAT | SRYNHKMIITPIN (SEQ ID NO: 5397)*SLSHP (SEQ |
| | AAATTGATCATTATCACACCCA | ID NO: 5372) |
| 107 | AGTGGATACAACCATAAAATGATAATTACACCCAT | SGYNHKMIITPIN (SEQ ID NO: 5398) *SLSHP (SEQ |
| | AAATTGATCATTATCACACCCA | ID NO: 5372) |
| 108 | AGTTCATACAACCATAAAATGATAATTACACCCAT | SSYNHKMIITPIN (SBQ ID NO: 5399) *SLSHP (SEQ |
| | AAATTGATCATTATCACACCCA | ID NO: 5372) |
| 109 | AGTTGATACAACCATAAAATGATAATTACACCCAT | S*YNHKMIITPINGSLSHP (SEQ ID NO: 5373) |
| | AAATGGATCATTATCACACCCA | |
| 110 | AGTTGATACAACCATAAAATGATAATTACACCCAT | S*YNHKMIITPINSSLSHP (SEQ ID NO: 5374) |
| | AAATTCATCATTATCACACCCA | |
| 111 | AGTCGATACAACCATAAAATGATAATTACACCCAT | SRYNHKMIITPINGSLSHP (SEQ ID NO: 5406) |
| | AAATGGATCATTATCACACCCA | |
| 112 | AGTCGATACAACCATAAAATGATAATTACACCCAT | SRYNHKMITPINSSLSHP (SEQ ID NO: 5407) |
| | AAATTCATCATTATCACACCCA | |
| 113 | AGTGGATACAACCATAAAATGATAATTACACCCAT | SGYNHKMIITPINGSLSHP (SEQ ID NO: 5408) |
| | AAATGGATCATTATCACACCCA | |
| 114 | AGTGGATACAACCATAAAATGATAATTACACCCAT | SGYNHKMIITPINSSLSHP (SEQ ID NO: 5409) |
| | AAATTCATCATTATCACACCCA | |
| 115 | AGTTCATACAACCATAAAATGATAATTACACCCAT | SSYNHKMIITPINGSLSHP (SEQ ID NO: 5410) |
| | AAATGGATCATTATCACACCCA | |
| 116 | AGTTCATACAACCATAAAATGATAATTACACCCAT | SSYNHKMIITPINSSLSHP (SEQ ID NO: 5411) |
| | AAATTCATCATTATCACACCCA | |
| 117 | TGTTGATACAACCCTAAAATGATAATTACACCCAT | C*YNPKMIITPIN (SEQ ID NO: 5412) **LSHP (SEQ |
| | AAATTGATAATTATCACACCCA | ID NO: 5353) |
| 118 | TGTCGATACAACCCTAAAATGATAATTACACCCAT | CRYNPKMIITPIN (SEQ ID NO: 5413) **LSHP (SEQ |
| | AAATTGATAATTATCACACCCA | ID NO: 5353) |
| 119 | TGTGGATACAACCCTAAAATGATAATTACACCCAT | CGYNPKMITPIN (SEQ ID NO: 5414) **LSHP (SEQ |
| | AAATTGATAATTATCACACCCA | ID NO: 5353) |
| 120 | TGTTCATACAACCCTAAAATGATAATTACACCCAT | CSYNPKMIITPIN (SEQ ID NO: 5415) **LSHP (SEQ |
| | AAATTGATAATTATCACACCCA | ID NO: 5353) |
| 121 | TGTTGATACAACCCTAAAATGATAATTACACCCAT | C*YNPKMIITPING (SEQ ID NO: 5416) *LSHP (SEQ |
| | AAATGGATAATTATCACACCCA | ID NO: 5353) |
| 122 | TGTTGATACAACCCTAAAATGATAATTACACCCAT | C*YNPKMIITPINS (SEQ ID NO: 5417) *LSHP (SEQ |
| | AAATTCATAATTATCACACCCA | ID NO: 5353) |
| 123 | TGTCGATACAACCCTAAAATGATAATTACACCCAT | CRYNPKMIITPING (SEQ ID NO: 5418) *LSHP (SEQ |
| | AAATGGATAATTATCACACCCA | ID NO: 5353) |
| 124 | TGTCGATACAACCCTAAAATGATAATTACACCCAT | CRYNPKMIITPINS (SEQ ID NO: 5419) *LSHP (SEQ |
| | AAATTCATAATTATCACACCCA | ID NO: 5353) |
| 125 | TGTGGATACAACCCTAAAATGATAATTACACCCAT | CGYNPKMIITPING (SEQ ID NO: 5420) *LSHP (SEQ |
| | AAATGGATAATTATCACACCCA | ID NO: 5353) |
| 126 | TGTGGATACAACCCTAAAATGATAATTACACCCAT | CGYNPKMIITPINS (SEQ ID NO: 5421) *LSHP (SEQ |
| | AAATTCATAATTATCACACCCA | ID NO: 5353) |
| 127 | TGTTCATACAACCCTAAAATGATAATTACACCCAT | CSYNPKMIITPING (SEQ ID NO: 5422) *LSHP (SEQ |
| | AAATGGATAATTATCACACCCA | ID NO: 5353) |
| 128 | TGTTCATACAACCCTAAAATGATAATTACACCCAT | CSYNPKMIITPINS (SEQ ID NO: 5423) *LSHP (SEQ |
| | AAATTCATAATTATCACACCCA | ID NO: 5353) |
| 129 | TGTTGATACAACCCTAAAATGATAATTACACCCAT | C*YNPKMIITPIN (SEQ ID NO: 5412) *SLSHP (SEQ |
| | AAATTGATCATTATCACACCCA | ID NO: 5372) |
| 130 | TGTCGATACAACCCTAAAATGATAATTACACCCAT | CRYNPKMIITPIN (SEQ ID NO: 5413) *SLSHP (SEQ |
| | AAATTGATCATTATCACACCCA | ID NO: 5372) |
| 131 | TGTGGATACAACCCTAAAATGATAATTACACCCAT | CGYNPKMIITPIN (SEQ ID NO: 5414) *SLSHP (SEQ |
| | AAATTGATCATTATCACACCCA | ID NO: 5372) |
| 132 | TGTTCATACAACCCTAAAATGATAATTACACCCAT | CSYNPKMITPIN (SEQ ID NO: 5415) *SLSHP (SEQ |
| | AAATTGATCATTATCACACCCA | ID NO: 5372) |
| 133 | TGTTGATACAACCCTAAAATGATAATTACACCCAT | C*YNPKMITPINGSLSHP (SEQ ID NO: 5424) |
| | AAATGGATCATTATCACACCCA | |
| 134 | TGTTGATACAACCCTAAAATGATAATTACACCCAT | C*YNPKMIITPINSSLSHP (SEQ ID NO: 5425) |
| | AAATTCATCATTATCACACCCA | |
| 135 | TGTCGATACAACCCTAAAATGATAATTACACCCAT | CRYNPKMIITPINGSLSHP (SEQ ID NO: 5426) |
| | AAATGGATCATTATCACACCCA | |
| 136 | TGTCGATACAACCCTAAAATGATAATTACACCCAT | CRYNPKMIITPINSSLSHP (SEQ ID NO: 5427) |
| | AAATTCATCATTATCACACCCA | |
| 137 | TGTGGATACAACCCTAAAATGATAATTACACCCAT | CGYNPKMITPINGSLSHP (SEQ ID NO: 5428) |
| | AAATGGATCATTATCACACCCA | |
| 138 | TGTGGATACAACCCTAAAATGATAATTACACCCAT | CGYNPKMIITPINSSLSHP (SEQ ID NO: 5429) |
| | AAATTCATCATTATCACACCCA | |
| 139 | TGTTCATACAACCCTAAAATGATAATTACACCCAT | CSYNPKMIITPINGSLSHP (SEQ ID NO: 5430) |
| | AAATGGATCATTATCACACCCA | |
| 140 | TGTTCATACAACCCTAAAATGATAATTACACCCAT | CSYNPKMIITPINSSLSHP (SEQ ID NO: 5431) |
| | AAATTCATCATTATCACACCCA | |
| 141 | TGTTGATACAACCATAAAATGATAATTACACCCAT | C*YNHKMIITPIN (SEQ ID NO: 5352)**LSPP (SEQ |
| | AAATTGATAATTATCACCCCCA | ID NO: 5432) |
| 142 | TGTCGATACAACCATAAAATGATAATTACACCCAT | CRYNHKMIITPIN(SEQ ID) NO: 5361) * *LSPP (SEQ |
| | AAATTGATAATTATCACCCCCA | ID NO: 5432) |
| 143 | TGTGGATACAACCATAAAATGATAATTACACCCAT | CGYNHKMIITPIN(SEQ ID NO: 5362) * *LSPP (SEQ |
| | AAATTGATAATTATCACCCCCA | ID NO: 5432) |
| 144 | TGTTCATACAACCATAAAATGATAATTACACCCAT | CSYNHKMIITPIN(SEQ ID NO: 5363) * *LSPP (SEQ |
| | AAATTGATAATTATCACCCCCA | ID NO: 5432) |
| 145 | TGTTGATACAACCATAAAATGATAATTACACCCAT | C*YNHKMIITPING(SEQ ID NO: 5364) * LSPP (SEQ |
| | AAATGGATAATTATCACCCCCA | DD NO: 5432) |
| 146 | TGTTGATACAACCATAAAATGATAATTACACCCAT | C*YNHKMIITPINS(SEQ ID NO: 5365) * LSPP (SEQ |
| | AAATTCATAATTATCACCCCCA | ID NO: 5432) |
| 147 | TGTCGATACAACCATAAAATGATAATTACACCCAT | CRYNHKMIITPING(SEQ ID NO: 5366) * LSPP (SEQ |
| | AAATGGATAATTATCACCCCCA | ID NO: 5432) |
| 148 | TGTCGATACAACCATAAAATGATAATTACACCCAT | CRYNHKMIITPINS(SEQ ID NO: 5367) * LSPP (SEQ |
| | AAATTCATAATTATCACCCCCA | ID NO: 5432) |
| 149 | TGTGGATACAACCATAAAATGATAATTACACCCAT | CGYNHKMIITPING(SEQ ID NO: 5368) * LSPP (SEQ |
| | AAATGGATAATTATCACCCCCA | ID NO: 5432) |
| 150 | TGTGGATACAACCATAAAATGATAATTACACCCAT | CGYNHKMIITPINS(SEQ ID NO: 5369) * LSPP (SEQ |
| | AAATTCATAATTATCACCCCCA | ID NO: 5432) |
| 151 | TGTTCATACAACCATAAAATGATAATTACACCCAT | CSYNHKMIITPING(SBQ ID NO: 5370) * LSPP (SEQ |
| | AAATGGATAATTATCACCCCCA | ID NO: 5432) |
| 152 | TGTTCATACAACCATAAAATGATAATTACACCCAT | CSYNHKMIITPINS(SEQ ID NO: 5371) * LSPP (SEQ |
| | AAATTCATAATTATCACCCCCA | ID NO: 5432) |
| 153 | TGTTGATACAACCATAAAATGATAATTACACCCAT | C*YNHKMIITPIN (SEQ ID NO: 5352)*SLSPP (SEQ |
| | AAATTGATCATTATCACCCCCA | ID NO: 5433) |
| 154 | TGTCGATACAACCATAAAATGATAATTACACCCAT | CRYNHKMIITPIN(SEQ ID NO: 5361) * SLSPP (SEQ |
| | AAATTGATCATTATCACCCCCA | ID NO: 5433) |
| 155 | TGTGGATACAACCATAAAATGATAATTACACCCAT | CGYNHKMIITPIN(SEQ ID NO: 5362) * SLSPP (SEQ |
| | AAATTGATCATTATCACCCCCA | ID NO: 5433) |
| 156 | TGTTCATACAACCATAAAATGATAATTACACCCAT | CSYNHKMIITPIN(SEQ ID NO: 5363) * SLSPP (SEQ |
| | AAATTGATCATTATCACCCCCA | ID NO: 5433) |
| 157 | TGTTGATACAACCATAAAATGATAATTACACCCAT | C*YNHKMIITPINGSLSPP (SEQ ID NO: 5434) |
| | AAATGGATCATTATCACCCCCA | |
| 158 | TGTTGATACAACCATAAAATGATAATTACACCCAT | C*YNHKMIITPINSSLSPP (SEQ ID NO: 5435) |
| | AAATTCATCATTATCACCCCCA | |
| 159 | TGTCGATACAACCATAAAATGATAATTACACCCAT | CRYNHKMIITPINGSLSPP (SEQ ID NO: 5436) |
| | AAATGGATCATTATCACCCCCA | |
| 160 | TGTCGATACAACCATAAAATGATAATTACACCCAT | CRYNHKMIITPINSSLSPP (SEQ ID NO: 5437) |
| | AAATTCATCATTATCACCCCCA | |
| 161 | TGTGGATACAACCATAAAATGATAATTACACCCAT | CGYNHKMIITPINGSLSPP (SEQ ID NO: 5354) |
| | AAATGGATCATTATCACCCCCA | |
| 162 | TGTGGATACAACCATAAAATGATAATTACACCCAT | CGYNHKMIITPINSSLSPP (SEQ ID NO: 5438) |
| | AAATTCATCATTATCACCCCCA | |
| 163 | TGTTCATACAACCATAAAATGATAATTACACCCAT | CSYNHKMIITPINGSLSPP (SEQ ID NO: 5439) |
| | AAATGGATCATTATCACCCCCA | |
| 164 | TGTTCATACAACCATAAAATGATAATTACACCCAT | CSYNHKMIITPINSSLSPP (SEQ ID NO: 5440) |
| | AAATTCATCATTATCACCCCCA | |
| 165 | TGTTGATACAACCCTAAAATGATAATTACACCCAT | C*YNPKMIITPIN (SEQ ID NO: 5412) **LSPP (SEQ |
| | AAATTGATAATTATCACCCCCA | ID NO: 5432) |
| 166 | TGTCGATACAACCCTAAAATGATAATTACACCCAT | CRYNPKMIITPIN (SEQ ID NO: 5413) **LSPP (SEQ |
| | AAATTGATAATTATCACCCCCA | ID NO: 5432) |
| 167 | TGTGGATACAACCCTAAAATGATAATTACACCCAT | CGYNPKMIITPIN (SEQ ID NO: 5414) **LSPP (SEQ |
| | AAATTGATAATTATCACCCCCA | ID NO: 5432) |
| 168 | TGTTCATACAACCCTAAAATGATAATTACACCCAT | CSYNPKMIITPIN (SEQ ID NO: 5415) **LSPP (SEQ |
| | AAATTGATAATTATCACCCCCA | ID NO: 5432) |
| 169 | TGTTGATACAACCCTAAAATGATAATTACACCCAT | C*YNPKMIITPING (SEQ ID NO: 5416) *LSPP (SEQ |
| | AAATGGATAATTATCACCCCCA | ID NO: 5432) |
| 170 | TGTTGATACAACCCTAAAATGATAATTACACCCAT | C*YNPKMIITPINS (SEQ ID NO: 5417) *LSPP (SEQ |
| | AAATTCATAATTATCACCCCCA | ID NO: 5432) |
| 171 | TGTCGATACAACCCTAAAATGATAATTACACCCAT | CRYNPKMIITPING (SEQ ID NO: 5418) *LSPP (SEQ |
| | AAATGGATAATTATCACCCCCA | ID NO: 5432) |
| 172 | TGTCGATACAACCCTAAAATGATAATTACACCCAT | CRYNPKMIITPINS (SEQ ID NO: 5419) *LSPP (SEQ |
| | AAATTCATAATTATCACCCCCA | ID NO: 5432) |
| 173 | TGTGGATACAACCCTAAAATGATAATTACACCCAT | CGYNPKMIITPING (SEQ ID NO: 5420) *LSPP (SEQ |
| | AAATGGATAATTATCACCCCCA | ID NO: 5432) |
| 174 | TGTGGATACAACCCTAAAATGATAATTACACCCAT | CGYNPKMIITPINS (SEQ ID NO: 5421) *LSPP (SEQ |
| | AAATTCATAATTATCACCCCCA | ID NO: 5432) |
| 175 | TGTTCATACAACCCTAAAATGATAATTACACCCAT | CSYNPKMITPING (SEQ ID NO: 5422) *LSPP (SEQ |
| | AAATGGATAATTATCACCCCCA | ID NO: 5432) |
| 176 | TGTTCATACAACCCTAAAATGATAATTACACCCAT | CSYNPKMIITPINS (SEQ ID NO: 5423) *LSPP (SEQ |
| | AAATTCATAATTATCACCCCCA | ID NO: 5432) |
| 177 | TGTTGATACAACCCTAAAATGATAATTACACCCAT | C*YNPKMIITPIN (SEQ ID NO: 5412) *SLSPP (SEQ |
| | AAATTGATCATTATCACCCCCA | ID NO: 5433) |
| 178 | TGTCGATACAACCCTAAAATGATAATTACACCCAT | CRYNPKMIITPIN (SEQ ID NO: 5413) *SLSPP (SEQ |
| | AAATTGATCATTATCACCCCCA | ID NO: 5433) |
| 179 | TGTGGATACAACCCTAAAATGATAATTACACCCAT | CGYNPKMIITPIN (SEQ ID NO: 5414) *SLSPP (SEQ |
| | AAATTGATCATTATCACCCCCA | ID NO: 5433) |
| 180 | TGTTCATACAACCCTAAAATGATAATTACACCCAT | CSYNPKMIITPIN (SEQ ID NO: 5415) *SLSPP (SEQ |
| | AAATTGATCATTATCACCCCCA | ID NO: 5433) |
| 181 | TGTTGATACAACCCTAAAATGATAATTACACCCAT | C*YNPKMIITPINGSLSPP (SEQ ID NO: 5441) |
| | AAATGGATCATTATCACCCCCA | |
| 182 | TGTTGATACAACCCTAAAATGATAATTACACCCAT | C*YNPKMIITPINSSLSPP (SEQ ID NO: 5442) |
| | AAATTCATCATTATCACCCCCA | |
| 183 | TGTCGATACAACCCTAAAATGATAATTACACCCAT | CRYNPKMIITPINGSLSPP (SEQ ID NO: 5443) |
| | AAATGGATCATTATCACCCCCA | |
| 184 | TGTCGATACAACCCTAAAATGATAATTACACCCAT | CRYNPKMITPINSSLSPP (SEQ ID NO: 5444) |
| | AAATTCATCATTATCACCCCCA | |
| 185 | TGTGGATACAACCCTAAAATGATAATTACACCCAT | CGYNPKMIITPINGSLSPP (SEQ ID NO: 5445) |
| | AAATGGATCATTATCACCCCCA | |
| 186 | TGTGGATACAACCCTAAAATGATAATTACACCCAT | CGYNPKMIITPINSSLSPP (SEQ ID NO: 5446) |
| | AAATTCATCATTATCACCCCCA | |
| 187 | TGTTCATACAACCCTAAAATGATAATTACACCCAT | CSYNPKMIITPINGSLSPP (SEQ ID NO: 5447) |
| | AAATGGATCATTATCACCCCCA | |
| 188 | TGTTCATACAACCCTAAAATGATAATTACACCCAT | CSYNPKMIITPINSSLSPP (SEQ ID NO: 5448) |
| | AAATTCATCATTATCACCCCCA | |
| 189 | TGTTGATACAACCATAAAACGATAATTACACCCAT | C*YNHKTIITPIN (SEQ ID NO: 5449) **LSHP (SEQ |
| | AAATTGATAATTATCACACCCA | ID NO: 5353) |
| 190 | TGTCGATACAACCATAAAACGATAATTACACCCAT | CRYNHKTIITPIN (SEQ ID NO: 5450) **LSHP (SEQ |
| | AAATTGATAATTATCACACCCA | ID NO: 5353) |
| 191 | TGTGGATACAACCATAAAACGATAATTACACCCAT | CGYNHKTIITPIN (SEQ ID NO: 5451) **LSHP (SEQ |
| | AAATTGATAATTATCACACCCA | ID NO: 5353) |
| 192 | TGTTCATACAACCATAAAACGATAATTACACCCAT | CSYNHKTIITPIN (SEQ ID NO: 5452) **LSHP (SEQ |
| | AAATTGATAATTATCACACCCA | ID NO: 5353) |
| 193 | TGTTGATACAACCATAAAACGATAATTACACCCAT | C*YNHKTIITPING (SEQ ID NO: 5453) *LSHP (SEQ |
| | AAATGGATAATTATCACACCCA | ID NO: 5353) |
| 194 | TGTTGATACAACCATAAAACGATAATTACACCCAT | C*YNHKTIITPINSLSHP (SEQ ID NO: 5353) |
| | AAATTCATAATTATCACACCCA | |
| 195 | TGTCGATACAACCATAAAACGATAATTACACCCAT | CRYNHKTIITPING (SEQ ID NO: 5454) *LSHP (SEQ |
| | AAATGGATAATTATCACACCCA | ID NO: 5353) |
| 196 | TGTCGATACAACCATAAAACGATAATTACACCCAT | CRYNHKTIITPINS (SEQ ID NO: 5455) *LSHP (SEQ |
| | AAATTCATAATTATCACACCCA | ID NO: 5353) |
| 197 | TGTGGATACAACCATAAAACGATAATTACACCCAT | CGYNHKTIITPING (SEQ ID NO: 5456) *LSHP (SEQ |
| | AAATGGATAATTATCACACCCA | ID NO: 5353) |
| 198 | TGTGGATACAACCATAAAACGATAATTACACCCAT | CGYNHKTIITPINS (SEQ ID NO: 5457) *LSHP (SEQ |
| | AAATTCATAATTATCACACCCA | ID NO: 5353) |
| 199 | TGTTCATACAACCATAAAACGATAATTACACCCAT | CSYNHKTIITPING (SEQ ID NO: 5458) *LSHP (SEQ |
| | AAATGGATAATTATCACACCCA | ID NO: 5353) |
| 200 | TGTTCATACAACCATAAAACGATAATTACACCCAT | CSYNHKTIITPINS (SEQ ID NO: 5459) *LSHP (SEQ |
| | AAATTCATAATTATCACACCCA | ID NO: 5353) |
| 201 | TGTTGATACAACCATAAAACGATAATTACACCCAT | C*YNHKTIITPIN (SEQ ID NO: 5449) *SLSHP (SEQ |
| | AAATTGATCATTATCACACCCA | ID NO: 5372) |
| 202 | TGTCGATACAACCATAAAACGATAATTACACCCAT | CRYNHKTIITPIN (SEQ ID NO: 5450) *SLSHP (SEQ |
| | AAATTGATCATTATCACACCCA | ID NO: 5372) |
| 203 | TGTGGATACAACCATAAAACGATAATTACACCCAT | CGYNHKTIITPIN (SEQ ID NO: 5451) *SLSHP (SEQ |
| | AAATTGATCATTATCACACCCA | ID NO: 5372) |
| 204 | TGTTCATACAACCATAAAACGATAATTACACCCAT | CSYNHKTIITPIN (SEQ ID NO: 5452) *SLSHP (SEQ |
| | AAATTGATCATTATCACACCCA | ID NO: 5372) |
| 205 | TGTTGATACAACCATAAAACGATAATTACACCCAT | C*YNHKTIITPINGSLSHP (SEQ ID NO: 5460) |
| | AAATGGATCATTATCACACCCA | |
| 206 | TGTTGATACAACCATAAAACGATAATTACACCCAT | C*YNHKTIITPINSSLSHP (SEQ ID NO: 5461) |
| | AAATTCATCATTATCACACCCA | |
| 207 | TGTCGATACAACCATAAAACGATAATTACACCCAT | CRYNHKTIITPINGSLSHP (SEQ ID NO: 5462) |
| | AAATGGATCATTATCACACCCA | |
| 208 | TGTCGATACAACCATAAAACGATAATTACACCCAT | CRYNHKTIITPINSSLSHP (SEQ ID NO: 5463) |
| | AAATTCATCATTATCACACCCA | |
| 209 | TGTGGATACAACCATAAAACGATAATTACACCCAT | CGYNHKTIITPINGSLSHP (SEQ ID NO: 5355) |
| | AAATGGATCATTATCACACCCA | |
| 210 | TGTGGATACAACCATAAAACGATAATTACACCCAT | CGYNHKTIITPINSSLSHP (SEQ ID NO: 5464) |
| | AAATTCATCATTATCACACCCA | |
| 211 | TGTTCATACAACCATAAAACGATAATTACACCCAT | CSYNHKTITPINGSLSHP (SEQ ID NO: 5465) |
| | AAATGGATCATTATCACACCCA | |
| 212 | TGTTCATACAACCATAAAACGATAATTACACCCAT | CSYNHKTITPINSSLSHP (SEQ ID NO: 5466) |
| | AAATTCATCATTATCACACCCA | |
| 213 | TGTTGATCCAACCATAAAATGATAATTACACCCAT | C*SNHKMIITPIN (SEQ ID NO: 5467) **LSHP (SEQ |
| | AAATTGATAATTATCACACCCA | ID NO: 5353) |
| 214 | TGTCGATCCAACCATAAAATGATAATTACACCCAT | CRSNHKMIITPIN (SEQ ID NO: 5468) **LSHP (SEQ |
| | AAATTGATAATTATCACACCCA | ID NO: 5353) |
| 215 | TGTGGATCCAACCATAAAATGATAATTACACCCAT | CGSNHKMIITPIN (SEQ ID NO: 5469) **LSHP (SEQ |
| | AAATTGATAATTATCACACCCA | ID NO: 5353) |
| 216 | TGTTCATCCAACCATAAAATGATAATTACACCCAT | CSSNHKMIITPIN (SEQ ID NO: 5470) **LSHP (SEQ |
| | AAATTGATAATTATCACACCCA | ID NO: 5353) |
| 217 | TGTTGATCCAACCATAAAATGATAATTACACCCAT | C*SNHKMIITPING (SEQ ID NO: 5471) *LSHP (SEQ |
| | AAATGGATAATTATCACACCCA | ID NO: 5353) |
| 218 | TGTTGATCCAACCATAAAATGATAATTACACCCAT | C*SNHKMIITPINS (SEQ ID NO: 5472) *LSHP (SEQ |
| | AAATTCATAATTATCACACCCA | ID NO: 5353) |
| 219 | TGTCGATCCAACCATAAAATGATAATTACACCCAT | CRSNHKMIITPING (SEQ ID NO: 5473) *LSHP (SEQ |
| | AAATGGATAATTATCACACCCA | ID NO: 5353) |
| 220 | TGTCGATCCAACCATAAAATGATAATTACACCCAT | CRSNHKMIITPINS (SEQ ID NO: 5474) *LSHP (SEQ |
| | AAATTCATAATTATCACACCCA | ID NO: 5353) |
| 221 | TGTGGATCCAACCATAAAATGATAATTACACCCAT | CGSNHKMIITPING (SEQ ID NO: 5475) *LSHP (SEQ |
| | AAATGGATAATTATCACACCCA | ID NO: 5353) |
| 222 | TGTGGATCCAACCATAAAATGATAATTACACCCAT | CGSNHKMIITPINS (SEQ ID NO: 5476) *LSHP (SEQ |
| | AAATTCATAATTATCACACCCA | ID NO: 5353) |
| 223 | TGTTCATCCAACCATAAAATGATAATTACACCCAT | CSSNHKMIITPING (SEQ ID NO: 5477) *LSHP (SEQ |
| | AAATGGATAATTATCACACCCA | ID NO: 5353) |
| 224 | TGTTCATCCAACCATAAAATGATAATTACACCCAT | CSSNHKMITPINS (SEQ ID NO: 5478) *LSHP (SEQ |
| | AAATTCATAATTATCACACCCA | ID NO: 5353) |
| 225 | TGTTGATCCAACCATAAAATGATAATTACACCCAT | C*SNHKMIITPIN (SEQ ID NO: 5467) *SLSHP (SEQ |
| | AAATTGATCATTATCACACCCA | ID NO: 5372) |
| 226 | TGTCGATCCAACCATAAAATGATAATTACACCCAT | CRSNHKMITPIN (SEQ ID NO: 5468) *SLSHP (SEQ |
| | AAATTGATCATTATCACACCCA | ID NO: 5372) |
| 227 | TGTGGATCCAACCATAAAATGATAATTACACCCAT | CGSNHKMIITPIN (SEQ ID NO: 5469) *SLSHP (SEQ |
| | AAATTGATCATTATCACACCCA | ID NO: 5372) |
| 228 | TGTTCATCCAACCATAAAATGATAATTACACCCAT | CSSNHKMIITPIN (SEQ ID NO: 5470) *SLSHP (SEQ |
| | AAATTGATCATTATCACACCCA | ID NO: 5372) |
| 229 | TGTTGATCCAACCATAAAATGATAATTACACCCAT | C*SNHKMIITPINGSLSHP (SEQ ID NO: 5372) |
| | AAATGGATCATTATCACACCCA | |
| 230 | TGTTGATCCAACCATAAAATGATAATTACACCCAT | C*SNHKMIITPINSSLSHP (SEQ ID NO: 5479) |
| | AAATTCATCATTATCACACCCA | |
| 231 | TGTCGATCCAACCATAAAATGATAATTACACCCAT | CRSNHKMIITPINGSLSHP (SEQ ID NO: 5480) |
| | AAATGGATCATTATCACACCCA | |
| 232 | TGTCGATCCAACCATAAAATGATAATTACACCCAT | CRSNHKMIITPINSSLSHP (SEQ ID NO: 5481) |
| | AAATTCATCATTATCACACCCA | |
| 233 | TGTGGATCCAACCATAAAATGATAATTACACCCAT | CGSNHKMIITPINGSLSHP (SEQ ID NO: 5356) |
| | AAATGGATCATTATCACACCCA | |
| 234 | TGTGGATCCAACCATAAAATGATAATTACACCCAT | CGSNHKMIITPINSSLSHP (SEQ ID NO: 5482) |
| | AAATTCATCATTATCACACCCA | |
| 235 | TGTTCATCCAACCATAAAATGATAATTACACCCAT | CSSNHKMIITPINGSLSHP (SEQ ID NO: 5483) |
| | AAATGGATCATTATCACACCCA | |
| 236 | TGTTCATCCAACCATAAAATGATAATTACACCCAT | CSSNHKMIITPINSSLSHP (SEQ ID NO: 5484) |
| | AAATTCATCATTATCACACCCA | |
| 237 | TGTTGATACAACCATAAAATGATAATTCACCCATA | C*YNHKMIIHP (SEQ ID NO: 5485) *IDNYHTP |
| | AATTGATAATTATCACACCCCA | (SEQ ID NO: 5486) |
| 238 | TGTTGATACAACCATAAAATGATAATTCACCCATC | C*YNHKMIIHPSIDNYHTP (SEQ ID NO: 5487) |
| | AATTGATAATTATCACACCCCA | |
| 239 | TGTTCATACAACCATAAAATGATAATTCACCCATA | CSYNHKMIIHP (SEQ ID NO: 5488) *IDNYHTP |
| | AATTGATAATTATCACACCCCA | (SEQ ID NO: 5486) |
| 240 | TGTTCATACAACCATAAAATGATAATTCACCCATC | CSYNHKMIIHPSIDNYHTP (SEQ ID NO: 5489) |
| | AATTGATAATTATCACACCCCA | |
| 241 | TGTGGATACAACCATAAAATGATAATTCACCCATA | CGYNHKMIIHP (SEQ ID NO: 5490) *IDNYHTP |
| | AATTGATAATTATCACACCCCA | (SEQ ID NO: 5486) |
| 242 | TGTGGATACAACCATAAAATGATAATTCACCCATC | CGYNHKMIIHPSIDNYHTP (SEQ ID NO: 5491) |
| | AATTGATAATTATCACACCCCA | |
| 243 | TGTCGATACAACCATAAAATGATAATTCACCCATA | CRYNHKMIIHP (SEQ ID NO: 5492) *IDNYHTP |
| | AATTGATAATTATCACACCCCA | (SEQ ID NO: 5486) |
| 244 | TGTCGATACAACCATAAAATGATAATTCACCCATC | CRYNHKMIIHPSIDNYHTP (SEQ ID NO: 5493) |
| | AATTGATAATTATCACACCCCA | |
| 245 | TGTTGATACAACCATAAAATGATAATTACCACCCA | C*YNHKMIITTHKLIIITP (SEQ ID NO: 5494) |
| | TAAATTGATAATTATCACACCC | |
| 246 | TGTTGATACAACCATAAAATGATAATTACCACCCC | C*YNHKMIITTPKLIIITP (SEQ ID NO: 5495) |
| | TAAATTGATAATTATCACACCC | |
| 247 | TGTTGATACAACCATAAAATGATAATTACCACCCA | C*YNHKMIITTHTLIIITP (SEQ ID NO: 5496) |
| | TACATTGATAATTATCACACCC | |
| 248 | TGTTGATACAACCATAAAATGATAATTACCACCCC | C*YNHKMIITTPTLIIITP (SEQ ID NO: 5497) |
| | TACATTGATAATTATCACACCC | |
| 249 | TGTTCATACAACCATAAAATGATAATTACCACCCA | CSYNHKMIITTHKLIITP (SEQ ID NO: 5498) |
| | TAAATTGATAATTATCACACCC | |
| 250 | TGTTCATACAACCATAAAATGATAATTACCACCCC | CSYNHKMITTPKLIIITP (SEQ ID NO: 5499) |
| | TAAATTGATAATTATCACACCC | |
| 251 | TGTTCATACAACCATAAAATGATAATTACCACCCA | CSYNHKMIITTHTLIIITP (SEQ ID NO: 5500) |
| | TACATTGATAATTATCACACCC | |
| 252 | TGTTCATACAACCATAAAATGATAATTACCACCCC | CSYNHKMIITTPTLIITP (SEQ ID NO: 5501) |
| | TACATTGATAATTATCACACCC | |
| 253 | TGTGGATACAACCATAAAATGATAATTACCACCCA | CGYNHKMIITTHKLIIITP (SEQ ID NO: 5502) |
| | TAAATTGATAATTATCACACCC | |
| 254 | TGTGGATACAACCATAAAATGATAATTACCACCCC | CGYNHKMIITTPKLIIITP (SEQ ID NO: 5503) |
| | TAAATTGATAATTATCACACCC | |
| 255 | TGTGGATACAACCATAAAATGATAATTACCACCCA | CGYNHKMIITTHTLIITP (SEQ ID NO: 5504) |
| | TACATTGATAATTATCACACCC | |
| 256 | TGTGGATACAACCATAAAATGATAATTACCACCCC | CGYNHKMIITTPTLIIITP (SEQ ID NO: 5505) |
| | TACATTGATAATTATCACACCC | |
| 257 | TGTCGATACAACCATAAAATGATAATTACCACCCA | CRYNHKMIITTHKLIIITP (SEQ ID NO: 5506) |
| | TAAATTGATAATTATCACACCC | |
| 258 | TGTCGATACAACCATAAAATGATAATTACCACCCC | CRYNHKMIITTPKLIIITP (SEQ ID NO: 5507) |
| | TAAATTGATAATTATCACACCC | |
| 259 | TGTCGATACAACCATAAAATGATAATTACCACCCA | CRYNHKMITTHTLIITP (SEQ ID NO: 5508) |
| | TACATTGATAATTATCACACCC | |
| 260 | TGTCGATACAACCATAAAATGATAATTACCACCCC | CRYNHKMIITTPTLIITP (SEQ ID NO: 5509) |
| | TACATTGATAATTATCACACCC | |
| 18 | TGTTGATACAACCATAAAATGATAATTACACCCAT | C*YNHKMIITPIN (SEQ ID NO: 5352)**LSHP (SEQ |
| | AAATTGATAATTATCACACCCA | ID NO: 5353) |
| 69 | TCTTGATACAACCATAAAATGATAATTACACCCAT | S*YNHKMIITPIN (SEQ ID NO: 5352)**LSHP (SEQ |
| | AAATTGATAATTATCACACCCA | ID NO: 5353) |
| 23 | TGTGGATACAACCATAAAATGATAATTACACCCAT | CGYNHKMIITPIN(SEQ ID NO: 5362) * *LSHP (SEQ |
| | AAATTGATAATTATCACACCCA | ID NO: 5353) |
| 264 | TGTTTATACAACCATAAAATGATAATTACACCCAT | CLYNHKMIITPIN (SEQ ID NO: 5510) **LSHP (SEQ |
| | AAATTGATAATTATCACACCCA | ID NO: 5353) |
| 24 | TGTTCATACAACCATAAAATGATAATTACACCCAT | CSYNHKMIITPIN(SEQ ID NO: 5363) * *LSHP (SEQ |
| | AAATTGATAATTATCACACCCA | ID NO: 5353) |
| 266 | TGTTGAAACAACCATAAAATGATAATTACACCCAT | C*NNHKMIITPIN (SEQ ID NO: 5511) **LSHP (SEQ |
| | AAATTGATAATTATCACACCCA | ID NO: 5353) |
| 213 | TGTTGATCCAACCATAAAATGATAATTACACCCAT | C*SNHKMIITPIN (SEQ ID NO: 5467) **LSHP (SEQ |
| | AAATTGATAATTATCACACCCA | ID NO: 5353) |
| 268 | TGTTGATACATCCATAAAATGATAATTACACCCAT | C*YIHKMITPIN (SEQ ID NO: 5512) **LSHP (SEQ |
| | AAATTGATAATTATCACACCCA | ID NO: 5353) |
| 269 | TGTTGATACACCCATAAAATGATAATTACACCCAT | C*YTHKMIITPIN (SEQ ID NO: 5513) **LSHP (SEQ |
| | AAATTGATAATTATCACACCCA | ID NO: 5353) |
| 270 | TGTTGATACAGCCATAAAATGATAATTACACCCAT | C*YSHKMIITPIN (SEQ ID NO: 5514) **LSHP (SEQ |
| | AAATTGATAATTATCACACCCA | ID NO: 5353) |
| 271 | TGTTGATACAACAATAAAATGATAATTACACCCAT | C*YNNKMIITPIN (SEQ ID NO: 5515) **LSHP (SEQ |
| | AAATTGATAATTATCACACCCA | ID NO: 5353) |
| 272 | TGTTGATACAACCTTAAAATGATAATTACACCCAT | C*YNLKMIITPIN (SEQ ID NO: 5516) **LSHP (SEQ |
| | AAATTGATAATTATCACACCCA | ID NO: 5353) |
| 117 | TGTTGATACAACCCTAAAATGATAATTACACCCAT | C*YNPKMITPIN (SEQ ID NO: 5412) **LSHP (SEQ |
| | AAATTGATAATTATCACACCCA | ID NO: 5353) |
| 274 | TGTTGATACAACCAAAAAATGATAATTACACCCAT | C*YNQKMIITPIN (SEQ ID NO: 5517) **LSHP (SEQ |
| | AAATTGATAATTATCACACCCA | ID NO: 5353) |
| 275 | TGTTGATACAACCAGAAAATGATAATTACACCCAT | C*YNQKMIITPIN (SEQ ID NO: 5517) **LSHP (SEQ |
| | AAATTGATAATTATCACACCCA | ID NO: 5353) |
| 276 | TGTTGATACAACCATATAATGATAATTACACCCAT | C*YNHIMIITPIN (SEQ ID NO: 5518) **LSHP (SEQ |
| | AAATTGATAATTATCACACCCA | ID NO: 5353) |
| 277 | TGTTGATACAACCATACAATGATAATTACACCCAT | C*YNHTMIITPIN (SEQ ID NO: 5519) **LSHP (SEQ |
| | AAATTGATAATTATCACACCCA | ID NO: 5353) |
| 278 | TGTTGATACAACCATAAATTGATAATTACACCCAT | C*YNHKLIITPIN (SEQ ID NO: 5520) **LSHP (SEQ |
| | AAATTGATAATTATCACACCCA | ID NO: 5353) |
| 279 | TGTTGATACAACCATAAACTGATAATTACACCCAT | C*YNHKLIITPIN (SEQ ID NO: 5520) **LSHP (SEQ |
| | AAATTGATAATTATCACACCCA | ID NO: 5353) |
| 280 | TGTTGATACAACCATAAAGTGATAATTACACCCAT | C*YNHKVIITPIN (SEQ ID NO: 5521) **LSHP (SEQ |
| | AAATTGATAATTATCACACCCA | ID NO: 5353) |
| 189 | TGTTGATACAACCATAAAACGATAATTACACCCAT | C*YNHKTIITPIN (SEQ ID NO: 5449) **LSHP (SEQ |
| | AAATTGATAATTATCACACCCA | ID NO: 5353) |
| 282 | TGTTGATACAACCATAAAATTATAATTACACCCAT | C*YNHKIIITPIN (SEQ ID NO: 5522) **LSHP (SEQ ID NO: |
| | AAATTGATAATTATCACACCCA | 5353) |
| 283 | TGTTGATACAACCATAAAATCATAATTACACCCAT | C*YNHKIIITPIN (SEQ ID NO: 5522) **LSHP (SEQ ID NO: |
| | AAATTGATAATTATCACACCCA | 5353) |
| 284 | TGTTGATACAACCATAAAATAATAATTACACCCAT | C*YNHKIIITPIN (SEQ ID NO: 5522) **LSHP (SEQ ID NO: |
| | AAATTGATAATTATCACACCCA | 5353) |
| 285 | TGTTGATACAACCATAAAATGATAATTACACCCAT | C*YNHKMIITPII (SEQ ID NO: 5523) **LSHP (SEQ ID NO: |
| | AATTTGATAATTATCACACCCA | 5353) |
| 286 | TGTTGATACAACCATAAAATGATAATTACACCCAT | C*YNHKMIITPIT (SEQ ID NO: 5524) **LSHP (SEQ |
| | AACTTGATAATTATCACACCCA | ID NO: 5353) |
| 287 | TGTTGATACAACCATAAAATGATAATTACACCCAT | C*YNHKMIITPIS (SEQ ID NO: 5526) **LSHP (SEQ |
| | AAGTTGATAATTATCACACCCA | ID NO: 5353) |
| 25 | TGTTGATACAACCATAAAATGATAATTACACCCAT | C*YNHKMIITPING(SEQ ID NO: 5364) * LSHP (SEQ |
| | AAATGGATAATTATCACACCCA | ID NO: 5353) |
| 289 | TGTTGATACAACCATAAAATGATAATTACACCCAT | C*YNHKMIITPINL (SEQ ID NO: 5527) *LSHP (SEQ |
| | AAATTTATAATTATCACACCCA | ID NO: 5353) |
| 26 | TGTTGATACAACCATAAAATGATAATTACACCCAT | C*YNHKMIITPINS(SEQ ID NO: 5365) * LSHP (SEQ |
| | AAATTCATAATTATCACACCCA | ID NO: 5353) |
| 291 | TGTTGATACAACCATAAAATGATAATTACACCCAT | C*YNHKMIITPIN (SEQ ID NO: 5352)*QLSHP (SEQ |
| | AAATTGACAATTATCACACCCA | ID NO: 5528) |
| 292 | TGTTGATACAACCATAAAATGATAATTACACCCAT | C*YNHKMIITPIN (SEQ ID NO: 5352)*LLSHP (SEQ |
| | AAATTGATTATTATCACACCCA | ID NO: 5529) |
| 33 | TGTTGATACAACCATAAAATGATAATTACACCCAT | C*YNHKMIITPIN (SEQ ID NO: 5352)*SLSHP (SEQ |
| | AAATTGATCATTATCACACCCA | ID NO: 5372) |
| 294 | TGTTGATACAACCATAAAATGATAATTACACCCAT | C*YNHKMIITPIN (SEQ ID NO: 5352)**LSNP (SEQ |
| | AAATTGATAATTATCAAACCCA | ID NO: 5543) |
| 298 | TGTTGATACAACCATAAAATGATAATTACACCCAT | C*YNHKMIITPIN (SEQ ID NO: 5352)**LSLP (SEQ |
| | AAATTGATAATTATCACTOCCA | ID NO: 5544) |
| 141 | TGTTGATACAACCATAAAATGATAATTACACCCAT | C*YNHKMIITPIN (SEQ ID NO: 5352)**LSPP (SEQ |
| | AAATTGATAATTATCACCCCCA | ID NO: 5432) |
| 29 | TGTGGATACAACCATAAAATGATAATTACACCCAT | CGYNHKMIITPING (SEQ ID NO: 5368) * LSHP |
| | AAATGGATAATTATCACACCCA | (SEQ ID NO: 5353) |
| 298 | TGTGGATACAACCATAAAATGATAATTACACCCAT | CGYNHKMIITPINL (SEQ ID NO: 5530) *LSHP (SEQ |
| | AAATTTATAATTATCACACCCA | ID NO: 5353) |
| 30 | TGTGGATACAACCATAAAATGATAATTACACCCAT | CGYNHKMIITPINS (SEQ ID NO: 5369) * LSHP |
| | AAATTCATAATTATCACACCCA | (SEQ ID NO: 5353) |
| 300 | TGTTTATACAACCATAAAATGATAATTACACCCAT | CLYNHKMITPING (SEQ ID NO: 5531) *LSHP (SEQ |
| | AAATGGATAATTATCACACCCA | ID NO: 5353) |
| 301 | TGTTTATACAACCATAAAATGATAATTACACCCAT | CLYNHKMIITPINL (SEQ ID NO: 5532) *LSHP (SEQ |
| | AAATTTATAATTATCACACCCA | ID NO: 5353) |
| 302 | TGTTTATACAACCATAAAATGATAATTACACCCAT | CLYNHKMIITPINS (SEQ ID NO: 5533) *LSHP (SEQ |
| | AAATTCATAATTATCACACCCA | ID NO: 5353) |
| 303 | TGTTGATACAACCATAAAATGATAATTACACCCAT | C*YNHKMIITPINGLLSHP (SEQ ID NO: 5534) |
| | AAATGGATTATTATCACACCCA | |
| 304 | TGTTGATACAACCATAAAATGATAATTACACCCAT | C*YNHKMIITPINLLLSHP (SEQ ID NO: 5535) |
| | AAATTTATTATTATCACACCCA | |
| 305 | TGTTGATACAACCATAAAATGATAATTACACCCAT | C*YNHKMIITPINSLLSHP (SEQ ID NO: 5536) |
| | AAATTCATTATTATCACACCCA | |
| 306 | TGTGGATACAACCATAAAATGATAATTACACCCAT | CGYNHKMIITPINGLLSHP (SEQ ID NO: 5537) |
| | AAATGGATTATTATCACACCCA | |
| 307 | TGTGGATACAACCATAAAATGATAATTACACCCAT | CGYNHKMIITPINLLLSHP (SEQ ID NO: 5538) |
| | AAATTTATTATTATCACACCCA | |
| 308 | TGTGGATACAACCATAAAATGATAATTACACCCAT | CGYNHKMIITPINSLLSHP (SEQ ID NO: 5539) |
| | AAATTCATTATTATCACACCCA | |
| 309 | TGTTTATACAACCATAAAATGATAATTACACCCAT | CLYNHKMIITPINGLLSHP (SEQ ID NO: 5540) |
| | AAATGGATTATTATCACACCCA | |
| 310 | TGTTTATACAACCATAAAATGATAATTACACCCAT | CLYNHKMIITPINLLLSHP (SEQ ID NO: 5541) |
| | AAATTTATTATTATCACACCCA | |
| 302 | TGTTTATACAACCATAAAATGATAATTACACCCAT | CLYNHKMIITPINSLLSHP (SEQ ID NO: 5542) |
| | AAATTCATTATTATCACACCCA | |

Frame 2

| | | |
|---|---|---|
| 18 | TGTTGATACAACCATAAAATGATAATTACACCCAT | [X]VDTTIK (SEQ ID NO: 5545) |
| | AAATTGATAATTATCACACCCA | **LHP*IDNYHT[Q/H] (SEQ ID NO: 5546) |
| 316 | TGTTGATACAACCATAAAAGGATAATTACACCCAT | [X]VDTTIKG (SEQ ID NO: 5547) |
| | AAATTGATAATTATCACACCCA | *LHP*IDNYHT[Q/H] (SEQ ID NO: 5546) |
| 283 | TGTTGATACAACCATAAAATCATAATTACACCCAT | [X]VDTTIKS (SEQ ID NO: 5548) |
| | AAATTGATAATTATCACACCCA | *LHP*IDNYHT[Q/H] (SEQ ID NO: 5546) |
| 318 | TGTTGATACAACCATAAAATGATCATTACACCCAT | [X]VDTTIK (SEQ ID NO: 5545) *SLHP (SEQ ID NO: |
| | AAATTGATAATTATCACACCCA | 5549) *IDNYHT[Q/H] (SEQ ID NO: 5546) |
| 319 | TGTTGATACAACCATAAAAGGATCATTACACCCAT | [X]VDTTIKGSLHP (SEQ ID NO: 5550) |
| | AAATTGATAATTATCACACCCA | *IDNYHT[Q/H] (SEQ ID NO: 5546) |
| 320 | TGTTGATACAACCATAAAATCATCATTACACCCAT | [X]VDTTIKSSLHP (SEQ ID NO: 5551) |
| | AAATTGATAATTATCACACCCA | *IDNYHT[Q/H] (SEQ ID NO: 5546) |
| 321 | TGTTGATACAACCATAAAATGATAATTACACCCAT | [X]VDTTIK (SEQ ID NO: 5545) |
| | CAATTGATAATTATCACACCCA | **LHPSIDNYHT[Q/H] (SEQ ID NO: 5552) |
| 322 | TGTTGATACAACCATAAAAGGATAATTACACCCAT | [X]VDTTIKG (SEQ ID NO: 5547) |
| | CAATTGATAATTATCACACCCA | *LHPSIDNYHT[Q/H] (SEQ ID NO: 5552) |
| 323 | TGTTGATACAACCATAAAATCATAATTACACCCAT | [X]VDTTIKS (SEQ ID NO: 5548) |
| | CAATTGATAATTATCACACCCA | *LHPSIDNYHT[Q/H] (SEQ ID NO: 5552) |
| 324 | TGTTGATACAACCATAAAATGATCATTACACCCAT | [X]VDTTIK (SEQ ID NO: 5545) |
| | CAATTGATAATTATCACACCCA | *SLHPSIDNYHT[Q/H] (SEQ ID NO: 5553) |
| 325 | TGTTGATACAACCATAAAAGGATCATTACACCCAT | [X]VDTTIKGSLHPSIDNYHT[Q/H] (SEQ ID NO: |
| | CAATTGATAATTATCACACCCA | 5554) |
| 326 | TGTTGATACAACCATAAAATCATCATTACACCCAT | [X]VDTTIKSSLHPSIDNYHT[Q/H] (SEQ ID NO: |
| | CAATTGATAATTATCACACCCA | 5555) |
| 327 | TGTTGATACAACCATAAAATGATAATTACCCCCAT | [X]VDTTIK (SEQ ID NO: 5545) |
| | AAATTGATAATTATCACACCCA | **LPP*IDNYHT[Q/H] (SEQ ID NO: 5546) |
| 328 | TGTTGATACAACCATAAAAGGATAATTACCCCCAT | [X]VDTTIKG (SEQ ID NO: 5547) |
| | AAATTGATAATTATCACACCCA | *LPP*IDNYHT[Q/H] (SEQ ID NO: 5546) |
| 329 | TGTTGATACAACCATAAAATCATAATTACCCCCAT | [X]VDTTIKS (SEQ ID NO: 5548) |
| | AAATTGATAATTATCACACCCA | *LPP*IDNYHT[Q/H] (SEQ ID NO: 5546) |
| 330 | TGTTGATACAACCATAAAATGATCATTACCCCCAT | [X]VDTTIK (SEQ ID NO: 5545) *SLPP (SEQ ID NO: |
| | AAATTGATAATTATCACACCCA | 5556) *IDNYHT[Q/H] (SEQ ID NO: 5546) |
| 331 | TGTTGATACAACCATAAAAGGATCATTACCCCCAT | [X]VDTTIKGSLPP (SEQ ID NO: 5557) |
| | AAATTGATAATTATCACACCCA | *IDNYHT[Q/H] (SEQ ID NO: 5546) |
| 332 | TGTTGATACAACCATAAAATCATCATTACCCCCAT | [X]VDTTIKSSLPP (SEQ ID NO: 5558) |
| | AAATTGATAATTATCACACCCA | *IDNYHT[Q/H] (SEQ ID NO: 5546) |
| 333 | TGTTGATACAACCATAAAATGATAATTACCCCCAT | [X]VDTTIK (SEQ ID NO: 5545) |
| | CAATTGATAATTATCACACCCA | **LPPSIDNYHT[Q/H] (SEQ ID NO: 5559) |
| 334 | TGTTGATACAACCATAAAAGGATAATTACCCCCAT | [X]VDTTIKG (SEQ ID NO: 5547) |
| | CAATTGATAATTATCACACCCA | *LPPSIDNYHT[Q/H] (SEQ ID NO: 5559) |
| 335 | TGTTGATACAACCATAAAATCATAATTACCCCCAT | [X]VDTTIKS (SEQ ID NO: 5548) |
| | CAATTGATAATTATCACACCCA | *LPPSIDNYHT[Q/H] (SEQ ID NO: 5559) |
| 336 | TGTTGATACAACCATAAAATGATCATTACCCCCAT | [X]VDTTIK (SEQ ID NO: 5545) |
| | CAATTGATAATTATCACACCCA | *SLPPSIDNYHT[Q/H] (SEQ ID NO: 5560) |
| 337 | TGTTGATACAACCATAAAAGGATCATTACCCCCAT | [X]VDTTIKGSLPPSIDNYHT[Q/H] (SEQ ID NO: |
| | CAATTGATAATTATCACACCCA | 5561) |
| 338 | TGTTGATACAACCATAAAATCATCATTACCCCCAT | [X]VDTTIKSSLPPSIDNYHT[Q/H] (SEQ ID NO: |
| | CAATTGATAATTATCACACCCA | 5562) |
| 339 | TGTTGATACAACCATAAAATGATAATTACACCCAT | [X]VDTTIK (SEQ ID NO: 5545) |
| | AAATTGATAATTATCCCACCCA | **LHP*IDNYPT[Q/H] (SEQ ID NO: 5563) |
| 340 | TGTTGATACAACCATAAAAGGATAATTACACCCAT | [X]VDTTIKG (SEQ ID NO: 5547) |
| | AAATTGATAATTATCCCACCCA | *LHP*IDNYPT[Q/H] (SEQ ID NO: 5563) |
| 341 | TGTTGATACAACCATAAAATCATAATTACACCCAT | [X]VDTTIKS (SEQ ID NO: 5548) |
| | AAATTGATAATTATCCCACCCA | *LHP*IDNYPT[Q/H] (SEQ ID NO: 5563) |
| 342 | TGTTGATACAACCATAAAATGATCATTACACCCAT | [X]VDTTIK (SEQ ID NO: 5545) *SLHP (SEQ ID NO: |
| | AAATTGATAATTATCCCACCCA | 5549) *IDNYPT[Q/H] (SEQ ID NO: 5563) |
| 343 | TGTTGATACAACCATAAAAGGATCATTACACCCAT | [X]VDTTIKGSLHP (SEQ ID NO: 5550) |
| | AAATTGATAATTATCCCACCCA | *IDNYPT[Q/H] (SEQ ID NO: 5563) |
| 344 | TGTTGATACAACCATAAAATCATCATTACACCCAT | [X]VDTTIKSSLHP (SEQ ID NO: 5551) |
| | AAATTGATAATTATCCCACCCA | *IDNYPT[Q/H] (SEQ ID NO: 5563) |
| 345 | TGTTGATACAACCATAAAATGATAATTACACCCAT | [X]VDTTIK (SEQ ID NO: 5545) |
| | CAATTGATAATTATCCCACCCA | **LHPSIDNYPT[Q/H] (SEQ ID NO: 5564) |
| 346 | TGTTGATACAACCATAAAAGGATAATTACACCCAT | [X]VDTTIKG (SEQ ID NO: 5547) |
| | CAATTGATAATTATCCCACCCA | *LHPSIDNYPT[Q/H] (SEQ ID NO: 5564) |
| 347 | TGTTGATACAACCATAAAATCATAATTACACCCAT | [X]VDTTIKS (SEQ ID NO: 5548) |
| | CAATTGATAATTATCCCACCCA | *LHPSIDNYPT[Q/H] (SEQ ID NO: 5564) |
| 348 | TGTTGATACAACCATAAAATGATCATTACACCCAT | [X]VDTTIK (SEQ ID NO: 5545) |
| | CAATTGATAATTATCCCACCCA | *SLHPSIDNYPT[Q/H] (SEQ ID NO: 5565) |
| 349 | TGTTGATACAACCATAAAAGGATCATTACACCCAT | [X]VDTTIKGSLHPSIDNYPT[Q/H] (SEQ ID NO: |
| | CAATTGATAATTATCCCACCCA | 5566) |
| 350 | TGTTGATACAACCATAAAATCATCATTACACCCAT | [X]VDTTIKSSLHPSIDNYPT[Q/H] (SEQ ID NO: |
| | CAATTGATAATTATCCCACCCA | 5567) |
| 351 | TGTTGATACAACCATAAAATGATAATTACACCCAT | [X]VDTTIK (SEQ ID NO: 5545) **LHP*IDNYHTP |
| | AAATTGATAATTATCACACCCC | (SEQ ID NO: 5486) |
| 352 | TGTTGATACAACCATAAAAGGATAATTACACCCAT | [X]VDTTIKG (SEQ ID NO: 5547) *LHP*IDNYHTP |
| | AAATTGATAATTATCACACCCC | (SEQ ID NO: 5486) |
| 353 | TGTTGATACAACCATAAAATCATAATTACACCCAT | [X]VDTTIKS (SEQ ID NO: 5548) *LHP*IDNYHTP |
| | AAATTGATAATTATCACACCCC | (SEQ ID NO: 5486) |
| 354 | TGTTGATACAACCATAAAATGATCATTACACCCAT | [X]VDTTIK (SEQ ID NO: 5545) *SLHP (SEQ ID NO: |
| | AAATTGATAATTATCACACCCC | 5549) *IDNYHTP (SEQ ID NO: 5486) |
| 355 | TGTTGATACAACCATAAAAGGATCATTACACCCAT | [X]VDTTIKGSLHP (SEQ ID NO: 5550) *IDNYHTP |
| | AAATTGATAATTATCACACCCC | (SEQ ID NO: 5486) |
| 356 | TGTTGATACAACCATAAAATCATCATTACACCCAT | [X]VDTTIKSSLHP (SEQ ID NO: 5551) *IDNYHTP |
| | AAATTGATAATTATCACACCCC | (SEQ ID NO: 5486) |
| 357 | TGTTGATACAACCATAAAATGATAATTACACCCAT | [X]VDTTIK (SEQ ID NO: 5545) **LHPSIDNYHTP |
| | CAATTGATAATTATCACACCCC | (SEQ ID NO: 5568) |
| 358 | TGTTGATACAACCATAAAAGGATAATTACACCCAT | [X]VDTTIKG (SEQ ID NO: 5547) *LHPSIDNYHTP |
| | CAATTGATAATTATCACACCCC | (SEQ ID NO: 5568) |
| 359 | TGTTGATACAACCATAAAATCATAATTACACCCAT | [X]VDTTIKS (SEQ ID NO: 5548) *LHPSIDNYHTP |
| | CAATTGATAATTATCACACCCC | (SEQ ID NO: 5568) |
| 360 | TGTTGATACAACCATAAAATGATCATTACACCCAT | [X]VDTTIK (SEQ ID NO: 5545) *SLHPSIDNYHTP |
| | CAATTGATAATTATCACACCCC | (SEQ ID NO: 5569) |
| 361 | TGTTGATACAACCATAAAAGGATCATTACACCCAT | [X]VDTTIKGSLHPSIDNYHTP (SEQ ID NO: 5570) |
| | CAATTGATAATTATCACACCCC | |
| 362 | TGTTGATACAACCATAAAATCATCATTACACCCAT | [X]VDTTIKSSLHPSIDNYHTP (SEQ ID NO: 5571) |
| | CAATTGATAATTATCACACCCC | |
| 363 | TGTTGATACAACCATAAAATGATAATTACCCCCAT | [X]VDTTIK (SEQ ID NO: 5545) |
| | AAATTGATAATTATCCCACCCA | **LPP*IDNYPT[Q/H] (SEQ ID NO: 5563) |
| 364 | TGTTGATACAACCATAAAAGGATAATTACCCCCAT | [X]VDTTIKG (SEQ ID NO: 5547) |
| | AAATTGATAATTATCCCACCCA | *LPP*IDNYPT[Q/H] (SEQ ID NO: 5563) |
| 365 | TGTTGATACAACCATAAAATCATAATTACCCCCAT | [X]VDTTIKS (SEQ ID NO: 5548) |
| | AAATTGATAATTATCCCACCCA | *LPP*IDNYPT[Q/H] (SEQ ID NO: 5563) |
| 366 | TGTTGATACAACCATAAAATGATCATTACCCCCAT | [X]VDTTIK (SEQ ID NO: 5545) *SLPP (SEQ ID NO: |
| | AAATTGATAATTATCCCACCCA | 5556) *IDNYPT[Q/H] (SEQ ID NO: 5563) |
| 367 | TGTTGATACAACCATAAAAGGATCATTACCCCCAT | [X]VDTTIKGSLPP (SEQ ID NO: 5557) |
| | AAATTGATAATTATCCCACCCA | *IDNYPT[Q/H] (SEQ ID NO: 5563) |
| 368 | TGTTGATACAACCATAAAATCATCATTACCCCCAT | [X]VDTTIKSSLPP (SEQ ID NO: 5558) |
| | AAATTGATAATTATCCCACCCA | *IDNYPT[Q/H] (SEQ ID NO: 5563) |
| 369 | TGTTGATACAACCATAAAATGATAATTACCCCCAT | [X]VDTTIK (SEQ ID NO: 5545) |
| | CAATTGATAATTATCCCACCCA | **LPPSIDNYPT[Q/H] (SEQ ID NO: 5572) |
| 370 | TGTTGATACAACCATAAAAGGATAATTACCCCCAT | [X]VDTTIKG (SEQ ID NO: 5547) |
| | CAATTGATAATTATCCCACCCA | *LPPSIDNYPT[Q/H] (SEQ ID NO: 5572) |
| 371 | TGTTGATACAACCATAAAATCATAATTACCCCCAT | [X]VDTTIKS (SEQ ID NO: 5548) |
| | CAATTGATAATTATCCCACCCA | *LPPSIDNYPT[Q/H] (SEQ ID NO: 5572) |
| 372 | TGTTGATACAACCATAAAATGATCATTACCCCCAT | [X]VDTTIK (SBQ ID NO: 5545) |
| | CAATTGATAATTATCCCACCCA | *SLPPSIDNYPT[Q/H] (SEQ ID NO: 5792) |
| 373 | TGTTGATACAACCATAAAAGGATCATTACCCCCAT | [X]VDTTIKGSLPPSIDNYPT[Q/H] (SEQ ID NO: |
| | CAATTGATAATTATCCCACCCA | 5793) |
| 374 | TGTTGATACAACCATAAAATCATCATTACCCCCAT | [X]VDTTIKSSLPPSIDNYPT[Q/H] (SEQ ID NO: |
| | CAATTGATAATTATCCCACCCA | 5794) |
| 375 | TGTTGATACAACCATAAAATGATAATTACCCCCAT | [X]VDTTIK (SEQ ID NO: 5545) **LPP*IDNYHTP |
| | AAATTGATAATTATCACACCCC | (SEQ ID NO: 5486) |
| 376 | TGTTGATACAACCATAAAAGGATAATTACCCCCAT | [X]VDTTIKG (SEQ ID NO: 5547) *LPP*IDNYHTP |
| | AAATTGATAATTATCACACCCC | (SEQ ID NO: 5486) |
| 377 | TGTTGATACAACCATAAAATCATAATTACCCCCAT | [X]VDTTIKS (SEQ ID NO: 5548) *LPP*IDNYHTP |
| | AAATTGATAATTATCACACCCC | (SEQ ID NO: 5486) |
| 378 | TGTTGATACAACCATAAAATGATCATTACCCCCAT | [X]VDTTIK (SEQ ID NO: 5545) *SLPP (SEQ ID NO: |
| | AAATTGATAATTATCACACCCC | 5556) *IDNYHTP (SEQ ID NO: 5486) |
| 379 | TGTTGATACAACCATAAAAGGATCATTACCCCCAT | [X]VDTTIKGSLPP (SEQ ID NO: 5557) *IDNYHTP |
| | AAATTGATAATTATCACACCCC | (SEQ ID NO: 5486) |
| 380 | TGTTGATACAACCATAAAATCATCATTACCCCCAT | [X]VDTTIKSSLPP (SEQ ID NO: 5558) *IDNYHTP |
| | AAATTGATAATTATCACACCCC | (SEQ ID NO: 5486) |
| 381 | TGTTGATACAACCATAAAATGATAATTACCCCCAT | [X]VDTTIK (SEQ ID NO: 5545) **LPPSIDNYHTP |
| | CAATTGATAATTATCACACCCC | (SEQ ID NO: 5795) |
| 382 | TGTTGATACAACCATAAAAGGATAATTACCCCCAT | [X]VDTTIKG (SEQ ID NO: 5547) |
| | CAATTGATAATTATCACACCCC | *LPPSIDNYHTP(SEQ ID NO: 5795) |
| 383 | TGTTGATACAACCATAAAATCATAATTACCCCCAT | [X]VDTTIKS (SEQ ID NO: 5548) |
| | CAATTGATAATTATCACACCCC | *LPPSIDNYHTP(SBQ ID NO: 5795) |
| 384 | TGTTGATACAACCATAAAATGATCATTACCCCCAT | [X]VDTTIK (SEQ ID NO: 5545) |
| | CAATTGATAATTATCACACCCC | *SLPPSIDNYHTP(SEQ ID NO: 5796) |
| 385 | TGTTGATACAACCATAAAAGGATCATTACCCCCAT | [X]VDTTIKGSLPPSIDNYHTP(SEQ ID NO: 5797) |
| | CAATTGATAATTATCACACCCC | |
| 386 | TGTTGATACAACCATAAAATCATCATTACCCCCAT | [X]VDTTIKSSLPPSIDNYHTP(SEQ ID NO: 5798) |
| | CAATTGATAATTATCACACCCC | |
| 387 | TGTTGATACAACCATAAAATGATAATTACACCCAT | [X]VDTTIK (SEQ ID NO: 5545) |
| | AAATTGATAATTATCCCACCCC | **LHP*IDNYPTP(SEQ ID NO: 5799) |
| 388 | TGTTGATACAACCATAAAAGGATAATTACACCCAT | [X]VDTTIKG (SEQ ID NO: 5547) |
| | AAATTGATAATTATCCCACCCC | *LHP*IDNYPTP(SEQ ID NO: 5799) |
| 389 | TGTTGATACAACCATAAAATCATAATTACACCCAT | [X]VDTTIKS (SEQ ID NO: 5548) |
| | AAATTGATAATTATCCCACCCC | *LHP*IDNYPTP(SEQ ID NO: 5799) |
| 390 | TGTTGATACAACCATAAAATGATCATTACACCCAT | [X]VDTTIK (SEQ ID NO: 5545) *SLHP (SEQ ID NO: |
| | AAATTGATAATTATCCCACCCC | 5549) *IDNYPTP(SEQ ID NO: 5799) |
| 391 | TGTTGATACAACCATAAAAGGATCATTACACCCAT | [X]VDTTIKGSLHP (SEQ ID NO: 5550) |
| | AAATTGATAATTATCCCACCCC | *IDNYPTP(SEQ ID NO: 5799) |
| 392 | TGTTGATACAACCATAAAATCATCATTACACCCAT | [X]VDTTIKSSLHP (SEQ ID NO: 5551) |
| | AAATTGATAATTATCCCACCCC | *IDNYPTP(SEQ ID NO: 5799) |
| 393 | TGTTGATACAACCATAAAATGATAATTACACCCAT | [X]VDTTIK (SEQ ID NO: 5545) **LHPSIDNYPTP |
| | CAATTGATAATTATCCCACCCC | (SEQ ID NO: 5800) |
| 394 | TGTTGATACAACCATAAAAGGATAATTACACCCAT | [X]VDTTIKG (SEQ ID NO: 5547) |
| | CAATTGATAATTATCCCACCCC | *LHPSIDNYPTP(SEQ ID NO: 5800) |
| 395 | TGTTGATACAACCATAAAATCATAATTACACCCAT | [X]VDTTIKS (SEQ ID NO: 5548) |
| | CAATTGATAATTATCCCACCCC | *LHPSIDNYPTP(SEQ ID NO: 5800) |
| 396 | TGTTGATACAACCATAAAATGATCATTACACCCAT | [X]VDTTIK (SEQ ID NO: 5545) |
| | CAATTGATAATTATCCCACCCC | *SLHPSIDNYPTP(SEQ ID NO: 5801) |
| 397 | TGTTGATACAACCATAAAAGGATCATTACACCCAT | [X]VDTTIKGSLHPSIDNYPTP (SEQ ID NO: 5802) |
| | CAATTGATAATTATCCCACCCC | |
| 398 | TGTTGATACAACCATAAAATCATCATTACACCCAT | [X]VDTTIKSSLHPSIDNYPTP (SEQ ID NO: 5803) |
| | CAATTGATAATTATCCCACCCC | |
| 399 | TGTTGATACAACCATAAAATGATAATTACCCCCAT | [X]VDTTIK (SEQ ID NO: 5545) |
| | AAATTGATAATTATCCCACCCC | **LPP*IDNYPTP(SEQ ID NO: 5799) |
| 400 | TGTTGATACAACCATAAAAGGATAATTACCCCCAT | [X]VDTTIKG (SEQ ID NO: 5547) |
| | AAATTGATAATTATCCCACCCC | *LPP*IDNYPTP(SEQ ID NO: 5799) |
| 401 | TGTTGATACAACCATAAAATCATAATTACCCCCAT | [X]VDTTIKS (SEQ ID NO: 5548) |
| | AAATTGATAATTATCCCACCCC | *LPP*IDNYPTP(SEQ ID NO: 5799) |
| 402 | TGTTGATACAACCATAAAATGATCATTACCCCCAT | [X]VDTTIK (SEQ ID NO: 5545) *SLPP (SEQ ID NO: |
| | AAATTGATAATTATCCCACCCC | 5556) *IDNYPTP(SEQ ID NO: 5799) |
| 403 | TGTTGATACAACCATAAAAGGATCATTACCCCCAT | [X]VDTTIKGSLPP (SEQ ID NO: 5557) |
| | AAATTGATAATTATCCCACCCC | *IDNYPTP(SEQ ID NO: 5799) |
| 404 | TGTTGATACAACCATAAAATCATCATTACCCCCAT | [X]VDTTIKSSLPP (SEQ ID NO: 5558) |
| | AAATTGATAATTATCCCACCCC | *IDNYPTP(SEQ ID NO: 5799) |
| 405 | TGTTGATACAACCATAAAATGATAATTACCCCCAT | [X]VDTTIK (SEQ ID NO: 5545) **LPPSIDNYPTP |
| | CAATTGATAATTATCCCACCCC | (SEQ ID NO: 5804) |
| 406 | TGTTGATACAACCATAAAAGGATAATTACCCCCAT | [X]VDTTIKG (SEQ ID NO: 5547) |
| | CAATTGATAATTATOCCACCCC | *LPPSIDNYPTP(SEQ ID NO: 5804) |
| 407 | TGTTGATACAACCATAAAATCATAATTACCCCCAT | [X]VDTTIKS (SEQ ID NO: 5548) |
| | CAATTGATAATTATCCCACCCC | *LPPSIDNYPTP(SEQ ID NO: 5804) |
| 408 | TGTTGATACAACCATAAAATGATCATTACCCCCAT | [X]VDTTIK (SEQ ID NO: 5545) |
| | CAATTGATAATTATCCCACCCC | *SLPPSIDNYPTP(SEQ ID NO: 5805) |
| 409 | TGTTGATACAACCATAAAAGGATCATTACCCCCAT | [X]VDTTIKGSLPPSIDNYPTP(SEQ ID NO: 5806) |
| | CAATTGATAATTATCCCACCCC | |
| 410 | TGTTGATACAACCATAAAATCATCATTACCCCCAT | [X]VDTTIKSSLPPSIDNYPTP (SEQ ID NO: 5807) |
| | CAATTGATAATTATCCCACCCC | |
| 411 | TGTTGATACAACCATAAAATGATAATTACACCCAT | [X]VDTTIK (SEQ ID NO: 5545) |
| | AAATTGATAATTCTCACACCCA | **LHP*IDNSHT[Q/H](SEQ ID NO: 5837) |
| 316 | TGTTGATACAACCATAAAAGGATAATTACACCCAT | [X]VDTTIKG (SEQ ID NO: 5547) |
| | AAATTGATAATTATCACACCCA | *LHP*IDNYHT[Q/H] (SEQ ID NO: 5546) |
| 283 | TGTTGATACAACCATAAAATCATAATTACACCCAT | [X]VDTTIKS (SEQ ID NO: 5548) |
| | AAATTGATAATTATCACACCCA | *LHP*IDNYHT[Q/H] (SEQ ID NO: 5546) |
| 318 | TGTTGATACAACCATAAAATGATCATTACACCCAT | [X]VDTTIK (SEQ ID NO: 5545) *SLHP (SEQ ID NO: |
| | AAATTGATAATTATCACACCCA | 5549) *IDNYHT[Q/H] (SEQ ID NO: 5546) |
| 319 | TGTTGATACAACCATAAAAGGATCATTACACCCAT | [X]VDTTIKGSLHP (SEQ ID NO: 5550) |
| | AAATTGATAATTATCACACCCA | *IDNYHT[Q/H] (SEQ ID NO: 5546) |
| 320 | TGTTGATACAACCATAAAATCATCATTACACCCAT | [X]VDTTIKSSLHP (SEQ ID NO: 5551) |
| | AAATTGATAATTATCACACCCA | *IDNYHT[Q/H] (SEQ ID NO: 5546) |
| 321 | TGTTGATACAACCATAAAATGATAATTACACCCAT | [X]VDTTIK (SEQ ID NO: 5545) |
| | CAATTGATAATTATCACACCCA | **LHPSIDNYHT[Q/H] (SEQ ID NO: 5552) |
| 322 | TGTTGATACAACCATAAAAGGATAATTACACCCAT | [X]VDTTIKG (SEQ ID NO: 5547) |
| | CAATTGATAATTATCACACCCA | *LHPSIDNYHT[Q/H] (SEQ ID NO: 5552) |
| 323 | TGTTGATACAACCATAAAATCATAATTACACCCAT | [X]VDTTIKS (SEQ ID NO: 5548) |
| | CAATTGATAATTATCACACCCA | *LHPSIDNYHT[Q/H] (SEQ ID NO: 5552) |
| 324 | TGTTGATACAACCATAAAATGATCATTACACCCAT | [X]VDTTIK (SEQ ID NO: 5545) |
| | CAATTGATAATTATCACACCCA | *SLHPSIDNYHT[Q/H] (SEQ ID NO: 5553) |
| 325 | TGTTGATACAACCATAAAAGGATCATTACACCCAT | [X]VDTTIKGSLHPSIDNYHT[Q/H] (SEQ ID NO: |
| | CAATTGATAATTATCACACCCA | 5554) |
| 326 | TGTTGATACAACCATAAAATCATCATTACACCCAT | [X]VDTTIKSSLHPSIDNYHT[Q/H] (SEQ ID NO: |
| | CAATTGATAATTATCACACCCA | 5555) |
| 327 | TGTTGATACAACCATAAAATGATAATTACCCCCAT | [X]VDTTIK (SEQ ID NO: 5545) |
| | AAATTGATAATTATCACACCCA | **LPP*IDNYHT[Q/H] (SEQ ID NO: 5546) |
| 328 | TGTTGATACAACCATAAAAGGATAATTACCCCCAT | [X]VDTTIKG (SEQ ID NO: 5547) |
| | AAATTGATAATTATCACACCCA | *LPP*IDNYHT[Q/H] (SEQ ID NO: 5546) |
| 329 | TGTTGATACAACCATAAAATCATAATTACCCCCAT | [X]VDTTIKS (SEQ ID NO: 5548) |
| | AAATTGATAATTATCACACCCA | *LPP*IDNYHT[Q/H] (SEQ ID NO: 5546) |
| 330 | TGTTGATACAACCATAAAATGATCATTACCCCCAT | [X]VDTTIK (SEQ ID NO: 5545) *SLPP (SEQ ID NO: |
| | AAATTGATAATTATCACACCCA | 5556) *IDNYHT[Q/H] (SEQ ID NO: 5546) |
| 331 | TGTTGATACAACCATAAAAGGATCATTACCCCCAT | [X]VDTTIKGSLPP (SEQ ID NO: 5557) |
| | AAATTGATAATTATCACACCCA | *IDNYHT[Q/H] (SEQ ID NO: 5546) |
| 332 | TGTTGATACAACCATAAAATCATCATTACCCCCAT | [X]VDTTIKSSLPP (SEQ ID NO: 5558) |
| | AAATTGATAATTATCACACCCA | *IDNYHT[Q/H] (SEQ ID NO: 5546) |
| 333 | TGTTGATACAACCATAAAATGATAATTACCCCCAT | [X]VDTTIK (SEQ ID NO: 5545) |
| | CAATTGATAATTATCACACCCA | **LPPSIDNYHT[Q/H] (SEQ ID NO: 5559) |
| 334 | TGTTGATACAACCATAAAAGGATAATTACCCCCAT | [X]VDTTIKG (SEQ ID NO: 5547) |
| | CAATTGATAATTATCACACCCA | *LPPSIDNYHT[Q/H] (SEQ ID NO: 5559) |
| 335 | TGTTGATACAACCATAAAATCATAATTACCCCCAT | [X]VDTTIKS (SEQ ID NO: 5548) |
| | CAATTGATAATTATCACACCCA | *LPPSIDNYHT[Q/H] (SEQ ID NO: 5559) |
| 336 | TGTTGATACAACCATAAAATGATCATTACCCCCAT | [X]VDTTIK (SEQ ID NO: 5545) |
| | CAATTGATAATTATCACACCCA | *SLPPSIDNYHT[Q/H] (SEQ ID NO: 5560) |
| 337 | TGTTGATACAACCATAAAAGGATCATTACCCCCAT | [X]VDTTIKGSLPPSIDNYHT[Q/H] (SEQ ID NO: |
| | CAATTGATAATTATCACACCCA | 5561) |
| 338 | TGTTGATACAACCATAAAATCATCATTACCCCCAT | [X]VDTTIKSSLPPSIDNYHT[Q/H] (SEQ ID NO: |
| | CAATTGATAATTATCACACCCA | 5562 |
| 339 | TGTTGATACAACCATAAAATGATAATTACACCCAT | [X]VDTTIK (SEQ ID NO: 5545) |
| | AAATTGATAATTATCCCACCCA | **LHP*IDNYPT[Q/H] (SEQ ID NO: 5563) |
| 340 | TGTTGATACAACCATAAAAGGATAATTACACCCAT | [X]VDTTIKG (SEQ ID NO: 5547) |
| | AAATTGATAATTATCCCACCCA | *LHP*IDNYPT[Q/H] (SEQ ID NO: 5563) |
| 341 | TGTTGATACAACCATAAAATCATAATTACACCCAT | [X]VDTTIKS (SEQ ID NO: 5548) |
| | AAATTGATAATTATCCCACCCA | *LHP*IDNYPT[Q/H] (SEQ ID NO: 5563) |
| 342 | TGTTGATACAACCATAAAATGATCATTACACCCAT | [X]VDTTIK (SEQ ID NO: 5545) *SLHP (SEQ ID NO: |
| | AAATTGATAATTATCCCACCCA | 5549) *IDNYPT[Q/H] (SEQ ID NO: 5563) |
| 343 | TGTTGATACAACCATAAAAGGATCATTACACCCAT | [X]VDTTIKGSLHP (SEQ ID NO: 5550) |
| | AAATTGATAATTATCCCACCCA | *IDNYPT[Q/H] (SEQ ID NO: 5563) |
| 344 | TGTTGATACAACCATAAAATCATCATTACACCCAT | [X]VDTTIKSSLHP (SEQ ID NO: 5551) |
| | AAATTGATAATTATCCCACCCA | *IDNYPT[Q/H] (SEQ ID NO: 5563) |
| 345 | TGTTGATACAACCATAAAATGATAATTACACCCAT | [X]VDTTIK (SEQ ID NO: 5545) |
| | CAATTGATAATTATCCCACCCA | **LHPSIDNYPT[Q/H] (SEQ ID NO: 5564) |
| 346 | TGTTGATACAACCATAAAAGGATAATTACACCCAT | [X]VDTTIKG (SEQ ID NO: 5547) |
| | CAATTGATAATTATCCCACCCA | *LHPSIDNYPT[Q/H] (SEQ ID NO: 5564) |
| 347 | TGTTGATACAACCATAAAATCATAATTACACCCAT | [X]VDTTIKS (SEQ ID NO: 5548) |
| | CAATTGATAATTATCCCACCCA | *LHPSIDNYPT[Q/H] (SEQ ID NO: 5564) |
| 348 | TGTTGATACAACCATAAAATGATCATTACACCCAT | [X]VDTTIK (SEQ ID NO: 5545) |
| | CAATTGATAATTATCCCACCCA | *SLHPSIDNYPT[Q/H] (SEQ ID NO: 5565) |
| 349 | TGTTGATACAACCATAAAAGGATCATTACACCCAT | [X]VDTTIKGSLHPSIDNYPT[Q/H] (SEQ ID NO: |
| | CAATTGATAATTATCCCACCCA | 5566) |
| 350 | TGTTGATACAACCATAAAATCATCATTACACCCAT | [X]VDTTIKSSLHPSIDNYPT[Q/H] (SEQ ID NO: |
| | CAATTGATAATTATCCCACCCA | 5567) |
| 351 | TGTTGATACAACCATAAAATGATAATTACACCCAT | [X]VDTTIK (SEQ ID NO: 5545) **LHP*IDNYHTP |
| | AAATTGATAATTATCACACCCC | (SEQ ID NO: 5486) |
| 352 | TGTTGATACAACCATAAAAGGATAATTACACCCAT | [X]VDTTIKG (SEQ ID NO: 5547) *LHP*IDNYHTP |
| | AAATTGATAATTATCACACCCC | (SEQ ID NO: 5486) |
| 353 | TGTTGATACAACCATAAAATCATAATTACACCCAT | [X]VDTTIKS (SEQ ID NO: 5548) *LHP*IDNYHTP |
| | AAATTGATAATTATCACACCCC | (SEQ ID NO: 5486) |
| 354 | TGTTGATACAACCATAAAATGATCATTACACCCAT | [X]VDTTIK (SEQ ID NO: 5545) *SLHP (SEQ ID NO: |
| | AAATTGATAATTATCACACCCC | 5549) *IDNYHTP (SEQ ID NO: 5486) |
| 355 | TGTTGATACAACCATAAAAGGATCATTACACCCAT | [X]VDTTIKGSLHP (SEQ ID NO: 5550) *IDNYHTP |
| | AAATTGATAATTATCACACCCC | (SEQ ID NO: 5486) |
| 356 | TGTTGATACAACCATAAAATCATCATTACACCCAT | [X]VDTTIKSSLHP (SEQ ID NO: 5551) *IDNYHTP |
| | AAATTGATAATTATCACACCCC | (SEQ ID NO: 5486) |
| 357 | TGTTGATACAACCATAAAATGATAATTACACCCAT | [X]VDTTIK (SEQ ID NO: 5545) **LHPSIDNYHTP |
| | CAATTGATAATTATCACACCCC | (SEQ ID NO: 5568) |
| 358 | TGTTGATACAACCATAAAAGGATAATTACACCCAT | [X]VDTTIKG (SEQ ID NO: 5547) *LHPSIDNYHTP |
| | CAATTGATAATTATCACACCCC | (SEQ ID NO: 5568) |
| 359 | TGTTGATACAACCATAAAATCATAATTACACCCAT | [X]VDTTIKS (SEQ ID NO: 5548) *LHPSIDNYHTP |
| | CAATTGATAATTATCACACCCC | (SEQ ID NO: 5568) |
| 360 | TGTTGATACAACCATAAAATGATCATTACACCCAT | [X]VDTTIK (SEQ ID NO: 5545) *SLHPSIDNYHTP |
| | CAATTGATAATTATCACACCCC | (SEQ ID NO: 5569) |
| 361 | TGTTGATACAACCATAAAAGGATCATTACACCCAT | [X]VDTTIKGSLHPSIDNYHTP (SEQ ID NO: 5570) |
| | CAATTGATAATTATCACACCCC | |
| 362 | TGTTGATACAACCATAAAATCATCATTACACCCAT | [X]VDTTIKSSLHPSIDNYHTP (SEQ ID NO: 5571) |
| | CAATTGATAATTATCACACCCC | |
| 363 | TGTTGATACAACCATAAAATGATAATTACCCCCAT | [X]VDTTIK (SEQ ID NO: 5545) |
| | AAATTGATAATTATCCCACCCA | **LPP*IDNYPT[Q/H] (SEQ ID NO: 5563) |
| 364 | TGTTGATACAACCATAAAAGGATAATTACCCCCAT | [X]VDTTIKG (SEQ ID NO: 5547) |
| | AAATTGATAATTATCCCACCCA | *LPP*IDNYPT[Q/H] (SEQ ID NO: 5563) |
| 365 | TGTTGATACAACCATAAAATCATAATTACCCCCAT | [X]VDTTIKS (SEQ ID NO: 5548) |
| | AAATTGATAATTATCCCACCCA | *LPP*IDNYPT[Q/H] (SEQ ID NO: 5563) |
| 366 | TGTTGATACAACCATAAAATGATCATTACCCCCAT | [X]VDTTIK (SEQ ID NO: 5545) *SLPP (SEQ ID NO: |
| | AAATTGATAATTATCCCACCCA | 5556) *IDNYPT[Q/H] (SEQ ID NO: 5563) |
| 367 | TGTTGATACAACCATAAAAGGATCATTACCCCCAT | [X]VDTTIKGSLPP (SEQ ID NO: 5557) |
| | AAATTGATAATTATCCCACCCA | *IDNYPT[Q/H] (SEQ ID NO: 5563) |
| 368 | TGTTGATACAACCATAAAATCATCATTACCCCCAT | [X]VDTTIKSSLPP (SEQ ID NO: 5558) |
| | AAATTGATAATTATCCCACCCA | *IDNYPT[Q/H] (SEQ ID NO: 5563) |
| 369 | TGTTGATACAACCATAAAATGATAATTACCCCCAT | [X]VDTTIK (SEQ ID NO: 5545) |
| | CAATTGATAATTATCCCACCCA | **LPPSIDNYPT[Q/H] (SEQ ID NO: 5572) |
| 370 | TGTTGATACAACCATAAAAGGATAATTACCCCCAT | [X]VDTTIKG (SEQ ID NO: 5547) |
| | CAATTGATAATTATCCCACCCA | *LPPSIDNYPT[Q/H] (SEQ ID NO: 5572) |
| 371 | TGTTGATACAACCATAAAATCATAATTACCCCCAT | [X]VDTTIKS (SEQ ID NO: 5548) |
| | CAATTGATAATTATCCCACCCA | *LPPSIDNYPT[Q/H] (SEQ ID NO: 5572) |
| 372 | TGTTGATACAACCATAAAATGATCATTACCCCCAT | [X]VDTTIK (SEQ ID NO: 5545) |
| | CAATTGATAATTATCCCACCCA | *SLPPSIDNYPT[Q/H] (SEQ ID NO: 5792) |
| 373 | TGTTGATACAACCATAAAAGGATCATTACCCCCAT | [X]VDTTIKGSLPPSIDNYPT[Q/H] (SEQ ID NO: |
| | CAATTGATAATTATCCCACCCA | 5793) |
| 374 | TGTTGATACAACCATAAAATCATCATTACCCCCAT | [X]VDTTIKSSLPPSIDNYPT[Q/H] (SEQ ID NO: |
| | CAATTGATAATTATCCCACCCA | 5794) |
| 375 | TGTTGATACAACCATAAAATGATAATTACCCCCAT | [X]VDTTIK (SEQ ID NO: 5545) **LPP*IDNYHTP |
| | AAATTGATAATTATCACACCCC | (SEQ ID NO: 5486) |
| 376 | TGTTGATACAACCATAAAAGGATAATTACCCCCAT | [X]VDTTIKG (SEQ ID NO: 5547) *LPP*IDNYHTP |
| | AAATTGATAATTATCACACCCC | (SEQ ID NO: 5486) |
| 377 | TGTTGATACAACCATAAAATCATAATTACCCCCAT | [X]VDTTIKS (SEQ ID NO: 5548) *LPP*IDNYHTP |
| | AAATTGATAATTATCACACCCC | (SEQ ID NO: 5486) |
| 378 | TGTTGATACAACCATAAAATGATCATTACCCCCAT | [X]VDTTIK (SEQ ID NO: 5545) *SLPP (SEQ ID NO: |
| | AAATTGATAATTATCACACCCC | 5556) *IDNYHTP (SEQ ID NO: 5486) |
| 379 | TGTTGATACAACCATAAAAGGATCATTACCCCCAT | [X]VDTTIKGSLPP (SEQ ID NO: 5557) *IDNYHTP |
| | AAATTGATAATTATCACACCCC | (SEQ ID NO: 5486) |
| 380 | TGTTGATACAACCATAAAATCATCATTACCCCCAT | [X]VDTTIKSSLPP (SEQ ID NO: 5558) *IDNYHTP |
| | AAATTGATAATTATCACACCCC | (SEQ ID NO: 5486) |
| 381 | TGTTGATACAACCATAAAATGATAATTACCCCCAT | [X]VDTTIK (SEQ ID NO: 5545) |
| | CAATTGATAATTATCACACCCC | **LPPSIDNYHTP(SEQ ID NO: 5795) |
| 382 | TGTTGATACAACCATAAAAGGATAATTACCCCCAT | [X]VDTTIKG (SEQ ID NO: 5547) |
| | CAATTGATAATTATCACACCCC | *LPPSIDNYHTP(SEQ ID NO: 5795) |
| 383 | TGTTGATACAACCATAAAATCATAATTACCCCCAT | [X]VDTTIKS (SEQ ID NO: 5548) |
| | CAATTGATAATTATCACACCCC | *LPPSIDNYHTP(SEQ ID NO: 5795) |
| 384 | TGTTGATACAACCATAAAATGATCATTACCCCCAT | [X]VDTTIK (SEQ ID NO: 5545) |
| | CAATTGATAATTATCACACCCC | *SLPPSIDNYHTP(SEQ ID NO: 5796) |
| 385 | TGTTGATACAACCATAAAAGGATCATTACCCCCAT | [X]VDTTIKGSLPPSIDNYHTP(SEQ ID NO: 5797) |
| | CAATTGATAATTATCACACCCC | |
| 386 | TGTTGATACAACCATAAAATCATCATTACCCCCAT | [X]VDTTIKSSLPPSIDNYHTP(SEQ ID NO: 5798) |
| | CAATTGATAATTATCACACCCC | |
| 387 | TGTTGATACAACCATAAAATGATAATTACACCCAT | [X]VDTTIK (SEQ ID NO: 5545) |
| | AAATTGATAATTATCCCACCCC | **LHP*IDNYPTP(SEQ ID NO: 5799) |
| 388 | TGTTGATACAACCATAAAAGGATAATTACACCCAT | [X]VDTTIKG (SEQ ID NO: 5547) |
| | AAATTGATAATTATCCCACCCC | *LHP*IDNYPTP(SEQ ID NO: 5799) |
| 389 | TGTTGATACAACCATAAAATCATAATTACACCCAT | [X]VDTTIKS (SEQ ID NO: 5548) |
| | AAATTGATAATTATCCCACCCC | *LHP*IDNYPTP(SEQ ID NO: 5799) |
| 390 | TGTTGATACAACCATAAAATGATCATTACACCCAT | [X]VDTTIK (SEQ ID NO: 5545) *SLHP (SEQ ID NO: |
| | AAATTGATAATTATCCCACCCC | 5549) *IDNYPTP(SEQ ID NO: 5799) |
| 391 | TGTTGATACAACCATAAAAGGATCATTACACCCAT | [X]VDTTIKGSLHP (SEQ ID NO: 5550) |
| | AAATTGATAATTATCCCACCCC | *IDNYPTP(SEQ ID NO: 5799) |
| 392 | TGTTGATACAACCATAAAATCATCATTACACCCAT | [X]VDTTIKSSLHP (SEQ ID NO: 5551) |
| | AAATTGATAATTATCCCACCCC | *IDNYPTP(SEQ ID NO: 5799) |
| 393 | TGTTGATACAACCATAAAATGATAATTACACCCAT | [X]VDTTIK (SEQ ID NO: 5545) |
| | CAATTGATAATTATCCCACCCC | **LHPSIDNYPTP(SEQ ID NO: 5800) |
| 394 | TGTTGATACAACCATAAAAGGATAATTACACCCAT | [X]VDTTIKG (SEQ ID NO: 5547) |
| | CAATTGATAATTATOCCACCCC | *LHPSIDNYPTP(SEQ ID NO: 5800) |
| 395 | TGTTGATACAACCATAAAATCATAATTACACCCAT | [X]VDTTIKS (SEQ ID NO: 5548) |
| | CAATTGATAATTATCCCACCCC | *LHPSIDNYPTP(SEQ ID NO: 5800) |
| 396 | TGTTGATACAACCATAAAATGATCATTACACCCAT | [X]VDTTIK (SEQ ID NO: 5545) |
| | CAATTGATAATTATCCCACCCC | *SLHPSIDNYPTP(SEQ ID NO: 5801) |
| 397 | TGTTGATACAACCATAAAAGGATCATTACACCCAT | [X]VDTTIKGSLHPSIDNYPTP (SEQ ID NO: 5802) |
| | CAATTGATAATTATCCCACCCC | |
| 398 | TGTTGATACAACCATAAAATCATCATTACACCCAT | [X]VDTTIKSSLHPSIDNYPTP (SEQ ID NO: 5803) |
| | CAATTGATAATTATCCCACCCC | |
| 399 | TGTTGATACAACCATAAAATGATAATTACCCCCAT | [X]VDTTIK (SEQ ID NO: 5545) |
| | AAATTGATAATTATCCCACCCC | **LPP*IDNYPTP(SEQ ID NO: 5799) |
| 400 | TGTTGATACAACCATAAAAGGATAATTACCCCCAT | [X]VDTTIKG (SEQ ID NO: 5547) |
| | AAATTGATAATTATCCCACCCC | *LPP*IDNYPTP(SEQ ID NO: 5799) |
| 401 | TGTTGATACAACCATAAAATCATAATTACCCCCAT | [X]VDTTIKS (SEQ ID NO: 5548) |
| | AAATTGATAATTATCCCACCCC | *LPP*IDNYPTP(SEQ ID NO: 5799) |
| 402 | TGTTGATACAACCATAAAATGATCATTACCCCCAT | [X]VDTTIK (SEQ ID NO: 5545) *SLPP (SEQ ID NO: |
| | AAATTGATAATTATCCCACCCC | 5556) *IDNYPTP(SEQ ID NO: 5799) |
| 403 | TGTTGATACAACCATAAAAGGATCATTACCCCCAT | [X]VDTTIKGSLPP (SEQ ID NO: 5557) |
| | AAATTGATAATTATCCCACCCC | *IDNYPTP(SEQ ID NO: 5799) |
| 404 | TGTTGATACAACCATAAAATCATCATTACCCCCAT | [X]VDTTIKSSLPP (SEQ ID NO: 5558) |
| | AAATTGATAATTATCCCACCCC | *IDNYPTP(SEQ ID NO: 5799) |
| 405 | TGTTGATACAACCATAAAATGATAATTACCCCCAT | [X]VDTTIK (SEQ ID NO: 5545) |
| | CAATTGATAATTATCCCACCCC | ** LPPSIDNYPTP(SEQ ID NO: 5804) |
| 406 | TGTTGATACAACCATAAAAGGATAATTACCCCCAT | [X]VDTTIKG (SEQ ID NO: 5547) |
| | CAATTGATAATTATCCCACCCC | *LPPSIDNYPTP(SEQ ID NO: 5804) |
| 407 | TGTTGATACAACCATAAAATCATAATTACCCCCAT | [X]VDTTIKS (SEQ ID NO: 5548) |
| | CAATTGATAATTATCCCACCCC | *LPPSIDNYPTP(SEQ ID NO: 5804) |
| 408 | TGTTGATACAACCATAAAATGATCATTACCCCCAT | [X]VDTTIK (SEQ ID NO: 5545) |
| | CAATTGATAATTATCCCACCCC | *SLPPSIDNYPTP(SEQ ID NO: 5805) |
| 409 | TGTTGATACAACCATAAAAGGATCATTACCCCCAT | [X]VDTTIKGSLPPSIDNYPTP (SEQ ID NO: 5806) |
| | CAATTGATAATTATCCCACCCC | |
| 410 | TGTTGATACAACCATAAAATCATCATTACCCCCAT | [X]VDTTIKSSLPPSIDNYPTP(SEQ ID NO: 5807) |
| | CAATTGATAATTATCCCACCCC | |
| 507 | TGTTGATACAACCATAAAATGATAATTCACCCATA | [X]VDTTIK (SEQ ID NO: 5545) **FTHKLIIITPT |
| | AATTGATAATTATCACACCCAC | (SEQ ID NO: 5808) |
| 508 | TGTTGATACAACCATAAAAGGATAATTCACCCATA | [X]VDTTIKG (SEQ ID NO: 5547) *FTHKLIIITPT |
| | AATTGATAATTATCACACCCAC | (SEQ ID NO: 5808) |
| 509 | TGTTGATACAACCATAAAATCATAATTCACCCATA | [X]VDTTIKS (SEQ ID NO: 5548) *FTHKLIITPT |
| | AATTGATAATTATCACACCCAC | (SEQ ID NO: 5808) |
| 510 | TGTTGATACAACCATAAAATGATCATTCACCCATA | [X]VDTTIK (SEQ ID NO: 5545) *SFTHKLIIITPT |
| | AATTGATAATTATCACACCCAC | (SEQ ID NO: 5809) |
| 511 | TGTTGATACAACCATAAAAGGATCATTCACCCATA | [X]VDTTIKGSFTHKLIIITPT (SEQ ID NO: 5810) |
| | AATTGATAATTATCACACCCAC | |
| 512 | TGTTGATACAACCATAAAATCATCATTCACCCATA | [X]VDTTIKSSFTHKLIIITPT (SEQ ID NO: 5811) |
| | AATTGATAATTATCACACCCAC | |
| 513 | TGTTGATACAACCATAAAATGATAATTCACCCCTA | [X]VDTTIK (SEQ ID NO: 5545) **FTPKLIITPT |
| | AATTGATAATTATCACACCCAC | (SEQ ID NO: 5812) |
| 514 | TGTTGATACAACCATAAAAGGATAATTCACCCCTA | [X]VDTTIKG (SEQ ID NO: 5547) *FTPKLIITPT |
| | AATTGATAATTATCACACCCAC | (SEQ ID NO: 5812) |
| 515 | TGTTGATACAACCATAAAATCATAATTCACCCCTA | [X]VDTTIKS (SEQ ID NO: 5548) *FTPKLIIITPT |
| | AATTGATAATTATCACACCCAC | (SEQ ID NO: 5812) |
| 516 | TGTTGATACAACCATAAAATGATCATTCACCCCTA | [X]VDTTIK (SEQ ID NO: 5545) *SFTPKLIIITPT |
| | AATTGATAATTATCACACCCAC | (SEQ ID NO: 5813) |
| 517 | TGTTGATACAACCATAAAAGGATCATTCACCCCTA | [X]VDTTIKGSFTPKLIHITPT (SEQ ID NO: 5814) |
| | AATTGATAATTATCACACCCAC | |
| 518 | TGTTGATACAACCATAAAATCATCATTCACCCCTA | [X]VDTTIKSSFTPKLIIITPT (SEQ ID NO: 5815) |
| | AATTGATAATTATCACACCCAC | |
| 18 | TGTTGATACAACCATAAAATGATAATTACACCCAT | [X]VDTTIK (SEQ ID NO: 5545) |
| | AAATTGATAATTATCACACCCA | **LHP*IDNYHT[Q/H] (SEQ ID NO: 5546) |
| 520 | TGTTAATACAACCATAAAATGATAATTACACCCAT | [X]VNTTIK(SEQ ID NO: |
| | AAATTGATAATTATCACACCCA | 5816)**LHP*IDNYHT[Q/H] (SEQ ID NO: 5546) |
| 521 | TGTTGTTACAACCATAAAATGATAATTACACCCAT | [X]VVTTIK(SEQ ID NO: |
| | AAATTGATAATTATCACACCCA | 5817)**LHP*IDNYHT[Q/H] (SEQ ID NO: 5546) |
| 522 | TGTTGCTACAACCATAAAATGATAATTACACCCAT | [X]VATTIK(SEQ ID NO: |
| | AAATTGATAATTATCACACCCA | 5818)**LHP*IDNYHT[Q/H] (SEQ ID NO: 5546) |
| 523 | TGTTGGTACAACCATAAAATGATAATTACACCCAT | [X]VGTTIK(SEQ ID NO: |
| | AAATTGATAATTATCACACCCA | 5819)**LHP*IDNYHT[Q/H] (SEQ ID NO: 5546) |
| 277 | TGTTGATACAACCATACAATGATAATTACACCCAT | [X]VDTTIQ(SEQ ID NO: |
| | AAATTGATAATTATCACACCCA | 5820)**LHP*IDNYHT[Q/H] (SEQ ID NO: 5546) |
| 525 | TGTTGATACAACCATAATATGATAATTACACCCAT | [X]VDTTII(SEQ ID NO: 5821)**LHP*IDNYHT[Q/H] |
| | AAATTGATAATTATCACACCCA | (SEQ ID NO: 5546) |
| 526 | TGTTGATACAACCATAACATGATAATTACACCCAT | [X]VDTTIT(SEQ ID NO: 5822)**LHP*IDNYHT[Q/H] |
| | AAATTGATAATTATCACACCCA | (SEQ ID NO: 5546) |
| 278 | TGTTGATACAACCATAAATTGATAATTACACCCAT | [X]VDTTIN(SEQ ID NO: |
| | AAATTGATAATTATCACACCCA | 5823)**LHP*IDNYHT[Q/H] (SEQ ID NO: 5546) |
| 279 | TGTTGATACAACCATAAACTGATAATTACACCCAT | [X]VDTTIN**LHP*IDNYHT[Q/H] (SEQ ID NO: |
| | AAATTGATAATTATCACACCCA | 5546) |
| 316 | TGTTGATACAACCATAAAAGGATAATTACACCCAT | [X]VDTTIKG (SEQ ID NO: 5547) |
| | AAATTGATAATTATCACACCCA | *LHP*IDNYHT[Q/H] (SEQ ID NO: 5546) |
| 282 | TGTTGATACAACCATAAAATTATAATTACACCCAT | [X]VDTTIKL(SEQ ID NO: |
| | AAATTGATAATTATCACACCCA | 5824)*LHP*IDNYHT[Q/H] (SEQ ID NO: 5546) |
| 283 | TGTTGATACAACCATAAAATCATAATTACACCCAT | [X]VDTTIKS (SEQ ID NO: 5548) |
| | AAATTGATAATTATCACACCCA | *LHP*IDNYHT[Q/H] (SEQ ID NO: 5546) |
| 532 | TGTTGATACAACCATAAAATGACAATTACACCCAT | [X]VDTTIK (SEQ ID NO: 5545) *QLHP(SEQ ID NO: |
| | AAATTGATAATTATCACACCCA | 5825)*IDNYHT[Q/H] (SEQ ID NO: 5546) |
| 533 | TGTTGATACAACCATAAAATGATTATTACACCCAT | [X]VDTTIK (SEQ ID NO: 5545) *LLHP(SEQ ID NO: |
| | AAATTGATAATTATCACACCCA | 5826)*IDNYHT[Q/H] (SEQ ID NO: 5546) |
| 318 | TGTTGATACAACCATAAAATGATCATTACACCCAT | [X]VDTTIK (SEQ ID NO: 5545) *SLHP (SEQ ID NO: |
| | AAATTGATAATTATCACACCCA | 5549) *IDNYHT[Q/H] (SEQ ID NO: 5546) |
| 535 | TGTTGATACAACCATAAAATGATAATTAAACCCAT | [X]VDTTIK (SEQ ID NO: 5545) |
| | AAATTGATAATTATCACACCCA | **LNP*IDNYHT[Q/H] (SEQ ID NO: 5546) |
| 536 | TGTTGATACAACCATAAAATGATAATTACTCCCAT | [X]VDTTIK (SEQ ID NO: 5545) |
| | AAATTGATAATTATCACACCCA | **LLP*IDNYHT[Q/H] (SEQ ID NO: 5546) |
| 327 | TGTTGATACAACCATAAAATGATAATTACCCCCAT | [X]VDTTIK (SEQ ID NO: 5545) |
| | AAATTGATAATTATCACACCCA | **LPP*IDNYHT[Q/H] (SEQ ID NO: 5546) |
| 538 | TGTTGATACAACCATAAAATGATAATTACAACCAT | [X]VDTTIK (SEQ ID NO: 5545) |
| | AAATTGATAATTATCACACCCA | **LQP*IDNYHT[Q/H] (SEQ ID NO: 5546) |
| 539 | TGTTGATACAACCATAAAATGATAATTACAGCCAT | [X]VDTTIK (SEQ ID NO: 5545) |
| | AAATTGATAATTATCACACCCA | **LQP*IDNYHT[Q/H] (SEQ ID NO: 5546) |
| 540 | TGTTGATACAACCATAAAATGATAATTACACCCAC | [X]VDTTIK (SEQ ID NO: 5545) |
| | AAATTGATAATTATCACACCCA | **LHPQIDNYHT[Q/H](SEQ ID NO: 5827) |
| 541 | TGTTGATACAACCATAAAATGATAATTACACCCAT | [X]VDTTIK (SEQ ID NO: 5545) |
| | TAATTGATAATTATCACACCCA | **LHPLIDNYHT[Q/H](SEQ ID NO: 5828) |
| 321 | TGTTGATACAACCATAAAATGATAATTACACCCAT | [X]VDTTIK (SEQ ID NO: 5545) |
| | CAATTGATAATTATCACACCCA | **LHPSIDNYHT[Q/H] (SEQ ID NO: 5552) |
| 543 | TGTTGATACAACCATAAAATGATAATTACACCCAT | [X]VDTTIK (SEQ ID NO: 5545) |
| | AAATTAATAATTATCACACCCA | **LHP*INNYHT[Q/H](SEQ ID NO: 5829) |
| 544 | TGTTGATACAACCATAAAATGATAATTACACCCAT | [X]VDTTIK (SEQ ID NO: 5545) |
| | AAATTGTTAATTATCACACCCA | **LHP*IVNYHT[Q/H](SEQ ID NO: 5830) |
| 545 | TGTTGATACAACCATAAAATGATAATTACACCCAT | [X]VDTTIK (SEQ ID NO: 5545) |
| | AAATTGCTAATTATCACACCCA | **LHP*IANYHT[Q/H](SEQ ID NO: 5831) |
| 546 | TGTTGATACAACCATAAAATGATAATTACACCCAT | [X]VDTTIK (SEQ ID NO: 5545) |
| | AAATTGGTAATTATCACACCCA | **LHP*IGNYHT[Q/H](SEQ ID NO: 5832) |
| 547 | TGTTGATACAACCATAAAATGATAATTACACCCAT | [X]VDTTIK (SEQ ID NO: 5545) |
| | AAATTGATATTTATCACACCCA | **LHP*IDIYHT[Q/H](SEQ ID NO: 5833) |
| 548 | TGTTGATACAACCATAAAATGATAATTACACCCAT | [X]VDTTIK (SEQ ID NO: 5545) |
| | AAATTGATACTTATCACACCCA | **LHP*IDTYHT[Q/H](SEQ ID NO: 5834) |
| 549 | TGTTGATACAACCATAAAATGATAATTACACCCAT | [X]VDTTIK (SEQ ID NO: 5545) |
| | AAATTGATAGTTATCACACCCA | **LHP*IDSYHT[Q/H](SEQ ID NO: 5835) |
| 550 | TGTTGATACAACCATAAAATGATAATTACACCCAT | [X]VDTTIK (SEQ ID NO: 5545) |
| | AAATTGATAATAATCACACCCA | **LHP*IDNNHT[Q/H](SEQ ID NO: 5836) |
| 411 | TGTTGATACAACCATAAAATGATAATTACACCCAT | [X]VDTTIK (SEQ ID NO: 5545) |
| | AAATTGATAATTCTCACACCCA | **LHP*IDNSHT[Q/H](SEQ ID NO: 5837) |
| 552 | TGTTGATACAACCATAAAATGATAATTACACCCAT | [X]VDTTIK (SEQ ID NO: 5545) |
| | AAATTGATAATTATAACACCCA | **LHP*IDNYNT[Q/H](SEQ ID NO: 5838) |
| 553 | TGTTGATACAACCATAAAATGATAATTACACCCAT | [X]VDTTIK (SEQ ID NO: 5545) |
| | AAATTGATAATTATCTCACCCA | **LHP*IDNYLT[Q/H](SEQ ID NO: 5839) |
| 339 | TGTTGATACAACCATAAAATGATAATTACACCCAT | [X]VDTTIK (SEQ ID NO: 5545) |
| | AAATTGATAATTATCCCACCCA | **LHP*IDNYPT[Q/H] (SEQ ID NO: 5563) |
| 294 | TGTTGATACAACCATAAAATGATAATTACACCCAT | [X]VDTTIK (SEQ ID NO: 5545) |
| | AAATTGATAATTATCAAACCCA | **LHP*IDNYQT[Q/H](SEQ ID NO: 5840) |
| 556 | TGTTGATACAACCATAAAATGATAATTACACCCAT | [X]VDTTIK (SEQ ID NO: 5545) |
| | AAATTGATAATTATCAGACCCA | **LHP*IDNYQT[Q/H](SEQ ID NO: 5840) |
| 557 | TGTTGATACAACCATAAAATGATTATTACACCCAT | [X]VDTTIK (SBQ ID NO: 5545) |
| | TAATTGATAATTATCACACCCA | *LLHPLIDNYHT[Q/H](SEQ ID NO: 5841) |
| 558 | TGTTGATACAACCATAAAAGGATTATTACACCCAT | [X]VDTTIKGLLHPLIDNYHT[Q/H] (SEQ ID NO: |
| | TAATTGATAATTATCACACCCA | 5842) |
| 559 | TGTTGATACAACCATAAAATTATTATTACACCCAT | [X]VDTTIKLLLHPLIDNYHT[Q/H] (SEQ ID NO: |
| | TAATTGATAATTATCACACCCA | 5843) |
| 560 | TGTTGATACAACCATAAAATCATTATTACACCCAT | [X]VDTTIKSLLHPLIDNYHT[Q/H] (SEQ ID NO: |
| | TAATTGATAATTATCACACCCA (551) | 5844) |

Frame 3

| | | |
|---|---|---|
| 18 | TGTTGATACAACCATAAAATGATAATTACACCCAT | [M/L/V]LIQP(SEQ ID NO: |
| | AAATTGATAATTATCACACCCA | 5724)*NDNYTHKLIIITP[X](SEQ ID NO: 5748) |
| 565 | TGTTGATACAACCATCAAATGATAATTACACCCAT | [M/L/V]LIQPSNDNYTHKLIIITP[X] (SEQ ID NO: |
| | AAATTGATAATTATCACACCCA | 5747) |
| 566 | TGTTGATACAACCATAAAATGATAATTACACCCCT | [M/L/V]LIQP(SEQ ID NO: |
| | AAATTGATAATTATCACACCCA | 5724)*NDNYTPKLIIITP[X] (SEQ ID NO: 5739) |
| 567 | TGTTGATACAACCATCAAATGATAATTACACCCCT | [M/L/V]LIQPSNDNYTPKLIIITP[X] (SEQ ID NO: |
| | AAATTGATAATTATCACACCCA | 5749) |
| 568 | TGTTGATACAACCATAAAATGATAATTCCACCCAT | [M/L/V]LIQP(SEQ ID NO: |
| | AAATTGATAATTATCACACCCA | 5724)*NDNSTHKLIIITP[X] (SEQ ID NO: 5736) |
| 569 | TGTTGATACAACCATCAAATGATAATTCCACCCAT | [M/L/V]LIQPSNDNSTHKLIIITP[X] (SEQ ID NO: |
| | AAATTGATAATTATCACACCCA | 5750) |
| 570 | TGTTGATACAACCATAAAATGATAATTCCACCCCT | [M/L/V]LIQP(SEQ ID NO: |
| | AAATTGATAATTATCACACCCA | 5724)*NDNSTPKLIIITP[X] (SEQ ID NO: 5751) |
| 571 | TGTTGATACAACCATCAAATGATAATTCCACCCCT | [M/L/V]LIQPSNDNSTPKLIIITP[X] (SEQ ID NO: |
| | AAATTGATAATTATCACACCCA | 5752) |
| 572 | TGTTGATACAACCATAAAATGGTAATTACACCCAT | [M/L/V]LIQP(SEQ ID NO: |
| | AAATTGATAATTATCACACCCA | 5724)*NGNYTHKLIIITP[X](SEQ ID NO: 5731) |
| 573 | TGTTGATACAACCATCAAATGGTAATTACACCCAT | [M/L/V]LIQPSNGNYTHKLIIITP[X] (SEQ ID NO: |
| | AAATTGATAATTATCACACCCA | 5358) |
| 574 | TGTTGATACAACCATAAAATGGTAATTACACCCCT | [M/L/V]LIQP(SEQ ID NO: |
| | AAATTGATAATTATCACACCCA | 5724)*NGNYTPKLIIITP[X] (SEQ ID NO: 5753) |
| 575 | TGTTGATACAACCATCAAATGGTAATTACACCCCT | [M/L/V]LIQPSNGNYTPKLIIITP[X] (SEQ ID NO: |
| | AAATTGATAATTATCACACCCA | 5754) |
| 576 | TGTTGATACAACCATAAAATGGTAATTCCACCCAT | [M/L/V]LIQP(SEQ ID NO: |
| | AAATTGATAATTATCACACCCA | 5724)*NGNSTHKLIIITP[X] (SEQ ID NO: 5755) |
| 577 | TGTTGATACAACCATCAAATGGTAATTCCACCCAT | [M/L/V]LIQPSNGNSTHKLIUITP[X] (SEQ ID NO: |
| | AAATTGATAATTATCACACCCA | 5756) |
| 578 | TGTTGATACAACCATAAAATGGTAATTCCACCCCT | [M/L/V]LIQP(SEQ ID NO: |
| | AAATTGATAATTATCACACCCA | 5724)*NGNSTPKLIIITP[X] (SEQ ID NO: 5757) |
| 579 | TGTTGATACAACCATCAAATGGTAATTCCACCCCT | [M/L/V]LIQPSNGNSTPKLIIITP[X] (SEQ ID NO: |
| | AAATTGATAATTATCACACCCA | 5758) |
| 580 | TGTTGATACAACCATAAAATGATAATTACACCCAT | [M/L/V]LIQP(SEQ ID NO: |
| | ACATTGATAATTATCACACCCA | 5724)*NDNYTHTLIIITP[X](SEQ ID NO: 5743) |
| 581 | TGTTGATACAACCATCAAATGATAATTACACCCAT | [M/L/V]LIQPSNDNYTHTLIIITP[X] (SEQ ID NO: |
| | ACATTGATAATTATCACACCCA | 5759) |
| 582 | TGTTGATACAACCATAAAATGATAATTACACCCCT | [M/L/V]LIQP(SEQ ID NO: |
| | ACATTGATAATTATCACACCCA | 5724)*NDNYTPTLIIITP[X] (SEQ ID NO: 5760) |
| 583 | TGTTGATACAACCATCAAATGATAATTACACCCCT | [M/L/V]LIQPSNDNYTPTLIIITP[X] (SEQ ID NO: |
| | ACATTGATAATTATCACACCCA | 5761) |
| 584 | TGTTGATACAACCATAAAATGATAATTCCACCCAT | [M/L/V]LIQP(SEQ ID NO: |
| | ACATTGATAATTATCACACCCA | 5724)*NDNSTHTLIIITP[X] (SEQ ID NO: 5762) |
| 585 | TGTTGATACAACCATCAAATGATAATTCCACCCAT | [M/L/V]LIQPSNDNSTHTLIITP[X] (SEQ ID NO: |
| | ACATTGATAATTATCACACCCA | 5763) |
| 586 | TGTTGATACAACCATAAAATGATAATTCCACCCCT | [M/L/V]LIQP(SEQ ID NO: |
| | ACATTGATAATTATCACACCCA | 5724)*NDNSTPTLIIITP[X] (SEQ ID NO: 5764) |
| 587 | TGTTGATACAACCATCAAATGATAATTCCACCCCT | [M/L/V]LIQPSNDNSTPTLIIITP[X] (SEQ ID NO: |
| | ACATTGATAATTATCACACCCA | 5765) |
| 588 | TGTTGATACAACCATAAAATGGTAATTACACCCAT | [M/L/V]LIQP(SEQ ID NO: |
| | ACATTGATAATTATCACACCCA | 5724)*NGNYTHTLIIITP[X] (SEQ ID NO: 5766) |
| 589 | TGTTGATACAACCATCAAATGGTAATTACACCCAT | [M/L/V]LIQPSNGNYTHTLIIITP[X] (SEQ ID NO: |
| | ACATTGATAATTATCACACCCA | 5767) |
| 590 | TGTTGATACAACCATAAAATGGTAATTACACCCCT | [M/L/V]LIQP(SEQ ID NO: |
| | ACATTGATAATTATCACACCCA | 5724)*NGNYTPTLIIITP[X] (SEQ ID NO: 5768) |
| 591 | TGTTGATACAACCATCAAATGGTAATTACACCCCT | [M/L/V]LIQPSNGNYTPTLIIITP[X] (SEQ ID NO: |
| | ACATTGATAATTATCACACCCA | 5769) |
| 592 | TGTTGATACAACCATAAAATGGTAATTCCACCCAT | [M/L/V]LIQP(SEQ ID NO: |
| | ACATTGATAATTATCACACCCA | 5724)*NGNSTHTLIIITP[X] (SEQ ID NO: 5770) |
| 593 | TGTTGATACAACCATCAAATGGTAATTCCACCCAT | [M/L/V]LIQPSNGNSTHTLIIITP[X] (SEQ ID NO: |
| | ACATTGATAATTATCACACCCA | 5771) |
| 594 | TGTTGATACAACCATAAAATGGTAATTCCACCCCT | [M/L/V]LIQP(SEQ ID NO: |
| | ACATTGATAATTATCACACCCA | 5724)*NGNSTPTLIIITP[X] (SEQ ID NO: 5772) |
| 595 | TGTTGATACAACCATCAAATGGTAATTCCACCCCT | [M/L/V]LIQPSNGNSTPTLIIITP[X] (SEQ ID NO: |
| | ACATTGATAATTATCACACCCA | 5773) |
| 596 | TGTTGATACCACCATAAAATGATAATTACACCCAT | [M/L/V]LIPP(SEQ ID NO: |
| | AAATTGATAATTATCACACCCA | 5774)*NDNYTHKLIIITP[X](SEQ ID NO: 5748) |
| 597 | TGTTGATACCACCATCAAATGATAATTACACCCAT | [M/L/V]LIPPSNDNYTHKLIIITP[X] (SEQ ID NO: |
| | AAATTGATAATTATCACACCCA | 5775) |
| 598 | TGTTGATACCACCATAAAATGATAATTACACCCCT | [M/L/V]LIPP(SEQ ID NO: |
| | AAATTGATAATTATCACACCCA | 5774)*NDNYTPKLIIITP[X](SEQ ID NO: 5739) |
| 599 | TGTTGATACCACCATCAAATGATAATTACACCCCT | [M/L/V]LIPPSNDNYTPKLIIITP[X] (SEQ ID NO: |
| | AAATTGATAATTATCACACCCA | 5776) |
| 600 | TGTTGATACCACCATAAAATGATAATTCCACCCAT | [M/L/V]LIPP(SEQ ID NO: |
| | AAATTGATAATTATCACACCCA | 5774)*NDNSTHKLIIITP[X](SEQ ID NO: 5736) |
| 601 | TGTTGATACCACCATCAAATGATAATTCCACCCAT | [M/L/V]LIPPSNDNSTHKLIIITP[X] (SEQ ID NO: |
| | AAATTGATAATTATCACACCCA | 5777) |
| 602 | TGTTGATACCACCATAAAATGATAATTCCACCCCT | [M/L/V]LIPP(SEQ ID NO: |
| | AAATTGATAATTATCACACCCA | 5774)*NDNSTPKLIIITP[X] (SEQ ID NO: 5751) |
| 603 | TGTTGATACCACCATCAAATGATAATTCCACCCCT | [M/L/V]LIPPSNDNSTPKLIIITP[X] (SEQ ID NO: |
| | AAATTGATAATTATCACACCCA | 5778) |
| 604 | TGTTGATACCACCATAAAATGGTAATTACACCCAT | [M/L/V]LIPP(SEQ ID NO: |
| | AAATTGATAATTATCACACCCA | 5774)*NGNYTHKLIIITP[X](SEQ ID NO: 5731) |
| 605 | TGTTGATACCACCATCAAATGGTAATTACACCCAT | [M/L/V]LIPPSNGNYTHKLIIITP[X] (SEQ ID NO: |
| | AAATTGATAATTATCACACCCA | 5779) |
| 606 | TGTTGATACCACCATAAAATGGTAATTACACCCCT | [M/L/V]LIPP(SEQ ID NO: |
| | AAATTGATAATTATCACACCCA | 5774)*NGNYTPKLIIITP[X] (SEQ ID NO: 5753) |
| 607 | TGTTGATACCACCATCAAATGGTAATTACACCCCT | [M/L/V]LIPPSNGNYTPKLIIITP[X] (SEQ ID NO: |
| | AAATTGATAATTATCACACCCA | 5780) |
| 608 | TGTTGATACCACCATAAAATGGTAATTCCACCCAT | [M/L/V]LIPP(SEQ ID NO: |
| | AAATTGATAATTATCACACCCA | 5774)*NGNSTHKLIIITP[X] (SEQ ID NO: 5755) |
| 609 | TGTTGATACCACCATCAAATGGTAATTCCACCCAT | [M/L/V]LIPPSNGNSTHKLIIITP[X] (SEQ ID NO: |
| | AAATTGATAATTATCACACCCA | 5781) |
| 610 | TGTTGATACCACCATAAAATGGTAATTCCACCCCT | [M/L/V]LIPP(SEQ ID NO: |
| | AAATTGATAATTATCACACCCA | 5774)*NGNSTPKLIIITP[X] |
| 611 | TGTTGATACCACCATCAAATGGTAATTCCACCCCT | [M/L/V]LIPPSNGNSTPKLIIITP[X] (SEQ ID NO: |
| | AAATTGATAATTATCACACCCA | 5782) |
| 612 | TGTTGATACCACCATAAAATGATAATTACACCCAT | [M/L/V]LIPP(SEQ ID NO: |
| | ACATTGATAATTATCACACCCA | 5774)*NDNYTHTLIIITP[X](SEQ ID NO: 5743) |
| 613 | TGTTGATACCACCATCAAATGATAATTACACCCAT | [M/L/V]LIPPSNDNYTHTLIIITP[X] (SEQ ID NO: |
| | ACATTGATAATTATCACACCCA | 5783) |
| 614 | TGTTGATACCACCATAAAATGATAATTACACCCCT | [M/L/V]LIPP(SEQ ID NO: |
| | ACATTGATAATTATCACACCCA | 5774)*NDNYTPTLIIITP[X] (SEQ ID NO: 5760) |
| 615 | TGTTGATACCACCATCAAATGATAATTACACCCCT | [M/L/V]LIPPSNDNYTPTLIIITP[X] (SEQ ID NO: |
| | ACATTGATAATTATCACACCCA | 5784) |
| 616 | TGTTGATACCACCATAAAATGATAATTCCACCCAT | [M/L/V]LIPP(SEQ ID NO: |
| | ACATTGATAATTATCACACCCA | 5774)*NDNSTHTLIIITP[X] (SEQ ID NO: 5762) |
| 617 | TGTTGATACCACCATCAAATGATAATTCCACCCAT | [M/L/V]LIPPSNDNSTHTLIIITP[X] (SEQ ID NO: |
| | ACATTGATAATTATCACACCCA | 5785) |
| 618 | TGTTGATACCACCATAAAATGATAATTCCACCCCT | [M/L/V]LIPP(SEQ ID NO: 5774)*NDNSTPTLIIITP[X] |
| | ACATTGATAATTATCACACCCA | (SEQ ID NO: 5764) |
| 619 | TGTTGATACCACCATCAAATGATAATTCCACCCCT | [M/L/V]LIPPSNDNSTPTLIIITP[X] (SEQ ID NO: |
| | ACATTGATAATTATCACACCCA | 5786) |
| 620 | TGTTGATACCACCATAAAATGGTAATTACACCCAT | [M/L/V]LIPP(SEQ ID NO: |
| | ACATTGATAATTATCACACCCA | 5774)*NGNYTHTLIIITP[X] (SEQ ID NO: 5766) |
| 621 | TGTTGATACCACCATCAAATGGTAATTACACCCAT | [M/L/V]LIPPSNGNYTHTLIIITP[X] (SEQ ID NO: |
| | ACATTGATAATTATCACACCCA | 5787) |
| 622 | TGTTGATACCACCATAAAATGGTAATTACACCCCT | [M/L/V]LIPP(SEQ ID NO: |
| | ACATTGATAATTATCACACCCA | 5774)*NGNYTPTLIIITP[X] (SEQ ID NO: 5768) |
| 623 | TGTTGATACCACCATCAAATGGTAATTACACCCCT | [M/L/V]LIPPSNGNYTPTLIIITP[X] (SEQ ID NO: |
| | ACATTGATAATTATCACACCCA | 5788) |
| 624 | TGTTGATACCACCATAAAATGGTAATTCCACCCAT | [M/L/V]LIPP(SEQ ID NO: |
| | ACATTGATAATTATCACACCCA | 5774)*NGNSTHTLIIITP[X] (SEQ ID NO: 5770) |
| 625 | TGTTGATACCACCATCAAATGGTAATTCCACCCAT | [M/L/V]LIPPSNGNSTHTLIIITP[X] (SEQ ID NO: |
| | ACATTGATAATTATCACACCCA | 5789) |
| 626 | TGTTGATACCACCATAAAATGGTAATTCCACCCCT | [M/L/V]LIPP(SEQ ID NO: 5774)*NGNSTPTLIIITP[X] |
| | ACATTGATAATTATCACACCCA | (SEQ ID NO: 5772) |
| 627 | TGTTGATACCACCATCAAATGGTAATTCCACCCCT | [M/L/V]LIPPSNGNSTPTLIIITP[X] (SEQ ID NO: |
| | ACATTGATAATTATCACACCCA | 5790) |
| 18 | TGTTGATACAACCATAAAATGATAATTACACCCAT | [M/L/V]LIQP(SEQ ID NO: |
| | AAATTGATAATTATCACACCCA | 5724)*NDNYTHKLIIITP[X](SEQ ID NO: 5748) |
| 629 | TGTTGATACTACCATAAAATGATAATTACACCCAT | [M/L/V]LILP(SEQ ID NO: |
| | AAATTGATAATTATCACACCCA | 5791)*NDNYTHKLIIITP[X](SEQ ID NO: 5748) |
| 596 | TGTTGATACCACCATAAAATGATAATTACACCCAT | [M/L/V]LIPP(SEQ ID NO: |
| | AAATTGATAATTATCACACCCA | 5774)*NDNYTHKLIIITP[X](SEQ ID NO: 5748) |
| 631 | TGTTGATACAACCACAAAATGATAATTACACCCAT | [M/L/V]LIQPQNDNYTHKLIIITP[X] (SEQ ID NO: |
| | AAATTGATAATTATCACACCCA | 5745) |
| 632 | TGTTGATACAACCATTAAATGATAATTACACCCAT | [M/L/V]LIQPLNDNYTHKLIIITP[X] (SEQ ID NO: |
| | AAATTGATAATTATCACACCCA | 5746) |
| 565 | TGTTGATACAACCATCAAATGATAATTACACCCAT | [M/L/V]LIQPSNDNYTHKLIIITP[X] (SEQ ID NO: |
| | AAATTGATAATTATCACACCCA | 5747) |
| 278 | TGTTGATACAACCATAAATTGATAATTACACCCAT | [M/L/V]LIQP(SEQ ID NO: |
| | AAATTGATAATTATCACACCCA | 5724)*IDNYTHKLIIITP[X](SEQ ID NO: 5725) |
| 279 | TGTTGATACAACCATAAACTGATAATTACACCCAT | [M/L/V]LIQP(SEQ ID NO: |
| | AAATTGATAATTATCACACCCA | 5724)*TDNYTHKLIIITP[X](SEQ ID NO: 5726) |
| 280 | TGTTGATACAACCATAAAGTGATAATTACACCCAT | [M/L/V]LIQP(SEQ ID NO: |
| | AAATTGATAATTATCACACCCA | 5724)*SDNYTHKLIIITP[X](SEQ ID NO: 5727) |
| 284 | TGTTGATACAACCATAAAATAATAATTACACCCAT | [M/L/V]LIQP(SEQ ID NO: |
| | AAATTGATAATTATCACACCCA | 5724)*NNNYTHKLIIITP[X](SEQ ID NO: 5728) |
| 638 | TGTTGATACAACCATAAAATGTTAATTACACCCAT | [M/L/V]LIQP(SEQ ID NO: |
| | AAATTGATAATTATCACACCCA | 5724)*NVNYTHKLIIITP[X](SEQ ID NO: 5729) |
| 639 | TGTTGATACAACCATAAAATGCTAATTACACCCAT | [M/L/V]LIQP(SEQ ID NO: |
| | AAATTGATAATTATCACACCCA | 5724)*NANYTHKLIIITP[X](SEQ ID NO: 5730) |
| 572 | TGTTGATACAACCATAAAATGGTAATTACACCCAT | [M/L/V]LIQP(SEQ ID NO: |
| | AAATTGATAATTATCACACCCA | 5724)*NGNYTHKLIIITP[X](SEQ ID NO: 5731) |
| 641 | TGTTGATACAACCATAAAATGATATTTACACCCAT | [M/L/V]LIQP(SEQ ID NO: |
| | AAATTGATAATTATCACACCCA | 5724)*NDIYTHKLIIITP[X](SEQ ID NO: 5732) |
| 642 | TGTTGATACAACCATAAAATGATACTTACACCCAT | [M/L/V]LIQP(SEQ ID NO: |
| | AAATTGATAATTATCACACCCA | 5724)*NDTYTHKLIIITP[X](SEQ ID NO: 5733) |
| 643 | TGTTGATACAACCATAAAATGATAGTTACACCCAT | [M/L/V]LIQP(SEQ ID NO: |
| | AAATTGATAATTATCACACCCA | 5724)*NDSYTHKLIIITP[X](SEQ ID NO: 5734) |
| 644 | TGTTGATACAACCATAAAATGATAATAACACCCAT | [M/L/V]LIQP(SEQ ID NO: |
| | AAATTGATAATTATCACACCCA | 5724)*NDNNTHKLIIITP[X](SEQ ID NO: 5735) |
| 568 | TGTTGATACAACCATAAAATGATAATTCCACCCAT | [M/L/V]LIQP(SEQ ID NO: |
| | AAATTGATAATTATCACACCCA | 5724)*NDNSTHKLIIITP[X](SEQ ID NO: 5736) |
| 646 | TGTTGATACAACCATAAAATGATAATTACACCAAT | [M/L/V]LIQP(SEQ ID NO: |
| | AAATTGATAATTATCACACCCA | 5724)*NDNYTNKLIIITP[X](SEQ ID NO: 5737) |
| 647 | TGTTGATACAACCATAAAATGATAATTACACCCTT | [M/L/V]LIQP(SEQ ID NO: |
| | AAATTGATAATTATCACACCCA | 5724)*NDNYTLKLIIITP[X](SEQ ID NO: 5738) |
| 566 | TGTTGATACAACCATAAAATGATAATTACACCCCT | [M/L/V]LIQP(SEQ ID NO: |
| | AAATTGATAATTATCACACCCA | 5724)*NDNYTPKLIIITP[X](SEQ ID NO: 5739) |
| 649 | TGTTGATACAACCATAAAATGATAATTACACCCAA | [M/L/V]LIQP(SEQ ID NO: |
| | AAATTGATAATTATCACACCCA | 5724)*NDNYTQKLIIITP[X](SEQ ID NO: 5740) |
| 650 | TGTTGATACAACCATAAAATGATAATTACACCCAG | [M/L/V]LIQP(SEQ ID NO: |
| | AAATTGATAATTATCACACCCA | 5724)*NDNYTQKLIIITP[X](SEQ ID NO: 5740) |
| 321 | TGTTGATACAACCATAAAATGATAATTACACCCAT | [M/L/V]LIQP(SEQ ID NO: |
| | CAATTGATAATTATCACACCCA | 5724)*NDNYTHQLIIITP[X](SEQ ID NO: 5741) |
| 652 | TGTTGATACAACCATAAAATGATAATTACACCCAT | [M/L/V]LIQP(SEQ ID NO: |
| | ATATTGATAATTATCACACCCA | 5724)*NDNYTHILIIITP[X](SEQ ID NO: 5742) |
| 580 | TGTTGATACAACCATAAAATGATAATTACACCCAT | [M/L/V]LIQP(SEQ ID NO: |
| | ACATTGATAATTATCACACCCA | 5724)*NDNYTHTLIIITP[X](SEQ ID NO: 5743) |
| 285 | TGTTGATACAACCATAAAATGATAATTACACCCAT | [M/L/V]LIQP(SEQ ID NO: |
| | AATTTGATAATTATCACACCCA | 5724)*NDNYTHNLIIITP[X](SEQ ID NO: 5744) |
| 286 | TGTTGATACAACCATAAAATGATAATTACACCCAT | [M/L/V]LIQP(SEQ ID NO: |
| | AACTTGATAATTATCACACCCA | 5724)*NDNYTHNLIIITP[X](SEQ ID NO: 5744) |
| 656 | TGTTGATACAACCATTAAATGGTAATTACACCCAT | [M/L/V]LIQPLNGNYTHKLIIITP[X] (SEQ ID NO: |
| | AAATTGATAATTATCACACCCA | 5703) |
| 657 | TGTTGATACAACCATTAAATGATAATTCCACCCAT | [M/L/V]LIQPLNDNSTHKLIIITP[X] (SEQ ID NO: |
| | AAATTGATAATTATCACACCCA | 5704) |
| 658 | TGTTGATACAACCATTAAATGATAATTACACCCAA | [M/L/V]LIQPLNDNYTQKLIIITP[X] (SEQ ID NO: |
| | AAATTGATAATTATCACACCCA | 5705) |
| 659 | TGTTGATACAACCATTAAATGATAATTACACCCAT | [M/L/V]LIQPLNDNYTHILIIITP[X] (SEQ ID NO: |
| | ATATTGATAATTATCACACCCA | 5706) |
| 660 | TGTTGATACAACCATTAAATGATAATTACACCCAT | [M/L/V]LIQPLNDNYTHNLIIITP[X] (SEQ ID NO: |
| | AATTTGATAATTATCACACCCA | 5707) |
| 661 | TGTTGATACAACCATTAAATAATAATTCCACCCAT | [M/L/V]LIQPLNNNSTHKLIIITP[X] (SEQ ID NO: |
| | AAATTGATAATTATCACACCCA | 5708) |
| 662 | TGTTGATACAACCATTAAATGTTAATTCCACCCAT | [M/L/V]LIQPLNVNSTHKLIIITP[X] (SEQ ID NO: |
| | AAATTGATAATTATCACACCCA | 5709) |
| 663 | TGTTGATACAACCATTAAATGCTAATTCCACCCAT | [M/L/V]LIQPLNANSTHKLIIITP[X] (SEQ ID NO: |
| | AAATTGATAATTATCACACCCA | 5710) |
| 664 | TGTTGATACAACCATTAAATGGTAATTCCACCCAT | [M/L/V]LIQPLNGNSTHKLIIITP[X] (SEQ ID NO: |
| | AAATTGATAATTATCACACCCA | 5711) |
| 665 | TGTTGATACAACCATTAAATGATAATTCCACCAAT | [M/L/V]LIQPLNDNSTNKLIIITP[X] (SEQ ID NO: |
| | AAATTGATAATTATCACACCCA | 5712) |
| 666 | TGTTGATACAACCATTAAATGATAATTCCACCCTT | [M/L/V]LIQPLNDNSTLKLIIITP[X] (SEQ ID NO: |
| | AAATTGATAATTATCACACCCA | 5713) |
| 667 | TGTTGATACAACCATTAAATGATAATTCCACCCCT | [M/L/V]LIQPLNDNSTPKLIIITP[X] (SEQ ID NO: |
| | AAATTGATAATTATCACACCCA | 5714) |
| 668 | TGTTGATACAACCATTAAATGATAATTCCACCCAA | [M/L/V]LIQPLNDNSTQKLIIITP[X] (SEQ ID NO: |
| | AAATTGATAATTATCACACCCA | 5715) |
| 669 | TGTTGATACAACCATTAAATGATAATTCCACCCAG | [M/L/V]LIQPLNDNSTQKLIIITP[X] (SEQ ID NO: |
| | AAATTGATAATTATCACACCCA | 5715) |
| 670 | TGTTGATACAACCATTAAATGATAATTCCACCCAT | [M/L/V]LIQPLNDNSTHQLIIITP[X] (SEQ ID NO: |
| | CAATTGATAATTATCACACCCA | 5716) |
| 671 | TGTTGATACAACCATTAAATGATAATTCCACCCAT | [M/L/V]LIQPLNDNSTHILIIITP[X] (SEQ ID NO: |
| | ATATTGATAATTATCACACCCA | 5717) |
| 672 | TGTTGATACAACCATTAAATGATAATTCCACCCAT | [M/L/V]LIQPLNDNSTHTLIIITP[X] (SEQ ID NO: |
| | ACATTGATAATTATCACACCCA | 5718) |
| 673 | TGTTGATACAACCATTAAATGATAATTCCACCCAT | [M/L/V]LIQPLNDNSTHNLIIITP[X] (SEQ ID NO: |
| | AATTTGATAATTATCACACCCA | 5719) |
| 674 | TGTTGATACAACCATTAAATGATAATTCCACCCAT | [M/L/V]LIQPLNDNSTHNLIIITP[X] (SEQ ID NO: |
| | AACTTGATAATTATCACACCCA | 5719) |
| 664 | TGTTGATACAACCATTAAATGGTAATTCCACCCAT | [M/L/V]LIQPLNGNSTHKLIIITP[X] (SEQ ID NO: |
| | AAATTGATAATTATCACACCCA | 5711) |
| 668 | TGTTGATACAACCATTAAATGATAATTCCACCCAA | [M/L/V]LIQPLNDNSTQKLIIITP[X] (SEQ ID NO: |
| | AAATTGATAATTATCACACCCA | 5715) |
| 671 | TGTTGATACAACCATTAAATGATAATTCCACCCAT | [M/L/V]LIQPLNDNSTHILIIITP[X] (SEQ ID NO: |
| | ATATTGATAATTATCACACCCA | 5717) |
| 678 | TGTTGATACAACCATTAAATGGTAATTCCACCCAT | [M/L/V]LIQPLNGNSTHILIIITP[X] (SEQ ID NO: |
| | ATATTGATAATTATCACACCCA | 5720) |
| 679 | TGTTGATACAACCATTAAATGATAATTCCACCCAA | [M/L/V]LIQPLNDNSTQILIIITP[X] (SEQ ID NO: |
| | ATATTGATAATTATCACACCCA | 5721) |
| 680 | TGTTGATACAACCATTAAATGGTAATTCCACCCAA | [M/L/V]LIQPLNGNSTQKLIIITP[X] (SEQ ID NO: |
| | AAATTGATAATTATCACACCCA | 5722 |
| 681 | TGTTGATACAACCATTAAATGGTAATTCCACCCAA | [M/L/V]LIQPLNGNSTQILIIITP[X] (SEQ ID NO: |
| | ATATTGATAATTATCACACCCA | 5360) |

Frame 4

| | | |
|---|---|---|
| 682 | TGGGTGTGATAATTATCAATTTATGGGTGTAATTA | WV**LSIYGCNYHFMVVST(SEQ ID NO: 5678) |
| | TCATTTTATGGTTGTATCAACA | |
| 683 | GGGGTGTGATAATTATCAATTTATGGGTGTAATTA | GV**LSIYGCNYHFMVVST(SEQ ID NO: 5678) |
| | TCATTTTATGGTTGTATCAACA | |
| 684 | TTGGTGTGATAATTATCAATTTATGGGTGTAATTAT | LV**LSIYGCNYHFMVVST(SEQ ID NO: 5678) |
| | CATTTTATGGTTGTATCAACA | |
| 685 | TCGGTGTGATAATTATCAATTTATGGGTGTAATTA | SV**LSIYGCNYHFMVVST(SEQ ID NO: 5678) |
| | TCATTTTATGGTTGTATCAACA | |
| 686 | TGGGTGGGATAATTATCAATTTATGGGTGTAATTA | WVG*LSIYGCNYHEMVVST(SEQ ID NO: 5678) |
| | TCATTTTATGGTTGTATCAACA | |
| 687 | TGGGTGTTATAATTATCAATTTATGGGTGTAATTAT | WVL*LSIYGCNYHFMVVST(SEQ ID NO: 5678) |
| | CATTTTATGGTTGTATCAACA | |
| 688 | TGGGTGTCATAATTATCAATTTATGGGTGTAATTA | WVS*LSIYGCNYHFMVVST(SEQ ID NO: 5678) |
| | TCATTTTATGGTTGTATCAACA | |
| 689 | TGGGTGTGACAATTATCAATTTATGGGTGTAATTA | WV*QLSIYGCNYHFMVVST(SEQ ID NO: 5679) |
| | TCATTTTATGGTTGTATCAACA | |
| 690 | TGGGTGTGATTATTATCAATTTATGGGTGTAATTAT | WV*LLSIYGCNYHFMVVST(SEQ ID NO: 5680) |
| | CATTTTATGGTTGTATCAACA | |
| 691 | TGGGTGTGATCATTATCAATTTATGGGTGTAATTA | WV*SLSIYGCNYHFMVVST(SEQ ID NO: 5681) |
| | TCATTTTATGGTTGTATCAACA | |
| 692 | TGGGTGTGATAATTATCAATTAATGGGTGTAATTA | WV**LSINGCNYHFMVVST(SEQ ID NO: 5682) |
| | TCATTTTATGGTTGTATCAACA | |
| 693 | TGGGTGTGATAATTATCAATTTCTGGGTGTAATTA | WV**LSISGCNYHFMVVST (SEQ ID NO: 5683) |
| | TCATTTTATGGTTGTATCAACA | |
| 694 | TGGGTGTGATAATTATCAATTTATGGGAGTAATTA | WV**LSIYGSNYHFMVVST(SEQ ID NO: 5684) |
| | TCATTTTATGGTTGTATCAACA | |
| 695 | TGGGTGTGATAATTATCAATTTATGGGGGTAATTA | WV**LSIYGGNYHFMVVST(SEQ ID NO: 5685) |
| | TCATTTTATGGTTGTATCAACA | |
| 696 | TGGGTGTGATAATTATCAATTTATGGGTCTAATTA | WV**LSIYGSNYHFMVVST(SEQ ID NO: 5684) |
| | TCATTTTATGGTTGTATCAACA | |
| 697 | TGGGTGTGATAATTATCAATTTATGGGTGTATTTAT | WV**LSIYGCIYHFMVVST(SEQ ID NO: 5686) |
| | CATTTTATGGTTGTATCAACA | |
| 698 | TGGGTGTGATAATTATCAATTTATGGGTGTACTTA | WV**LSIYGCTYHFMVVST(SEQ ID NO: 5687) |
| | TCATTTTATGGTTGTATCAACA | |
| 699 | TGGGTGTGATAATTATCAATTTATGGGTGTAGTTA | WV**LSIYGCSYHFMVVST(SEQ ID NO: 5688) |
| | TCATTTTATGGTTGTATCAACA | |
| 700 | TGGGTGTGATAATTATCAATTTATGGGTGTAATAA | WV**LSIYGCNNHPMVVST(SEQ ID NO: 5689) |
| | TCATTTTATGGTTGTATCAACA | |
| 701 | TGGGTGTGATAATTATCAATTTATGGGTGTAATTC | WV**LSIYGCNSHFMVVST(SEQ ID NO: 5690) |
| | TCATTTTATGGTTGTATCAACA | |
| 702 | TGGGTGTGATAATTATCAATTTATGGGTGTAATTA | WV**LSIYGCNYNFMVVST(SEQ ID NO: 5691) |
| | TAATTTTATGGTTGTATCAACA | |
| 703 | TGGGTGTGATAATTATCAATTTATGGGTGTAATTA | WV**LSIYGCNYLFMVVST(SEQ ID NO: 5692) |
| | TCTTTTTATGGTTGTATCAACA | |
| 704 | TGGGTGTGATAATTATCAATTTATGGGTGTAATTA | WV**LSIYGCNYPFMVVST(SEQ ID NO: 5693) |
| | TCCTTTTATGGTTGTATCAACA | |
| 705 | TGGGTGTGATAATTATCAATTTATGGGTGTAATTA | WV**LSIYGCNYQFMVVST(SEQ ID NO: 5694) |
| | TCAATTTATGGTTGTATCAACA | |
| 706 | TGGGTGTGATAATTATCAATTTATGGGTGTAATTA | WV**LSIYGCNYQFMVVST(SEQ ID NO: 5694) |
| | TCAGTTTATGGTTGTATCAACA | |
| 707 | TGGGTGTGATAATTATCAATTTATGGGTGTAATTA | WV**LSIYGCNYHLMVVST(SEQ ID NO: 5695) |
| | TCATCTTATGGTTGTATCAACA | |
| 708 | TGGGTGTGATAATTATCAATTTATGGGTGTAATTA | WV**LSIYGCNYHIMVVST(SEQ ID NO: 5696) |
| | TCATATTATGGTTGTATCAACA | |
| 709 | TGGGTGTGATAATTATCAATTTATGGGTGTAATTA | WV**LSIYGCNYHVMVVST(SEQ ID NO: 5697) |
| | TCATGTTATGGTTGTATCAACA | |
| 710 | TGGGTGTGATAATTATCAATTTATGGGTGTAATTA | WV**LSIYGCNYHSMVVST(SEQ ID NO: 5698) |
| | TCATTCTATGGTTGTATCAACA | |
| 711 | TGGGTGTGATAATTATCAATTTATGGGTGTAATTA | WV**LSIYGCNYHLMVVST(SEQ ID NO: 5695) |
| | TCATTTAATGGTTGTATCAACA | |
| 712 | TGGGTGTGATAATTATCAATTTATGGGTGTAATTA | WV**LSIYGCNYHLMVVST(SEQ ID NO: 5695) |
| | TCATTTGATGGTTGTATCAACA | |
| 713 | TGGGTGTGATAATTATCAATTTATGGGTGTAATTA | WV**LSIYGCNYHFLVVST(SEQ ID NO: 5699) |
| | TCATTTTTTGGTTGTATCAACA | |
| 714 | TGGGTGTGATAATTATCAATTTATGGGTGTAATTA | WV**LSIYGCNYHFLVVST(SEQ ID NO: 5699) |
| | TCATTTTCTGGTTGTATCAACA | |
| 715 | TGGGTGTGATAATTATCAATTTATGGGTGTAATTA | WV**LSIYGCNYHFVVVST(SEQ ID NO: 5700) |
| | TCATTTTGTGGTTGTATCAACA | |
| 716 | TGGGTGTGATAATTATCAATTTATGGGTGTAATTA | WV**LSIYGCNYHFTVVST(SEQ ID NO: 5701) |
| | TCATTTTACGGTTGTATCAACA | |
| 717 | TGGGTGTGATAATTATCAATTTATGGGTGTAATTA | WV**LSIYGCNYHFIVVST(SEQ ID NO: 5702) |
| | TCATTTTATTGTTGTATCAACA | |
| 718 | TGGGTGTGATAATTATCAATTTATGGGTGTAATTA | WV**LSIYGCNYHFIVVST(SEQ ID NO: 5702) |
| | TCATTTTATCGTTGTATCAACA | |
| 719 | TGGGTGTGATAATTATCAATTTATGGGTGTAATTA | WV**LSIYGCNYHFIVVST(SEQ ID NO: 5702) |
| | TCATTTTATAGTTGTATCAACA | |
| 685 | TCGGTGTGATAATTATCAATTTATGGGTGTAATTA | SV**LSIYGCNYHFMVVST(SEQ ID NO: 5678) |
| | TCATTTTATGGTTGTATCAACA | |
| 721 | TCGGTGTCATAATTATCAATTTATGGGTGTAATTAT | SVS*LSIYGCNYHFMVVST(SEQ ID NO: 5678) |
| | CATTTTATGGTTGTATCAACA | |
| 722 | TCGGTGTCACAATTATCAATTTATGGGTGTAATTA | SVSQLSIYGCNYHFMVVST(SEQ ID NO: 5644) |
| | TCATTTTATGGTTGTATCAACA | |
| 723 | TCGGTGTCATTATTATCAATTTATGGGTGTAATTAT | SVSLLSIYGCNYHFMVVST (SEQ ID NO: 5645) |
| | CATTTTATGGTTGTATCAACA | |
| 724 | TCGGTGTCATCATTATCAATTTATGGGTGTAATTAT | SVSSLSIYGCNYHFMVVST(SEQ ID NO: 5646) |
| | CATTTTATGGTTGTATCAACA | |
| 725 | TCGGTGTCACAATTATCAATTTATGGGTGTAATTA | SVSQLSIYGCNYHFLVVST(SEQ ID NO: 5647) |
| | TCATTTTTTGGTTGTATCAACA | |
| 726 | TCGGTGTCATTATTATCAATTTATGGGTGTAATTAT | SVSLLSIYGCNYHFLVVST(SEQ ID NO: 5648) |
| | CATTTTTTGGTTGTATCAACA | |
| 727 | TCGGTGTCATCATTATCAATTTATGGGTGTAATTAT | SVSSLSIYGCNYHFLVVST(SEQ ID NO: 5649) |
| | CATTTTTTGGTTGTATCAACA | |
| 728 | TCGGTGTCACAATTATCAATTTATGGGTGTAATTA | SVSQLSIYGCNYHLMVVST(SEQ ID NO: 5650) |
| | TCATTTAATGGTTGTATCAACA | |
| 729 | TCGGTGTCATTATTATCAATTTATGGGTGTAATTAT | SVSLLSIYGCNYHLMVVST(SEQ ID NO: 5651) |
| | CATTTAATGGTTGTATCAACA | |
| 730 | TCGGTGTCATCATTATCAATTTATGGGTGTAATTAT | SVSSLSIYGCNYHLMVVST(SEQ ID NO: 5652) |
| | CATTTAATGGTTGTATCAACA | |
| 731 | TCGGTGTCACAATTATCAATTTATGGGTGTAATTA | SVSQLSIYGCNYLFMVVST(SEQ ID NO: 5653) |
| | TCTTTTTATGGTTGTATCAACA | |
| 732 | TCGGTGTCATTATTATCAATTTATGGGTGTAATTAT | SVSLLSIYGCNYLFMVVST(SEQ ID NO: 5654) |
| | CTTTTTATGGTTGTATCAACA | |
| 733 | TCGGTGTCATCATTATCAATTTATGGGTGTAATTAT | SVSSLSIYGCNYLFMVVST(SEQ ID NO: 5655) |
| | CTTTTTATGGTTGTATCAACA | |
| 734 | TCGGTGTCACAATTATCAATTTATGGGTGTAATAA | SVSQLSIYGCNNHFMVVST(SEQ ID NO: 5656) |
| | TCATTTTATGGTTGTATCAACA | |
| 735 | TCGGTGTCATTATTATCAATTTATGGGTGTAATAAT | SVSLLSIYGCNNHFMVVST(SEQ ID NO: 5657) |
| | CATTTTATGGTTGTATCAACA | |
| 736 | TCGGTGTCATCATTATCAATTTATGGGTGTAATAA | SVSSLSIYGCNNHFMVVST(SEQ ID NO: 5658) |
| | TCATTTTATGGTTGTATCAACA | |
| 737 | TCGGTGTCACAATTATCAATTTATGGGTGTATTTAT | SVSQLSIYGCIYHFMVVST(SEQ ID NO: 5659) |
| | CATTTTATGGTTGTATCAACA | |
| 738 | TCGGTGTCATTATTATCAATTTATGGGTGTATTTAT | SVSLLSIYGCIYHFMVVST(SEQ ID NO: 5660) |
| | CATTTTATGGTTGTATCAACA | |
| 739 | TCGGTGTCATCATTATCAATTTATGGGTGTATTTAT | SVSSLSIYGCIYHFMVVST(SEQ ID NO: 5661) |
| | CATTTTATGGTTGTATCAACA | |
| 740 | TCGGTGTCACAATTATCAATTTATGGGGGTAATTA | SVSQLSIYGGNYHFMVVST(SEQ ID NO: 5662) |
| | TCATTTTATGGTTGTATCAACA | |
| 741 | TCGGTGTCATTATTATCAATTTATGGGGGTAATTAT | SVSLLSIYGGNYHFMVVST(SEQ ID NO: 5663) |
| | CATTTTATGGTTGTATCAACA | |
| 742 | TCGGTGTCATCATTATCAATTTATGGGGGTAATTA | SVSSLSIYGGNYHFMVVST(SEQ ID NO: 5664) |
| | TCATTTTATGGTTGTATCAACA | |
| 743 | TCGGTGTCACAATTATCAATTAATGGGTGTAATTA | SVSQLSINGCNYHFMVVST(SEQ ID NO: 5665) |
| | TCATTTTATGGTTGTATCAACA | |
| 744 | TCGGTGTCATTATTATCAATTAATGGGTGTAATTAT | SVSLLSINGCNYHFMVVST(SEQ ID NO: 5666) |
| | CATTTTATGGTTGTATCAACA | |
| 745 | TCGGTGTCATCATTATCAATTAATGGGTGTAATTA | SVSSLSINGCNYHFMVVST (SEQ ID NO: 5667) |
| | TCATTTTATGGTTGTATCAACA | |
| 728 | TCGGTGTCACAATTATCAATTTATGGGTGTAATTA | SVSQLSIYGCNYHLMVVST(SEQ ID NO: 5650) |
| | TCATTTAATGGTTGTATCAACA | |
| 728 | TCGGTGTCATTATTATCAATTTATGGGTGTAATTAT | SVSLLSIYGCNYHLMVVST(SEQ ID NO: 5651) |
| | CATTTAATGGTTGTATCAACA | |
| 730 | TCGGTGTCATCATTATCAATTTATGGGTGTAATTAT | SVSSLSIYGCNYHLMVVST(SEQ ID NO: 5652) |
| | CATTTAATGGTTGTATCAACA | |
| 749 | TCGGTGTCACAATTATCAATTTATGGGTGTAATTA | SVSQLSIYGCNYHILLVVST(SEQ ID NO: 5668) |
| | TCATTTATTGGTTGTATCAACA | |
| 750 | TCGGTGTCATTATTATCAATTTATGGGTGTAATTAT | SVSLLSIYGCNYHLLVVST(SEQ ID NO: 5669) |
| | CATTTATTGGTTGTATCAACA | |
| 751 | TCGGTGTCATCATTATCAATTTATGGGTGTAATTAT | SVSSLSIYGCNYHLLVVST(SEQ ID NO: 5670) |
| | CATTTATTGGTTGTATCAACA | |
| 752 | TCGGTGTCACAATTATCAATTTATGGGTGTAATTA | SVSQLSIYGCNYLLLVVST(SEQ ID NO: 5671) |
| | TCTTTTATTGGTTGTATCAACA | |
| 753 | TCGGTGTCATTATTATCAATTTATGGGTGTAATAAT | SVSLLSIYGCNNHLLVVST(SEQ ID NO: 5672) |
| | CATTTATTGGTTGTATCAACA | |
| 754 | TCGGTGTCATCATTATCAATTTATGGGTGTATTTAT | SVSSLSIYGCIYHLLVVST(SEQ ID NO: 5673) |
| | CATTTATTGGTTGTATCAACA | |
| 755 | TCGGTGTCACAATTATCAATTTATGGGGGTAATTA | SVSQLSIYGGNYHLLVVST(SEQ ID NO: 5674) |
| | TCATTTATTGGTTGTATCAACA | |
| 756 | TCGGTGTCATTATTATCAATTAATGGGTGTAATTAT | SVSLLSINGCNYHLLVVST(SEQ ID NO: 5675) |
| | CATTTATTGGTTGTATCAACA | |
| 757 | TCGGTGTCATCATTATCAATTAATGGGGGTAATAA | SVSSLSINGGNNLLLVVST(SEQ ID NO: 5676) |
| | TCTTTTATTGGTTGTATCAACA | |
| 758 | TCGGTGTCACAATTATCAATTAATGGGGGTATTAA | SVSQLSINGGINLLLVVST(SEQ ID NO: 5677) |
| | TCTTTTATTGGTTGTATCAACA | |

Frame 5

| | | |
|---|---|---|
| 682 | TGGGTGTGATAATTATCAATTTATGGGTGTAATTA | [X]GCDNYQFMGVIIILWLYQ[Q/H] (SEQ ID NO: |
| | TCATTTTATGGTTGTATCAACA | 5613) |
| 760 | TGGGAGTGATAATTATCAATTTATGGGTGTAATTA | [X]GSDNYQFMGVIIILWLYQ[Q/H] (SEQ ID NO: |
| | TCATTTTATGGTTGTATCAACA | 5614) |
| 761 | TGGGGGTGATAATTATCAATTTATGGGTGTAATTA | [X]GGDNYQFMGVIIILWLYQ[Q/H] (SEQ ID NO: |
| | TCATTTTATGGTTGTATCAACA | 5615) |
| 762 | TGGGTCTGATAATTATCAATTTATGGGTGTAATTA | [X]GSDNYQFMGVIIILWLYQ[Q/H] (SEQ ID NO: |
| | TCATTTTATGGTTGTATCAACA | 5614) |
| 763 | TGGGTGTAATAATTATCAATTTATGGGTGTAATTA | [X]GCNNYQFMGVIIILWLYQ[Q/H] (SEQ ID NO: |
| | TCATTTTATGGTTGTATCAACA | 5616) |
| 764 | TGGGTGTGTTAATTATCAATTTATGGGTGTAATTAT | [X]GCVNYQFMGVIIILWLYQ[Q/H] (SEQ ID NO: |
| | CATTTTATGGTTGTATCAACA | 5617) |
| 765 | TGGGTGTGCTAATTATCAATTTATGGGTGTAATTA | [X]GCANYQFMGVIIILWLYQ[Q/H] (SEQ ID NO: |
| | TCATTTTATGGTTGTATCAACA | 5618) |
| 766 | TGGGTGTGGTAATTATCAATTTATGGGTGTAATTA | [X]GCGNYQFMGVIIILWLYQ[Q/H] (SEQ ID NO: |
| | TCATTTTATGGTTGTATCAACA | 5619) |
| 767 | TGGGTGTGATATTTATCAATTTATGGGTGTAATTAT | [X]GCDIYQFMGVIIILWLYQ[Q/H] (SEQ ID NO: |
| | CATTTTATGGTTGTATCAACA | 5620) |
| 768 | TGGGTGTGATACTTATCAATTTATGGGTGTAATTA | [X]GCDTYQFMGVIIILWLYQ[Q/H] (SEQ ID NO: |
| | TCATTTTATGGTTGTATCAACA | 5621) |
| 769 | TGGGTGTGATAGTTATCAATTTATGGGTGTAATTA | [X]GCDSYQFMGVIIILWLYQ[Q/H] (SEQ ID NO: |
| | TCATTTTATGGTTGTATCAACA | 5622) |
| 770 | TGGGTGTGATAATAATCAATTTATGGGTGTAATTA | [X]GCDNNQFMGVIIILWLYQ[Q/H] (SEQ ID NO: |
| | TCATTTTATGGTTGTATCAACA | 5623) |
| 771 | TGGGTGTGATAATTCTCAATTTATGGGTGTAATTA | [X]GCDNSQFMGVIIILWLYQ[Q/H] (SEQ ID NO: |
| | TCATTTTATGGTTGTATCAACA | 5624) |
| 772 | TGGGTGTGATAATTATCTATTTATGGGTGTAATTAT | [X]GCDNYLFMGVIIILWLYQ[Q/H] (SEQ ID NO: |
| | CATTTTATGGTTGTATCAACA | 5625} |
| 773 | TGGGTGTGATAATTATCCATTTATGGGTGTAATTA | [X]GCDNYPFMGVIIILWLYQ[Q/H] (SEQ ID NO: |
| | TCATTTTATGGTTGTATCAACA | 5626) |
| 774 | TGGGTGTGATAATTATCAACTTATGGGTGTAATTA | [X]GCDNYQLMGVIIILWLYQ[Q/H] (SEQ ID NO: |
| | TCATTTTATGGTTGTATCAACA | 5627) |
| 775 | TGGGTGTGATAATTATCAAATTATGGGTGTAATTA | [X]GCDNYQIMGVIIILWLYQ[Q/H] (SEQ ID NO: |
| | TCATTTTATGGTTGTATCAACA | 5628) |
| 776 | TGGGTGTGATAATTATCAAGTTATGGGTGTAATTA | [X]GCDNYQVMGVIIILWLYQ[Q/H] (SEQ ID NO: |
| | TCATTTTATGGTTGTATCAACA | 5629) |
| 777 | TGGGTGTGATAATTATCAATCTATGGGTGTAATTA | [X]GCDNYQSMGVIIILWLYQ[Q/H] (SEQ ID NO: |
| | TCATTTTATGGTTGTATCAACA | 5630) |
| 692 | TGGGTGTGATAATTATCAATTAATGGGTGTAATTA | [X]GCDNYQLMGVIIILWLYQ[Q/H] (SEQ ID NO: |
| | TCATTTTATGGTTGTATCAACA | 5627) |
| 779 | TGGGTGTGATAATTATCAATTGATGGGTGTAATTA | [X]GCDNYQLMGVIIILWLYQ[Q/H] (SEQ ID NO: |
| | TCATTTTATGGTTGTATCAACA | 5627) |
| 780 | TGGGTGTGATAATTATCAATTTTTGGGTGTAATTAT | [X]GCDNYQFLGVIIILWLYQ[Q/H] (SEQ ID NO: |
| | CATTTTATGGTTGTATCAACA | 5631) |
| 693 | TGGGTGTGATAATTATCAATTTCTGGGTGTAATTA | [X]GCDNYQFLGVIIILWLYQ[Q/H] (SEQ ID NO: |
| | TCATTTTATGGTTGTATCAACA | 5631) |
| 782 | TGGGTGTGATAATTATCAATTTGTGGGTGTAATTA | [X]GCDNYQFVGVIIILWLYQ[Q/H] (SEQ ID NO: |
| | TCATTTTATGGTTGTATCAACA | 5632) |
| 783 | TGGGTGTGATAATTATCAATTTACGGGTGTAATTA | [X]GCDNYQFTGVIIILWLYQ[Q/H] (SEQ ID NO: |
| | TCATTTTATGGTTGTATCAACA | 5633) |
| 784 | TGGGTGTGATAATTATCAATTTATTGGTGTAATTAT | [X]GCDNYQFIGVIIILWLYQ[Q/H] (SEQ ID NO: |
| | CATTTTATGGTTGTATCAACA | 5634) |
| 785 | TGGGTGTGATAATTATCAATTTATCGGTGTAATTA | [X]GCDNYQFIGVIIILWLYQ[Q/H] (SEQ ID NO: |
| | TCATTTTATGGTTGTATCAACA | 5634) |
| 786 | TGGGTGTGATAATTATCAATTTATAGGTGTAATTA | [X]GCDNYQFIGVIIILWLYQ[Q/H] (SEQ ID NO: |
| | TCATTTTATGGTTGTATCAACA | 5634) |
| 787 | TGGGTGTGATAATTATCAATTTATGGGTGTAATTA | [X]GCDNYQFMGVIIILGLYQ[Q/H] (SEQ ID NO: |
| | TCATTTTAGGGTTGTATCAACA | 5635) |
| 717 | TGGGTGTGATAATTATCAATTTATGGGTGTAATTA | [X]GCDNYQFMGVIIILLLYQ[Q/H] (SEQ ID NO: |
| | TCATTTTATTGTTGTATCAACA | 5636) |
| 789 | TGGGTGTGATAATTATCAATTTATGGGTGTAATTA | [X]GCDNYQFMGVIIILSLYQ[Q/H] (SEQ ID NO: |
| | TCATTTTATCGTTGTATCAACA | 5637) |
| 790 | TGGGTGTGATAATTATCAATTTATGGGTGTAATTA | [X]GCDNYQFMGVIIILWLNQ[Q/H] (SEQ ID NO: |
| | TCATTTTATGGTTGAATCAACA | 5638) |
| 791 | TGGGTGTGATAATTATCAATTTATGGGTGTAATTA | [X]GCDNYQFMGVIIILWLSQ[Q/H] (SEQ ID NO: |
| | TCATTTTATGGTTGTCTCAACA | 5639) |
| 792 | TGGGTGTGATAATTATCAATTTATGGGTGTAATTA | [X]GCDNYQFMGVIIILWLYL[Q/H] (SEQ ID NO: |
| | TCATTTTATGGTTGTATCTACA | 5640) |
| 793 | TGGGTGTGATAATTATCAATTTATGGGTGTAATTA | [X]GCDNYQFMGVIIILWLYP[Q/H] (SEQ ID NO: |
| | TCATTTTATGGTTGTATCCACA | 5641) |
| 760 | TGGGAGTGATAATTATCAATTTATGGGTGTAATTA | [X]GSDNYQFMGVIIILWLYQ[Q/H] (SEQ ID NO: |
| | TCATTTTATGGTTGTATCAACA | 5614) |
| 795 | TGGGAGTGGTAATTATCAATTTATGGGTGTAATTA | [X]GSGNYQFMGVIIILWLYQ[Q/H] (SEQ ID NO: |
| | TCATTTTATGGTTGTATCAACA | 5642) |
| 796 | TGGGAGTGGTAATTATCAAATTATGGGTGTAATTA | [X]GSGNYQIMGVIIILWLYQ[Q/H] (SEQ ID NO: |
| | TCATTTTATGGTTGTATCAACA | 5643) |

Frame 6

| | | |
|---|---|---|
| 682 | TGGGTGTGATAATTATCAATTTATGGGTGTAATTA | [M/L/V]GVIIINLWV (SEQ ID NO: |
| | TCATTTTATGGTTGTATCAACA | 5591)*LSFYGCIN[X](SEQ ID NO: 5592) |
| 798 | TGGGTGTGATAATTATCATTTTATGGGTGTAATTAT | [M/L/V]GVIIIILWV(SEQ ID NO: |
| | CATTTTATGGTTGTATCAACA | 5593)*LSFYGCIN[X](SEQ ID NO: 5592) |
| 799 | TGGGTGTGATAATTATCACTTTATGGGTGTAATTA | [M/L/V]GVIIITLWV(SEQ ID NO: |
| | TCATTTTATGGTTGTATCAACA | 5594)*LSFYGCIN[X](SEQ ID NO: 5592) |
| 800 | TGGGTGTGATAATTATCAGTTTATGGGTGTAATTA | [M/L/V]GVIIISLWV(SEQ ID NO: |
| | TCATTTTATGGTTGTATCAACA | 5595)*LSFYGCIN[X](SEQ ID NO: 5592) |
| 801 | TGGGTGTGATAATTATCAATTTAGGGGTGTAATTA | [M/L/V]GVIIINLGV(SEQ ID NO: |
| | TCATTTTATGGTTGTATCAACA | 5596)*LSFYGCIN[X](SEQ ID NO: 5592) |
| 784 | TGGGTGTGATAATTATCAATTTATTGGTGTAATTAT | [M/L/V]GVIIINLLV(SEQ ID NO: |
| | CATTTTATGGTTGTATCAACA | 5597)*LSFYGCIN[X](SEQ ID NO: 5592) |
| 785 | TGGGTGTGATAATTATCAATTTATCGGTGTAATTA | [M/L/V]GVIIINLSV(SEQ ID NO: |
| | TCATTTTATGGTTGTATCAACA | 5598)*LSFYGCIN[X](SEQ ID NO: 5592) |
| 804 | TGGGTGTGATAATTATCAATTTATGGGTGCAATTA | [M/L/V]GVIIINLWVQLSFYGCIN[X] (SEQ ID NO: |
| | TCATTTTATGGTTGTATCAACA | 5599) |
| 805 | TGGGTGTGATAATTATCAATTTATGGGTGTTATTAT | [M/L/V]GVIIINLWVLLSFYGCIN[X] (SEQ ID NO: |
| | CATTTTATGGTTGTATCAACA | 5600) |
| 806 | TGGGTGTGATAATTATCAATTTATGGGTGTCATTA | [M/L/V]GVIIINLWVSLSFYGCIN[X] (SEQ ID NO: |
| | TCATTTTATGGTTGTATCAACA | 5601) |
| 807 | TGGGTGTGATAATTATCAATTTATGGGTGTAATTA | [M/L/V]GVIIINLWV(SEQ ID NO: |
| | TCACTTTATGGTTGTATCAACA | 5591)*LSLYGCIN[X](SEQ ID NO: 5602) |
| 705 | TGGGTGTGATAATTATCAATTTATGGGTGTAATTA | [M/L/V]GVIIINLWV (SEQ ID NO: |
| | TCAATTTATGGTTGTATCAACA | 5591)*LSIYGCIN[X](SEQ ID NO: 5603) |
| 706 | TGGGTGTGATAATTATCAATTTATGGGTGTAATTA | [M/L/V]GVIIINLWV (SEQ ID NO: |
| | TCAGTTTATGGTTGTATCAACA | 5591)*LSVYGCIN[X](SEQ ID NO: 5604) |
| 707 | TGGGTGTGATAATTATCAATTTATGGGTGTAATTA | [M/L/V]GVIIINLWV (SEQ ID NO: |
| | TCATCTTATGGTTGTATCAACA | 5591)*LSSYGCIN[X](SEQ ID NO: 5605) |
| 811 | TGGGTGTGATAATTATCAATTTATGGGTGTAATTA | [M/L/V]GVIIINLWV(SEQ ID NO: |
| | TCATTATATGGTTGTATCAACA | 5591)*LSLYGCIN[X](SEQ ID NO: 5602) |
| 812 | TGGGTGTGATAATTATCAATTTATGGGTGTAATTA | [M/L/V]GVIIINLWV(SEQ ID NO: |
| | TCATTGTATGGTTGTATCAACA | 5591)*LSLYGCIN[X](SEQ ID NO: 5602) |
| 711 | TGGGTGTGATAATTATCAATTTATGGGTGTAATTA | [M/L/V]GVIIINLWV(SBQ ID NO: |
| | TCATTTAATGGTTGTATCAACA | 5591)*LSFNGCIN[X](SEQ ID NO: 5606) |
| 714 | TGGGTGTGATAATTATCAATTTATGGGTGTAATTA | [M/L/V]GVIIINLWV(SEQ ID NO: |
| | TCATTTTCTGGTTGTATCAACA | 5591)*LSFSGCIN[X](SEQ ID NO: 5607) |
| 815 | TGGGTGTGATAATTATCAATTTATGGGTGTAATTA | [M/L/V]GVIIINLWV(SEQ ID NO: |
| | TCATTTTATGGTAGTATCAACA | 5591)*LSFYGSIN[X] (SEQ ID NO: 5612) |
| 816 | TGGGTGTGATAATTATCAATTTATGGGTGTAATTA | [M/L/V]GVIIINLWV(SEQ ID NO: |
| | TCATTTTATGGTGGTATCAACA | 5591)*LSFYGGIN[X](SEQ ID NO: 5608) |
| 817 | TGGGTGTGATAATTATCAATTTATGGGTGTAATTA | [M/L/V]GVIIINLWV(SEQ ID NO: |
| | TCATTTTATGGTTCTATCAACA | 5591)*LSFYGSIN[X] (SEQ ID NO: 5612) |
| 818 | TGGGTGTGATAATTATCAATTTATGGGTGTAATTA | [M/L/V]GVIIINLWV (SEQ ID NO: |
| | TCATTTTATGGTTGTATCATCA | 5591)*LSFYGCII[X ](SEQ ID NO: 5609) |
| 819 | TGGGTGTGATAATTATCAATTTATGGGTGTAATTA | [M/L/V]GVIIINLWV (SEQ ID NO: |
| | TCATTTTATGGTTGTATCACCA | 5591)*LSFYGCIT[X](SEQ ID NO: 5610) |
| 820 | TGGGTGTGATAATTATCAATTTATGGGTGTAATTA | [M/L/V]GVIIINLWV(SEQ ID NO: |
| | TCATTTTATGGTTGTATCAGCA | 5591)*LSFYGCIS[X](SEQ ID NO: 5610) |
| 798 | TGGGTGTGATAATTATCATTTTATGGGTGTAATTAT | [M/L/V]GVIIIILWV(SEQ ID NO: |
| | CATTTTATGGTTGTATCAACA | 5593)*LSFYGCIN[X](SEQ ID NO: 5592) |
| 822 | TGGGTGTGATAATTATCATTTTATGGGTGTTATTAT | [M/L/V]GVIIIILWVLLSFYGCIN[X] (SEQ ID NO: |
| | CATTTTATGGTTGTATCAACA | 5575) |
| 823 | TGGGTGTGATAATTATCATTTTATGGGTGTTATTAT | [M/L/V]GVIIIILWVLLSLYGCIN[X] (SEQ ID NO: |
| | CATTATATGGTTGTATCAACA | 5576) |
| 824 | TGGGTGTGATAATTATCATTTTATGGGTGTTATTAT | [M/L/V]GVIIIILWVLLSLNGCIN[X] (SEQ ID NO: |
| | CATTAAATGGTTGTATCAACA | 5577) |
| 825 | TGGGTGTGATAATTATCATTTTATGGGTGTTATTAT | [M/L/V]GVIIIILWVLLSLYGSIN[X] (SEQ ID NO: |
| | CATTATATGGTAGTATCAACA | 5578) |
| 826 | TGGGTGTGATAATTATCATTTTATGGGTGTTATTAT | [M/L/V]GVIIIILWVLLSLYGSIN[X] (SEQ ID NO: |
| | CATTATATGGTTCTATCAACA | 5578) |
| 827 | TGGGTGTGATAATTATCATTTTATGGGTGTTATTAT | [M/L/V]GVIIIILWVLLSLYGCII[X] (SEQ ID NO: |
| | CATTATATGGTTGTATCATCA | 5579) |
| 828 | TGGGTGTGATAATTATCATTTTATGGGTGTTATTAT | [M/L/V]GVIIIILWVLLSLYGCIT[X] (SEQ ID NO: |
| | CATTATATGGTTGTATCACCA | 5580) |
| 829 | TGGGTGTGATAATTATCATTTTATGGGTGTTATTAT | [M/L/V]GVIIIILWVLLSLYGCIS[X] (SEQ ID NO: |
| | CATTATATGGTTGTATCAGCA | 5581) |
| 830 | TGGGTGTGATAATTATCATTTTAGGGGTGTTATTAT | [M/L/V]GVIIIILGVLLSLYGCIN[X] (SEQ ID NO: |
| | CATTATATGGTTGTATCAACA | 5582) |
| 831 | TGGGTGTGATAATTATCATTTTATTGGTGTTATTAT | [M/L/V]GVIIIILLVLLSLYGCIN[X] (SEQ ID NO: |
| | CATTATATGGTTGTATCAACA | 5583) |
| 832 | TGGGTGTGATAATTATCATTTTATCGGTGTTATTAT | [M/L/V]GVIIIILSVLLSLYGCIN[X] (SEQ ID NO: |
| | CATTATATGGTTGTATCAACA | 5584) |
| 833 | TGGGTGTGATAATTATCATTTTATGGGTGTTATTAT | [M/L/V]GVIIIILWVLLSLNGSIN[X] (SEQ ID NO: |
| | CATTAAATGGTAGTATCAACA | 5585) |
| 834 | TGGGTGTGATAATTATCATTTTATGGGTGTTATTAT | [M/L/V]GVIIIILWVLLSLNGSIN[X] (SEQ ID NO: |
| | CATTAAATGGTTCTATCAACA | 5585) |
| 835 | TGGGTGTGATAATTATCATTTTATGGGTGTTATTAT | [M/L/V]GVIIIILWVLLSLNGSII[X] (SEQ ID NO: |
| | CATTAAATGGTAGTATCATCA | 5586) |
| 836 | TGGGTGTGATAATTATCATTTTATGGGTGTTATTAT | [M/L/V]GVIIIILWVLLSLNGSII[X] (SEQ ID NO: |
| | CATTAAATGGTTCTATCATCA | 5586) |
| 837 | TGGGTGTGATAATTATCATTTTATGGGTGTTATTAT | [M/L/V]GVIIIILWVLLSLNGSIS[X] (SEQ ID NO: |
| | CATTAAATGGTAGTATCAGCA | 5587) |
| 838 | TGGGTGTGATAATTATCATTTTATGGGTGTTATTAT | [M/L/V]GVIIIILWVLLSLNGSIS[X] (SEQ ID NO: |
| | CATTAAATGGTTCTATCAGCA | 5587) |
| 839 | TGGGTGTGATAATTATCATTTTATGGGTGTTATTAT | [M/L/V]GVIIIILWVLLSLYGSIS[X] (SEQ ID NO: |
| | CATTATATGGTAGTATCAGCA | 5588) |
| 840 | TGGGTGTGATAATTATCATTTTATGGGTGTTATTAT | [M/L/V]GVIIIILWVLLSLYGSIS[X] (SEQ ID NO: |
| | CATTATATGGTTCTATCAGCA | 5588) |
| 841 | TGGGTGTGATAATTATCATTTTAGGGGTGTTATTAT | [M/L/V]GVIIIILGVLLSLYGSIS[X] (SEQ ID NO: |
| | CATTATATGGTAGTATCAGCA | 5589) |
| 842 | TGGGTGTGATAATTATCATTTTATTGGTGTTATTAT | [M/L/V]GVIIIILLVLLSLYGSIS[X] (SEQ ID NO: |
| | CATTATATGGTTCTATCAGCA | 5590) |
| 843 | TGGGTGTGATAATTATCATTTTAGGGGTGTTATTAT | [M/L/V]GVIIIILGVLLSLNGSIS[X] (SEQ ID NO: |
| | CATTAAATGGTAGTATCAGCA | 5574) |
| 844 | TGGGTGTGATAATTATCATTTTATTGGTGTTATTAT | [M/L/V]GVIIIILLVLLSLNGSIS[X] (SEQ ID NO: |
| | CATTAAATGGTTCTATCAGCA | 5573) |

TABLE 2
Selected engineered (right) transposon end variant sequences

| | | | 57-bp | |
|---|---|---|---|---|
| | | | transposon | |
| ID | Alias | Description | right end | Amino acid sequence |
| WT | WT_minimal_ | 57-bp | TGTTGATACAACC | C*YNHKMIITPIN**LSHP |
| | pDonor, | WT | ATAAAATGATAAT | (in frame 1) |
| | Linker v1 | TnR | TACACCCATAAAT | (SEQ ID NOs: |
| | | | TGATAATTATCAC | 5352-5353) |
| | | | ACCCA | |
| | | | (SEQ ID NO: 1) | |
| ORF1a | Linker v2 | TnR | TGTgGATACAACC | CGYNHKMITPINGSLSPP |
| | | variant | ATAAAATGATAAT | (SEQ ID NOs: 5354) |
| | | ORF1a | TACACCCATAAAT | |
| | | | gGATcATTATCAC | |
| | | | cCCCA | |
| | | | (SEQ ID NO: 2) | |
| ORF1b | Linker v3 | TnR | TGTgGATACAACC | CGYNHKTIITPINGSLSHP |
| | | variant | ATAAAAcGATAAT | (SEQ ID NOs: 5355) |
| | | ORF1b | TACACCCATAAAT | |
| | | | gGATcATTATCAC | |
| | | | ACCCA (SEQ ID | |
| | | | NO: 3) | |
| ORF1c | Linker v4 | TnR | TGTgGATcCAACC | CGSNHKMITPINGSLSHP |
| | | variant | ATAAAATGATAAT | (SEQ ID NOs: 5356) |
| | | ORF1c | TACACCCATAAAT | |
| | | | gGATcATTATCAC | |
| | | | ACCCA (SEQ ID | |
| | | | NO: 4) | |
| ORF2a | Linker v5 | TR | TGTTGATACAACC | [X]VDTTIKGLLHPLIDNYHT |
| | | variant | ATAAAAgGATtAT | [Q/H](SEQ ID NO: 5357) |
| | | ORF2a | TACACCCATIAAT | |
| | | | TGATAATTATCAC | |
| | | | ACCCA (SEQ ID | |
| | | | NO: 5) | |
| ORF3a | Linker v6 | TnR | TGTTGATACAACC | [M/L/V]LIQPSNGNYTHKLIIITP |
| | | variant | ATcAAATGgTAAT | [X](SEQ ID NO: 5358) |
| | | ORF3a | TACACCCATAAAT | |
| | | | TGATAATTATCAC | |
| | | | ACCCA (SEQ ID | |
| | | | NO: 6) | |
| ORF3b | Linker v7 | TnR | TGTTGATACAACC | [M/L/V]LIQPLNDNSTHNLIITP |
| | | variant | ATAAATGATAATT | [X](SEQ ID NO: 5359) |
| | | ORF3b | CCACCCATAAtTT | |
| | | | GATAATTATCACA | |
| | | | CCCA (SEQ ID | |
| | | | NO: 7) | |
| ORF3c | Linker v8 | TnR | TGTTGATACAACC | [M/L/V]LIQPLNGNSTQILIITP |
| | | variant | ATAAATGgTAATT | [X](SEQ ID NO: 5360) |
| | | ORF3c | CCACCCAaAtATT | |
| | | | GATAATTATCACA | |
| | | | CCCA | |
| | | | (SEQ ID NO: 8) | |

TABLE 3
IHF protein constructs

| | | SEQ ID |
|---|---|---|
| Protein name | Protein sequence | NO |
| hCO dcIHFA-NLS | MALTKAEMSEYLFDKLGLSKRDAKELVELFFEEIRRALEN | 5136 |
| | GEQVKLSGFGNFDLRDKNQRPGRNPKTGEDIPITARRVVT | |
| | FRPGQKLKSRVENASPKDEGSGKRTADGSEFESPKKKRKV | |
| | * | |
| hCO NLS-dcIHFA | MGKRTADGSEFESPKKKRKVGSGMALTKAEMSEYLFDKLG | 5137 |
| | LSKRDAKELVELFFEEIRRALENGEQVKLSGFGNFDLRDK | |
| | NQRPGRNPKTGEDIPITARRVVTFRPGQKLKSRVENASPK | |
| | DE* | |
| hCO dcIHFB-NLS | MGTKSELIERLATQQSHIPAKTVEDAVKEMLEHMASTLAQ | 5138 |
| | GERIEIRGFGSFSLHYRAPRTGRNPKTGDKVELEGKYVPH | |
| | FKPGKELRDRANIYGGSGKRTADGSEFESPKKKRKV* | |
| hCO NLS-dcIHFB | MGKRTADGSEFESPKKKRKVGSGMGTKSELIERLATQQSH | 5139 |
| | IPAKTVEDAVKEMLEHMASTLAQGERIEIRGFGSFSLHYR | |
| | APRTGRNPKTGDKVELEGKYVPHFKPGKELRDRANIYG* | |
| hCO NLS-scIHF2 | MGTKSELIERLATQQSHIPAKTVEDAVKEMLEHMASTLAQ | 5140 |
| | GGSGGLTKAEMSEYLFDKLGLSKRDAKELVELFFEEIRRA | |
| | LENGEQVKLSGFGNFDLRDKNQRPGRNPKTGEDIPITARR | |
| | VVTFRPGQKLKSRVENAGGGERIEIRGFGSFSLHYRAPRT | |
| | GRNPKTGDKVELEGKYVPHFKPGKELRDRANIYG* | |
| hCO scIHF2 | MGTKSELIERLATQQSHIPAKTVEDAVKEMLEHMASTLAQ | 5141 |
| | GGSGGLTKAEMSEYLFDKLGLSKRDAKELVELFFEEIRRA | |
| | LENGEQVKLSGFGNFDLRDKNQRPGRNPKTGEDIPITARR | |
| | VVTFRPGQKLKSRVENAGGGERIEIRGFGSFSLHYRAPRT | |
| | GRNPKTGDKVELEGKYVPHFKPGKELRDRANIYG* | |
| TnsA-NLS- | MYIRNLRKPSPNKNVFKFASTKVSSVVMCESSLEFDACFH | 5142 |
| GSGSGG-IHF- | HEYNDLIESFGSQPEGFKYEFMGKSLPYTPDALISYTDKT | |
| XTEN-GS-TasB | QKYHEYKPYSKIASPLFRABFAAKRAASLKLGIDLVLVTD | |
| | RQIRVNPILNNLKLLHRYSGVYGISGIQKELLSFIHKSGV | |
| | IKLNDISSQVGIPIGETRSFLFGLMHKGLVKADLGCDDLT | |
| | NNPTLWATPGSGSGKRTADGSEFESPKKKRKVGSGSGGMG | |
| | TKSELIERLATQQSHIPAKTVEDAVKEMLEHMASTLAQGG | |
| | SGGLTKAEMSEYLFDKLGLSKRDAKELVELFFEEIRRALE | |
| | NGEQVKLSGFGNFDLRDKNQRPGRNPKTGEDIPITARRVV | |
| | TFRPGQKLKSRVENAGGGERIEIRGFGSFSLHYRAPRTGR | |
| | NPKTGDKVELEGKYVPHFKPGKELRDRANIYGSGSETPGT | |
| | SESATPESGGSGSSGGSGSSGGMTDFFNEFDESLVPLKPQ | |
| | TPTQYVKLDDANLIQRDLDTFSDTFKNQALQRYKLISTID | |
| | KKLSRGWTQRNLDPILDELFKGGDVVRPNWRTVARWRKKY | |
| | IESNGDIASLADKNHKMGNRTNRIKGDDKFEDKALERFLD | |
| | AKRPTIATAYQYYKDLIVIENESIVEGKIPIISYNAFNKR | |
| | IKAIPPYAVAVARHGKFKADQWFAYCAAHVPPTRILERVE | |
| | IDHTPLDLILLDDELLIPIGRPYLTLLIDVFSGCVLGFHL | |
| | SYKSPSYVSAAKAITHAIKPKSLDALNIELQNDWPCFGKF | |
| | ENLVVDNGAEFWSKNLEHACQSAGINIQYNPVRKPWLKPF | |
| | IERFFGVMNEYFLPELPGKTFSNILEKEEYKPEKDAIMRF | |
| | STFVEEFHRWIADVYHQDSNSRETRIPIKRWQQGFDAYPP | |
| | LTMNEEEETRESMLMRISDSRTLTRNGFKYQELMYDSTAL | |
| | ADYRKHYPQTKETVKKLIKVDPDDISKIYVYLEELESYLE | |
| | VPCTDPTGYTDGLSIYEHKTIKKINREVIRESKDSLGLAK | |
| | ARMAIHERVKQEQEVFIESKTKAKITAVKKQAQIADVSNT | |
| | GTSTIKVSEESAAPVQKHISNDNSDDWDDDLEAFE* | |
| TnsA-NLS- | MYIRNLRKPSPNKNVFKFASTKVSSVVMCESSLEFDACFH | 5143 |
| GSGSGG-XTEN- | HEYNDLIESFGSQPEGFKYEFMGKSLPYTPDALISYTDKT | |
| IHF-XTEN-GS- | QKYHEYKPYSKIASPLFRAEFAAKRAASLKLGIDLVLVTD | |
| TnsB | RQIRVNPILNNLKLLHRYSGVYGISGIQKELLSFIHKSGV | |
| | IKLNDISSQVGIPIGETRSFLFGLMHKGLVKADLGCDDLT | |
| | NNPTLWATPGSGSGKRTADGSEFESPKKKRKVGSSGSETP | |
| | GTSESATPESSGGSSGGSSTMGTKSELIERLATQQSHIPA | |
| | KTVEDAVKEMLEHMASTLAQGGSGGLTKAEMSEYLEDKLG | |
| | LSKRDAKELVELFFEEIRRALENGEQVKLSGFGNFDLRDK | |
| | NQRPGRNPKTGEDIPITARRVVTFRPGQKLKSRVENAGGG | |
| | ERIEIRGFGSFSLHYRAPRTGRNPKTGDKVELEGKYVPHF | |
| | KPGKELRDRANIYGSGSETPGTSESATPESGGSGSSGGSG | |
| | SSGGMTDFFNEFDESLVPLKPQTPTQYVKLDDANLIQRDL | |
| | DTFSDTFKNQALQRYKLISTIDKKLSRGWTQRNLDPILDE | |
| | LFKGGDVVRPNWRTVARWRKKYIESNGDIASLADKNHKMG | |
| | NRTNRIKGDDKFFDKALERFLDAKRPTIATAYQYYKDLIV | |
| | IENESIVEGKIPIISYNAFNKRIKAIPPYAVAVARHGKFK | |
| | ADQWFAYCAAHVPPTRILERVEIDHTPLDLILLDDELLIP | |
| | IGRPYLTLLIDVFSGCVLGFHLSYKSPSYVSAAKAITHAI | |
| | KPKSLDALNIELQNDWPCFGKFENLVVDNGAEFWSKNLEH | |
| | ACQSAGINIQYNPVRKPWLKPFIERFFGVMNEYFLPELPG | |
| | KTESNILEKEEYKPEKDAIMRESTFVEEFHRWIADVYHQD | |
| | SNSRETRIPIKRWQQGFDAYPPLTMNEEEETRFSMLMRIS | |
| | DSRTLTRNGFKYQELMYDSTALADYRKHYPQTKETVKKLI | |
| | KVDPDDISKIYVYLEELESYLEVPCTDPTGYTDGLSIYEH | |
| | KTIKKINREVIRESKDSLGLAKARMAIHERVKQEQEVFIE | |
| | SKTKAKITAVKKQAQIADVSNTGTSTIKVSEESAAPVQKH | |
| | ISNDNSDDWDDDLEAFE* | |
| TnsA-NLS-(GGS)6- | MYIRNLRKPSPNKNVFKFASTKVSSVVMCESSLEFDACFH | 5144 |
| IHF-(XTEN)3-TnsB | HEYNDLIESFGSQPEGFKYEFMGKSLPYTPDALISYTDKT | |
| | QKYHEYKPYSKIASPLFRAEFAAKRAASLKLGIDLVLVTD | |
| | RQIRVNPILNNLKLLHRYSGVYGISGIQKELLSFIHKSGV | |
| | IKLNDISSQVGIPIGETRSFLFGLMHKGLVKADLGCDDLT | |
| | NNPTLWATPGSGSGKRTADGSEFESPKKKRKVGSGGSGGS | |
| | GGSGGSGGSGGSMGTKSELIERLATQQSHIPAKTVEDAVK | |
| | EMLEHMASTLAQGGSGGLTKAEMSEYLFDKLGLSKRDAKE | |
| | LVELFFEBIRRALENGEQVKLSGFGNFDLRDKNQRPGRNP | |
| | KTGEDIPITARRVVTFRPGQKLKSRVENAGGGERIBIRGF | |
| | GSFSLHYRAPRTGRNPKTGDKVELEGKYVPHFKPGKELRD | |
| | RANIYGSGGSSGGSSGSETPGTSESATPESSGSETPGTSE | |
| | SATPESSGSETPGTSESATPESSGGSSGGSSTMTDFFNEF | |
| | DESLVPLKPQTPTQYVKLDDANLIQRDLDTFSDTFKNQAL | |
| | QRYKLISTIDKKLSRGWTQRNLDPILDELFKGGDVVRPNW | |
| | RTVARWRKKYIESNGDIASLADKNHKMGNRTNRIKGDDKF | |
| | FDKALERFLDAKRPTIATAYQYYKDLIVIENESIVEGKIP | |
| | IISYNAFNKRIKAIPPYAVAVARHGKFKADQWFAYCAAHV | |
| | PPTRILERVEIDHTPLDLILLDDELLIPIGRPYLTLLIDV | |
| | FSGCVLGFHLSYKSPSYVSAAKAITHAIKPKSLDALNIEL | |
| | QNDWPCFGKFENLVVDNGAEFWSKNLEHACQSAGINIQYN | |
| | PVRKPWLKPFIERFFGVMNEYFLPELPGKTFSNILEKEEY | |
| | KPEKDAIMRESTFVEEFHRWIADVYHQDSNSRETRIPIKR | |
| | WQQGFDAYPPLTMNEEEETRESMLMRISDSRTLTRNGFKY | |
| | QELMYDSTALADYRKHYPQTKETVKKLIKVDPDDISKIYV | |
| | YLEELESYLEVPCTDPTGYTDGLSIYEHKTIKKINREVIR | |
| | ESKDSLGLAKARMAIHERVKQEQEVFIESKTKAKITAVKK | |
| | QAQIADVSNTGTSTIKVSEESAAPVQKHISNDNSDDWDDD | |
| | LEAFE* | |
| TnsA-NLS- | MYIRNLRKPSPNKNVFKFASTKVSSVVMCESSLEFDACFH | 5145 |
| (XTEN)3-IHF- | HEYNDLIESFGSQPEGFKYEFMGKSLPYTPDALISYTDKT | |
| (GGS)6-TnsB | QKYHEYKPYSKIASPLFRAEFAAKRAASLKLGIDLVLVTD | |
| | RQIRVNPILNNLKLLHRYSGVYGISGIQKELLSFIHKSGV | |
| | IKLNDISSQVGIPIGETRSFLFGLMHKGLVKADLGCDDLT | |
| | NNPTLWATPGSGSGKRTADGSEFESPKKKRKVGSSGGSSG | |
| | GSSGSETPGTSESATPESSGSETPGTSESATPESSGSETP | |
| | GTSESATPESSGGSSGGSSTMGTKSELIERLATQQSHIPA | |
| | KTVEDAVKEMLEHMASTLAQGGSGGLTKAEMSEYLFDKLG | |
| | LSKRDAKELVELFFEEIRRALENGEQVKLSGFGNFDLRDK | |
| | NQRPGRNPKTGEDIPITARRVVTFRPGQKLKSRVENAGGG | |
| | ERIEIRGFGSFSLHYRAPRTGRNPKTGDKVELEGKYVPHF | |
| | KPGKELRDRANIYGGGSGGSGGSGGSGGSGGSMTDFFNEF | |
| | DESLVPLKPQTPTQYVKLDDANLIQRDLDTFSDTFKNQAL | |
| | QRYKLISTIDKKLSRGWTQRNLDPILDELFKGGDVVRPNW | |
| | RTVARWRKKYIESNGDIASLADKNHKMGNRTNRIKGDDKF | |
| | FDKALERFLDAKRPTIATAYQYYKDLIVIENESIVEGKIP | |
| | IISYNAFNKRIKAIPPYAVAVARHGKFKADQWFAYCAAHV | |
| | PPTRILERVEIDHTPLDLILLDDELLIPIGRPYLTLLIDV | |
| | FSGCVLGFHLSYKSPSYVSAAKAITHAIKPKSLDALNIEL | |
| | QNDWPCFGKFENLVVDNGAEFWSKNLEHACQSAGINIQYN | |
| | PVRKPWLKPFIERFFGVMNEYFLPELPGKTFSNILEKEEY | |
| | KPEKDAIMRFSTFVEEFHRWIADVYHQDSNSRETRIPIKR | |
| | WQQGFDAYPPLTMNEEEETRESMLMRISDSRTLTRNGFKY | |
| | QELMYDSTALADYRKHYPQTKETVKKLIKVDPDDISKIYV | |
| | YLEELESYLEVPCTDPTGYTDGLSIYEHKTIKKINREVIR | |
| | ESKDSLGLAKARMAIHERVKQEQEVFIESKTKAKITAVKK | |
| | QAQIADVSNTGTSTIKVSEESAAPVQKHISNDNSDDWDDD | |
| | LEAFE* | |
| IHF-dCas9 | MPKKKRKVGGSGGSMGTKSELIERLATQQSHIPAKTVEDA | 5146 |
| | VKEMLEHMASTLAQGGSGGLTKAEMSEYLFDKLGLSKRDA | |
| | KELVELFFEEIRRALENGEQVKLSGFGNFDLRDKNQRPGR | |
| | NPKTGEDIPITARRVVTFRPGQKLKSRVENAGGGERIEIR | |
| | GFGSFSLHYRAPRTGRNPKTGDKVELEGKYVPHFKPGKEL | |
| | RDRANIYGGGGSGGGSGTGGSGGSGGSGGSGGSGRPMDKK | |
| | YSIGLAIGTNSVGWAVITDEYKVPSKKFKVLGNTDRHSIK | |
| | KNLIGALLEDSGETAEATRLKRTARRRYTRRKNRICYLQE | |
| | IFSNEMAKVDDSFFHRLEESFLVEEDKKHERHPIFGNIVD | |
| | EVAYHEKYPTIYHLRKKLVDSTDKADLRLIYLALAHMIKF | |
| | RGHFLIEGDLNPDNSDVDKLFIQLVQTYNQLFEENPINAS | |
| | GVDAKAILSARLSKSRRLENLIAQLPGEKKNGLFGNLIAL | |
| | SLGLTPNEKSNFDLAEDAKLQLSKDTYDDDLDNLLAQIGD | |
| | QYADLFLAAKNLSDAILLSDILRVNTEITKAPLSASMIKR | |
| | YDEHHQDLTLLKALVRQQLPEKYKEIFFDQSKNGYAGYID | |
| | GGASQEEFYKFIKPILEKMDGTEELLVKLNREDLLRKQRT | |
| | FDNGSIPHQIHLGELHAILRRQEDFYPFLKDNREKIEKIL | |
| | TFRIPYYVGPLARGNSRFAWMTRKSEETITPWNFEEVVDK | |
| | GASAQSFIERMTNFDKNLPNEKVLPKHSLLYEYFTVYNEL | |
| | TKVKYVTEGMRKPAFLSGEQKKAIVDLLFKTNRKVTVKQL | |
| | KEDYFKKIECFDSVEISGVEDRENASLGTYHDLLKIIKDK | |
| | DFLDNEENEDILEDIVLTLTLFEDREMIEERLKTYAHLFD | |
| | DKVMKQLKRRRYTGWGRLSRKLINGIRDKQSGKTILDFLK | |
| | SDGFANRNFMQLIHDDSLTFKEDIQKAQVSGQGDSLHEHI | |
| | ANLAGSPAIKKGILQTVKVVDELVKVMGRHKPENIVIEMA | |
| | RENQTTQKGQKNSRERMKRIEEGIKELGSQILKEHPVENT | |
| | QLQNEKLYLYYLQNGRDMYVDQELDINRLSDYDVDAIVPQ | |
| | SFLKDDSIDNKVLTRSDKNRGKSDNVPSEEVVKKMKNYWR | |
| | QLLNAKLITQRKFDNLTKAERGGLSELDKAGFIKRQLVET | |
| | RQITKHVAQILDSRMNTKYDENDKLIREVKVITLKSKLVS | |
| | DFRKDFQFYKVREINNYHHAHDAYLNAVVGTALIKKYPKL | |
| | ESEFVYGDYKVYDVRKMIAKSEQEIGKATAKYFFYSNIMN | |
| | FFKTEITLANGEIRKRPLIETNGETGEIVWDKGRDFATVR | |
| | KVLSMPQVNIVKKTEVQTGGFSKESILPKRNSDKLIARKK | |
| | DWDPKKYGGFDSPTVAYSVLVVAKVEKGKSKKLKSVKELL | |
| | GITIMERSSFEKNPIDFLEAKGYKEVKKDLIIKLPKYSLF | |
| | ELENGRKRMLASAGELQKGNELALPSKYVNFLYLASHYEK | |
| | LKGSPEDNEQKQLFVEQHKHYLDEIIEQISEFSKRVILAD | |
| | ANLDKVLSAYNKHRDKPIREQAENIIHLFTLTNLGAPAAF | |
| | KYEDTTIDRKRYTSTKEVLDATLIHQSITGLYETRIDLSQ | |
| | LGGDGGSGGSGGSGGSGGSASGGGSGGGSKRPAATKKAGQ | |
| | AKKKKGGSGSGATNFSLLKQAGDVEENPGPAAA* | |
| Integration host | MALTKAEMSEYLEDKLGLSKRDAKELVELFFEEIRRALEN | 5147 |
| factor subunit alpha | GEQVKLSGFGNFDLRDKNQRPGRNPKTGEDIPITARRVVT | |
| (E. coli) | FRPGQKLKSRVENASPKDE | |
| Integration host | MTKSELIERLATQQSHIPAKTVEDAVKEMLEHMASTLAQG | 5148 |
| factor subunit beta | ERIEIRGFGSFSLHYRAPRTGRNPKTGDKVELEGKYVPHF | |
| (E. coli) | KPGKELRDRANIYG | |
| Integration host | MALTKAELAEALFEQLGMSKRDAKDTVEVFFEEIRKALES | 5149 |
| factor subunit alpha | GEQVKLSGFGNFDLRDKNERPGRNPKTGEDIPITARRVVT | |
| (Vibrio cholerae | FRPGQKLKARVENIKVEK | |
| HE-45) | | |
| Integration host | MTKSELIERLCAEQTHLSAKEIEDAVKNILEHMASTLEAG | 5150 |
| factor subunit beta | ERIEIRGFGSFSLHYREPRVGRNPKTGDKVELEGKYVPHF | |
| (Vibrio cholerae | KPGKELRERVNL | |
| HE-45) | | |
| Integration host | MALTKADIAEHLFEKLGINKKDAKDLVEAFFEEIRSALEK | 5151 |
| factor subunit alpha | GEQVKLSGFGNFDLRDKKERPGRNPKTGEDIPISARRVVT | |
| (Psuedoalteromonas | FRPGQKLKTRVEVGTSKAK | |
| sp. S983) | | |
| Integration host | MTKSELIETLAEQHAHVPVKDVENAVKEILEQMAGSLSTS | 5152 |
| factor subunit beta | DRIEIRGFGSFSLHYRAPRTGRNPKTGDTVELDGKHVPHF | |
| (Psuedoalteromonas | KPGKELRDRVNESIA | |
| sp. S983) | | |

TABLE 4
Selected Variant Transposition

| | | | | | | | | | normalized | | |
|---|---|---|---|---|---|---|---|---|---|---|---|
| | | | | | | | | | log2 fold- | | |
| | | | | | | | | | change | | |
| | | | | | | | log2 fold-change | | log2 | | |
| | | | | abundance (Ab) = | | | log2(AbOutput/ | | (foldchange/ | | |
| | Read count | | | (count/total_counts) | | | AbInput) | | average WT foldchange) | | Read count |
| ID | input | LR | RL | input | LR | RL | LR | RL | LR | RL | input |
| ORF1a | 1420 | 585 | 715 | 0.0426 | 0.0597 | 0.0323 | 0.4861 | −0.3989 | 1.22 | −1.47 | 1420 |
| ORF1b | 2578 | 672 | 883 | 0.0773 | 0.0685 | 0.0399 | −0.1742 | −0.9548 | 0.56 | −2.03 | 2578 |
| ORF1c | 2368 | 729 | 791 | 0.0710 | 0.0744 | 0.0357 | 0.0658 | −0.9910 | 0.80 | −2.06 | 2368 |
| ORF2a | 2365 | 973 | 708 | 0.0710 | 0.0992 | 0.0320 | 0.4842 | −1.1491 | 1.22 | −2.22 | 2365 |
| ORF3a | 778 | 621 | 695 | 0.0233 | 0.0633 | 0.0314 | 1.4403 | 0.4282 | 2.18 | −0.64 | 778 |
| ORF3b | 1124 | 1170 | 877 | 0.0337 | 0.1193 | 0.0396 | 1.8233 | 0.2330 | 2.56 | −0.84 | 1124 |
| ORF3c | 2525 | 903 | 629 | 0.0758 | 0.0921 | 0.0284 | 0.2820 | −1.4142 | 1.02 | −2.49 | 2525 |

| | | | | | | | | normalized | |
|---|---|---|---|---|---|---|---|---|---|
| | | | | | | | | log2 fold- | |
| | | | | | | | | change | |
| | | | | | | log2 fold-change | | log2 | |
| | | | abundance (Ab) = | | | log2(AbOutput/ | | (foldchange/ | |
| | Read count | | (count/total_counts) | | | AbInput) | | average_WT_foldchange) | |
| ID | LR | RL | input | LR | RL | LR | RL | LR | RL |
| ORF1a | 677 | 955 | 0.0426 | 0.0378 | 0.0765 | 0.8443 | −0.1720 | 1.49 | −1.08 |
| ORF1b | 822 | 1209 | 0.0773 | 0.0479 | 0.0929 | 0.2639 | −0.6922 | 0.91 | −1.60 |
| ORF1c | 742 | 1183 | 0.0710 | 0.0468 | 0.0838 | 0.2388 | −0.6009 | 0.89 | −1.51 |
| ORF2a | 999 | 953 | 0.071 | 0.0377 | 0.1129 | 0.6696939 | −0.9110151 | 1.32 | −1.82 |
| ORF3a | 651 | 1066 | 0.0233 | 0.0422 | 0.0736 | 1.6558648 | 0.8546424 | 2.30 | −0.05 |
| ORF3b | 1058 | 1021 | 0.0337 | 0.0404 | 0.1195 | 1.825675 | 0.2616178 | 2.47 | −0.65 |
| ORF3c | 645 | 972 | 0.0758 | 0.0385 | 0.0729 | −0.0559349 | −0.9769782 | 0.59 | −1.89 |

TABLE 5
Tn6677 hyperactive transposon right end variants.

| | | | | | | | Enrichment score = |
|---|---|---|---|---|---|---|---|
| | | | | Ab = Abundance = | | | Log2 (FC = Fold Change = |
| Variant | Read count | | | (count/total_counts) | | | (Ab_Output/Ab_Input)) |
| SEQ ID NO: | Input | RL | LR | input | RL | LR | RL |
| 2691 | 144 | 534 | 62 | 0.00833458 | 0.04369821 | 0.01347144 | 2.39039236 |
| 2692 | 165 | 522 | 74 | 0.00955004 | 0.04271623 | 0.01607881 | 2.16120521 |
| 2693 | 502 | 1453 | 170 | 0.02905528 | 0.11890169 | 0.03693781 | 2.03289686 |
| 2694 | 497 | 1323 | 170 | 0.02876589 | 0.10826354 | 0.03693781 | 1.91211673 |
| 2695 | 343 | 880 | 147 | 0.01985251 | 0.07201203 | 0.03194034 | 1.85891638 |
| 2696 | 591 | 1530 | 183 | 0.03420652 | 0.12520274 | 0.03976247 | 1.87192305 |
| 2697 | 416 | 1192 | 165 | 0.02407768 | 0.09754357 | 0.03585141 | 2.01835023 |
| 2698 | 661 | 1483 | 194 | 0.03825805 | 0.12135664 | 0.04215256 | 1.66541785 |
| 2699 | 873 | 2177 | 301 | 0.05052841 | 0.17814795 | 0.06540166 | 1.81790928 |
| 2700 | 528 | 1279 | 190 | 0.03056014 | 0.10466294 | 0.04128344 | 1.77602786 |
| 2701 | 711 | 1706 | 186 | 0.041152 | 0.13960514 | 0.04041431 | 1.76231761 |
| 2702 | 680 | 1639 | 217 | 0.03935775 | 0.13412241 | 0.04715003 | 1.76883063 |

| | Enrichment score = | | | | |
|---|---|---|---|---|---|
| | Log2 (FC = Fold Change = | Normalized enrichment | | Normalized FC = | |
| Variant | (Ab_Output/Ab_Input)) | Log2 (Normalized FC) | | Normalized enrichment \hat{ } 2 | |
| SEQ ID NO: | LR | RL | LR | RL | LR |
| 2691 | 0.69272194 | 1.39407206 | 1.12302665 | 2.628194525 | 2.178034264 |
| 2692 | 0.75158178 | 1.16488491 | 1.18188649 | 2.242153283 | 2.268732461 |
| 2693 | 0.34629801 | 1.03657656 | 0.77660272 | 2.051354117 | 1.713092104 |
| 2694 | 0.36073952 | 0.91579643 | 0.79104424 | 1.886610275 | 1.730326441 |
| 2695 | 0.68605821 | 0.86259607 | 1.11636292 | 1.818307337 | 2.16799724 |
| 2696 | 0.21713614 | 0.87560274 | 0.64744086 | 1.834774472 | 1.566387177 |
| 2697 | 0.57433312 | 1.02202993 | 1.00463784 | 2.030774332 | 2.006439757 |
| 2698 | 0.13985701 | 0.66909755 | 0.57016172 | 1.590078014 | 1.484689989 |
| 2699 | 0.37223246 | 0.82158898 | 0.80253717 | 1.767351477 | 1.744165777 |
| 2700 | 0.43391212 | 0.77970756 | 0.86421683 | 1.716782839 | 1.820351217 |
| 2701 | −0.0260963 | 0.76599731 | 0.4042084 | 1.700545149 | 1.323362588 |
| 2702 | 0.26061092 | 0.77251033 | 0.69091564 | 1.708239584 | 1.614307751 |

TABLE 6
Tn6677 hyperactive transposon left end variants.

| | | | | | | | Enrichment score = |
|---|---|---|---|---|---|---|---|
| | | | | Ab = Abundance = | | | Log2 (FC = Fold Change = |
| Variant | Read count | | | (count/total_counts) | | | (Ab_Output/Ab_Input)) |
| SEQ ID NO: | Input | RL | LR | input | RL | LR | RL |
| 4666 | 366 | 1970 | 638 | 0.029638 | 0.12776461 | 0.12956682 | 2.10796813 |
| 4667 | 778 | 3348 | 727 | 0.063001 | 0.21713499 | 0.14764119 | 1.78514552 |
| 4668 | 613 | 2242 | 687 | 0.04963961 | 0.14540521 | 0.13951788 | 1.55051536 |
| 4669 | 565 | 1992 | 494 | 0.04575266 | 0.12919143 | 0.1003229 | 1.49758293 |
| 4670 | 596 | 2077 | 444 | 0.04826298 | 0.13470411 | 0.09016876 | 1.48080504 |
| 4671 | 774 | 2499 | 666 | 0.06267709 | 0.16207298 | 0.13525314 | 1.37063349 |
| 4672 | 875 | 2802 | 448 | 0.07085588 | 0.18172408 | 0.09098109 | 1.35879009 |
| 4673 | 655 | 2086 | 623 | 0.05304069 | 0.13528781 | 0.12652058 | 1.3508604 |

| | Enrichment score = | | | | |
|---|---|---|---|---|---|
| | Log2 (FC = Fold Change = | Normalized enrichment | | Normalized FC = | |
| Variant | (Ab_Output/Ab_Input)) | Log2 (Normalized FC) | | Normalized enrichment \hat{ } 2 | |
| SEQ ID NO: | LR | RL | LR | RL | LR |
| 4666 | 2.1281762 | 1.36927774 | 1.54104242 | 2.583411998 | 2.910046931 |
| 4667 | 1.22864863 | 1.04645514 | 0.64151485 | 2.065448574 | 1.559966286 |
| 4668 | 1.49088645 | 0.81182497 | 0.90375267 | 1.755430611 | 1.870926224 |
| 4669 | 1.1327236 | 0.75889254 | 0.54558982 | 1.692191145 | 1.45961696 |
| 4670 | 0.90171077 | 0.74211465 | 0.31457699 | 1.672625717 | 1.243646952 |
| 4671 | 1.10965203 | 0.6319431 | 0.52251825 | 1.549650742 | 1.436460428 |
| 4672 | 0.36067914 | 0.6200997 | −0.2264546 | 1.536981393 | 0.854732805 |
| 4673 | 1.25420068 | 0.61217001 | 0.6670669 | 1.528556638 | 1.587841491 |

TABLE 7
Transposon Left end Variants SEQ ID NOs: 3120-4665

| | Normalized | Normalized |
|---|---|---|
| | enrichment = Log2 | enrichment = Log2 |
| SEQ | (Normalized | (Normalized |
| ID | FC) − RL | FC) − RL |
| NO | (1^{st} Replicate) | (2^{nd} Replicate) |
| 3120 | −0.0920 | −0.1196 |
| 3121 | −0.1589 | −0.1221 |
| 3122 | −0.3028 | −0.1583 |
| 3123 | −0.1687 | −0.0930 |
| 3124 | 0.3025 | 0.7183 |
| 3125 | −0.0271 | 0.0739 |
| 3126 | −0.0323 | −0.1814 |
| 3127 | −0.0960 | 0.0584 |
| 3128 | 0.2132 | −0.0754 |
| 3129 | −0.0700 | −0.1073 |
| 3130 | −0.1346 | −0.1114 |
| 3131 | −0.1020 | −0.0508 |
| 3132 | −0.4968 | −0.3233 |
| 3133 | 0.0804 | 0.0462 |
| 3134 | 0.0547 | 0.1251 |
| 3135 | −0.0254 | −0.1459 |
| 3136 | −0.4439 | −0.0746 |
| 3137 | 0.3111 | 0.1639 |
| 3138 | 0.4355 | 0.3983 |
| 3139 | −0.3595 | −0.2221 |
| 3140 | −0.1083 | −0.0183 |
| 3141 | −0.5151 | −0.3808 |
| 3142 | −0.3106 | −0.2442 |
| 3143 | −0.1557 | 0.0135 |
| 3144 | 0.1481 | 0.3777 |
| 3145 | −0.4356 | −0.1720 |
| 3146 | −0.7206 | −0.5393 |
| 3147 | −0.3510 | −0.1659 |
| 3148 | −0.1876 | −0.0795 |
| 3149 | −0.3738 | −0.0162 |
| 3150 | −0.2141 | −0.0705 |
| 3151 | −0.0857 | 0.0537 |
| 3152 | −0.6942 | −0.3374 |
| 3153 | −0.4993 | −0.3103 |
| 3154 | −0.0674 | 0.4852 |
| 3155 | −0.7764 | −0.5471 |
| 3156 | −0.4871 | 0.1020 |
| 3157 | −0.5977 | 0.0836 |
| 3158 | −0.9712 | −0.3965 |
| 3159 | −0.4368 | 0.0634 |
| 3160 | −1.1051 | −0.4840 |
| 3161 | −0.7405 | −0.3679 |
| 3162 | −0.6708 | −0.1799 |
| 3163 | −1.1210 | −0.4550 |
| 3164 | −1.0947 | −0.4992 |
| 3165 | −2.2545 | −1.3719 |
| 3166 | −1.2450 | −0.4252 |
| 3167 | −1.5277 | −0.8136 |
| 3168 | −0.7241 | −0.4416 |
| 3169 | −0.5053 | −0.1762 |
| 3170 | −0.2774 | 0.1031 |
| 3171 | −0.2312 | −0.2834 |
| 3172 | 0.3464 | 0.2509 |
| 3173 | −0.0615 | −0.1089 |
| 3174 | −0.0863 | −0.0523 |
| 3175 | −0.2961 | 0.0501 |
| 3176 | −1.2165 | −0.4332 |
| 3177 | −1.4467 | −0.7979 |
| 3178 | −0.7622 | −0.4362 |
| 3179 | −1.5470 | −0.8870 |
| 3180 | −1.6309 | −0.9422 |
| 3181 | 0.3110 | 1.0091 |
| 3182 | −1.2189 | −0.1928 |
| 3183 | −1.6377 | −0.9052 |
| 3184 | −1.2608 | −0.4924 |
| 3185 | −0.4089 | −0.1479 |
| 3186 | −1.6843 | −0.7709 |
| 3187 | −0.4196 | 0.2258 |
| 3188 | −0.2757 | −0.0805 |
| 3189 | −0.6227 | −0.3051 |
| 3190 | −0.4993 | 0.0297 |
| 3191 | −0.4146 | −0.2337 |
| 3192 | −0.6172 | −0.2926 |
| 3193 | −0.4065 | 0.2214 |
| 3194 | −0.9305 | −0.4781 |
| 3195 | 0.7222 | 0.7256 |
| 3196 | −0.1765 | 0.1603 |
| 3197 | −1.1174 | −0.5726 |
| 3198 | −0.3624 | −0.2735 |
| 3199 | −0.4419 | −0.0944 |
| 3200 | −0.9599 | −0.3779 |
| 3201 | −1.3623 | −0.5538 |
| 3202 | −0.7134 | −0.2438 |
| 3203 | −0.2653 | −0.1483 |
| 3204 | −0.4222 | 0.1532 |
| 3205 | −0.0904 | 0.0000 |
| 3206 | −0.6912 | −0.4357 |
| 3207 | −0.4444 | −0.1814 |
| 3208 | 0.1603 | −0.0410 |
| 3209 | 0.2512 | −0.0580 |
| 3210 | 0.4014 | −0.1544 |
| 3211 | −0.5184 | −0.2894 |
| 3212 | −0.3019 | −0.3769 |
| 3213 | 0.3582 | −0.1970 |
| 3214 | 0.0402 | 0.0218 |
| 3215 | −0.1361 | −0.1799 |
| 3216 | −0.2938 | −0.3356 |
| 3217 | −0.0483 | 0.0935 |
| 3218 | −0.3091 | −0.1700 |
| 3219 | −0.2061 | −0.0907 |
| 3220 | −0.3132 | −0.2378 |
| 3221 | −0.0732 | 0.0267 |
| 3222 | −0.1509 | −0.1241 |
| 3223 | 0.6388 | 0.6740 |
| 3224 | −0.1398 | −0.0764 |
| 3225 | −0.3265 | −0.1962 |
| 3226 | 0.0372 | −0.0264 |
| 3227 | 0.0936 | 0.1591 |
| 3228 | −0.1101 | 0.0757 |
| 3229 | −0.0869 | −0.0397 |
| 3230 | −0.8036 | −0.3790 |
| 3231 | −0.1971 | −0.1186 |
| 3232 | −0.2289 | −0.1483 |
| 3233 | −0.1512 | 0.1710 |
| 3234 | −0.1102 | 0.2761 |
| 3235 | −0.6459 | 0.4398 |
| 3236 | −0.8987 | −0.2954 |
| 3237 | 0.3283 | 0.7435 |
| 3238 | −0.8013 | 0.0096 |
| 3239 | −0.6420 | −0.2563 |
| 3240 | −0.6780 | −0.2798 |
| 3241 | −1.0339 | −0.5114 |
| 3242 | −0.7362 | −0.1432 |
| 3243 | −2.5286 | −1.5930 |
| 3244 | −0.8623 | −0.3292 |
| 3245 | −1.0364 | −0.3680 |
| 3246 | −0.7861 | −0.4232 |
| 3247 | 0.1000 | 0.0769 |
| 3248 | −0.1173 | −0.0413 |
| 3249 | −0.1518 | −0.0069 |
| 3250 | −0.2585 | −0.1758 |
| 3251 | −0.2599 | −0.2256 |
| 3252 | 0.3003 | 0.3741 |
| 3253 | 0.0931 | 0.0056 |
| 3254 | −1.2538 | −0.5527 |
| 3255 | −1.3846 | −0.7316 |
| 3256 | −0.3987 | −0.1798 |
| 3257 | −1.1606 | −0.5933 |
| 3258 | −0.2277 | 0.6652 |
| 3259 | −0.4728 | −0.1899 |
| 3260 | −1.9077 | −0.7874 |
| 3261 | −1.7705 | −0.8829 |
| 3262 | −0.8518 | −0.2210 |
| 3263 | −0.1836 | 0.2494 |
| 3264 | −1.5606 | −0.7171 |
| 3265 | −0.8294 | −0.1546 |
| 3266 | −0.1933 | 0.0878 |
| 3267 | −0.5022 | 0.1007 |
| 3268 | −0.6472 | −0.2745 |
| 3269 | −0.5700 | −0.2469 |
| 3270 | −0.5368 | 0.0313 |
| 3271 | −0.3058 | 0.0662 |
| 3272 | −0.9554 | −0.3724 |
| 3273 | 0.0478 | 0.1511 |
| 3274 | −0.5997 | −0.4512 |
| 3275 | −0.7102 | −0.1341 |
| 3276 | −0.4344 | −0.2913 |
| 3277 | −0.6564 | −0.2087 |
| 3278 | −0.9131 | −0.3402 |
| 3279 | −1.0881 | −0.5730 |
| 3280 | −0.3899 | −0.0133 |
| 3281 | −0.3274 | −0.2099 |
| 3282 | −0.1650 | −0.0922 |
| 3283 | 0.2040 | −0.0634 |
| 3284 | 0.0921 | 0.0261 |
| 3285 | −0.1805 | −0.1135 |
| 3286 | 0.1486 | 0.0127 |
| 3287 | 0.0990 | 0.3949 |
| 3288 | 0.2114 | 0.2833 |
| 3289 | −0.3099 | −0.1482 |
| 3290 | −0.4794 | −0.2858 |
| 3291 | −0.1380 | 0.0129 |
| 3292 | 0.2457 | 0.2287 |
| 3293 | 0.0376 | 0.1233 |
| 3294 | 0.2507 | 0.1959 |
| 3295 | 0.9627 | 1.3754 |
| 3296 | 0.1975 | 0.4383 |
| 3297 | −0.0636 | −0.0953 |
| 3298 | −0.0051 | 0.1238 |
| 3299 | −0.1011 | −0.1423 |
| 3300 | −0.2047 | −0.1775 |
| 3301 | 0.1215 | 0.0730 |
| 3302 | −0.0021 | −0.0055 |
| 3303 | −0.1110 | −0.0867 |
| 3304 | 0.1024 | 0.0244 |
| 3305 | −0.0874 | −0.0071 |
| 3306 | 0.1709 | −0.0268 |
| 3307 | −0.1435 | −0.0171 |
| 3308 | −0.1460 | −0.0564 |
| 3309 | −0.3433 | −0.1020 |
| 3310 | 0.0335 | 0.2450 |
| 3311 | −1.7266 | −0.9350 |
| 3312 | −0.7628 | −0.3291 |
| 3313 | −0.1637 | −0.1701 |
| 3314 | −0.1959 | −0.0276 |
| 3315 | −0.1566 | −0.2424 |
| 3316 | 0.0087 | 0.1330 |
| 3317 | −0.2125 | −0.0344 |
| 3318 | 0.1286 | 0.2256 |
| 3319 | 0.1407 | 0.3080 |
| 3320 | 0.0017 | 0.0141 |
| 3321 | −0.1934 | −0.1629 |
| 3322 | 0.0123 | 0.1363 |
| 3323 | −0.0615 | −0.0465 |
| 3324 | −0.3329 | −0.2224 |
| 3325 | 0.2673 | 0.2911 |
| 3326 | 0.2965 | 0.1062 |
| 3327 | −0.1085 | 0.1041 |
| 3328 | 0.2418 | 0.3614 |
| 3329 | −0.1223 | 0.1349 |
| 3330 | −0.5580 | −0.2829 |
| 3331 | −0.2965 | −0.0249 |
| 3332 | −0.1642 | −0.1712 |
| 3333 | −0.1894 | −0.0493 |
| 3334 | 0.0089 | 0.3469 |
| 3335 | −0.0443 | 0.1667 |
| 3336 | −0.1064 | 0.3599 |
| 3337 | 0.0058 | 0.3173 |
| 3338 | 0.1012 | 0.3787 |
| 3339 | −0.3249 | −0.1124 |
| 3340 | 0.0041 | 0.1095 |
| 3341 | −0.2369 | −0.0245 |
| 3342 | −0.1359 | 0.0205 |
| 3343 | 0.0239 | −0.1785 |
| 3344 | −0.2065 | 0.0451 |
| 3345 | −0.3686 | −0.2144 |
| 3346 | −0.0556 | 0.0326 |
| 3347 | −0.0034 | −0.0777 |
| 3348 | −0.3918 | −0.2598 |
| 3349 | 0.1169 | 0.0718 |
| 3350 | 0.2011 | 0.1090 |
| 3351 | 0.1952 | 0.0993 |
| 3352 | −0.0069 | 0.0273 |
| 3353 | −0.3104 | −0.2498 |
| 3354 | −0.3173 | −0.1654 |
| 3355 | 0.4345 | 0.5069 |
| 3356 | −0.2530 | 0.0221 |
| 3357 | 0.3429 | 0.3979 |
| 3358 | 0.1199 | 0.0532 |
| 3359 | −0.1156 | −0.1410 |
| 3360 | 0.1217 | 0.4870 |
| 3361 | 0.2641 | 0.2933 |
| 3362 | −0.2793 | −0.2661 |
| 3363 | −0.0905 | −0.0442 |
| 3364 | 0.0124 | 0.1125 |
| 3365 | −0.0289 | 0.0489 |
| 3366 | 0.0567 | 0.0723 |
| 3367 | −0.0845 | −0.1613 |
| 3368 | −0.0775 | −0.1692 |
| 3369 | −0.0821 | 0.0659 |
| 3370 | −0.1032 | −0.0300 |
| 3371 | 0.1267 | 0.1648 |
| 3372 | −0.2152 | −0.2285 |
| 3373 | −0.0131 | 0.0466 |
| 3374 | −0.4187 | −0.0983 |
| 3375 | −0.3211 | −0.1103 |
| 3376 | 0.1035 | 0.0352 |
| 3377 | 0.2394 | 0.0875 |
| 3378 | −0.2170 | 0.0367 |
| 3379 | 0.6321 | 0.7869 |
| 3380 | −0.0529 | 0.0122 |
| 3381 | 0.2605 | 0.1699 |
| 3382 | −0.0120 | 0.2825 |
| 3383 | 0.3446 | 0.2396 |
| 3384 | 0.2301 | 0.1138 |
| 3385 | 0.1555 | 0.2009 |
| 3386 | −0.2475 | −0.0450 |
| 3387 | −0.1752 | −0.0094 |
| 3388 | −0.2281 | −0.0269 |
| 3389 | −0.3734 | −0.0695 |
| 3390 | −0.1926 | 0.0413 |
| 3391 | 0.2229 | 0.2883 |
| 3392 | −0.8778 | −0.3497 |
| 3393 | −0.0055 | 0.1300 |
| 3394 | 0.1687 | 0.3511 |
| 3395 | −0.5498 | 0.0737 |
| 3396 | −0.5859 | −0.0805 |
| 3397 | −0.5661 | −0.1748 |
| 3398 | −0.7318 | −0.3400 |
| 3399 | −0.3771 | −0.1687 |
| 3400 | −0.9319 | −0.1577 |
| 3401 | −0.8696 | −0.3717 |
| 3402 | −0.5608 | −0.2061 |
| 3403 | −0.7624 | −0.0910 |
| 3404 | −0.6219 | −0.0357 |
| 3405 | −1.8231 | −0.9065 |
| 3406 | −0.5600 | −0.1328 |
| 3407 | −0.7157 | −0.1934 |
| 3408 | −0.2219 | −0.0521 |
| 3409 | 0.3523 | 0.2468 |
| 3410 | 0.5827 | 0.8577 |
| 3411 | −0.1792 | −0.1491 |
| 3412 | −0.0789 | 0.2508 |
| 3413 | −0.0919 | 0.0581 |
| 3414 | −0.6321 | −0.3010 |
| 3415 | −0.0199 | 0.2168 |
| 3416 | −1.3578 | −0.1074 |
| 3417 | −1.4147 | −0.7307 |
| 3418 | −0.2736 | −0.0535 |
| 3419 | −0.7568 | −0.0674 |
| 3420 | −1.2528 | −0.4814 |
| 3421 | −0.4604 | −0.2260 |
| 3422 | −1.2567 | −0.4108 |
| 3423 | −1.4159 | −0.7229 |
| 3424 | −0.5361 | 0.0017 |
| 3425 | −0.4040 | −0.2998 |
| 3426 | −1.8369 | −1.1365 |
| 3427 | −0.1699 | −0.1922 |
| 3428 | 0.1036 | 0.2169 |
| 3429 | 0.2003 | 0.3173 |
| 3430 | −0.1211 | 0.2013 |
| 3431 | −0.1378 | 0.0908 |
| 3432 | −0.5235 | −0.1654 |
| 3433 | −0.0093 | 0.0811 |
| 3434 | −0.3919 | −0.0171 |
| 3435 | −0.3491 | −0.1702 |
| 3436 | −0.2486 | −0.0474 |
| 3437 | −0.3026 | 0.0605 |
| 3438 | 0.0117 | 0.1561 |
| 3439 | −0.7688 | −0.3225 |
| 3440 | −0.4209 | −0.1694 |
| 3441 | −0.3198 | 0.1408 |
| 3442 | 0.1465 | 0.2436 |
| 3443 | 0.0376 | 0.0952 |
| 3444 | −0.0460 | 0.1093 |
| 3445 | −0.1387 | −0.0593 |
| 3446 | −1.3196 | −0.5134 |
| 3447 | −0.0984 | −0.0301 |
| 3448 | −0.0306 | 0.0171 |
| 3449 | −0.1654 | 0.2429 |
| 3450 | −0.6395 | −0.1631 |
| 3451 | −0.1463 | −0.0198 |
| 3452 | −0.6391 | −0.2697 |
| 3453 | −0.4937 | −0.2199 |
| 3454 | −0.0693 | 0.0612 |
| 3455 | 0.3442 | 0.2163 |
| 3456 | 0.2390 | 0.2447 |
| 3457 | −0.3429 | −0.0750 |
| 3458 | −7.3745 | −5.9937 |
| 3459 | −1.7851 | −0.9890 |
| 3460 | −0.2636 | −0.0484 |
| 3461 | −3.0977 | −2.0777 |
| 3462 | −0.2877 | 0.0686 |
| 3463 | −6.7372 | −6.6300 |
| 3464 | −7.0578 | −6.9688 |
| 3465 | −2.9142 | −2.3904 |
| 3466 | −0.2604 | −0.1799 |
| 3467 | −0.0770 | 0.1278 |
| 3468 | −0.3130 | −0.3243 |
| 3469 | −0.1209 | −0.0712 |
| 3470 | −0.2249 | −0.1187 |
| 3471 | −0.1566 | −0.1059 |
| 3472 | −0.1984 | −0.0975 |
| 3473 | 0.1227 | 0.0603 |
| 3474 | 0.2934 | 0.2998 |
| 3475 | −0.1029 | −0.1788 |
| 3476 | 0.2117 | 0.2663 |
| 3477 | −0.0304 | −0.0821 |
| 3478 | 0.0310 | 0.0949 |
| 3479 | 0.0662 | 0.1999 |
| 3480 | −0.0961 | 0.0490 |
| 3481 | −0.1820 | −0.0806 |
| 3482 | 0.0491 | −0.0610 |
| 3483 | 0.1072 | 0.0660 |
| 3484 | −0.3046 | −0.2405 |
| 3485 | 0.2195 | 0.2661 |
| 3486 | −0.6998 | −0.2431 |
| 3487 | −0.2508 | −0.1478 |
| 3488 | −0.5263 | −0.3600 |
| 3489 | −0.7341 | −0.5099 |
| 3490 | −1.6229 | −0.7069 |
| 3491 | −1.3102 | −0.5617 |
| 3492 | −1.5812 | −0.7399 |
| 3493 | −1.4361 | −0.5874 |
| 3494 | −2.0186 | −0.8282 |
| 3495 | −1.7076 | −0.6839 |
| 3496 | −1.8013 | −0.8024 |
| 3497 | −0.5524 | −0.3481 |
| 3498 | −1.2646 | −0.6822 |
| 3499 | −1.9382 | −1.1565 |
| 3500 | −2.6612 | −1.6674 |
| 3501 | −2.4133 | −1.3353 |
| 3502 | −2.1137 | −1.3538 |
| 3503 | −2.2442 | −1.1592 |
| 3504 | −1.7341 | −0.9315 |
| 3505 | −0.6662 | −0.1042 |
| 3506 | −0.7563 | −0.1913 |
| 3507 | −0.8098 | −0.2350 |
| 3508 | −0.5032 | −0.1595 |
| 3509 | −0.2582 | 0.0262 |
| 3510 | −0.3906 | 0.0897 |
| 3511 | −0.2851 | −0.0764 |
| 3512 | −0.0261 | 0.0610 |
| 3513 | −0.1052 | 0.0931 |
| 3514 | −0.3196 | −0.1720 |
| 3515 | −0.3190 | −0.2229 |
| 3516 | −0.6221 | −0.1924 |
| 3517 | −1.2209 | −0.6594 |
| 3518 | −1.8688 | −0.9356 |
| 3519 | −1.1462 | −0.7795 |
| 3520 | −2.0041 | −1.0151 |
| 3521 | −1.7712 | −0.7010 |
| 3522 | −0.2104 | 0.9835 |
| 3523 | 0.4923 | 0.7954 |
| 3524 | −1.1093 | −0.4603 |
| 3525 | −1.9721 | −1.2051 |
| 3526 | −2.8726 | −1.5430 |
| 3527 | −2.9835 | −1.7603 |
| 3528 | −2.8047 | −1.5229 |
| 3529 | −1.5927 | −0.4979 |
| 3530 | −1.6275 | −0.8578 |
| 3531 | −0.7395 | −0.3344 |
| 3532 | −0.4259 | −0.1069 |
| 3533 | −0.2840 | 0.0671 |
| 3534 | −0.0872 | −0.0409 |
| 3535 | 0.0044 | 0.1133 |
| 3536 | −0.3071 | −0.0952 |
| 3537 | −0.1912 | −0.0794 |
| 3538 | 0.1357 | 0.2275 |
| 3539 | 0.4049 | 0.4807 |
| 3540 | 0.3191 | 0.3877 |
| 3541 | −0.0670 | −0.0732 |
| 3542 | −0.0357 | −0.1296 |
| 3543 | −1.9226 | −0.9763 |
| 3544 | −2.0594 | −1.1209 |
| 3545 | −0.1766 | 0.3661 |
| 3546 | −0.1285 | −0.0182 |
| 3547 | −0.0085 | 0.1802 |
| 3548 | −0.2488 | −0.1044 |
| 3549 | 0.0240 | 0.2932 |
| 3550 | −0.3492 | −0.0432 |
| 3551 | −0.9267 | −0.2694 |
| 3552 | −1.0129 | −0.3493 |
| 3553 | −0.9017 | −0.4108 |
| 3554 | −0.4854 | −0.2118 |
| 3555 | −0.3588 | −0.0785 |
| 3556 | −0.1686 | 0.0217 |
| 3557 | −0.1206 | −0.0503 |
| 3558 | −0.2258 | −0.0206 |
| 3559 | −0.0992 | 0.2240 |
| 3560 | −0.0296 | −0.0301 |
| 3561 | −0.6978 | −0.3727 |
| 3562 | −0.1886 | −0.1335 |
| 3563 | −0.2675 | −0.2049 |
| 3564 | 0.1400 | 0.1143 |
| 3565 | −0.3684 | −0.2679 |
| 3566 | −0.0912 | −0.1272 |
| 3567 | 0.1647 | 0.1701 |
| 3568 | 0.2549 | 0.4486 |
| 3569 | −0.4684 | 0.0186 |
| 3570 | −1.2810 | −0.6467 |
| 3571 | −1.5004 | −0.7471 |
| 3572 | −1.6255 | −0.8515 |
| 3573 | −1.6050 | −0.9353 |
| 3574 | −2.1740 | −1.1812 |
| 3575 | −0.9718 | −0.5020 |
| 3576 | −0.8027 | −0.3189 |
| 3577 | −0.1506 | −0.0606 |
| 3578 | −1.7236 | −1.1256 |
| 3579 | −2.1236 | −1.5141 |
| 3580 | −2.7275 | −1.6364 |
| 3581 | −3.2774 | −2.1109 |
| 3582 | −2.2367 | −1.1165 |
| 3583 | −1.6533 | −0.6486 |
| 3584 | −1.0185 | −0.4380 |
| 3585 | −0.1122 | 0.2742 |
| 3586 | −0.2750 | 0.2113 |
| 3587 | −0.6347 | −0.1979 |
| 3588 | −0.3931 | −0.1860 |
| 3589 | −0.6175 | −0.2544 |
| 3590 | −1.8680 | −1.2824 |
| 3591 | −0.0024 | 0.1526 |
| 3592 | −7.4639 | −6.2221 |
| 3593 | −7.9885 | −6.4635 |
| 3594 | −8.3930 | −9.6810 |
| 3595 | −7.3423 | −10.9782 |
| 3596 | −0.3903 | −0.3416 |
| 3597 | 0.0917 | 0.1494 |
| 3598 | −0.0758 | −0.0618 |
| 3599 | −0.2396 | −0.2441 |
| 3600 | −0.2948 | −0.0989 |
| 3601 | −0.2653 | −0.1860 |
| 3602 | −0.1487 | −0.1682 |
| 3603 | 0.2530 | 0.2161 |
| 3604 | 0.1562 | 0.1653 |
| 3605 | 0.0556 | −0.0740 |
| 3606 | −0.3582 | −0.3250 |
| 3607 | −0.6016 | −0.2498 |
| 3608 | −0.4632 | −0.1732 |
| 3609 | −0.7017 | −0.2571 |
| 3610 | −0.6669 | −0.3597 |
| 3611 | −0.4609 | −0.0806 |
| 3612 | −1.1828 | −0.5130 |
| 3613 | −1.3720 | −0.5901 |
| 3614 | −2.2294 | −1.3354 |
| 3615 | −2.6956 | −1.6841 |
| 3616 | −0.9745 | −0.3097 |
| 3617 | −0.4727 | −0.4828 |
| 3618 | −0.2409 | −0.0700 |
| 3619 | −0.7012 | −0.3244 |
| 3620 | −0.7103 | −0.0184 |
| 3621 | −1.5205 | −0.8541 |
| 3622 | −1.9097 | −1.1174 |
| 3623 | −1.4267 | −0.7179 |
| 3624 | −0.9269 | −0.5197 |
| 3625 | −0.5775 | −0.1805 |
| 3626 | −1.6068 | −0.8562 |
| 3627 | −1.8065 | −0.8846 |
| 3628 | −0.6095 | −0.2797 |
| 3629 | 0.0616 | 0.1545 |
| 3630 | −0.4946 | −0.1218 |
| 3631 | 0.2111 | 0.5455 |
| 3632 | 0.1446 | 0.2120 |
| 3633 | −0.0361 | 0.2558 |
| 3634 | 0.2105 | 0.3505 |
| 3635 | −0.5164 | −0.2773 |
| 3636 | −0.3142 | −0.1470 |
| 3637 | −0.3062 | −0.0815 |
| 3638 | −1.2010 | −0.5013 |
| 3639 | −1.3650 | −0.6889 |
| 3640 | −2.7044 | −1.5048 |
| 3641 | −2.4767 | −1.2990 |
| 3642 | −0.3075 | 0.1915 |
| 3643 | 0.0668 | 0.2320 |
| 3644 | 0.1260 | 0.1088 |
| 3645 | −0.2224 | −0.0350 |
| 3646 | −0.4072 | −0.0328 |
| 3647 | −1.2555 | −0.6835 |
| 3648 | −1.9614 | −0.9218 |
| 3649 | −1.7198 | −0.9177 |
| 3650 | −1.1859 | −0.6095 |
| 3651 | −0.6788 | −0.1107 |
| 3652 | −1.3040 | −0.7265 |
| 3653 | −0.8762 | −0.2100 |
| 3654 | −0.0706 | −0.0031 |
| 3655 | 0.1123 | 0.1012 |
| 3656 | 0.4096 | 0.5286 |
| 3657 | 0.1043 | 0.1039 |
| 3658 | 0.2247 | 0.3261 |
| 3659 | −0.4147 | −0.3101 |
| 3660 | −0.3476 | −0.0225 |
| 3661 | −0.1968 | −0.0928 |
| 3662 | −0.1174 | 0.1090 |
| 3663 | −1.1018 | −0.4812 |
| 3664 | −0.3143 | −0.0155 |
| 3665 | 0.0859 | 0.0522 |
| 3666 | −0.5218 | −0.1111 |
| 3667 | −0.2070 | 0.0362 |
| 3668 | 0.1721 | 0.1037 |
| 3669 | −0.1819 | −0.0326 |
| 3670 | −1.0517 | −0.4065 |
| 3671 | −0.9464 | −0.0715 |
| 3672 | 0.2220 | 0.4830 |
| 3673 | 0.3636 | 0.3177 |
| 3674 | 0.4927 | 0.4113 |
| 3675 | 0.1753 | 0.2096 |
| 3676 | 0.2912 | 0.1367 |
| 3677 | −0.0267 | −0.0441 |
| 3678 | −0.0622 | 0.0076 |
| 3679 | 0.8972 | 0.8312 |
| 3680 | −0.0575 | 0.0481 |
| 3681 | 0.2716 | 0.2170 |
| 3682 | 0.3474 | 0.6009 |
| 3683 | 0.1165 | 0.2648 |
| 3684 | 0.0354 | 0.1638 |
| 3685 | 0.1521 | 0.1534 |
| 3686 | 0.1855 | 0.2316 |
| 3687 | 0.0816 | 0.1675 |
| 3688 | −0.2594 | −0.0299 |
| 3689 | −0.1511 | −0.1117 |
| 3690 | −0.0788 | 0.2959 |
| 3691 | 0.0219 | 0.4376 |
| 3692 | −0.6198 | 0.0010 |
| 3693 | −0.9111 | 0.0283 |
| 3694 | −2.7198 | −1.5187 |
| 3695 | −2.3341 | −1.1304 |
| 3696 | −0.8652 | −0.3453 |
| 3697 | −0.1374 | 0.0439 |
| 3698 | −0.6141 | −0.2269 |
| 3699 | −0.3952 | 0.0503 |
| 3700 | −0.7037 | −0.2627 |
| 3701 | −1.5155 | −0.7281 |
| 3702 | −1.4859 | −0.7755 |
| 3703 | −0.5488 | −0.0770 |
| 3704 | −0.1624 | −0.0285 |
| 3705 | −0.4721 | 0.0344 |
| 3706 | −0.9048 | −0.3789 |
| 3707 | −0.4851 | 0.0210 |
| 3708 | −0.1538 | −0.0593 |
| 3709 | −0.4584 | −0.1921 |
| 3710 | 0.9657 | 0.7010 |
| 3711 | −0.5900 | −0.3670 |
| 3712 | −0.3751 | −0.2187 |
| 3713 | −6.6103 | −6.4389 |
| 3714 | −6.6670 | −5.8711 |
| 3715 | −6.5462 | −6.3747 |
| 3716 | 0.1479 | −0.0674 |
| 3717 | 0.0185 | 0.1212 |
| 3718 | 0.1261 | 0.2565 |
| 3719 | 0.2649 | 0.1810 |
| 3720 | 0.1467 | 0.0603 |
| 3721 | −0.2376 | −0.1994 |
| 3722 | −0.4029 | −0.3422 |
| 3723 | 0.0912 | 0.2361 |
| 3724 | −0.0448 | 0.0067 |
| 3725 | −7.1208 | −6.5508 |
| 3726 | −7.8909 | −7.3410 |
| 3727 | −7.9173 | −6.3129 |
| 3728 | −6.3232 | −6.8299 |
| 3729 | −6.5660 | −6.6576 |
| 3730 | −6.5578 | −6.5881 |
| 3731 | −1.9897 | −1.1980 |
| 3732 | −1.3420 | −0.1297 |
| 3733 | −0.8629 | −0.2536 |
| 3734 | −0.6479 | −0.0343 |
| 3735 | −1.3591 | −0.6919 |
| 3736 | −1.6052 | −0.9177 |
| 3737 | −1.2376 | −0.4101 |
| 3738 | −2.1335 | −1.0905 |
| 3739 | −2.2272 | −0.9022 |
| 3740 | −1.7603 | −0.9709 |
| 3741 | −1.5292 | −0.8834 |
| 3742 | −0.9992 | −0.5166 |
| 3743 | −1.1001 | −0.4502 |
| 3744 | −1.6066 | −0.8165 |
| 3745 | −3.1501 | −2.0481 |
| 3746 | −3.0733 | −1.6838 |
| 3747 | −2.7858 | −1.7811 |
| 3748 | −2.0530 | −1.1040 |
| 3749 | −1.0257 | −0.4796 |
| 3750 | −1.4423 | −0.7901 |
| 3751 | −0.8159 | −0.1649 |
| 3752 | −0.5485 | −0.1426 |
| 3753 | −0.2077 | 0.0230 |
| 3754 | −0.1692 | 0.0353 |
| 3755 | 0.2299 | 0.3595 |
| 3756 | 0.1226 | 0.2551 |
| 3757 | 0.3669 | 0.3405 |
| 3758 | 0.0226 | 0.1316 |
| 3759 | 0.1025 | 0.1716 |
| 3760 | 0.0937 | 0.2427 |
| 3761 | 0.4615 | 0.4891 |
| 3762 | −0.0471 | 0.1277 |
| 3763 | 0.0771 | 0.3126 |
| 3764 | 0.1602 | 0.3812 |
| 3765 | 0.1168 | 0.1601 |
| 3766 | −0.1229 | −0.0133 |
| 3767 | 0.0377 | 0.1236 |
| 3768 | −0.1936 | 0.2087 |
| 3769 | −1.1037 | −0.3663 |
| 3770 | −2.7142 | −1.8371 |
| 3771 | −2.2169 | −0.9507 |
| 3772 | −0.8267 | −0.2781 |
| 3773 | −0.5995 | −0.0810 |
| 3774 | −0.1158 | 0.1724 |
| 3775 | −0.7023 | −0.1228 |
| 3776 | −1.2452 | −0.4765 |
| 3777 | −1.2617 | −0.7049 |
| 3778 | −2.0607 | −0.9821 |
| 3779 | −0.4303 | 0.9917 |
| 3780 | −0.4639 | −0.0857 |
| 3781 | −0.0586 | 0.0715 |
| 3782 | −0.1455 | 0.0497 |
| 3783 | −0.2681 | 0.1055 |
| 3784 | 0.3717 | 0.3063 |
| 3785 | 0.2207 | 0.2138 |
| 3786 | −0.1653 | 0.1069 |
| 3787 | −0.5095 | 0.0159 |
| 3788 | −0.6963 | −0.1975 |
| 3789 | −2.0648 | −1.0341 |
| 3790 | −2.5705 | −1.2881 |
| 3791 | −2.3167 | −1.2264 |
| 3792 | −2.8388 | −1.5957 |
| 3793 | −2.8725 | −1.4941 |
| 3794 | −2.3413 | −1.1979 |
| 3795 | −1.3551 | −0.4832 |
| 3796 | −0.9870 | −0.5305 |
| 3797 | −0.7153 | −0.1909 |
| 3798 | −0.8816 | −0.3397 |
| 3799 | −2.8112 | −1.5518 |
| 3800 | −1.8032 | −0.9632 |
| 3801 | −2.5367 | −1.1941 |
| 3802 | −2.4224 | −1.3335 |
| 3803 | −1.2752 | −0.2312 |
| 3804 | −1.3238 | −0.6241 |
| 3805 | −0.8582 | −0.3499 |
| 3806 | −0.2521 | 0.1802 |
| 3807 | −0.2855 | −0.0192 |
| 3808 | −0.2604 | −0.0882 |
| 3809 | −0.2029 | 0.0489 |
| 3810 | −0.6620 | −0.2028 |
| 3811 | −0.1029 | 0.0875 |
| 3812 | −0.8157 | −0.2993 |
| 3813 | −1.8832 | −0.9071 |
| 3814 | −2.1965 | −1.0238 |
| 3815 | −2.2303 | −1.4157 |
| 3816 | −1.4450 | −0.0178 |
| 3817 | −2.5140 | −1.3709 |
| 3818 | −2.6814 | −1.4115 |
| 3819 | −1.9138 | −0.7965 |
| 3820 | −2.5097 | −1.4805 |
| 3821 | −1.3987 | −0.7231 |
| 3822 | −1.0824 | −0.4571 |
| 3823 | −0.3037 | 0.1448 |
| 3824 | −0.6652 | 0.0205 |
| 3825 | −2.1865 | −1.2341 |
| 3826 | −2.5509 | −1.5239 |
| 3827 | −2.1358 | −1.0643 |
| 3828 | −1.8494 | −0.8651 |
| 3829 | −1.1086 | −0.4576 |
| 3830 | −1.3151 | −0.5997 |
| 3831 | −1.5385 | −0.8916 |
| 3832 | −0.8832 | −0.2915 |
| 3833 | −0.9407 | −0.3956 |
| 3834 | −0.0136 | 0.1605 |
| 3835 | −0.0252 | 0.1395 |
| 3836 | −0.1409 | 0.1251 |
| 3837 | −0.1000 | −0.1398 |
| 3838 | 0.0507 | −0.0084 |
| 3839 | 0.2537 | 0.2821 |
| 3840 | −0.4245 | −0.2666 |
| 3841 | −0.4470 | −0.5817 |
| 3842 | −7.7951 | −7.7392 |
| 3843 | −6.6152 | −6.5507 |
| 3844 | −7.3824 | −6.3879 |
| 3845 | −6.6939 | −8.0370 |
| 3846 | −8.2173 | −7.4610 |
| 3847 | −7.1848 | −7.6763 |
| 3848 | −0.8947 | −0.3952 |
| 3849 | −1.7841 | −1.0846 |
| 3850 | −1.0333 | −0.3654 |
| 3851 | −0.4336 | −0.0154 |
| 3852 | −0.4770 | −0.3016 |
| 3853 | −1.8007 | −0.9576 |
| 3854 | −2.3532 | −1.4240 |
| 3855 | −2.7858 | −1.7034 |
| 3856 | −3.2892 | −2.0858 |
| 3857 | −3.6335 | −2.7189 |
| 3858 | −4.4349 | −2.5291 |
| 3859 | −3.8024 | −2.5555 |
| 3860 | −1.5494 | −0.5047 |
| 3861 | −3.4732 | −2.3159 |
| 3862 | −1.4722 | −0.7757 |
| 3863 | −2.9028 | −1.7105 |
| 3864 | −3.2498 | −1.8531 |
| 3865 | −2.7448 | −1.0525 |
| 3866 | −3.0800 | −1.7711 |
| 3867 | −2.5465 | −1.4106 |
| 3868 | −1.1538 | −0.2917 |
| 3869 | −1.4123 | −0.7039 |
| 3870 | −0.8210 | −0.3623 |
| 3871 | −0.4194 | −0.3083 |
| 3872 | −0.1116 | −0.0424 |
| 3873 | 0.0020 | 0.3486 |
| 3874 | 0.1383 | 0.2687 |
| 3875 | −0.3176 | −0.1227 |
| 3876 | −0.1069 | 0.0395 |
| 3877 | −0.3039 | −0.0814 |
| 3878 | 0.1043 | 0.2540 |
| 3879 | 0.0634 | 0.2149 |
| 3880 | −0.2647 | −0.1246 |
| 3881 | −0.1912 | −0.0254 |
| 3882 | −0.6951 | −0.3121 |
| 3883 | −0.3633 | −0.0565 |
| 3884 | −0.7923 | −0.1811 |
| 3885 | −1.8221 | −1.0672 |
| 3886 | −1.8377 | −1.1566 |
| 3887 | −1.9299 | −1.1633 |
| 3888 | −1.1371 | −0.2829 |
| 3889 | −0.2420 | 0.1446 |
| 3890 | −0.1731 | −0.8035 |
| 3891 | −0.0754 | 0.2170 |
| 3892 | −0.8371 | −0.3460 |
| 3893 | −2.2494 | −1.1168 |
| 3894 | −2.5760 | −1.7004 |
| 3895 | −2.2619 | −1.3856 |
| 3896 | −1.1496 | −0.4850 |
| 3897 | 1.0843 | 1.0330 |
| 3898 | 0.0194 | 0.0300 |
| 3899 | 0.4735 | 0.3391 |
| 3900 | 0.0364 | 0.2375 |
| 3901 | −0.2936 | −0.3552 |
| 3902 | −0.1969 | −0.1804 |
| 3903 | −0.4510 | −0.1950 |
| 3904 | −0.6307 | −0.3177 |
| 3905 | −0.9492 | −0.4200 |
| 3906 | −0.8309 | −0.2319 |
| 3907 | −2.1130 | −1.2277 |
| 3908 | −2.3936 | −1.3696 |
| 3909 | −2.7245 | −1.5341 |
| 3910 | −3.2207 | −1.8024 |
| 3911 | −2.7229 | −1.8116 |
| 3912 | −2.7452 | −1.7118 |
| 3913 | −2.6558 | −1.2485 |
| 3914 | −2.2330 | −1.3629 |
| 3915 | −2.8029 | −1.7141 |
| 3916 | −1.9187 | −1.2206 |
| 3917 | −2.9916 | −1.8797 |
| 3918 | −3.3533 | −2.0066 |
| 3919 | −3.3050 | −1.9791 |
| 3920 | −2.3746 | −1.4180 |
| 3921 | −2.3163 | −1.0773 |
| 3922 | −0.7157 | −0.6205 |
| 3923 | −0.0575 | 0.2491 |
| 3924 | −0.1865 | −0.1292 |
| 3925 | 0.0081 | 0.1496 |
| 3926 | 0.0834 | 0.4999 |
| 3927 | −0.6296 | −0.1794 |
| 3928 | −0.7982 | −0.2766 |
| 3929 | −0.5029 | −0.0635 |
| 3930 | −0.9681 | −0.1932 |
| 3931 | −1.6209 | −0.5626 |
| 3932 | −1.4860 | −0.6959 |
| 3933 | −2.4215 | −1.3238 |
| 3934 | −2.7021 | −1.5688 |
| 3935 | −2.8460 | −1.6477 |
| 3936 | −3.3087 | −1.8582 |
| 3937 | −2.9202 | −1.6205 |
| 3938 | −2.4528 | −1.1929 |
| 3939 | −3.0506 | −1.8012 |
| 3940 | −2.4127 | −1.5068 |
| 3941 | −2.6009 | −1.3663 |
| 3942 | −1.6939 | −0.4220 |
| 3943 | −2.5743 | −1.6360 |
| 3944 | −2.4953 | −1.5113 |
| 3945 | −2.5817 | −1.4304 |
| 3946 | −2.1973 | −1.0842 |
| 3947 | −2.1175 | −1.1388 |
| 3948 | −1.6923 | −1.1109 |
| 3949 | −1.8457 | −0.9609 |
| 3950 | −1.0391 | −0.5563 |
| 3951 | −0.4103 | −0.3085 |
| 3952 | −0.1065 | −0.1810 |
| 3953 | −0.2992 | −0.3197 |
| 3954 | 0.0080 | −0.0273 |
| 3955 | 0.1571 | 0.0018 |
| 3956 | 0.1187 | −0.0014 |
| 3957 | −4.2770 | −2.5534 |
| 3958 | −3.2419 | −2.3449 |
| 3959 | −0.6152 | −0.3664 |
| 3960 | −0.7793 | −0.3715 |
| 3961 | −0.9565 | −0.4858 |
| 3962 | −0.8604 | −0.5104 |
| 3963 | −0.8511 | −0.4197 |
| 3964 | −1.7232 | −0.9323 |
| 3965 | −2.1794 | −1.1737 |
| 3966 | −3.7282 | −2.4069 |
| 3967 | −3.6827 | −2.2868 |
| 3968 | −3.9633 | −2.5074 |
| 3969 | −3.6147 | −2.3600 |
| 3970 | −5.7595 | −4.3250 |
| 3971 | −5.1180 | −3.4529 |
| 3972 | −3.0156 | −1.7906 |
| 3973 | −4.5720 | −2.8892 |
| 3974 | −5.7903 | −3.8463 |
| 3975 | −5.8893 | −4.4369 |
| 3976 | −4.2119 | −2.5864 |
| 3977 | −3.5761 | −1.8017 |
| 3978 | −4.2974 | −2.8132 |
| 3979 | −3.3266 | −2.1330 |
| 3980 | −3.1987 | −2.0847 |
| 3981 | −4.7911 | −3.0516 |
| 3982 | −2.8451 | −1.8240 |
| 3983 | −3.4318 | −2.2881 |
| 3984 | −1.5112 | −1.0627 |
| 3985 | −4.7825 | −3.3026 |
| 3986 | −2.4516 | −1.3520 |
| 3987 | −3.5690 | −2.8254 |
| 3988 | −6.9564 | −6.4631 |
| 3989 | −3.5961 | −2.5577 |
| 3990 | −4.8013 | −3.7384 |
| 3991 | −4.9157 | −3.7048 |
| 3992 | −2.5321 | −1.8533 |
| 3993 | −4.9343 | −3.3363 |
| 3994 | −6.3558 | −3.7381 |
| 3995 | −5.0854 | −3.3609 |
| 3996 | −3.4805 | −2.0429 |
| 3997 | −3.3227 | −2.2239 |
| 3998 | −4.2238 | −2.8769 |
| 3999 | −3.2264 | −1.5717 |
| 4000 | −5.5634 | −4.3255 |
| 4001 | −5.0848 | −4.2353 |
| 4002 | −6.8895 | −6.4243 |
| 4003 | −8.6461 | −7.6267 |
| 4004 | −4.0559 | −2.9363 |
| 4005 | −6.5983 | −5.1467 |
| 4006 | −15.0000 | −6.1749 |
| 4007 | −5.1914 | −3.8524 |
| 4008 | −3.8071 | −2.5748 |
| 4009 | −4.6349 | −2.8150 |
| 4010 | −3.6521 | −2.1964 |
| 4011 | −4.6661 | −2.7536 |
| 4012 | −5.0217 | −3.1467 |
| 4013 | −3.7002 | −2.1919 |
| 4014 | −4.0916 | −2.5890 |
| 4015 | −2.8705 | −1.4735 |
| 4016 | −3.8946 | −2.2092 |
| 4017 | −5.2073 | −3.4416 |
| 4018 | −3.9015 | −2.7375 |
| 4019 | −5.2557 | −4.5279 |
| 4020 | −3.2722 | −1.8281 |
| 4021 | −2.9829 | −1.7571 |
| 4022 | −7.9421 | −5.0862 |
| 4023 | −3.4527 | −2.2656 |
| 4024 | −5.3789 | −4.4139 |
| 4025 | −5.6407 | −4.0497 |
| 4026 | −4.8675 | −3.3783 |
| 4027 | −4.0998 | −3.2053 |
| 4028 | −4.5055 | −2.8720 |
| 4029 | −3.2422 | −2.5015 |
| 4030 | −5.9397 | −4.6163 |
| 4031 | −4.3377 | −2.3926 |
| 4032 | −7.8855 | −5.3218 |
| 4033 | −6.6498 | −5.3354 |
| 4034 | −5.6591 | −3.7222 |
| 4035 | −4.2866 | −2.3426 |
| 4036 | −4.1994 | −2.6555 |
| 4037 | −0.5867 | −0.1385 |
| 4038 | −0.4394 | 0.0374 |
| 4039 | −0.7508 | −0.3047 |
| 4040 | 0.0941 | 0.2647 |
| 4041 | −0.6200 | −0.2123 |
| 4042 | −0.0213 | 0.3888 |
| 4043 | −0.9611 | −0.4077 |
| 4044 | 0.0388 | 0.3442 |
| 4045 | −1.1953 | −0.7035 |
| 4046 | −0.2093 | 0.0686 |
| 4047 | −0.3223 | −0.2660 |
| 4048 | −0.4455 | −0.1354 |
| 4049 | −0.1302 | −0.0055 |
| 4050 | −0.2372 | −0.1959 |
| 4051 | −0.1709 | −0.2292 |
| 4052 | 0.8826 | 0.5588 |
| 4053 | −0.0737 | 0.3054 |
| 4054 | −0.6169 | 0.6621 |
| 4055 | −0.1614 | 0.2482 |
| 4056 | −0.5976 | −0.2513 |
| 4057 | −0.7230 | −0.2977 |
| 4058 | −0.5139 | −0.0925 |
| 4059 | −0.6313 | −0.4020 |
| 4060 | −0.6281 | −0.2748 |
| 4061 | −0.6786 | −0.4674 |
| 4062 | −0.5161 | −0.2033 |
| 4063 | −0.6426 | −0.4339 |
| 4064 | −0.0836 | −0.0126 |
| 4065 | −0.1998 | −0.0269 |
| 4066 | −0.1543 | −0.2367 |
| 4067 | −0.4159 | −0.2229 |
| 4068 | −0.3122 | 0.0737 |
| 4069 | −0.6880 | −0.3456 |
| 4070 | −0.7606 | −0.3950 |
| 4071 | −0.5957 | −0.3650 |
| 4072 | −0.2804 | −0.1135 |
| 4073 | −1.2219 | −0.5064 |
| 4074 | −0.9748 | −0.4882 |
| 4075 | −0.6093 | −0.3414 |
| 4076 | −1.6243 | −0.7931 |
| 4077 | −2.4821 | −1.3362 |
| 4078 | −7.5388 | −4.9923 |
| 4079 | −7.5635 | −5.7196 |
| 4080 | −3.5944 | −2.5715 |
| 4081 | −4.7852 | −2.6058 |
| 4082 | −0.2801 | −0.1887 |
| 4083 | −0.6145 | −0.0889 |
| 4084 | −3.5454 | −1.9086 |
| 4085 | −3.6858 | −1.9000 |
| 4086 | −1.8691 | −0.7719 |
| 4087 | −2.9235 | −1.6448 |
| 4088 | −2.0571 | −1.2492 |
| 4089 | −2.4869 | −1.3495 |
| 4090 | −1.2984 | −0.6806 |
| 4091 | −0.8234 | −0.5493 |
| 4092 | −0.0748 | −0.0459 |
| 4093 | −1.0120 | −0.6734 |
| 4094 | −1.4180 | −0.7176 |
| 4095 | −3.1132 | −1.9571 |
| 4096 | −4.6135 | −2.7551 |
| 4097 | −7.3866 | −5.5293 |
| 4098 | −9.3999 | −6.3354 |
| 4099 | −4.4924 | −3.1233 |
| 4100 | −0.7228 | −0.3319 |
| 4101 | −4.8721 | −2.8886 |
| 4102 | −9.1119 | −6.8701 |
| 4103 | −9.1708 | −7.4144 |
| 4104 | −10.3534 | −9.4451 |
| 4105 | −8.1852 | −10.0138 |
| 4106 | −3.3964 | −1.5781 |
| 4107 | −1.6429 | −0.9812 |
| 4108 | −1.1936 | −0.5794 |
| 4109 | −1.8197 | −0.7503 |
| 4110 | −1.3845 | −0.5896 |
| 4111 | −2.8410 | −1.5967 |
| 4112 | −1.4996 | −0.7388 |
| 4113 | −1.5744 | −1.0508 |
| 4114 | 0.1891 | 0.3493 |
| 4115 | −0.2963 | −0.1535 |
| 4116 | −0.5279 | −0.2648 |
| 4117 | −2.4876 | −1.2704 |
| 4118 | −8.0899 | −5.7091 |
| 4119 | −3.9825 | −2.0838 |
| 4120 | −1.4939 | −0.6887 |
| 4121 | −0.5932 | −0.2624 |
| 4122 | −0.5714 | −0.2354 |
| 4123 | −0.9800 | −0.1823 |
| 4124 | −0.1340 | −0.0585 |
| 4125 | −0.2942 | −0.0188 |
| 4126 | −0.6273 | −0.3336 |
| 4127 | −1.0036 | 0.0136 |
| 4128 | −1.6053 | −0.7043 |
| 4129 | −2.7858 | −1.5551 |
| 4130 | −5.2728 | −3.0045 |
| 4131 | −5.5197 | −3.8659 |
| 4132 | −0.5016 | −0.1254 |
| 4133 | −0.8077 | −0.4151 |
| 4134 | −0.4514 | 0.0587 |
| 4135 | −2.4424 | −1.6148 |
| 4136 | −3.9106 | −2.0814 |
| 4137 | −3.2782 | −1.7224 |
| 4138 | −7.6419 | −7.1810 |
| 4139 | −8.9810 | −7.2247 |
| 4140 | −8.1018 | −7.4449 |
| 4141 | −0.3868 | −0.0189 |
| 4142 | −1.7988 | −0.8596 |
| 4143 | −6.9247 | −6.8845 |
| 4144 | −0.8343 | −0.5536 |
| 4145 | −7.0229 | −5.8863 |
| 4146 | −1.3126 | −0.9575 |
| 4147 | −5.7091 | −6.9609 |
| 4148 | −8.0609 | −5.6524 |
| 4149 | −2.6741 | −1.7829 |
| 4150 | −4.7610 | −4.0261 |
| 4151 | −0.2993 | −0.3204 |
| 4152 | −1.7133 | −1.0255 |
| 4153 | −2.4119 | −1.3197 |
| 4154 | −2.5067 | −1.8653 |
| 4155 | −3.4838 | −2.1707 |
| 4156 | −3.7982 | −2.3365 |
| 4157 | −3.8067 | −2.4165 |
| 4158 | −3.2825 | −2.0355 |
| 4159 | −2.3802 | −1.2325 |
| 4160 | −0.6672 | −0.1661 |
| 4161 | −2.7418 | −1.7506 |
| 4162 | −5.4951 | −4.1905 |
| 4163 | −6.9981 | −4.7910 |
| 4164 | −7.4257 | −5.4888 |
| 4165 | −7.1841 | −6.5102 |
| 4166 | −8.9882 | −5.5437 |
| 4167 | −6.3946 | −5.6668 |
| 4168 | −7.2991 | −6.1277 |
| 4169 | −6.5179 | −4.6685 |
| 4170 | −3.5445 | −2.4481 |
| 4171 | −4.4952 | −4.4211 |
| 4172 | −4.6834 | −4.0124 |
| 4173 | −5.6052 | −3.6136 |
| 4174 | −5.6899 | −3.8132 |
| 4175 | −6.3603 | −5.2273 |
| 4176 | −6.0123 | −4.3598 |
| 4177 | −5.7591 | −3.3904 |
| 4178 | −5.0021 | −3.6083 |
| 4179 | −3.9751 | −2.1187 |
| 4180 | −3.6606 | −2.1447 |
| 4181 | −4.3178 | −2.8447 |
| 4182 | −5.8759 | −3.3404 |
| 4183 | −3.6016 | −2.3733 |
| 4184 | −2.7036 | −1.5494 |
| 4185 | −2.3867 | −1.3141 |
| 4186 | −0.4156 | −0.0812 |
| 4187 | −2.8466 | −1.7705 |
| 4188 | −5.0731 | −3.4696 |
| 4189 | −5.6188 | −4.0734 |
| 4190 | −5.9523 | −4.5810 |
| 4191 | −8.2778 | −5.8168 |
| 4192 | −6.2424 | −4.1052 |
| 4193 | −4.5717 | −2.6943 |
| 4194 | −1.1539 | −0.4645 |
| 4195 | −1.3774 | −0.7380 |
| 4196 | −4.4779 | −2.8896 |
| 4197 | −6.8045 | −6.0767 |
| 4198 | −8.5362 | −6.9053 |
| 4199 | −10.0015 | −7.3707 |
| 4200 | −8.1663 | −7.2579 |
| 4201 | −8.6062 | −8.1129 |
| 4202 | −8.5856 | −7.8292 |
| 4203 | −7.3784 | −6.1481 |
| 4204 | −7.2603 | −5.0533 |
| 4205 | −7.1605 | −5.7411 |
| 4206 | −6.5793 | −5.4410 |
| 4207 | −6.7388 | −5.5674 |
| 4208 | −6.5820 | −5.0287 |
| 4209 | −8.3013 | −5.5719 |
| 4210 | −10.5450 | −7.1256 |
| 4211 | −8.5315 | −7.4532 |
| 4212 | −9.2981 | −10.1267 |
| 4213 | −11.0468 | −7.7054 |
| 4214 | −9.2545 | −6.3282 |
| 4215 | −6.2852 | −4.7993 |
| 4216 | −6.0291 | −3.6251 |
| 4217 | −4.3772 | −2.3729 |
| 4218 | −3.4461 | −2.0312 |
| 4219 | −3.3663 | −1.8611 |
| 4220 | −2.1677 | −1.2129 |
| 4221 | −4.1013 | −2.7563 |
| 4222 | −3.9280 | −2.5983 |
| 4223 | −4.0370 | −2.7000 |
| 4224 | −4.2518 | −3.0081 |
| 4225 | −4.7767 | −2.6367 |
| 4226 | −2.5234 | −1.4724 |
| 4227 | −3.1750 | −2.1840 |
| 4228 | −3.0882 | −1.9811 |
| 4229 | −3.4769 | −2.1071 |
| 4230 | −4.2505 | −2.7741 |
| 4231 | −2.7144 | −1.7127 |
| 4232 | −1.3113 | −0.4433 |
| 4233 | −5.9920 | −4.4421 |
| 4234 | −8.3474 | −6.2584 |
| 4235 | −8.6983 | −6.7196 |
| 4236 | −7.3016 | −6.3417 |
| 4237 | −1.7008 | −0.7549 |
| 4238 | −3.2926 | −1.9782 |
| 4239 | −4.9849 | −3.1806 |
| 4240 | −4.6550 | −3.4998 |
| 4241 | −5.0363 | −3.6425 |
| 4242 | 0.0887 | 0.0823 |
| 4243 | −0.0654 | −0.1674 |
| 4244 | −0.4098 | −0.2030 |
| 4245 | −0.4623 | −0.0904 |
| 4246 | −0.8455 | −0.3030 |
| 4247 | −0.9395 | −0.5686 |
| 4248 | −5.7367 | −4.4057 |
| 4249 | −1.4331 | −0.5754 |
| 4250 | −1.2159 | −0.4522 |
| 4251 | −8.6796 | −5.1390 |
| 4252 | −6.1987 | −5.0590 |
| 4253 | −6.8613 | −6.8419 |
| 4254 | −4.2232 | −3.2601 |
| 4255 | −2.0656 | −1.0923 |
| 4256 | −1.8605 | −0.9405 |
| 4257 | −5.3148 | −4.5783 |
| 4258 | −3.0681 | −1.8346 |
| 4259 | −4.9889 | −3.0140 |
| 4260 | −7.1633 | −4.4339 |
| 4261 | −4.9989 | −3.5511 |
| 4262 | −6.9439 | −5.8396 |
| 4263 | −6.6411 | −5.3745 |
| 4264 | −6.4560 | −4.2666 |
| 4265 | −2.7336 | −1.8293 |
| 4266 | −0.0882 | 0.0523 |
| 4267 | −0.3627 | −0.2952 |
| 4268 | −0.2184 | −0.1239 |
| 4269 | 0.1647 | 0.0885 |
| 4270 | 0.5787 | 0.7525 |
| 4271 | 0.1581 | 0.1731 |
| 4272 | −0.2887 | −0.3198 |
| 4273 | 0.1893 | −0.0354 |
| 4274 | 0.1482 | 0.0429 |
| 4275 | −0.0734 | −0.1184 |
| 4276 | 0.0515 | −0.0510 |
| 4277 | 0.3467 | 0.2425 |
| 4278 | −0.0413 | −0.0977 |
| 4279 | −0.1681 | −0.0926 |
| 4280 | −0.4317 | −0.2283 |
| 4281 | −1.2357 | −0.3548 |
| 4282 | −1.3706 | −0.6826 |
| 4283 | −1.7344 | −0.9155 |
| 4284 | −2.3167 | −1.1726 |
| 4285 | −2.8847 | −1.5462 |
| 4286 | −2.5894 | −1.5156 |
| 4287 | −2.5242 | −1.2164 |
| 4288 | −2.5027 | −1.3552 |
| 4289 | −2.9463 | −1.5414 |
| 4290 | −2.5669 | −1.4039 |
| 4291 | −2.8592 | −1.5864 |
| 4292 | −2.8346 | −1.6958 |
| 4293 | −3.1266 | −1.8493 |
| 4294 | −2.9827 | −1.5558 |
| 4295 | −3.5692 | −1.6892 |
| 4296 | −2.9646 | −1.6460 |
| 4297 | −3.1527 | −1.6093 |
| 4298 | −3.1710 | −1.9017 |
| 4299 | −3.1493 | −2.1030 |
| 4300 | −3.0906 | −1.8630 |
| 4301 | −3.3345 | −1.9880 |
| 4302 | −3.2093 | −1.9537 |
| 4303 | −3.0574 | −1.8476 |
| 4304 | −3.1651 | −1.7519 |
| 4305 | −3.0836 | −1.7950 |
| 4306 | −3.2920 | −1.6702 |
| 4307 | −3.9095 | −2.2168 |
| 4308 | −5.1460 | −2.8286 |
| 4309 | −5.1286 | −3.2552 |
| 4310 | −5.3711 | −3.8778 |
| 4311 | −6.9313 | −5.0754 |
| 4312 | −7.0657 | −5.2280 |
| 4313 | −6.3463 | −4.0456 |
| 4314 | 6.7799 | −5.3943 |
| 4315 | −7.3159 | −5.1026 |
| 4316 | −8.6164 | −6.0824 |
| 4317 | −8.2670 | −6.0314 |
| 4318 | −9.2838 | −6.5274 |
| 4319 | −9.3181 | −5.8836 |
| 4320 | −6.6746 | −5.4658 |
| 4321 | −9.1124 | −4.8749 |
| 4322 | −8.0568 | −6.1077 |
| 4323 | −8.9872 | −6.1153 |
| 4324 | −6.9138 | −5.7728 |
| 4325 | −7.1361 | −4.5153 |
| 4326 | −7.2192 | −5.2784 |
| 4327 | −7.0939 | −6.0220 |
| 4328 | −7.1006 | −4.7979 |
| 4329 | −7.6235 | −4.9840 |
| 4330 | −6.8873 | −4.7544 |
| 4331 | −6.6799 | −5.0611 |
| 4332 | −6.5452 | −4.7704 |
| 4333 | −5.2359 | −3.4795 |
| 4334 | −7.0448 | −5.6510 |
| 4335 | −7.9245 | −5.6461 |
| 4336 | −7.4728 | −5.6493 |
| 4337 | −8.2866 | −6.1152 |
| 4338 | −7.3930 | −5.4281 |
| 4339 | −7.8563 | −5.2698 |
| 4340 | −6.8064 | −5.2364 |
| 4341 | −7.7156 | −5.1905 |
| 4342 | −7.3397 | −5.6593 |
| 4343 | −7.4485 | −5.7971 |
| 4344 | −7.3961 | −5.5120 |
| 4345 | −7.8495 | −6.0260 |
| 4346 | −7.8940 | −6.1377 |
| 4347 | −8.3984 | −6.1395 |
| 4348 | −7.1395 | −6.3120 |
| 4349 | −7.7741 | −5.9550 |
| 4350 | −7.5347 | −6.5978 |
| 4351 | −7.8147 | −6.3732 |
| 4352 | −7.9215 | −6.1651 |
| 4353 | −8.1969 | −5.9000 |
| 4354 | −7.8234 | −5.9857 |
| 4355 | −7.6703 | −5.8208 |
| 4356 | −7.7217 | −6.1810 |
| 4357 | −7.1485 | −6.6445 |
| 4358 | −7.4051 | −5.8913 |
| 4359 | −8.0773 | −6.2108 |
| 4360 | −7.6286 | −6.7314 |
| 4361 | −8.5862 | −6.2994 |
| 4362 | −9.4599 | −7.0661 |
| 4363 | −8.5372 | −7.7284 |
| 4364 | −10.0959 | −8.4651 |
| 4365 | −10.8159 | −9.4745 |
| 4366 | −11.5988 | −11.4273 |
| 4367 | −10.9058 | −9.4124 |
| 4368 | −12.5255 | −12.3540 |
| 4369 | −10.2981 | −12.1267 |
| 4370 | −11.6616 | −15.0000 |
| 4371 | −12.3976 | −11.2262 |
| 4372 | −9.4886 | −11.9021 |
| 4373 | −10.7323 | −11.5609 |
| 4374 | −11.0354 | −11.8640 |
| 4375 | −15.0000 | −8.7991 |
| 4376 | −9.6562 | −10.0697 |
| 4377 | −15.0000 | −9.7136 |
| 4378 | −0.0928 | 0.1697 |
| 4379 | −0.0251 | 0.2248 |
| 4380 | 0.0893 | 0.2538 |
| 4381 | −0.1069 | 0.1382 |
| 4382 | −0.2477 | 0.0638 |
| 4383 | −0.4002 | 0.0274 |
| 4384 | −0.5858 | −0.1915 |
| 4385 | 0.0916 | 0.4503 |
| 4386 | 0.2029 | 0.4486 |
| 4387 | −0.2848 | −0.0601 |
| 4388 | −0.2576 | 0.1287 |
| 4389 | −0.1256 | 0.1868 |
| 4390 | −0.5482 | −0.1718 |
| 4391 | −0.5317 | 0.0013 |
| 4392 | 0.2628 | 0.3622 |
| 4393 | 0.4406 | 0.9444 |
| 4394 | −0.3541 | −0.1041 |
| 4395 | −0.6106 | −0.4553 |
| 4396 | −0.9846 | −0.1801 |
| 4397 | −0.1535 | 0.2033 |
| 4398 | −0.1171 | 0.2453 |
| 4399 | −0.0859 | 0.2548 |
| 4400 | −0.8444 | −0.0676 |
| 4401 | −0.4879 | 0.0232 |
| 4402 | −0.5299 | −0.2444 |
| 4403 | −0.5009 | −0.1025 |
| 4404 | 0.0003 | 0.1529 |
| 4405 | 0.2422 | 0.2800 |
| 4406 | −0.5640 | −0.0561 |
| 4407 | −0.4763 | −0.2973 |
| 4408 | −0.2658 | −0.2175 |
| 4409 | −0.0299 | 0.1849 |
| 4410 | 0.2592 | 0.3388 |
| 4411 | −0.0641 | 0.1207 |
| 4412 | 0.1802 | 0.3054 |
| 4413 | 0.2669 | −0.0556 |
| 4414 | −0.3153 | 0.1862 |
| 4415 | −0.6194 | −0.0127 |
| 4416 | −0.3937 | 0.2567 |
| 4417 | 0.1141 | 0.6298 |
| 4418 | −0.6399 | −0.1898 |
| 4419 | 0.5921 | −0.2310 |
| 4420 | −0.2786 | 0.2818 |
| 4421 | −0.2468 | 0.2338 |
| 4422 | 0.1563 | 0.3611 |
| 4423 | −0.4409 | −0.2822 |
| 4424 | −0.4965 | 0.2816 |
| 4425 | −0.4324 | 0.2135 |
| 4426 | −1.0210 | −0.5078 |
| 4427 | −0.4199 | −0.0059 |
| 4428 | −0.6114 | −0.2296 |
| 4429 | −0.1407 | 0.1940 |
| 4430 | −0.3626 | 0.0942 |
| 4431 | −0.9248 | −0.3784 |
| 4432 | −1.2743 | −0.5287 |
| 4433 | −0.3835 | 0.2901 |
| 4434 | −0.4619 | 0.3310 |
| 4435 | −0.8648 | −0.2030 |
| 4436 | −1.3171 | −0.6714 |
| 4437 | −0.8105 | −0.1554 |
| 4438 | −0.9386 | −0.1473 |
| 4439 | −0.3446 | −0.0717 |
| 4440 | −0.7553 | −0.5013 |
| 4441 | −0.4459 | −0.1539 |
| 4442 | −1.2869 | −0.4868 |
| 4443 | −0.3469 | −0.0234 |
| 4444 | −0.1531 | 0.0689 |
| 4445 | 0.0781 | 0.2614 |
| 4446 | −0.3784 | −0.0855 |
| 4447 | −0.0805 | 0.1194 |
| 4448 | −0.4057 | −0.0809 |
| 4449 | −0.2716 | 0.1137 |
| 4450 | −0.8925 | −0.5200 |
| 4451 | −0.4112 | 0.0335 |
| 4452 | −0.6856 | −0.2091 |
| 4453 | −0.8151 | −0.3294 |
| 4454 | −0.5609 | −0.2483 |
| 4455 | −0.5952 | 0.0039 |
| 4456 | −1.2291 | −0.4587 |
| 4457 | −0.4150 | 0.2526 |
| 4458 | −0.4832 | 0.0149 |
| 4459 | −0.6890 | −0.0668 |
| 4460 | −1.1077 | −0.5728 |
| 4461 | −1.3577 | −0.5443 |
| 4462 | −1.1894 | −0.3917 |
| 4463 | −0.8944 | −0.3646 |
| 4464 | −0.8404 | −0.6107 |
| 4465 | −0.9863 | −0.4765 |
| 4466 | −1.0465 | −0.6295 |
| 4467 | −1.2109 | −0.5278 |
| 4468 | −1.7276 | −1.0368 |
| 4469 | −0.8500 | −0.2302 |
| 4470 | −0.8042 | −0.1729 |
| 4471 | 0.9338 | −0.2728 |
| 4472 | −1.5495 | −0.6623 |
| 4473 | −1.9489 | −1.0847 |
| 4474 | −1.2539 | −0.6980 |
| 4475 | −0.5823 | −0.1411 |
| 4476 | −0.7785 | −0.6120 |
| 4477 | −0.4596 | −0.2037 |
| 4478 | −1.1086 | −0.3935 |
| 4479 | −0.9981 | −0.5321 |
| 4480 | −0.4792 | 0.2191 |
| 4481 | −0.4174 | −0.0481 |
| 4482 | −0.4710 | −0.2168 |
| 4483 | 0.2574 | 0.7933 |
| 4484 | −0.4876 | −0.2359 |
| 4485 | −0.8438 | −0.5064 |
| 4486 | −0.0597 | 0.2937 |
| 4487 | −0.0668 | 0.4310 |
| 4488 | −0.0075 | 0.2802 |
| 4489 | −0.1956 | 0.2812 |
| 4490 | −0.0306 | 0.2927 |
| 4491 | −0.5503 | 0.0206 |
| 4492 | −0.2502 | 0.1515 |
| 4493 | −0.2575 | 0.2769 |
| 4494 | 0.0050 | 0.2684 |
| 4495 | −0.2255 | 0.2695 |
| 4496 | −0.0617 | 0.3281 |
| 4497 | −0.3405 | 0.0236 |
| 4498 | −0.7789 | −0.3568 |
| 4499 | −0.2473 | 0.3700 |
| 4500 | −0.3735 | −0.3020 |
| 4501 | −0.2268 | −0.0706 |
| 4502 | −0.5106 | −0.2395 |
| 4503 | −0.3317 | 0.1984 |
| 4504 | −1.2502 | −0.5835 |
| 4505 | −0.8524 | −0.1787 |
| 4506 | −0.3267 | −0.0736 |
| 4507 | −0.5326 | 0.0119 |
| 4508 | −1.2168 | −0.4554 |
| 4509 | −0.5952 | −0.0832 |
| 4510 | −0.5800 | −0.2684 |
| 4511 | −0.7149 | −0.3167 |
| 4512 | −0.3789 | −0.0173 |
| 4513 | −0.1780 | −0.0380 |
| 4514 | −1.2420 | −0.6912 |
| 4515 | −0.2325 | 0.0433 |
| 4516 | −0.0582 | 0.2535 |
| 4517 | 0.7464 | 1.0213 |
| 4518 | 0.2554 | 0.3988 |
| 4519 | 0.0968 | 0.2240 |
| 4520 | −0.3460 | −0.1079 |
| 4521 | −0.1834 | 0.1124 |
| 4522 | 0.4059 | 0.0557 |
| 4523 | −0.5902 | −0.1390 |
| 4524 | −0.4918 | −0.0294 |
| 4525 | −0.7562 | −0.2440 |
| 4526 | −0.7892 | −0.1810 |
| 4527 | −0.9519 | −0.2061 |
| 4528 | −0.7984 | −0.3081 |
| 4529 | −0.8061 | −0.2020 |
| 4530 | −0.5900 | −0.0788 |
| 4531 | 0.8137 | −0.1631 |
| 4532 | −0.4139 | 0.0775 |
| 4533 | −1.1626 | −0.4566 |
| 4534 | −1.1039 | −0.4323 |
| 4535 | −0.8349 | −0.4786 |
| 4536 | −0.4696 | −0.1873 |
| 4537 | −0.7593 | −0.4944 |
| 4538 | −1.0887 | −0.5375 |
| 4539 | −1.2355 | −0.4869 |
| 4540 | −1.1856 | −0.4518 |
| 4541 | −1.1643 | −0.3410 |
| 4542 | −0.9590 | −0.3608 |
| 4543 | −0.8976 | −0.3216 |
| 4544 | −1.0774 | −0.2992 |
| 4545 | −1.8020 | −1.0098 |
| 4546 | −0.9263 | −0.4451 |
| 4547 | −0.9774 | −0.6273 |
| 4548 | −0.9892 | −0.5318 |
| 4549 | −0.1300 | 0.0608 |
| 4550 | −1.2471 | −0.4383 |
| 4551 | −0.7886 | −0.4694 |
| 4552 | −0.5649 | −0.2256 |
| 4553 | −0.5073 | −0.1512 |
| 4554 | −0.3131 | −0.0067 |
| 4555 | 0.1132 | 0.8137 |
| 4556 | −0.7470 | −0.5433 |
| 4557 | −0.9315 | −0.4381 |
| 4558 | −0.1049 | 0.1342 |
| 4559 | 0.2612 | 0.3757 |
| 4560 | −0.1107 | 0.1276 |
| 4561 | −0.1799 | 0.1240 |
| 4562 | 0.1302 | 0.3577 |
| 4563 | −0.3948 | −0.0947 |
| 4564 | −0.3401 | 0.0482 |
| 4565 | −0.1454 | 0.2078 |
| 4566 | −0.2097 | −0.0351 |
| 4567 | −0.1966 | 0.0107 |
| 4568 | −0.1539 | 0.4095 |
| 4569 | 0.4565 | 0.9229 |
| 4570 | −0.5050 | −0.1828 |
| 4571 | −0.3595 | −0.1506 |
| 4572 | −0.2938 | 0.0304 |
| 4573 | −0.2609 | −0.0050 |
| 4574 | −0.5506 | −0.2852 |
| 4575 | −0.3034 | 0.0140 |
| 4576 | −0.9504 | −0.4560 |
| 4577 | −0.7329 | −0.3105 |
| 4578 | −0.4157 | 0.0093 |
| 4579 | −0.4979 | 0.0034 |
| 4580 | −0.8566 | −0.4617 |
| 4581 | −0.9013 | −0.3083 |
| 4582 | −0.7945 | −0.5184 |
| 4583 | 0.2312 | 0.2493 |
| 4584 | −0.3961 | −0.2553 |
| 4585 | −0.5433 | 0.0358 |
| 4586 | −0.3481 | −0.0839 |
| 4587 | −0.4486 | −0.1328 |
| 4588 | −0.1857 | 0.0088 |
| 4589 | 0.2189 | 0.1929 |
| 4590 | 0.4798 | 0.5300 |
| 4591 | −0.1226 | −0.0760 |
| 4592 | −0.4566 | −0.1437 |
| 4593 | −8.3927 | −9.3367 |
| 4594 | −4.5421 | −2.6510 |
| 4595 | −3.1208 | −1.8725 |
| 4596 | −8.7566 | −11.5852 |
| 4597 | −15.0000 | −8.2338 |
| 4598 | −8.1118 | −5.8335 |
| 4599 | −11.8058 | −11.6344 |
| 4600 | −7.7253 | −5.3663 |
| 4601 | −3.4032 | −1.8883 |
| 4602 | −3.0625 | −1.9392 |
| 4603 | −7.3621 | −9.1907 |
| 4604 | −8.2431 | −8.6566 |
| 4605 | −7.6721 | −4.5938 |
| 4606 | −8.3143 | −8.7279 |
| 4607 | −3.4931 | −1.6561 |
| 4608 | −2.7917 | −1.2435 |
| 4609 | −4.0705 | −2.1201 |
| 4610 | −2.8478 | −1.6354 |
| 4611 | −3.3968 | −1.7887 |
| 4612 | −2.9980 | −1.6567 |
| 4613 | −3.6085 | −2.1055 |
| 4614 | −3.3854 | −2.3520 |
| 4615 | −4.6649 | −2.6861 |
| 4616 | −3.8048 | −2.2184 |
| 4617 | −3.5524 | −1.9691 |
| 4618 | −3.7235 | −2.1103 |
| 4619 | −4.1942 | −2.0228 |
| 4620 | −4.5216 | −2.6159 |
| 4621 | −4.9591 | −3.6842 |
| 4622 | −4.5748 | −2.3500 |
| 4623 | −4.1324 | −2.8369 |
| 4624 | −3.9852 | −2.5375 |
| 4625 | −3.3849 | −1.7604 |
| 4626 | −3.2030 | −1.5389 |
| 4627 | −4.3883 | −2.5741 |
| 4628 | −4.2066 | −2.1698 |
| 4629 | −3.4566 | −1.8250 |
| 4630 | −3.0797 | −1.7268 |
| 4631 | −4.2604 | −2.1355 |
| 4632 | −4.5075 | −2.6036 |
| 4633 | −4.9475 | −3.1968 |
| 4634 | −4.5765 | −2.5791 |
| 4635 | −4.4585 | −2.5478 |
| 4636 | −4.0757 | −2.4245 |
| 4637 | −3.4836 | −1.8664 |
| 4638 | −3.3376 | −2.1104 |
| 4639 | −3.8391 | −2.3916 |
| 4640 | −3.2730 | −1.7013 |
| 4641 | −3.5346 | −1.5280 |
| 4642 | −4.2236 | −3.0402 |
| 4643 | −6.3577 | −4.0488 |
| 4644 | −6.8833 | −10.1714 |
| 4645 | −7.6141 | −4.4427 |
| 4646 | −8.2277 | −15.0000 |
| 4647 | −7.3042 | −8.1328 |
| 4648 | −11.1685 | −10.9971 |
| 4649 | −2.0207 | −0.7528 |
| 4650 | −9.0291 | −8.5358 |
| 4651 | −3.6645 | −2.5888 |
| 4652 | −2.9125 | −2.2111 |
| 4653 | −5.4147 | −3.6583 |
| 4654 | −5.9467 | −4.5798 |
| 4655 | −5.4437 | −3.8747 |
| 4656 | −3.1928 | −2.1734 |
| 4657 | −4.8176 | −3.4793 |
| 4658 | −9.0656 | −6.8942 |
| 4659 | −4.1729 | −2.8270 |
| 4660 | −4.6152 | −2.9950 |
| 4661 | −7.0611 | −5.7966 |
| 4662 | −6.6003 | −4.8189 |
| 4663 | −7.8195 | −4.8180 |
| 4664 | −6.6203 | −5.9964 |
| 4665 | −7.0256 | −5.3181 |

TABLE 8
Transposon Right end Variants SEQ ID NOs: 845-2690

| | Normalized | Normalized |
|---|---|---|
| | enrichment = | enrichment = |
| | Log2 (Normalized | Log2 (Normalized |
| SEQ ID | FC) - RL (1^{st} | FC) - RL (2nd |
| NO | Replicate) | Replicate) |
| 845 | −0.0972 | −0.0917 |
| 846 | 0.1133 | 0.0709 |
| 847 | 0.1499 | 0.2162 |
| 848 | −0.1129 | −0.0245 |
| 849 | −0.2200 | −0.0967 |
| 850 | −0.0175 | 0.0266 |
| 851 | −0.0715 | −0.0576 |
| 852 | −0.5016 | −0.2900 |
| 853 | 0.0165 | 0.0605 |
| 854 | −0.0212 | 0.0131 |
| 855 | −0.1773 | −0.0457 |
| 856 | −0.0125 | −0.0060 |
| 857 | 0.0242 | 0.0000 |
| 858 | 0.1188 | 0.1580 |
| 859 | 0.1927 | 0.0611 |
| 860 | 0.0026 | 0.0011 |
| 861 | −0.2103 | −0.0695 |
| 862 | −0.0484 | 0.0157 |
| 863 | −0.2294 | −0.0776 |
| 864 | −0.1197 | −0.0652 |
| 865 | 0.1329 | 0.0411 |
| 866 | 0.0305 | 0.0139 |
| 867 | 0.0908 | 0.1321 |
| 868 | −0.0285 | −0.0594 |
| 869 | −0.0538 | −0.1167 |
| 870 | −0.0690 | −0.0491 |
| 871 | −0.0171 | −0.0639 |
| 872 | −0.0248 | 0.0467 |
| 873 | −0.2783 | −0.0966 |
| 874 | −0.3390 | −0.3395 |
| 875 | −0.1778 | −0.1568 |
| 876 | −0.3871 | −0.3062 |
| 877 | −0.0506 | 0.0656 |
| 878 | −0.2168 | −0.1973 |
| 879 | −0.4239 | −0.2132 |
| 880 | −0.4862 | −0.2364 |
| 881 | −0.3112 | 0.0123 |
| 882 | −0.2186 | −0.2249 |
| 883 | −0.1887 | −0.1332 |
| 884 | −0.1433 | −0.1747 |
| 885 | 0.0795 | 0.0436 |
| 886 | −0.0144 | 0.0158 |
| 887 | −0.1188 | −0.0667 |
| 888 | 0.1965 | 0.1410 |
| 889 | −0.0110 | −0.0979 |
| 890 | 0.1291 | 0.0906 |
| 891 | 0.1271 | 0.1828 |
| 892 | −0.6427 | −0.4707 |
| 893 | −0.7523 | −0.5963 |
| 894 | −0.0641 | −0.0799 |
| 895 | −0.5287 | −0.6630 |
| 896 | 0.0538 | 0.1945 |
| 897 | −0.0415 | −0.0249 |
| 898 | −0.5239 | −0.3676 |
| 899 | −0.7044 | −0.5808 |
| 900 | −0.5191 | −0.3504 |
| 901 | −0.1370 | 0.0589 |
| 902 | −0.7140 | −0.7370 |
| 903 | −0.1906 | −0.2914 |
| 904 | −0.1037 | −0.0799 |
| 905 | 0.1294 | 0.1549 |
| 906 | −0.4808 | −0.4430 |
| 907 | −0.2015 | −0.1620 |
| 908 | 0.0207 | 0.0031 |
| 909 | −0.0069 | 0.1029 |
| 910 | −0.0639 | −0.0663 |
| 911 | −0.1108 | −0.1709 |
| 912 | −0.3188 | −0.2430 |
| 913 | −0.2313 | −0.2410 |
| 914 | −0.5408 | −0.3811 |
| 915 | −0.1487 | −0.0595 |
| 916 | −0.2023 | −0.2057 |
| 917 | 0.0030 | −0.1030 |
| 918 | −0.4366 | −0.3627 |
| 919 | −0.3066 | −0.1887 |
| 920 | −0.1731 | 0.1401 |
| 921 | −0.8055 | −0.7116 |
| 922 | −0.4143 | −0.1711 |
| 923 | −0.2934 | 0.0030 |
| 924 | −0.4469 | −0.2285 |
| 925 | 0.0162 | 0.1070 |
| 926 | −0.0903 | −0.1001 |
| 927 | −0.3575 | −0.3030 |
| 928 | −0.5593 | −0.2101 |
| 929 | −0.5401 | −0.2754 |
| 930 | −0.2162 | −0.0815 |
| 931 | −0.6106 | −0.4018 |
| 932 | −0.4470 | −0.2328 |
| 933 | −0.5047 | −0.4051 |
| 934 | −0.5751 | −0.4384 |
| 935 | −0.1005 | 0.1509 |
| 936 | −0.6109 | −0.2998 |
| 937 | −0.1516 | 0.0573 |
| 938 | −0.3121 | −0.3291 |
| 939 | −0.5857 | −0.3550 |
| 940 | −0.5489 | −0.4308 |
| 941 | −0.6264 | −0.4099 |
| 942 | 0.0565 | 0.3352 |
| 943 | −0.1753 | −0.0650 |
| 944 | −0.4000 | −0.4042 |
| 945 | −0.7127 | −0.4350 |
| 946 | 0.0251 | −0.2093 |
| 947 | −0.4439 | −0.3738 |
| 948 | −0.4076 | −0.3305 |
| 949 | −0.4771 | −0.3682 |
| 950 | −0.9827 | −0.8885 |
| 951 | −0.2673 | −0.3466 |
| 952 | −0.4213 | −0.5371 |
| 953 | −0.1103 | 0.0186 |
| 954 | 0.0836 | 0.0535 |
| 955 | 0.0757 | −0.0860 |
| 956 | −0.1355 | −0.0196 |
| 957 | −0.1310 | −0.1235 |
| 958 | 0.0618 | −0.1443 |
| 959 | −0.1458 | −0.1082 |
| 960 | 0.0306 | 0.0718 |
| 961 | −0.8817 | −0.7317 |
| 962 | −0.9083 | −0.9826 |
| 963 | −0.0536 | −0.0798 |
| 964 | 0.2161 | −0.4247 |
| 965 | −0.7814 | −0.8480 |
| 966 | 0.0470 | −0.1558 |
| 967 | −0.8321 | −0.9597 |
| 968 | −0.6211 | −0.5975 |
| 969 | −0.1929 | −0.2793 |
| 970 | −0.0364 | −0.1358 |
| 971 | −1.2181 | −1.2482 |
| 972 | −0.1452 | −0.0881 |
| 973 | 0.3667 | 0.3038 |
| 974 | 0.1401 | −0.0801 |
| 975 | −0.4893 | −0.5730 |
| 976 | −0.2694 | −0.1883 |
| 977 | −0.2019 | −0.0446 |
| 978 | 0.4096 | 0.4099 |
| 979 | −0.2269 | −0.1175 |
| 980 | −0.4636 | −0.4742 |
| 981 | −0.2837 | −0.2560 |
| 982 | −0.6064 | −0.5119 |
| 983 | −0.0641 | −0.0852 |
| 984 | −0.0505 | −0.0569 |
| 985 | −1.4198 | −1.2264 |
| 986 | −0.8098 | −0.6498 |
| 987 | −0.2677 | −0.2607 |
| 988 | 0.0986 | 0.1762 |
| 989 | −0.0479 | 0.0529 |
| 990 | 0.8539 | 1.0127 |
| 991 | −1.3086 | −1.2200 |
| 992 | −0.5181 | −0.3570 |
| 993 | −0.5095 | −0.3630 |
| 994 | −0.6538 | −0.4741 |
| 995 | −0.0613 | −0.0722 |
| 996 | −0.5768 | −0.3511 |
| 997 | −0.8888 | −0.7021 |
| 998 | −0.7110 | −0.5351 |
| 999 | −0.3799 | −0.3643 |
| 1000 | −0.2306 | −0.1073 |
| 1001 | −0.8315 | −0.7541 |
| 1002 | −0.3623 | −0.0686 |
| 1003 | −0.4499 | −0.5013 |
| 1004 | −0.3464 | −0.2103 |
| 1005 | −1.0335 | −0.7974 |
| 1006 | −0.5741 | −0.4152 |
| 1007 | −0.8813 | −0.6250 |
| 1008 | −0.6577 | −0.4492 |
| 1009 | −0.5500 | −0.3941 |
| 1010 | −1.9146 | −1.5090 |
| 1011 | −0.5733 | −0.4754 |
| 1012 | −0.2210 | −0.2784 |
| 1013 | −0.1405 | −0.1524 |
| 1014 | −0.1962 | −0.0545 |
| 1015 | 0.2993 | 0.2331 |
| 1016 | 0.1498 | 0.3270 |
| 1017 | 1.3602 | 1.4722 |
| 1018 | 0.0515 | −0.1304 |
| 1019 | 0.3793 | 0.3130 |
| 1020 | −0.2447 | −0.1861 |
| 1021 | −0.7838 | −0.7585 |
| 1022 | −0.9265 | −0.8223 |
| 1023 | 0.1286 | −0.0335 |
| 1024 | −0.4673 | −0.6612 |
| 1025 | −0.2544 | −0.3110 |
| 1026 | −0.2247 | −0.3731 |
| 1027 | −1.3635 | −1.3215 |
| 1028 | −0.5855 | −0.3110 |
| 1029 | −0.5955 | −0.5531 |
| 1030 | −0.0980 | −0.2128 |
| 1031 | −1.2607 | −1.0972 |
| 1032 | −0.0756 | −0.2187 |
| 1033 | 0.2025 | 0.0598 |
| 1034 | −0.1733 | −0.2244 |
| 1035 | −0.0949 | −0.1540 |
| 1036 | 0.0484 | −0.0482 |
| 1037 | −0.0613 | −0.0422 |
| 1038 | −0.6332 | −0.5313 |
| 1039 | −0.0677 | −0.1704 |
| 1040 | −0.0583 | −0.0652 |
| 1041 | −0.2022 | −0.2329 |
| 1042 | −0.3770 | −0.3858 |
| 1043 | 0.1607 | 0.4341 |
| 1044 | −0.1388 | −0.1000 |
| 1045 | −0.0984 | 0.0814 |
| 1046 | −0.5026 | −0.4517 |
| 1047 | 0.1214 | 0.1101 |
| 1048 | 0.2508 | 0.1662 |
| 1049 | −0.1773 | −0.2940 |
| 1050 | −0.1284 | −0.0849 |
| 1051 | −0.1162 | −0.1769 |
| 1052 | −0.2483 | −0.2220 |
| 1053 | 0.2150 | 0.1375 |
| 1054 | −0.0343 | −0.0760 |
| 1055 | 0.3564 | 0.3269 |
| 1056 | 0.1272 | 0.1034 |
| 1057 | −0.7125 | −0.7111 |
| 1058 | −0.4534 | −0.3559 |
| 1059 | −0.1197 | 0.0009 |
| 1060 | 0.1311 | 0.0775 |
| 1061 | 0.2255 | 0.2426 |
| 1062 | −0.9085 | −0.7188 |
| 1063 | −6.4750 | −5.3052 |
| 1064 | −1.0147 | −0.6019 |
| 1065 | −0.1005 | −0.0524 |
| 1066 | −2.4832 | −1.9162 |
| 1067 | −0.6184 | −0.4395 |
| 1068 | −6.1709 | −5.4441 |
| 1069 | −6.6114 | −6.7160 |
| 1070 | −1.9540 | −1.6726 |
| 1071 | 0.0191 | 0.1691 |
| 1072 | −0.2429 | −0.2170 |
| 1073 | −0.2817 | −0.1854 |
| 1074 | −0.1426 | −0.1629 |
| 1075 | −0.1736 | −0.1749 |
| 1076 | −0.0508 | −0.0561 |
| 1077 | −0.1196 | −0.1088 |
| 1078 | −0.0218 | 0.0241 |
| 1079 | −0.0763 | −0.1423 |
| 1080 | −0.0041 | 0.0526 |
| 1081 | −0.0071 | −0.0316 |
| 1082 | 0.0937 | 0.0666 |
| 1083 | −0.0049 | −0.0924 |
| 1084 | −0.1870 | −0.0910 |
| 1085 | −0.1287 | −0.1056 |
| 1086 | −0.0072 | 0.1001 |
| 1087 | 0.3397 | 0.3328 |
| 1088 | 0.1505 | 0.1116 |
| 1089 | 0.0103 | −0.0031 |
| 1090 | 0.0763 | 0.1241 |
| 1091 | −0.2111 | −0.1293 |
| 1092 | 0.0470 | 0.0065 |
| 1093 | −0.2505 | −0.1845 |
| 1094 | −0.2878 | −0.2396 |
| 1095 | −0.5054 | −0.4204 |
| 1096 | −0.6403 | −0.4591 |
| 1097 | −0.0201 | 0.0192 |
| 1098 | −0.2590 | −0.2499 |
| 1099 | −0.0900 | −0.0843 |
| 1100 | 1.2979 | 1.4027 |
| 1101 | −0.7546 | −0.5592 |
| 1102 | −0.5416 | −0.4750 |
| 1103 | −0.7140 | −0.4936 |
| 1104 | −0.5335 | −0.4716 |
| 1105 | −1.2368 | −0.9783 |
| 1106 | −0.1365 | −0.0905 |
| 1107 | 0.5732 | 0.7451 |
| 1108 | −0.8582 | −0.6030 |
| 1109 | −0.4895 | −0.2336 |
| 1110 | −0.8601 | −0.5282 |
| 1111 | −0.3335 | −0.0574 |
| 1112 | −0.5970 | −0.5052 |
| 1113 | −0.5596 | −0.4172 |
| 1114 | −0.7238 | −0.6062 |
| 1115 | −0.6959 | −0.7126 |
| 1116 | −1.1843 | −0.7625 |
| 1117 | −0.5295 | −0.5207 |
| 1118 | −0.3534 | −0.3543 |
| 1119 | −1.0659 | −0.8250 |
| 1120 | −0.5712 | −0.5344 |
| 1121 | −0.4024 | −0.4923 |
| 1122 | 0.5495 | 0.4422 |
| 1123 | −0.7704 | −0.6445 |
| 1124 | −1.2678 | −1.1422 |
| 1125 | −2.2415 | −2.1220 |
| 1126 | −3.2710 | −3.0021 |
| 1127 | −1.8651 | −1.6938 |
| 1128 | −1.1325 | −0.8602 |
| 1129 | −0.0103 | −0.0302 |
| 1130 | 0.1811 | −0.0566 |
| 1131 | −0.8755 | −0.7693 |
| 1132 | 0.1435 | 0.0482 |
| 1133 | −0.6978 | −0.5524 |
| 1134 | −0.9802 | −0.9319 |
| 1135 | −0.8680 | −0.7714 |
| 1136 | −1.1320 | −0.9297 |
| 1137 | −0.3590 | −0.4287 |
| 1138 | −1.1004 | −0.7888 |
| 1139 | −1.9804 | −1.7244 |
| 1140 | −1.0642 | −0.8442 |
| 1141 | −0.6490 | −0.6346 |
| 1142 | −0.3133 | −0.3279 |
| 1143 | −1.3951 | −1.2839 |
| 1144 | −1.4158 | −1.2992 |
| 1145 | −2.8650 | −2.5688 |
| 1146 | −3.8628 | −3.7285 |
| 1147 | −1.9540 | −1.7465 |
| 1148 | −0.4532 | −0.2019 |
| 1149 | −0.5588 | −0.5654 |
| 1150 | −0.8854 | −0.7707 |
| 1151 | −0.3970 | −0.4226 |
| 1152 | −0.1217 | −0.1515 |
| 1153 | −0.2943 | −0.2989 |
| 1154 | −0.4198 | −0.4741 |
| 1155 | −1.1598 | −0.9642 |
| 1156 | −0.1363 | 0.1526 |
| 1157 | −6.3693 | −6.4833 |
| 1158 | −6.7049 | −6.3689 |
| 1159 | −6.1630 | −6.5027 |
| 1160 | −6.2235 | −6.6506 |
| 1161 | −0.2925 | −0.2750 |
| 1162 | −0.0191 | 0.0153 |
| 1163 | −0.2720 | −0.1613 |
| 1164 | −0.1078 | −0.1073 |
| 1165 | −0.1771 | −0.1057 |
| 1166 | 0.0599 | 0.0633 |
| 1167 | −0.4888 | −0.2902 |
| 1168 | −0.0771 | −0.0866 |
| 1169 | −0.0023 | −0.0596 |
| 1170 | −0.0355 | −0.0004 |
| 1171 | −0.3142 | −0.1313 |
| 1172 | −0.2688 | −0.2331 |
| 1173 | −0.1434 | −0.1321 |
| 1174 | −0.2966 | −0.0615 |
| 1175 | −0.1991 | −0.0429 |
| 1176 | −0.5519 | −0.4188 |
| 1177 | −0.8182 | −0.4566 |
| 1178 | −0.0872 | −0.0421 |
| 1179 | −0.0327 | −0.1015 |
| 1180 | −0.0860 | −0.1599 |
| 1181 | 0.0660 | −0.0014 |
| 1182 | −0.1228 | −0.1465 |
| 1183 | −0.6048 | −0.3343 |
| 1184 | −0.6977 | −0.5532 |
| 1185 | −0.1949 | −0.2821 |
| 1186 | −0.0167 | −0.1439 |
| 1187 | −0.1985 | −0.2580 |
| 1188 | −0.4633 | −0.4195 |
| 1189 | −0.4746 | −0.3123 |
| 1190 | −0.8289 | −0.6101 |
| 1191 | −0.4667 | −0.1463 |
| 1192 | −0.2454 | 0.1051 |
| 1193 | −0.4368 | −0.1116 |
| 1194 | −0.4125 | −0.2973 |
| 1195 | −0.4247 | −0.2010 |
| 1196 | −0.8292 | −0.4289 |
| 1197 | −1.2878 | −0.9080 |
| 1198 | −0.6738 | −0.5303 |
| 1199 | −1.5567 | −1.3018 |
| 1200 | −0.8928 | −0.6332 |
| 1201 | −0.1547 | −0.1702 |
| 1202 | −0.3243 | −0.2689 |
| 1203 | −0.1981 | −0.1769 |
| 1204 | −0.3098 | −0.3504 |
| 1205 | −0.2624 | −0.2906 |
| 1206 | −0.7014 | −0.7713 |
| 1207 | −0.6682 | −0.5498 |
| 1208 | −0.1719 | −0.3577 |
| 1209 | −0.0287 | 0.0170 |
| 1210 | −0.2438 | 0.0253 |
| 1211 | −0.9559 | −0.8861 |
| 1212 | −0.8260 | −0.5582 |
| 1213 | −0.1419 | −0.2345 |
| 1214 | −0.4096 | −0.2837 |
| 1215 | −0.1997 | −0.0628 |
| 1216 | −0.5405 | −0.4261 |
| 1217 | −0.8223 | −0.7210 |
| 1218 | −0.7911 | −0.6729 |
| 1219 | −1.9800 | −1.9652 |
| 1220 | −1.6845 | −1.2165 |
| 1221 | −0.6741 | −0.6091 |
| 1222 | −0.3867 | −0.2421 |
| 1223 | −0.2994 | −0.2956 |
| 1224 | −0.2320 | −0.1583 |
| 1225 | −0.3325 | −0.4582 |
| 1226 | −0.6108 | −0.6203 |
| 1227 | −0.9205 | −0.9350 |
| 1228 | −0.1911 | −0.2540 |
| 1229 | −0.3531 | −0.2884 |
| 1230 | −0.6742 | −0.6006 |
| 1231 | −0.4128 | −0.3220 |
| 1232 | −0.1489 | −0.1944 |
| 1233 | 0.4748 | 0.2843 |
| 1234 | −0.2267 | −0.1731 |
| 1235 | −0.9208 | −0.7290 |
| 1236 | −0.3935 | −0.2296 |
| 1237 | −0.1188 | −0.1454 |
| 1238 | −5.4023 | −4.2914 |
| 1239 | −6.2608 | −6.6389 |
| 1240 | −6.6528 | −6.5892 |
| 1241 | 0.1282 | 0.3011 |
| 1242 | 0.1017 | 0.2009 |
| 1243 | −0.0179 | 0.0286 |
| 1244 | −0.0006 | 0.0162 |
| 1245 | −0.1826 | −0.0935 |
| 1246 | 0.0299 | 0.1719 |
| 1247 | −0.2954 | −0.2584 |
| 1248 | −0.0437 | −0.0372 |
| 1249 | −0.1558 | −0.0856 |
| 1250 | −6.2442 | −7.0622 |
| 1251 | −6.2726 | −6.9032 |
| 1252 | −5.1789 | −6.2329 |
| 1253 | −5.8702 | −5.9493 |
| 1254 | −6.2998 | −6.1229 |
| 1255 | −5.6230 | −4.5139 |
| 1256 | −1.6586 | −1.2090 |
| 1257 | −1.6549 | −1.4101 |
| 1258 | −0.7977 | −0.7664 |
| 1259 | −0.8858 | −0.7854 |
| 1260 | −1.4355 | −1.1652 |
| 1261 | −1.1663 | −0.9791 |
| 1262 | −1.1320 | −0.8006 |
| 1263 | −1.6870 | −1.5627 |
| 1264 | −2.3376 | −2.1798 |
| 1265 | −2.0035 | −1.8135 |
| 1266 | −0.9422 | −0.8684 |
| 1267 | −0.2612 | −0.3539 |
| 1268 | −0.6164 | −0.6386 |
| 1269 | −0.6856 | −0.4844 |
| 1270 | −2.4235 | −2.2130 |
| 1271 | −2.3696 | −2.1270 |
| 1272 | −1.2745 | −1.1873 |
| 1273 | −1.7032 | −1.4048 |
| 1274 | −0.9630 | −0.9131 |
| 1275 | −0.8272 | −0.5738 |
| 1276 | −0.3831 | −0.3797 |
| 1277 | −0.6403 | −0.5088 |
| 1278 | −0.5832 | −0.3960 |
| 1279 | −1.4093 | −1.1704 |
| 1280 | −1.8790 | −1.5266 |
| 1281 | −2.0245 | −1.7991 |
| 1282 | −0.4959 | −0.4118 |
| 1283 | −1.5819 | −1.4164 |
| 1284 | −1.7800 | −1.9283 |
| 1285 | −1.8205 | −1.6594 |
| 1286 | −0.7893 | −0.6376 |
| 1287 | −0.6421 | −0.4161 |
| 1288 | 2.2631 | 3.3117 |
| 1289 | −0.6097 | −0.6117 |
| 1290 | −2.1778 | −2.0476 |
| 1291 | −0.7624 | −0.5950 |
| 1292 | −1.7240 | −1.4001 |
| 1293 | −2.0112 | −1.9930 |
| 1294 | −0.9688 | −0.8796 |
| 1295 | −1.6290 | −1.6665 |
| 1296 | −0.8800 | −0.7230 |
| 1297 | −0.3333 | −0.1335 |
| 1298 | −0.4204 | −0.3192 |
| 1299 | −0.4270 | −0.2141 |
| 1300 | −0.4963 | −0.3980 |
| 1301 | −0.5979 | −0.3694 |
| 1302 | −0.8924 | −0.5570 |
| 1303 | −0.7206 | −0.3899 |
| 1304 | −0.4632 | −0.2903 |
| 1305 | −0.4186 | −0.3804 |
| 1306 | −0.5212 | −0.3291 |
| 1307 | −0.3875 | −0.3266 |
| 1308 | −0.7989 | −0.4965 |
| 1309 | −0.3785 | −0.2717 |
| 1310 | −0.3116 | −0.1766 |
| 1311 | −0.0443 | −0.1059 |
| 1312 | −0.5944 | −0.4761 |
| 1313 | −1.0815 | −0.8558 |
| 1314 | −0.4181 | −0.3187 |
| 1315 | −0.5631 | −0.1885 |
| 1316 | −0.7481 | −0.4902 |
| 1317 | −0.3174 | −0.1767 |
| 1318 | −0.6081 | −0.5006 |
| 1319 | −0.2492 | −0.0642 |
| 1320 | −0.1930 | −0.2224 |
| 1321 | 0.2232 | 0.2194 |
| 1322 | −0.0202 | −0.0266 |
| 1323 | −0.1394 | −0.0830 |
| 1324 | −0.3632 | −0.2088 |
| 1325 | −0.3346 | −0.2052 |
| 1326 | −0.1587 | −0.1470 |
| 1327 | −6.4665 | −7.1605 |
| 1328 | −7.1385 | −7.3766 |
| 1329 | −6.7434 | −6.9995 |
| 1330 | −5.4555 | −5.7622 |
| 1331 | −5.9898 | −5.9362 |
| 1332 | −6.4308 | −7.9428 |
| 1333 | −0.8973 | −0.9353 |
| 1334 | −1.9011 | −1.5292 |
| 1335 | −0.9008 | −0.7775 |
| 1336 | −0.5444 | −0.5008 |
| 1337 | −0.8960 | −0.7826 |
| 1338 | −0.8754 | −0.7413 |
| 1339 | −2.7074 | −2.4673 |
| 1340 | −3.2654 | −2.9906 |
| 1341 | −3.8603 | −4.0416 |
| 1342 | −4.3731 | −4.3634 |
| 1343 | −4.4177 | −4.1405 |
| 1344 | −4.2119 | −4.1793 |
| 1345 | −2.0898 | −2.1689 |
| 1346 | −3.9532 | −3.5811 |
| 1347 | −1.5566 | −1.2616 |
| 1348 | −2.7740 | −2.6573 |
| 1349 | −2.8774 | −2.5098 |
| 1350 | −3.1250 | −2.7771 |
| 1351 | −1.4608 | −1.3436 |
| 1352 | −0.5113 | −0.2069 |
| 1353 | −1.3287 | −1.1224 |
| 1354 | −0.7777 | −0.7775 |
| 1355 | −0.5907 | −0.5901 |
| 1356 | −1.4064 | −1.0720 |
| 1357 | −0.2079 | −0.2277 |
| 1358 | −0.4067 | −0.3810 |
| 1359 | −0.8437 | −0.7015 |
| 1360 | −2.1790 | −2.3925 |
| 1361 | −3.3607 | −3.6427 |
| 1362 | −3.0680 | −3.0051 |
| 1363 | −3.6556 | −3.3395 |
| 1364 | −2.0201 | −1.9637 |
| 1365 | −1.8558 | −1.6892 |
| 1366 | −2.2051 | −2.2177 |
| 1367 | −1.0293 | −0.9951 |
| 1368 | −1.8634 | −1.6804 |
| 1369 | −1.8889 | −1.7740 |
| 1370 | −2.0082 | −1.8146 |
| 1371 | −1.5300 | −1.2530 |
| 1372 | −1.0915 | −0.9600 |
| 1373 | −1.0420 | −0.8164 |
| 1374 | −0.6743 | −0.5696 |
| 1375 | −0.4950 | −0.3779 |
| 1376 | −0.3242 | −0.2599 |
| 1377 | −0.6377 | −0.4441 |
| 1378 | −0.6059 | −0.3460 |
| 1379 | −1.2272 | −0.9050 |
| 1380 | −0.5863 | −0.4034 |
| 1381 | −1.0569 | −0.7652 |
| 1382 | −0.2362 | 0.0128 |
| 1383 | −0.6351 | −0.4266 |
| 1384 | −1.0605 | −0.8107 |
| 1385 | −0.7557 | −0.5987 |
| 1386 | −1.0498 | −0.6704 |
| 1387 | −0.5410 | −0.3941 |
| 1388 | −0.7122 | −0.5841 |
| 1389 | −0.5807 | −0.5413 |
| 1390 | −0.6753 | −0.4423 |
| 1391 | −0.6652 | −0.6053 |
| 1392 | −0.7209 | −0.6220 |
| 1393 | −0.7501 | −0.6532 |
| 1394 | −0.7056 | −0.5295 |
| 1395 | −0.1582 | −0.1418 |
| 1396 | −0.2646 | −0.2887 |
| 1397 | −0.2260 | −0.2113 |
| 1398 | −0.2210 | −0.0953 |
| 1399 | 0.0013 | −0.0024 |
| 1400 | 0.3407 | 0.3379 |
| 1401 | −0.0032 | 0.0436 |
| 1402 | −15.0000 | −8.8948 |
| 1403 | −7.4280 | −7.4546 |
| 1404 | −8.2717 | −9.8832 |
| 1405 | −0.7771 | −0.1201 |
| 1406 | −4.9847 | −4.9695 |
| 1407 | −15.0000 | −15.0000 |
| 1408 | −7.4951 | −8.8436 |
| 1409 | −8.1122 | −10.7237 |
| 1410 | −6.2941 | −5.9987 |
| 1411 | −0.3884 | −0.2739 |
| 1412 | 0.2663 | 0.2334 |
| 1413 | −2.7730 | −2.5333 |
| 1414 | −3.4387 | −3.5539 |
| 1415 | −0.5907 | −0.2066 |
| 1416 | −1.7891 | −1.4545 |
| 1417 | 0.1146 | −0.0182 |
| 1418 | −0.4913 | −0.0943 |
| 1419 | −9.0703 | −7.1900 |
| 1420 | −6.4981 | −7.1877 |
| 1421 | −6.8547 | −7.4662 |
| 1422 | −5.7807 | −7.4771 |
| 1423 | −6.7507 | −7.1923 |
| 1424 | −7.1338 | −6.8385 |
| 1425 | −5.8752 | −5.7443 |
| 1426 | −6.4936 | −7.2401 |
| 1427 | −7.0052 | −7.2123 |
| 1428 | −5.9967 | −5.8751 |
| 1429 | −6.6941 | −6.5507 |
| 1430 | −6.5594 | −6.6304 |
| 1431 | −6.3757 | −6.5828 |
| 1432 | −6.8453 | −6.2870 |
| 1433 | −6.3822 | −6.7498 |
| 1434 | −5.7589 | −5.5001 |
| 1435 | −6.1881 | −5.9293 |
| 1436 | −3.0811 | −3.1780 |
| 1437 | −3.0895 | −2.8425 |
| 1438 | −3.3071 | −3.2159 |
| 1439 | −1.2011 | −0.9876 |
| 1440 | −1.1118 | −0.9891 |
| 1441 | −3.6448 | −3.5323 |
| 1442 | −0.9287 | −0.8481 |
| 1443 | −3.4847 | −3.4804 |
| 1444 | −1.2100 | −0.6500 |
| 1445 | −2.0982 | −1.7234 |
| 1446 | 0.0221 | 0.2485 |
| 1447 | −0.3289 | 0.0972 |
| 1448 | −0.8643 | −0.3434 |
| 1449 | −0.0196 | 0.3471 |
| 1450 | 0.0714 | 0.5092 |
| 1451 | 0.1894 | 0.6446 |
| 1452 | −0.5919 | −0.1541 |
| 1453 | −0.3533 | 0.0156 |
| 1454 | −0.4238 | −0.0323 |
| 1455 | −0.0870 | 0.2232 |
| 1456 | −0.1576 | 0.1996 |
| 1457 | −0.4935 | 0.0316 |
| 1458 | −0.1038 | 0.2150 |
| 1459 | 0.6302 | 0.9431 |
| 1460 | 0.0495 | 0.2278 |
| 1461 | 0.0120 | 0.2304 |
| 1462 | 0.0921 | 0.2967 |
| 1463 | 0.5990 | 0.8271 |
| 1464 | −0.3096 | −0.2101 |
| 1465 | 0.0296 | 0.2530 |
| 1466 | −0.4406 | −0.3171 |
| 1467 | −0.3182 | −0.1457 |
| 1468 | −0.1583 | −0.0457 |
| 1469 | −0.3955 | −0.1701 |
| 1470 | −0.3171 | 0.1276 |
| 1471 | −0.4759 | −0.3844 |
| 1472 | −0.1222 | −0.0686 |
| 1473 | −0.1232 | 0.0349 |
| 1474 | −4.3036 | −5.0869 |
| 1475 | −2.8035 | −3.1780 |
| 1476 | −3.1850 | −3.0775 |
| 1477 | −1.1415 | −0.7737 |
| 1478 | −0.8517 | −0.4744 |
| 1479 | −2.2767 | −1.9738 |
| 1480 | −1.0685 | −0.5542 |
| 1481 | −1.4460 | −1.0334 |
| 1482 | −0.6205 | −0.3478 |
| 1483 | −0.6330 | −0.2327 |
| 1484 | −0.4403 | −0.0702 |
| 1485 | −1.5754 | −1.0219 |
| 1486 | −0.4861 | −0.2156 |
| 1487 | −0.5874 | −0.1697 |
| 1488 | −0.0805 | 0.1963 |
| 1489 | −0.3504 | −0.0641 |
| 1490 | 0.0258 | 0.2504 |
| 1491 | −0.3106 | −0.0365 |
| 1492 | 1.1926 | 1.4901 |
| 1493 | −0.1134 | 0.1560 |
| 1494 | −0.1202 | −0.0123 |
| 1495 | 0.3423 | 0.7625 |
| 1496 | −0.2646 | 0.0320 |
| 1497 | −0.2071 | −0.0776 |
| 1498 | −0.9520 | −0.3652 |
| 1499 | −0.2542 | 0.0033 |
| 1500 | 0.5712 | 0.8422 |
| 1501 | −0.5000 | −0.0896 |
| 1502 | 0.1312 | 0.3586 |
| 1503 | −0.4072 | 0.1070 |
| 1504 | −0.1311 | 0.1476 |
| 1505 | −0.1298 | 0.1228 |
| 1506 | −0.5973 | −0.3471 |
| 1507 | 0.2570 | 0.4720 |
| 1508 | −0.8475 | −0.5664 |
| 1509 | −1.5604 | −1.2550 |
| 1510 | −0.7856 | −0.6668 |
| 1511 | −4.7581 | −4.6055 |
| 1512 | −5.0827 | −5.0772 |
| 1513 | −1.9160 | −1.5486 |
| 1514 | −3.1906 | −2.7051 |
| 1515 | −0.3309 | −0.3317 |
| 1516 | 0.4344 | 0.3008 |
| 1517 | −1.1501 | −1.0586 |
| 1518 | −1.4402 | −1.0133 |
| 1519 | −1.6438 | −1.4626 |
| 1520 | −1.1336 | −0.9546 |
| 1521 | −1.2119 | −1.2170 |
| 1522 | −1.0062 | −0.9686 |
| 1523 | −0.3704 | −0.3289 |
| 1524 | −0.2841 | −0.1923 |
| 1525 | −0.6341 | −0.4925 |
| 1526 | −0.9848 | −0.7491 |
| 1527 | −1.6261 | −1.2224 |
| 1528 | −1.7354 | −1.3407 |
| 1529 | −3.0235 | −2.4425 |
| 1530 | −4.2933 | −4.1750 |
| 1531 | −4.9938 | −5.3320 |
| 1532 | −3.3184 | −3.0897 |
| 1533 | −0.5272 | −0.4993 |
| 1534 | −4.3766 | −4.0946 |
| 1535 | −6.0703 | −5.9071 |
| 1536 | −6.5033 | −6.9043 |
| 1537 | −7.0385 | −6.3732 |
| 1538 | −8.9694 | −7.1029 |
| 1539 | −1.7789 | −1.5003 |
| 1540 | −0.8205 | −0.7889 |
| 1541 | −0.9020 | −0.8005 |
| 1542 | −0.3904 | −0.3920 |
| 1543 | −0.0454 | −0.2096 |
| 1544 | −1.4908 | −1.1834 |
| 1545 | −0.7450 | −0.5924 |
| 1546 | −0.5293 | −0.5001 |
| 1547 | 0.1606 | 0.0061 |
| 1548 | −0.2372 | −0.2860 |
| 1549 | −0.2588 | 0.0197 |
| 1550 | −1.4255 | −1.1720 |
| 1551 | −5.3244 | −4.5145 |
| 1552 | −2.5337 | −2.1278 |
| 1553 | −1.3184 | −0.9478 |
| 1554 | −1.0100 | −0.8103 |
| 1555 | −0.3928 | −0.1208 |
| 1556 | −0.5473 | −0.3392 |
| 1557 | −0.4941 | −0.2841 |
| 1558 | −0.4247 | −0.2461 |
| 1559 | −0.4666 | −0.4763 |
| 1560 | −1.1110 | −0.9875 |
| 1561 | −1.1179 | −0.9110 |
| 1562 | −1.2551 | −0.9968 |
| 1563 | −2.4427 | −2.2461 |
| 1564 | −4.3354 | −4.1376 |
| 1565 | −0.5779 | −0.5747 |
| 1566 | −0.7596 | −0.5430 |
| 1567 | −0.8413 | −0.6010 |
| 1568 | −1.7788 | −1.4492 |
| 1569 | −2.4874 | −2.0469 |
| 1570 | −2.0435 | −1.7373 |
| 1571 | −6.7224 | −7.3339 |
| 1572 | −6.9795 | −6.5421 |
| 1573 | −7.2145 | −7.3785 |
| 1574 | −6.4574 | −7.7377 |
| 1575 | −7.1751 | −7.0090 |
| 1576 | −8.7860 | −7.7251 |
| 1577 | 0.0868 | −0.1387 |
| 1578 | −6.5239 | −6.5505 |
| 1579 | −2.2915 | −1.9710 |
| 1580 | −6.4079 | −6.2645 |
| 1581 | −7.1110 | −6.5781 |
| 1582 | −6.5821 | −6.7893 |
| 1583 | −7.3396 | −6.7381 |
| 1584 | −0.0346 | −0.1164 |
| 1585 | −0.6484 | −0.4964 |
| 1586 | −1.9590 | −1.8755 |
| 1587 | −3.7592 | −3.1882 |
| 1588 | −3.3737 | −2.9487 |
| 1589 | −2.8843 | −2.6501 |
| 1590 | −2.8111 | −2.5610 |
| 1591 | −2.9799 | −3.2554 |
| 1592 | −3.4575 | −3.1749 |
| 1593 | −3.9894 | −3.9629 |
| 1594 | −4.1455 | −3.4496 |
| 1595 | −2.9401 | −2.7609 |
| 1596 | −2.9819 | −3.4362 |
| 1597 | −3.5228 | −3.6763 |
| 1598 | −3.3291 | −3.0517 |
| 1599 | −3.1343 | −2.7116 |
| 1600 | −2.9580 | −3.2228 |
| 1601 | −3.4447 | −3.3088 |
| 1602 | −3.3658 | −3.2190 |
| 1603 | −3.5878 | −3.1213 |
| 1604 | −4.0870 | −4.2429 |
| 1605 | −3.3420 | −3.8731 |
| 1606 | −3.0817 | −3.4302 |
| 1607 | −3.8198 | −3.1313 |
| 1608 | −3.1066 | −3.5550 |
| 1609 | −4.7343 | −5.9535 |
| 1610 | −3.8131 | −3.4246 |
| 1611 | −3.2231 | −3.1204 |
| 1612 | −3.3658 | −2.7571 |
| 1613 | −4.3988 | −3.9848 |
| 1614 | −4.0281 | −2.9807 |
| 1615 | −3.1978 | −2.9728 |
| 1616 | −4.7016 | −3.1877 |
| 1617 | −6.5423 | −4.8684 |
| 1618 | −5.3396 | −3.8637 |
| 1619 | −0.2825 | −0.2919 |
| 1620 | −0.9262 | −0.5155 |
| 1621 | −2.1224 | −1.5337 |
| 1622 | −2.7993 | −2.4108 |
| 1623 | −2.6394 | −2.3948 |
| 1624 | −2.9905 | −2.5527 |
| 1625 | −2.1101 | −1.8062 |
| 1626 | −2.3825 | −1.9822 |
| 1627 | −3.6960 | −3.4204 |
| 1628 | −2.9079 | −2.3495 |
| 1629 | −3.2164 | −2.6039 |
| 1630 | −2.6875 | −2.3834 |
| 1631 | −0.9275 | −0.3806 |
| 1632 | −3.4579 | −2.7728 |
| 1633 | −3.0041 | −2.2636 |
| 1634 | −3.1906 | −2.9727 |
| 1635 | −2.4844 | −2.2589 |
| 1636 | −2.2874 | −1.8962 |
| 1637 | −2.3119 | −1.7450 |
| 1638 | −2.1170 | −1.5522 |
| 1639 | −2.5377 | −2.3128 |
| 1640 | −3.1255 | −3.2345 |
| 1641 | −2.4643 | −2.1194 |
| 1642 | −2.7854 | −1.7245 |
| 1643 | −2.5229 | −1.7860 |
| 1644 | −3.7617 | −5.1806 |
| 1645 | −3.4139 | −3.3932 |
| 1646 | −3.3181 | −2.9041 |
| 1647 | −3.2329 | −2.2837 |
| 1648 | −3.4540 | −2.1674 |
| 1649 | −3.4816 | −2.9463 |
| 1650 | −2.9328 | −2.3744 |
| 1651 | −3.5134 | −3.1379 |
| 1652 | −3.8653 | −3.1370 |
| 1653 | −4.1475 | −4.7046 |
| 1654 | −1.0180 | −0.6236 |
| 1655 | −0.5055 | −0.3908 |
| 1656 | −0.5311 | −0.3351 |
| 1657 | −0.3865 | −0.3128 |
| 1658 | −0.2852 | −0.2194 |
| 1659 | −0.6381 | −0.3901 |
| 1660 | −5.4944 | −5.1584 |
| 1661 | −5.1005 | −4.0275 |
| 1662 | −5.8392 | −4.8657 |
| 1663 | −4.6855 | −5.5194 |
| 1664 | −5.2630 | −3.9676 |
| 1665 | −6.2630 | −15.0000 |
| 1666 | −1.2496 | −1.2866 |
| 1667 | −4.2602 | −4.4029 |
| 1668 | −4.1285 | −3.8444 |
| 1669 | −4.6242 | −4.4528 |
| 1670 | −4.3145 | −3.8386 |
| 1671 | −0.8851 | −0.6482 |
| 1672 | −1.1907 | −0.9356 |
| 1673 | −1.5427 | −1.1059 |
| 1674 | −1.2775 | −0.8334 |
| 1675 | −1.6623 | −1.3183 |
| 1676 | 0.1396 | 0.1121 |
| 1677 | −0.0768 | −0.0984 |
| 1678 | −0.0585 | −0.0394 |
| 1679 | −0.0064 | 0.0376 |
| 1680 | −0.2236 | −0.1941 |
| 1681 | −3.5215 | −3.7279 |
| 1682 | −2.6714 | −2.7883 |
| 1683 | −4.3396 | −4.4402 |
| 1684 | −4.3866 | −4.1667 |
| 1685 | −4.9527 | −4.9523 |
| 1686 | −4.7655 | −4.2597 |
| 1687 | −3.8468 | −3.6130 |
| 1688 | −4.5620 | −4.6425 |
| 1689 | −4.6168 | −4.4387 |
| 1690 | −3.6832 | −3.5723 |
| 1691 | −3.2583 | −3.2024 |
| 1692 | −3.4480 | −2.7883 |
| 1693 | −2.7541 | −2.7806 |
| 1694 | −2.7379 | −2.4426 |
| 1695 | −0.8040 | −0.5576 |
| 1696 | −2.1977 | −1.8931 |
| 1697 | −2.2510 | −1.8655 |
| 1698 | −2.7760 | −2.1742 |
| 1699 | −3.5901 | −3.4720 |
| 1700 | −3.5304 | −3.1541 |
| 1701 | −2.9476 | −2.2792 |
| 1702 | −1.8694 | −1.5500 |
| 1703 | −0.4505 | 0.0106 |
| 1704 | −3.2837 | −2.9155 |
| 1705 | −1.9120 | −1.1677 |
| 1706 | −2.9507 | −2.9248 |
| 1707 | −2.4326 | −1.8728 |
| 1708 | −2.7768 | −1.9866 |
| 1709 | −0.9301 | −1.1666 |
| 1710 | −1.4375 | −1.3610 |
| 1711 | −1.2607 | −1.0543 |
| 1712 | −1.3637 | −1.3520 |
| 1713 | −1.6181 | −1.4154 |
| 1714 | −6.0565 | −5.6680 |
| 1715 | −3.4589 | −3.4855 |
| 1716 | 0.3802 | 0.2708 |
| 1717 | −0.1080 | 0.0978 |
| 1718 | 0.1550 | 0.2024 |
| 1719 | −0.1074 | −0.1139 |
| 1720 | −0.2674 | −0.2956 |
| 1721 | −0.4742 | −0.3674 |
| 1722 | −0.4035 | −0.4526 |
| 1723 | −0.3845 | −0.4161 |
| 1724 | −0.8176 | −0.6196 |
| 1725 | −0.6776 | −0.6652 |
| 1726 | −0.8598 | −0.7793 |
| 1727 | −0.8377 | −0.6657 |
| 1728 | −0.6022 | −0.5586 |
| 1729 | −0.7275 | −0.5694 |
| 1730 | −0.4579 | −0.2293 |
| 1731 | −0.5697 | −0.4937 |
| 1732 | −0.6096 | −0.4834 |
| 1733 | −0.7603 | −0.6312 |
| 1734 | −0.5497 | −0.3249 |
| 1735 | −0.6043 | −0.4064 |
| 1736 | −0.5104 | −0.3909 |
| 1737 | −0.8486 | −0.7422 |
| 1738 | −1.8384 | −1.1466 |
| 1739 | −0.6876 | −1.3885 |
| 1740 | 0.5184 | 0.7528 |
| 1741 | −0.5656 | −0.4049 |
| 1742 | −0.9772 | −0.7864 |
| 1743 | −0.8193 | −0.6319 |
| 1744 | −1.0203 | −0.8631 |
| 1745 | −1.1808 | −0.8542 |
| 1746 | −1.5349 | −1.2158 |
| 1747 | −2.1553 | −1.9371 |
| 1748 | −1.9378 | −1.5153 |
| 1749 | −2.4626 | −1.9388 |
| 1750 | −2.5176 | −2.1816 |
| 1751 | −2.6813 | −2.5262 |
| 1752 | −2.7281 | −2.5510 |
| 1753 | −2.0822 | −1.8569 |
| 1754 | −2.2898 | −2.3760 |
| 1755 | −2.2529 | −2.3692 |
| 1756 | −2.5857 | −2.2538 |
| 1757 | −2.2001 | −1.8941 |
| 1758 | −2.1374 | −1.7523 |
| 1759 | −2.5012 | −2.0892 |
| 1760 | −2.0021 | −1.8029 |
| 1761 | −2.1852 | −2.1243 |
| 1762 | −2.4707 | −2.0964 |
| 1763 | −2.2986 | −2.1000 |
| 1764 | −2.5959 | −2.7652 |
| 1765 | −3.5686 | −3.2621 |
| 1766 | −3.6923 | −3.8027 |
| 1767 | −3.9323 | −4.9588 |
| 1768 | −5.0990 | −5.5850 |
| 1769 | −6.2662 | −7.1229 |
| 1770 | −8.4684 | −7.0799 |
| 1771 | −6.5319 | −6.4330 |
| 1772 | −7.5296 | −7.4407 |
| 1773 | −6.6200 | −8.4946 |
| 1774 | −7.9993 | −9.0258 |
| 1775 | −6.3060 | −6.5390 |
| 1776 | −6.7990 | −8.2406 |
| 1777 | −8.2639 | −7.5129 |
| 1778 | −6.8024 | −6.1851 |
| 1779 | −6.3710 | −6.1081 |
| 1780 | −0.1884 | −0.1974 |
| 1781 | 0.0630 | 0.0212 |
| 1782 | −0.1593 | 0.0161 |
| 1783 | −0.1986 | 0.1778 |
| 1784 | −0.3875 | 0.0668 |
| 1785 | −0.2767 | −0.0735 |
| 1786 | −0.0521 | 0.0535 |
| 1787 | −0.0411 | 0.0422 |
| 1788 | 0.0279 | 0.0954 |
| 1789 | −0.2818 | −0.1797 |
| 1790 | −0.1612 | −0.0269 |
| 1791 | −0.7223 | −0.3408 |
| 1792 | −0.5762 | −0.2876 |
| 1793 | −0.4251 | −0.3620 |
| 1794 | −0.5902 | −0.3558 |
| 1795 | −0.8636 | −0.6166 |
| 1796 | −0.4896 | −0.3574 |
| 1797 | −0.4587 | −0.1573 |
| 1798 | −0.2438 | −0.0925 |
| 1799 | −0.4909 | −0.2061 |
| 1800 | −0.1442 | 0.2151 |
| 1801 | −0.2613 | 0.0109 |
| 1802 | 0.1319 | 0.4170 |
| 1803 | −0.3857 | −0.1277 |
| 1804 | −0.7922 | −0.5104 |
| 1805 | −0.5321 | −0.3149 |
| 1806 | −0.5105 | −0.2379 |
| 1807 | −0.6972 | −0.4511 |
| 1808 | −0.6894 | −0.3097 |
| 1809 | −0.3541 | −0.1616 |
| 1810 | −0.1030 | 0.1145 |
| 1811 | −0.2913 | 0.0354 |
| 1812 | −0.1528 | −0.0261 |
| 1813 | −0.4516 | −0.2099 |
| 1814 | −0.0415 | 0.0676 |
| 1815 | −0.8562 | −0.4200 |
| 1816 | −0.5293 | −0.3130 |
| 1817 | −0.5385 | −0.4124 |
| 1818 | −0.8875 | −0.7021 |
| 1819 | −0.8401 | −0.3512 |
| 1820 | −0.9603 | −0.5111 |
| 1821 | −0.5072 | −0.2250 |
| 1822 | −0.7101 | −0.4904 |
| 1823 | −0.1407 | −0.0903 |
| 1824 | −0.4841 | −0.2374 |
| 1825 | −0.7444 | −0.4468 |
| 1826 | −0.4685 | −0.4209 |
| 1827 | −1.1487 | −0.6876 |
| 1828 | −0.9775 | −0.4930 |
| 1829 | −0.6419 | −0.4336 |
| 1830 | −0.9713 | −0.6332 |
| 1831 | −1.2048 | −0.8363 |
| 1832 | −0.9414 | −0.6845 |
| 1833 | −0.8650 | −0.4227 |
| 1834 | −0.7330 | −0.5104 |
| 1835 | −0.8299 | −0.5851 |
| 1836 | −0.6756 | −0.4104 |
| 1837 | −0.7582 | −0.4932 |
| 1838 | −0.7064 | −0.5193 |
| 1839 | −0.9313 | −0.5635 |
| 1840 | −0.9115 | −0.5955 |
| 1841 | −0.7666 | −0.5850 |
| 1842 | 0.3330 | 0.6989 |
| 1843 | −1.1336 | −0.7636 |
| 1844 | −0.9073 | −0.4203 |
| 1845 | −0.7322 | −0.4273 |
| 1846 | −0.6067 | −0.3803 |
| 1847 | −0.6621 | −0.2915 |
| 1848 | −0.5684 | −0.2679 |
| 1849 | −0.2442 | 0.1767 |
| 1850 | −0.6885 | −0.2766 |
| 1851 | −0.2024 | −0.0443 |
| 1852 | −0.7836 | −0.5007 |
| 1853 | −0.9128 | −0.8126 |
| 1854 | −0.6203 | −0.3000 |
| 1855 | −1.0918 | −0.8300 |
| 1856 | −0.6731 | −0.5059 |
| 1857 | −0.4437 | −0.3213 |
| 1858 | −0.7793 | −0.4156 |
| 1859 | −0.8957 | −0.7888 |
| 1860 | −0.7864 | −0.5661 |
| 1861 | −1.2462 | −1.0430 |
| 1862 | −0.8125 | −0.6872 |
| 1863 | −0.9854 | −0.7621 |
| 1864 | −1.3139 | −1.1053 |
| 1865 | −1.0066 | −0.8724 |
| 1866 | −1.1595 | −0.9364 |
| 1867 | −1.2108 | −0.8956 |
| 1868 | −0.9622 | −0.6591 |
| 1869 | −0.5790 | −0.2467 |
| 1870 | −0.8475 | −0.5533 |
| 1871 | −0.8507 | −0.6949 |
| 1872 | −0.7625 | −0.6283 |
| 1873 | −1.0223 | −0.7750 |
| 1874 | −0.7589 | −0.4061 |
| 1875 | −1.3819 | −1.0563 |
| 1876 | −1.2122 | −0.9972 |
| 1877 | −1.1499 | −1.0649 |
| 1878 | −0.1130 | 0.2623 |
| 1879 | −1.5241 | −1.0467 |
| 1880 | −1.3777 | −1.0445 |
| 1881 | −0.6467 | −0.3516 |
| 1882 | −0.7509 | −0.5474 |
| 1883 | −0.8189 | −0.6615 |
| 1884 | −0.7177 | −0.5154 |
| 1885 | −0.8996 | −0.6612 |
| 1886 | −0.7738 | −0.5940 |
| 1887 | −0.0443 | 0.0295 |
| 1888 | −0.1631 | −0.1600 |
| 1889 | 0.3001 | 0.4333 |
| 1890 | −0.2806 | −0.1864 |
| 1891 | −0.1069 | −0.0491 |
| 1892 | −0.0120 | 0.0387 |
| 1893 | −0.0968 | 0.0120 |
| 1894 | 2.2330 | 2.6485 |
| 1895 | 0.0577 | 0.1408 |
| 1896 | −0.2540 | −0.1755 |
| 1897 | −0.7489 | −0.5314 |
| 1898 | −0.3929 | −0.2966 |
| 1899 | −0.7261 | −0.5368 |
| 1900 | −0.7723 | −0.5430 |
| 1901 | −0.5906 | −0.3447 |
| 1902 | −0.3956 | −0.1664 |
| 1903 | −1.0014 | −0.8067 |
| 1904 | −0.4138 | −0.3394 |
| 1905 | 0.2333 | 0.2220 |
| 1906 | 0.0273 | 0.0660 |
| 1907 | 0.3920 | 0.3043 |
| 1908 | 0.2698 | 0.1577 |
| 1909 | −0.0868 | −0.1166 |
| 1910 | 0.1876 | 0.2034 |
| 1911 | −0.6276 | −0.4523 |
| 1912 | −0.5701 | −0.3586 |
| 1913 | −0.3595 | −0.3673 |
| 1914 | −0.2319 | −0.0034 |
| 1915 | −0.3561 | −0.2030 |
| 1916 | −0.4266 | −0.2215 |
| 1917 | −0.0909 | −0.0486 |
| 1918 | −0.1586 | −0.0592 |
| 1919 | 0.0130 | −0.0771 |
| 1920 | 0.0714 | 0.1665 |
| 1921 | −0.1455 | −0.1258 |
| 1922 | 0.0258 | 0.1298 |
| 1923 | −0.1644 | −0.1790 |
| 1924 | −0.0984 | −0.0910 |
| 1925 | −0.1995 | −0.2254 |
| 1926 | −0.5606 | −0.4220 |
| 1927 | −0.5821 | −0.5888 |
| 1928 | −0.1674 | −0.0622 |
| 1929 | −0.2583 | −0.2485 |
| 1930 | −0.3176 | −0.3128 |
| 1931 | −0.3263 | −0.3692 |
| 1932 | −0.3893 | −0.2425 |
| 1933 | −0.2021 | −0.0572 |
| 1934 | −0.3504 | −0.3393 |
| 1935 | −0.6217 | −0.4515 |
| 1936 | −0.7025 | −0.5324 |
| 1937 | −0.5436 | −0.5048 |
| 1938 | −0.5724 | −0.5725 |
| 1939 | −0.7523 | −0.6730 |
| 1940 | −0.6490 | −0.5514 |
| 1941 | −0.3388 | −0.2195 |
| 1942 | −0.6093 | −0.3722 |
| 1943 | −0.3920 | −0.2775 |
| 1944 | −0.0366 | −0.0747 |
| 1945 | −0.1956 | −0.1438 |
| 1946 | −0.2370 | −0.2130 |
| 1947 | −0.5926 | −0.5318 |
| 1948 | −0.4041 | −0.3099 |
| 1949 | −0.7922 | −0.5728 |
| 1950 | −0.7085 | −0.4343 |
| 1951 | −0.9558 | −0.7619 |
| 1952 | −0.5265 | −0.3775 |
| 1953 | −0.8583 | −0.5741 |
| 1954 | −0.5944 | −0.5618 |
| 1955 | −0.4876 | −0.3517 |
| 1956 | 0.1224 | 0.4003 |
| 1957 | −0.6369 | −0.3938 |
| 1958 | −0.4816 | −0.3366 |
| 1959 | 0.1016 | 0.2466 |
| 1960 | 0.2920 | 0.3992 |
| 1961 | −0.0340 | 0.0227 |
| 1962 | 0.0892 | 0.1377 |
| 1963 | −0.3507 | −0.1711 |
| 1964 | −0.0673 | 0.1250 |
| 1965 | 0.2510 | 0.2611 |
| 1966 | 0.0619 | 0.1618 |
| 1967 | −0.2646 | −0.1503 |
| 1968 | −0.4728 | −0.1537 |
| 1969 | −0.4412 | −0.3232 |
| 1970 | −0.1844 | −0.1264 |
| 1971 | −0.3877 | −0.1207 |
| 1972 | −0.3151 | −0.1486 |
| 1973 | −0.3772 | −0.2890 |
| 1974 | −0.2296 | −0.0463 |
| 1975 | −0.6615 | −0.4068 |
| 1976 | −0.3579 | −0.1165 |
| 1977 | −0.1260 | 0.0405 |
| 1978 | −0.3375 | −0.0468 |
| 1979 | −0.0578 | −0.0330 |
| 1980 | 0.2785 | 0.4414 |
| 1981 | 0.0003 | 0.1884 |
| 1982 | −0.0715 | 0.1504 |
| 1983 | −0.4546 | −0.2193 |
| 1984 | 0.0270 | 0.1030 |
| 1985 | −0.5039 | −0.2524 |
| 1986 | −0.1878 | 0.1550 |
| 1987 | −0.6160 | −0.2139 |
| 1988 | −0.5063 | −0.2076 |
| 1989 | −0.0546 | 0.1704 |
| 1990 | 0.0593 | 0.0710 |
| 1991 | −0.0966 | 0.0191 |
| 1992 | −0.0087 | 0.1861 |
| 1993 | −0.0221 | 0.2525 |
| 1994 | −0.0541 | 0.0957 |
| 1995 | −7.1129 | −8.0140 |
| 1996 | −3.4969 | −3.0592 |
| 1997 | −7.2803 | −8.3069 |
| 1998 | −7.5205 | −7.0877 |
| 1999 | −2.1667 | −1.7954 |
| 2000 | −7.4835 | −9.0950 |
| 2001 | −7.9785 | −6.5104 |
| 2002 | −3.7134 | −3.8140 |
| 2003 | −6.2523 | −6.1914 |
| 2004 | −2.2890 | −2.4537 |
| 2005 | −5.1505 | −6.2702 |
| 2006 | −6.8435 | −6.4107 |
| 2007 | −0.4196 | −0.1150 |
| 2008 | −1.0520 | −0.7302 |
| 2009 | −0.6689 | −0.3614 |
| 2010 | −1.1280 | −0.7370 |
| 2011 | −0.7297 | −0.4421 |
| 2012 | −0.2258 | −0.2086 |
| 2013 | −0.3872 | −0.4002 |
| 2014 | −1.4100 | −1.0541 |
| 2015 | −0.3620 | 0.1070 |
| 2016 | −1.3658 | −0.9739 |
| 2017 | −0.9439 | −0.5450 |
| 2018 | −1.4644 | −0.9636 |
| 2019 | −1.5257 | −1.3231 |
| 2020 | −1.8990 | −1.4184 |
| 2021 | −1.5937 | −1.0264 |
| 2022 | −1.9251 | −1.4781 |
| 2023 | −1.2324 | −1.0090 |
| 2024 | −1.0463 | −0.8770 |
| 2025 | −1.2407 | −0.7602 |
| 2026 | −1.2775 | −1.1026 |
| 2027 | −0.3669 | −0.2368 |
| 2028 | −1.3166 | −0.9760 |
| 2029 | −0.7655 | −0.6093 |
| 2030 | −1.3221 | −0.9845 |
| 2031 | −1.1173 | −0.9110 |
| 2032 | −1.3259 | −1.0798 |
| 2033 | −1.0403 | −0.6767 |
| 2034 | −1.6979 | −1.2635 |
| 2035 | −1.1355 | −0.7183 |
| 2036 | −0.9044 | −0.5234 |
| 2037 | −0.9799 | −0.6716 |
| 2038 | −0.9124 | −0.6817 |
| 2039 | −0.8085 | −0.5564 |
| 2040 | −1.6072 | −1.0276 |
| 2041 | −0.6837 | −0.3112 |
| 2042 | 0.1938 | 0.2889 |
| 2043 | −3.4340 | −2.9613 |
| 2044 | −3.6451 | −3.4329 |
| 2045 | −3.0815 | −2.1385 |
| 2046 | −6.2508 | −6.2774 |
| 2047 | −2.9912 | −2.5729 |
| 2048 | −6.1049 | −6.4116 |
| 2049 | 0.2738 | 0.3285 |
| 2050 | −3.4583 | −2.4849 |
| 2051 | −0.7885 | −0.5992 |
| 2052 | −0.6760 | −0.2805 |
| 2053 | −0.8185 | −0.5969 |
| 2054 | −0.7079 | −0.4781 |
| 2055 | −0.9373 | −0.5756 |
| 2056 | −0.5923 | −0.4757 |
| 2057 | −1.3648 | −0.9257 |
| 2058 | −1.3537 | −0.9649 |
| 2059 | −1.9742 | −1.6101 |
| 2060 | −1.1791 | −0.9268 |
| 2061 | −2.0230 | −1.8174 |
| 2062 | −0.8160 | −0.3559 |
| 2063 | −2.2503 | −1.5521 |
| 2064 | −1.7615 | −1.3870 |
| 2065 | −2.7534 | −2.2951 |
| 2066 | −1.5942 | −1.3235 |
| 2067 | −1.4763 | −1.1494 |
| 2068 | −1.6811 | −1.2483 |
| 2069 | −1.7062 | −1.2011 |
| 2070 | −1.8639 | −1.3988 |
| 2071 | −2.7005 | −2.0227 |
| 2072 | −1.7197 | −1.4910 |
| 2073 | −2.4276 | −2.0984 |
| 2074 | −1.6913 | −1.3619 |
| 2075 | −2.3759 | −1.6772 |
| 2076 | −1.9170 | −1.6590 |
| 2077 | −2.4998 | −2.1910 |
| 2078 | −6.1930 | −6.3979 |
| 2079 | −8.2315 | −7.3511 |
| 2080 | −6.6046 | −7.1547 |
| 2081 | −7.5199 | −7.3241 |
| 2082 | −8.4808 | −7.5074 |
| 2083 | −6.3439 | −6.4346 |
| 2084 | −7.7945 | −6.5315 |
| 2085 | −7.9254 | −7.9519 |
| 2086 | −7.6212 | −7.4362 |
| 2087 | −5.5354 | −6.8421 |
| 2088 | −7.9551 | −7.5342 |
| 2089 | −6.6892 | −6.6487 |
| 2090 | −7.2609 | −7.0811 |
| 2091 | −7.0093 | −7.0359 |
| 2092 | −6.6746 | −7.2668 |
| 2093 | −7.0587 | −7.2165 |
| 2094 | −7.2409 | −7.8525 |
| 2095 | −7.6750 | −7.1869 |
| 2096 | −7.3981 | −7.0096 |
| 2097 | −6.9914 | −7.0793 |
| 2098 | −7.1053 | −7.3856 |
| 2099 | −6.8415 | −7.3826 |
| 2100 | −7.2186 | −7.5822 |
| 2101 | −7.3201 | −6.9316 |
| 2102 | −1.5074 | −1.2811 |
| 2103 | −1.8092 | −1.3219 |
| 2104 | −2.2420 | −1.6432 |
| 2105 | −4.0394 | −3.3114 |
| 2106 | −2.1639 | −1.8367 |
| 2107 | −2.9623 | −2.6276 |
| 2108 | −2.1558 | −1.8272 |
| 2109 | −2.0790 | −1.8225 |
| 2110 | −2.5318 | −2.1639 |
| 2111 | −3.5708 | −3.2029 |
| 2112 | −4.2836 | −4.0471 |
| 2113 | −5.6754 | −4.8089 |
| 2114 | −2.2872 | −1.5584 |
| 2115 | −2.0962 | −1.3329 |
| 2116 | −3.0533 | −2.4936 |
| 2117 | −4.8962 | −3.9814 |
| 2118 | −2.4944 | −2.1846 |
| 2119 | −3.1702 | −2.5134 |
| 2120 | −2.4887 | −2.1038 |
| 2121 | −3.3787 | −2.7220 |
| 2122 | −3.3570 | −2.8306 |
| 2123 | −3.9780 | −3.2089 |
| 2124 | −4.9082 | −4.3916 |
| 2125 | −4.9938 | −4.6135 |
| 2126 | −6.3501 | −7.6176 |
| 2127 | −8.3811 | −6.6707 |
| 2128 | −6.1743 | −5.7942 |
| 2129 | −8.0430 | −7.9176 |
| 2130 | −6.2265 | −6.6819 |
| 2131 | −6.5847 | −7.1556 |
| 2132 | −6.9461 | −6.5804 |
| 2133 | −6.7787 | −7.9428 |
| 2134 | −7.8370 | −6.3192 |
| 2135 | −7.9526 | −7.2422 |
| 2136 | −6.6305 | −6.8415 |
| 2137 | −7.1300 | −6.4561 |
| 2138 | −7.4703 | −7.2904 |
| 2139 | −7.7484 | −6.7750 |
| 2140 | −7.5993 | −7.3914 |
| 2141 | −7.7304 | −6.7570 |
| 2142 | −6.1621 | −5.8805 |
| 2143 | −7.7433 | −7.9073 |
| 2144 | −7.7440 | −8.6775 |
| 2145 | −7.0123 | −7.6239 |
| 2146 | −7.2439 | −6.5824 |
| 2147 | −7.5752 | −6.8497 |
| 2148 | −7.3769 | −7.3421 |
| 2149 | −7.7776 | −7.5978 |
| 2150 | −2.0575 | −1.6267 |
| 2151 | −2.3368 | −1.8651 |
| 2152 | −1.9120 | −1.5449 |
| 2153 | −2.3980 | −2.0917 |
| 2154 | −3.2663 | −2.8623 |
| 2155 | −4.8796 | −4.3079 |
| 2156 | −3.3002 | −3.0325 |
| 2157 | −4.6416 | −4.4574 |
| 2158 | −3.9163 | −3.3601 |
| 2159 | −5.4142 | −4.7176 |
| 2160 | −4.1644 | −3.6385 |
| 2161 | −5.9788 | −4.6361 |
| 2162 | −3.2222 | −2.7782 |
| 2163 | −3.2154 | −2.7747 |
| 2164 | −3.5469 | −2.8251 |
| 2165 | −3.5743 | −3.2476 |
| 2166 | −4.0971 | −3.6427 |
| 2167 | −4.4595 | −4.7168 |
| 2168 | −4.1276 | −3.9375 |
| 2169 | −4.9133 | −4.8633 |
| 2170 | −4.2037 | −3.6701 |
| 2171 | −5.0278 | −4.8134 |
| 2172 | −5.1401 | −4.8641 |
| 2173 | −6.2487 | −5.4403 |
| 2174 | −0.7818 | −0.2572 |
| 2175 | −1.1647 | −0.7065 |
| 2176 | −0.6972 | −0.5144 |
| 2177 | −1.2337 | −0.5908 |
| 2178 | −1.4268 | −1.1690 |
| 2179 | −2.1819 | −1.7161 |
| 2180 | −1.5017 | −1.2484 |
| 2181 | −2.1866 | −1.4674 |
| 2182 | −1.3127 | −0.9854 |
| 2183 | −1.9032 | −1.3824 |
| 2184 | −1.2514 | −0.7584 |
| 2185 | −1.9248 | −1.3184 |
| 2186 | −1.5015 | −1.1899 |
| 2187 | −1.4431 | −1.1738 |
| 2188 | −1.3871 | −1.1524 |
| 2189 | −1.8664 | −1.3155 |
| 2190 | −1.5750 | −1.3127 |
| 2191 | −1.9796 | −1.6227 |
| 2192 | −1.8311 | −1.3821 |
| 2193 | −2.1552 | −1.8222 |
| 2194 | −1.4715 | −1.0805 |
| 2195 | −2.1865 | −1.5765 |
| 2196 | −2.0339 | −1.6387 |
| 2197 | −2.2254 | −1.7144 |
| 2198 | −1.7795 | −1.4328 |
| 2199 | −2.6067 | −2.0439 |
| 2200 | −2.4026 | −1.9950 |
| 2201 | −3.0365 | −2.4475 |
| 2202 | −3.4011 | −3.0782 |
| 2203 | −4.4363 | −3.8729 |
| 2204 | −3.5271 | −3.3537 |
| 2205 | −4.7059 | −4.6285 |
| 2206 | −4.0901 | −3.5182 |
| 2207 | −4.7711 | −4.9980 |
| 2208 | −4.1653 | −3.6234 |
| 2209 | −4.9909 | −5.6024 |
| 2210 | −3.1495 | −2.4609 |
| 2211 | −2.8322 | −2.1713 |
| 2212 | −3.0604 | −2.6731 |
| 2213 | −3.0516 | −2.2141 |
| 2214 | −4.6305 | −3.8080 |
| 2215 | −4.8833 | −4.5610 |
| 2216 | −4.3300 | −3.7308 |
| 2217 | −4.6819 | −4.4296 |
| 2218 | −4.6073 | −4.1158 |
| 2219 | −4.5222 | −4.5383 |
| 2220 | −4.4873 | −4.6877 |
| 2221 | −5.4480 | −5.1367 |
| 2222 | −1.2439 | −0.8960 |
| 2223 | −1.5563 | −1.2030 |
| 2224 | −1.2838 | −0.9762 |
| 2225 | −0.8131 | −0.4098 |
| 2226 | −1.3225 | −1.0301 |
| 2227 | −2.5101 | −1.9090 |
| 2228 | −1.2945 | −1.0838 |
| 2229 | −2.6822 | −2.2227 |
| 2230 | −1.5998 | −1.3212 |
| 2231 | −2.2939 | −1.9974 |
| 2232 | −2.3573 | −1.9314 |
| 2233 | −3.3666 | −2.6318 |
| 2234 | −1.7897 | −1.5619 |
| 2235 | −2.0935 | −1.6687 |
| 2236 | −1.6048 | −1.2642 |
| 2237 | −2.3975 | −1.9737 |
| 2238 | −1.9837 | −1.5714 |
| 2239 | −2.4290 | −1.9829 |
| 2240 | −2.0479 | −1.7776 |
| 2241 | −2.3309 | −2.0609 |
| 2242 | −2.0274 | −1.6006 |
| 2243 | −2.4904 | −1.9721 |
| 2244 | −2.3668 | −1.9443 |
| 2245 | −2.5718 | −2.4181 |
| 2246 | −0.8184 | −0.5806 |
| 2247 | −1.1633 | −0.7744 |
| 2248 | −0.9278 | −0.6495 |
| 2249 | −1.6247 | −1.2168 |
| 2250 | −1.6170 | −1.4078 |
| 2251 | −2.2170 | −1.9320 |
| 2252 | −1.5645 | −1.2449 |
| 2253 | −2.8079 | −2.0905 |
| 2254 | −1.5100 | −1.2488 |
| 2255 | −2.3309 | −1.7567 |
| 2256 | −2.3471 | −1.8227 |
| 2257 | −3.1226 | −2.5655 |
| 2258 | −1.9112 | −1.4898 |
| 2259 | −1.7833 | −1.4333 |
| 2260 | −1.7458 | −1.4594 |
| 2261 | −2.6705 | −2.2130 |
| 2262 | −1.9613 | −1.5111 |
| 2263 | −2.7667 | −2.0650 |
| 2264 | −2.2105 | −1.9408 |
| 2265 | −2.7392 | −2.1935 |
| 2266 | −2.0635 | −1.5094 |
| 2267 | −2.4373 | −2.1211 |
| 2268 | −2.5504 | −2.1783 |
| 2269 | −3.2395 | −2.6058 |
| 2270 | −2.0031 | −1.7616 |
| 2271 | −3.2258 | −3.3167 |
| 2272 | −2.3862 | −2.1318 |
| 2273 | −4.6783 | −4.2810 |
| 2274 | −2.0372 | −1.7937 |
| 2275 | −3.1606 | −2.6225 |
| 2276 | −2.4579 | −2.0565 |
| 2277 | −3.6676 | −2.9314 |
| 2278 | −1.1501 | −0.8472 |
| 2279 | −3.2526 | −3.0015 |
| 2280 | −2.4979 | −2.3557 |
| 2281 | −3.2407 | −2.9977 |
| 2282 | −1.5486 | −1.2211 |
| 2283 | −4.2665 | −4.1530 |
| 2284 | −3.5851 | −3.0282 |
| 2285 | −4.9308 | −4.9574 |
| 2286 | −1.2799 | −0.9454 |
| 2287 | −2.7853 | −2.7768 |
| 2288 | −2.6621 | −2.3995 |
| 2289 | −3.2150 | −2.9177 |
| 2290 | −1.1698 | −1.0625 |
| 2291 | −3.0877 | −3.1558 |
| 2292 | −2.6007 | −2.1461 |
| 2293 | −3.2241 | −2.8990 |
| 2294 | −1.6521 | −1.2327 |
| 2295 | −3.1722 | −2.5055 |
| 2296 | −2.1402 | −1.5261 |
| 2297 | −2.1627 | −1.9035 |
| 2298 | −3.3346 | −2.9767 |
| 2299 | −1.6818 | −1.3810 |
| 2300 | −2.5294 | −2.1143 |
| 2301 | −4.1665 | −3.8541 |
| 2302 | −3.3350 | −2.6324 |
| 2303 | −3.6988 | −3.1743 |
| 2304 | −5.0074 | −4.4755 |
| 2305 | −0.8429 | −0.6314 |
| 2306 | −1.7300 | −1.3646 |
| 2307 | −3.3179 | −2.8547 |
| 2308 | −2.1262 | −1.7765 |
| 2309 | −2.4509 | −2.1063 |
| 2310 | −4.2183 | −3.8607 |
| 2311 | −2.5259 | −1.9417 |
| 2312 | −3.0899 | −2.6366 |
| 2313 | −5.2052 | −5.0339 |
| 2314 | −3.5200 | −3.1739 |
| 2315 | −4.5495 | −4.4087 |
| 2316 | −5.5160 | −5.3094 |
| 2317 | −1.2348 | −0.9428 |
| 2318 | −0.9673 | −0.6696 |
| 2319 | −2.2909 | −2.0971 |
| 2320 | −1.6534 | −1.3962 |
| 2321 | −2.0250 | −1.3635 |
| 2322 | −3.4271 | −2.7214 |
| 2323 | −2.1433 | −1.7101 |
| 2324 | −2.0710 | −1.7141 |
| 2325 | −4.2877 | −3.5465 |
| 2326 | −2.9483 | −2.3515 |
| 2327 | −3.1067 | −2.8808 |
| 2328 | −4.8101 | −4.5336 |
| 2329 | −1.2253 | −0.8411 |
| 2330 | −1.4536 | −1.1744 |
| 2331 | −3.0190 | −2.6746 |
| 2332 | −2.2468 | −1.8325 |
| 2333 | −2.5098 | −2.0612 |
| 2334 | −3.6436 | −3.1441 |
| 2335 | −1.8349 | −1.5164 |
| 2336 | −2.2984 | −2.0919 |
| 2337 | −4.2329 | −4.3872 |
| 2338 | −2.8997 | −2.6184 |
| 2339 | −3.4806 | −3.0253 |
| 2340 | −4.6965 | −5.0584 |
| 2341 | −1.2750 | −1.0242 |
| 2342 | −0.9919 | −0.5382 |
| 2343 | −1.1110 | −0.1115 |
| 2344 | −2.4636 | −1.7482 |
| 2345 | −2.3947 | −1.9788 |
| 2346 | −3.8343 | −3.7963 |
| 2347 | −2.5409 | −2.2272 |
| 2348 | −1.1621 | −0.4115 |
| 2349 | −5.4185 | −5.6056 |
| 2350 | −3.9129 | −3.4974 |
| 2351 | −3.8474 | −3.3321 |
| 2352 | −5.5369 | −5.4030 |
| 2353 | −0.9872 | −0.8463 |
| 2354 | −1.7729 | −1.1504 |
| 2355 | −3.4378 | −3.1656 |
| 2356 | −1.3882 | −1.0757 |
| 2357 | −2.7989 | −2.3021 |
| 2358 | −3.7588 | −3.6605 |
| 2359 | −2.5332 | −2.0110 |
| 2360 | −3.3817 | −3.0034 |
| 2361 | −5.4682 | −5.2183 |
| 2362 | −3.8863 | −3.5909 |
| 2363 | −5.1907 | −3.9884 |
| 2364 | −5.8138 | −5.7148 |
| 2365 | −1.5634 | −1.0344 |
| 2366 | −0.8701 | −0.6795 |
| 2367 | −2.8881 | −2.5794 |
| 2368 | −2.0596 | −1.6264 |
| 2369 | −2.3899 | −1.8587 |
| 2370 | −3.1556 | −2.4635 |
| 2371 | −2.7582 | −2.3304 |
| 2372 | −1.9147 | −1.6748 |
| 2373 | −4.2736 | −4.3109 |
| 2374 | −2.8749 | −2.4160 |
| 2375 | −3.4913 | −3.0880 |
| 2376 | −4.1777 | −4.5404 |
| 2377 | −1.4958 | −1.1395 |
| 2378 | −1.3546 | −1.0467 |
| 2379 | −3.5045 | −3.0544 |
| 2380 | −2.1692 | −1.6532 |
| 2381 | −2.4256 | −2.0734 |
| 2382 | −3.7444 | −3.6355 |
| 2383 | −3.1415 | −2.4233 |
| 2384 | −3.0577 | −2.6921 |
| 2385 | −5.3615 | −5.4827 |
| 2386 | −4.1305 | −3.8102 |
| 2387 | −4.6299 | −4.2336 |
| 2388 | −4.9895 | −5.1898 |
| 2389 | −1.5476 | −1.0863 |
| 2390 | −2.1864 | −1.8697 |
| 2391 | −2.6470 | −2.1051 |
| 2392 | −4.3671 | −4.7018 |
| 2393 | −3.4281 | −3.0664 |
| 2394 | −3.2244036 | −3.03416 |
| 2395 | −5.7428663 | −5.60897 |
| 2396 | −3.7571948 | −3.68972 |
| 2397 | −4.6622996 | −4.3086 |
| 2398 | −6.3901301 | −6.12124 |
| 2399 | −4.2512773 | −4.36734 |
| 2400 | −4.6003386 | −4.85087 |
| 2401 | −5.7540541 | −5.55823 |
| 2402 | −1.1998086 | −0.90182 |
| 2403 | −2.6938442 | −2.17817 |
| 2404 | −3.6247429 | −3.19543 |
| 2405 | −1.3007316 | −0.93601 |
| 2406 | −1.665633 | −1.39317 |
| 2407 | −3.3129689 | −2.89234 |
| 2408 | −3.5978822 | −3.70323 |
| 2409 | −1.6714951 | −1.18417 |
| 2410 | −0.6443582 | −0.05381 |
| 2411 | −3.3064288 | −2.8295 |
| 2412 | −5.0439452 | −4.53115 |
| 2413 | −1.6257667 | −1.27789 |
| 2414 | −2.2081452 | −1.91854 |
| 2415 | −3.7917037 | −3.40508 |
| 2416 | −5.2299972 | −5.45966 |
| 2417 | −2.0306575 | −1.60323 |
| 2418 | −3.1938703 | −2.59631 |
| 2419 | −3.1020778 | −2.63171 |
| 2420 | −4.0866251 | −4.16129 |
| 2421 | −2.6919731 | −2.15643 |
| 2422 | −3.0223734 | −3.08196 |
| 2423 | −3.5472507 | −2.81784 |
| 2424 | −4.3000111 | −4.05433 |
| 2425 | −2.7697687 | −2.39115 |
| 2426 | −3.9607477 | −4.11782 |
| 2427 | −3.6215616 | −3.71456 |
| 2428 | −5.4364436 | −5.56685 |
| 2429 | −3.3792833 | −2.91573 |
| 2430 | −4.9416692 | −4.724 |
| 2431 | −4.2123107 | −3.62946 |
| 2432 | −5.6371064 | −5.77471 |
| 2433 | −0.9552548 | −0.54186 |
| 2434 | −2.0904641 | −1.72769 |
| 2435 | −2.6361393 | −2.17521 |
| 2436 | −4.7644931 | −4.68502 |
| 2437 | −1.2263682 | −0.98215 |
| 2438 | −2.1259115 | −1.97525 |
| 2439 | −3.1396055 | −2.70105 |
| 2440 | −5.4744989 | −5.00099 |
| 2441 | −1.4783296 | −1.28835 |
| 2442 | −2.945823 | −2.64344 |
| 2443 | −3.6499764 | −3.18021 |
| 2444 | −6.746999 | −6.39917 |
| 2445 | −1.5437518 | −1.21134 |
| 2446 | −3.3531229 | −3.02631 |
| 2447 | −3.5907464 | −3.21483 |
| 2448 | −6.9172713 | −5.73439 |
| 2449 | −2.3640097 | −1.82885 |
| 2450 | −3.9405414 | −3.79419 |
| 2451 | −3.9522309 | −3.55349 |
| 2452 | −5.3619735 | −5.79863 |
| 2453 | −3.1092079 | −2.51519 |
| 2454 | −4.8881069 | −4.87883 |
| 2455 | −3.9529068 | −3.61109 |
| 2456 | −5.6247684 | −6.35178 |
| 2457 | −3.0368398 | −2.6967 |
| 2458 | −5.4937076 | −5.71061 |
| 2459 | −4.445899 | −4.01174 |
| 2460 | −3.7118987 | −2.58093 |
| 2461 | −3.5066298 | −2.80782 |
| 2462 | −6.4886684 | −6.73763 |
| 2463 | −4.791343 | −4.1585 |
| 2464 | −6.8496709 | −7.12778 |
| 2465 | −1.8442973 | −1.23897 |
| 2466 | −1.0662757 | −0.92975 |
| 2467 | −0.9172488 | −0.64154 |
| 2468 | −1.343801 | −1.13897 |
| 2469 | −1.0569375 | −1.0193 |
| 2470 | −0.9641409 | −0.84884 |
| 2471 | −1.6991112 | −1.35393 |
| 2472 | −1.1482473 | −0.99507 |
| 2473 | −2.2826271 | −1.71223 |
| 2474 | −0.7834575 | −0.60894 |
| 2475 | −1.9516205 | −1.47074 |
| 2476 | −0.8849024 | −0.61833 |
| 2477 | −1.0042589 | −0.75109 |
| 2478 | −0.768967 | −0.57988 |
| 2479 | −1.975371 | −1.51916 |
| 2480 | −1.4702561 | −1.07803 |
| 2481 | −1.0746524 | −0.90539 |
| 2482 | −0.8654849 | −0.73141 |
| 2483 | −0.8323908 | −0.61654 |
| 2484 | −1.5370961 | −0.9676 |
| 2485 | −1.4174268 | −1.06563 |
| 2486 | −1.5507766 | −1.05531 |
| 2487 | −0.740465 | −0.62047 |
| 2488 | −0.7281564 | −0.68761 |
| 2489 | −1.3858569 | −0.7984 |
| 2490 | −2.0566707 | −1.66902 |
| 2491 | −1.9862039 | −1.79892 |
| 2492 | −2.6676277 | −2.23875 |
| 2493 | −1.7611844 | −1.42816 |
| 2494 | −2.7157335 | −1.88194 |
| 2495 | −3.0882586 | −2.39104 |
| 2496 | −1.9364893 | −1.5058 |
| 2497 | −2.5334174 | −1.75483 |
| 2498 | −2.9649654 | −2.21136 |
| 2499 | −2.8221448 | −1.96575 |
| 2500 | −3.0827193 | −2.23837 |
| 2501 | −3.3226189 | −3.09228 |
| 2502 | −0.7451109 | −0.49476 |
| 2503 | −0.4172012 | −0.15043 |
| 2504 | −0.8026727 | −0.70425 |
| 2505 | −0.631294 | −0.50701 |
| 2506 | −0.8920533 | −0.72663 |
| 2507 | −1.2207223 | −1.09637 |
| 2508 | −1.468937 | −1.10591 |
| 2509 | −1.3182826 | −0.86912 |
| 2510 | −1.3150213 | −0.93068 |
| 2511 | −1.211751 | −0.79402 |
| 2512 | −0.6299349 | −0.55684 |
| 2513 | −1.3181023 | −1.00404 |
| 2514 | −1.9384823 | −1.49145 |
| 2515 | −0.7789869 | −0.56773 |
| 2516 | −1.5499598 | −1.17679 |
| 2517 | −1.348204 | −0.98181 |
| 2518 | −0.955361 | −0.4166 |
| 2519 | −0.7541713 | −0.60457 |
| 2520 | −1.5217027 | −1.05495 |
| 2521 | −1.6101307 | −1.10727 |
| 2522 | −1.8296628 | −1.40671 |
| 2523 | −1.4010485 | −1.12222 |
| 2524 | −1.8819491 | −1.38106 |
| 2525 | −1.1862519 | −0.63257 |
| 2526 | −1.2483563 | −0.65803 |
| 2527 | −1.6421789 | −1.15805 |
| 2528 | −2.22162 | −1.81946 |
| 2529 | −2.4782051 | −2.22973 |
| 2530 | −3.9579347 | −3.49781 |
| 2531 | −1.2096058 | −0.99288 |
| 2532 | −1.6775047 | −1.25703 |
| 2533 | −1.044021 | −0.91189 |
| 2534 | −2.2355167 | −1.62897 |
| 2535 | −1.6877766 | −1.28834 |
| 2536 | 0.02724083 | 0.784222 |
| 2537 | −1.4903288 | −0.9019 |
| 2538 | −1.5998599 | −1.41219 |
| 2539 | −1.7039935 | −1.30276 |
| 2540 | −1.1637701 | −0.91641 |
| 2541 | −1.4936506 | −1.19049 |
| 2542 | −1.1432239 | −0.87427 |
| 2543 | −2.2571491 | −2.10763 |
| 2544 | −0.8651703 | −0.74435 |
| 2545 | −1.4673999 | −1.20295 |
| 2546 | −1.1834394 | −0.94604 |
| 2547 | −1.1495841 | −0.81489 |
| 2548 | −0.8218145 | −0.79598 |
| 2549 | −0.7429876 | −0.77766 |
| 2550 | −1.8530822 | −1.56741 |
| 2551 | −1.7196975 | −1.3944 |
| 2552 | −1.1969268 | −0.74649 |
| 2553 | −1.3933533 | −1.12938 |
| 2554 | −1.6290546 | −1.39462 |
| 2555 | −2.3299007 | −1.8233 |
| 2556 | −2.93717 | −2.55668 |
| 2557 | −0.9019328 | −0.72441 |
| 2558 | −3.419818 | −3.08284 |
| 2559 | −1.9535045 | −1.62134 |
| 2560 | −1.349957 | −1.13037 |
| 2561 | −2.8695668 | −2.38107 |
| 2562 | −0.8395943 | −0.64683 |
| 2563 | −1.2455191 | −1.0339 |
| 2564 | −1.6914905 | −1.35227 |
| 2565 | −1.5440732 | −1.47301 |
| 2566 | −2.288956 | −1.98762 |
| 2567 | −2.4867525 | −1.88543 |
| 2568 | −0.7615909 | −0.55264 |
| 2569 | −0.7306614 | −0.53445 |
| 2570 | −1.5689512 | −1.16903 |
| 2571 | −1.6184381 | −1.13484 |
| 2572 | −1.5978315 | −1.01401 |
| 2573 | −1.8840855 | −1.62831 |
| 2574 | −1.5696043 | −1.06584 |
| 2575 | −1.6548671 | −1.21801 |
| 2576 | −1.0867712 | −0.77671 |
| 2577 | −1.7189417 | −1.32657 |
| 2578 | −1.2091111 | −1.04735 |
| 2579 | −1.056773 | −0.7984 |
| 2580 | −1.6086941 | −1.22806 |
| 2581 | −0.8908394 | −0.91648 |
| 2582 | −1.0966939 | −0.97929 |
| 2583 | −0.6758699 | −0.6938 |
| 2584 | −1.4888911 | −0.9447 |
| 2585 | −0.9416641 | −0.59133 |
| 2586 | 1.0872418 | 1.99433 |
| 2587 | −1.5358263 | −1.14766 |
| 2588 | −1.2510645 | −0.99248 |
| 2589 | −1.4260377 | −1.17687 |
| 2590 | −1.617369 | −1.23074 |
| 2591 | −1.3032917 | −1.10247 |
| 2592 | −1.138094 | −0.83178 |
| 2593 | −1.4258585 | −1.16598 |
| 2594 | −1.3568068 | −0.95222 |
| 2595 | −1.3538859 | −0.65488 |
| 2596 | −1.4728943 | −1.18834 |
| 2597 | −1.3440397 | −1.01486 |
| 2598 | −0.8687717 | −0.36232 |
| 2599 | −1.5591755 | −1.05773 |
| 2600 | −1.1094687 | −0.68192 |
| 2601 | −1.6662385 | −1.05614 |
| 2602 | −1.1622842 | −0.56222 |
| 2603 | −2.0571823 | −1.51761 |
| 2604 | −2.0460547 | −1.36318 |
| 2605 | −1.9547626 | −1.30995 |
| 2606 | −1.5522903 | −1.03328 |
| 2607 | −1.730117 | −1.28007 |
| 2608 | −1.6485331 | −1.15145 |
| 2609 | −1.5013049 | −1.08455 |
| 2610 | −1.6406066 | −1.24156 |
| 2611 | −1.715239 | −1.3998 |
| 2612 | −2.1452755 | −1.85154 |
| 2613 | −0.2313744 | 0.381126 |
| 2614 | −1.7688496 | −1.39891 |
| 2615 | −2.483645 | −1.88315 |
| 2616 | −1.6905582 | −1.27752 |
| 2617 | −4.9549009 | −4.28103 |
| 2618 | −5.0640567 | −5.18374 |
| 2619 | −1.4010726 | −0.90183 |
| 2620 | −1.276142 | −1.01086 |
| 2621 | −1.3836097 | −0.86505 |
| 2622 | −1.1953284 | −0.88708 |
| 2623 | −1.6628601 | −1.2592 |
| 2624 | −1.7796949 | −1.56974 |
| 2625 | −1.2053754 | −0.87014 |
| 2626 | −1.3221893 | −1.0371 |
| 2627 | −1.3017623 | −0.98456 |
| 2628 | −1.7778425 | −1.46748 |
| 2629 | −0.536355 | −0.45638 |
| 2630 | −0.9516366 | −0.7336 |
| 2631 | −1.0044283 | −0.79755 |
| 2632 | −1.188787 | −0.98523 |
| 2633 | −0.8366792 | −0.56098 |
| 2634 | −0.9221736 | −0.79839 |
| 2635 | −0.9532542 | −0.61156 |
| 2636 | −1.2654204 | −0.91689 |
| 2637 | −0.8353265 | −0.52821 |
| 2638 | −0.6644217 | −0.62686 |
| 2639 | −1.115754 | −0.78081 |
| 2640 | −1.1977378 | −0.74374 |
| 2641 | −2.062537 | −1.67286 |
| 2642 | −1.0535857 | −0.82071 |
| 2643 | −1.761709 | −1.3486 |
| 2644 | −6.3310061 | −5.80299 |
| 2645 | −2.0972254 | −1.49791 |
| 2646 | −0.253332 | 0.060851 |
| 2647 | −1.3806913 | −0.91729 |
| 2648 | −1.3265892 | −0.9581 |
| 2649 | −1.67489 | −1.21814 |
| 2650 | −1.9512512 | −1.48689 |
| 2651 | −1.8010266 | −1.42827 |
| 2652 | −6.5749452 | −6.12758 |
| 2653 | −2.4658991 | −1.68639 |
| 2654 | −3.2543849 | −2.73095 |
| 2655 | −1.319048 | −0.85289 |
| 2656 | −1.6601983 | −1.26504 |
| 2657 | −1.1338138 | −0.87975 |
| 2658 | −2.416775 | −1.81909 |
| 2659 | −2.3961048 | −1.87639 |
| 2660 | −3.1192493 | −2.38701 |
| 2661 | −2.6166433 | −1.99705 |
| 2662 | −3.9458821 | −3.36979 |
| 2663 | −2.5765975 | −1.96871 |
| 2664 | −3.1717375 | −2.70293 |
| 2665 | −4.5649517 | −3.68153 |
| 2666 | −3.6184972 | −3.385 |
| 2667 | −4.9749445 | −4.96835 |
| 2668 | −4.8768597 | −4.17473 |
| 2669 | −1.3871124 | −0.84097 |
| 2670 | −0.2228668 | 0.165601 |
| 2671 | −0.426376 | −0.12278 |
| 2672 | −0.2244834 | 0.146588 |
| 2673 | −0.2328573 | 0.12926 |
| 2674 | −0.5513359 | −0.12122 |
| 2675 | −0.4143876 | −0.01217 |
| 2676 | 0.72846811 | 1.022007 |
| 2677 | −0.0193746 | 0.331152 |
| 2678 | −0.178882 | 0.149153 |
| 2679 | −0.1030894 | 0.394592 |
| 2680 | 0.19396402 | 0.611923 |
| 2681 | −0.2898463 | 0.074417 |
| 2682 | −0.3675817 | −0.0785 |
| 2683 | −0.6233649 | −0.00046 |
| 2684 | −0.3323308 | −0.0602 |
| 2685 | −0.3873451 | −0.17892 |
| 2686 | −0.2770517 | −0.18569 |
| 2687 | −0.4180592 | −0.08773 |
| 2688 | −0.1241845 | 0.293618 |
| 2689 | −0.1850324 | 0.150474 |
| 2690 | −0.0353577 | 0.097671 |

TABLE 9
Plasmids

| ID | Description | DNA sequence |
|---|---|---|
| pSL3352 | pUC57_Tn6677_TnL_cargo_TnR (800 bp-miniTn) | 5153 |
| pSL1022 | pCDF_Vch_PT7_CRISPR(Target4)_QCascade_TnsABC_T7Term | 5154 |
| pSL1236 | pDonor | 5155 |
| pSL1022 | pEffector encoding the guide RNA that recognizes target 4 | 5156 |
| pSL4277 | pEffector encoding the guide RNA that recognizes target 5 | 5157 |
| pSL4278 | pEffector encoding the guide RNA that recognizes target 6 | 5158 |
| pSL4279 | pEffector encoding the guide RNA that recognizes target 7 | 5159 |
| pSL4196 | pTarget: Target 4_Downstream 4 | 5160 |
| pSL4200 | pTarget: Target 4_Downstream 5 | 5161 |
| pSL4201 | pTarget: Target 4_Downstream 6 | 5162 |
| pSL4202 | pTarget: Target 4_Downstream 7 | 5163 |
| pSL4197 | pTarget: Target 5_Downstream 5 | 5164 |
| pSL4203 | pTarget: Target 5_Downstream 4 | 5165 |
| pSL4198 | pTarget: Target 6_Downstream 6 | 5166 |
| pSL4204 | pTarget: Target 6_Downstream 4 | 5167 |
| pSL4199 | pTarget: Target 7_Downstream 7 | 5168 |
| pSL4205 | pTarget: Target 7_Downstream 4 | 5169 |
| pSL3937 | pCDF_Vch_PJ23119_CRISPR(tSL0004)_QCascade_TnsABC_300 | 5170 |
| | bp-miniTn(Tn7R(56 bp)_99 bp_Tn7L)_SmR | |
| pSL3524 | pDonor_TnR(WT_H.0001) | 5171 |
| pSL3567 | pDonor_TnR(ORF1a_H.0141) | 5172 |
| pSL3568 | pDonor_TnR(ORF1b_H.0189) | 5173 |
| pSL3569 | pDonor_TnR(ORF1c_H.0213) | 5174 |
| pSL3572 | pDonor_TnR(ORF2a_H.0452) | 5175 |
| pSL3570 | pDonor_TnR(ORF3a_H.0506) | 5176 |
| pSL3573 | pDonor_TnR(ORF3b_H.0597) | 5177 |
| pSL3574 | pDonor_TnR(ORF3c_H.0645) | 5178 |
| pSL3571 | oDonor_TnR(ORF3d_H.0653) | 5179 |
| pSL0001 | pUC19 | 5180 |
| pSL3496 | pCOLA_T7_sfGFP1-10_32AA_sfGFP11 | 5181 |
| pSL3497 | pCOLA_T7_sfGFP1-10_32AA | 5182 |
| pSL0008 | pCOLADuet-1 | 5183 |
| pSL3616 | pUC19_TnR(132 bp WT)_Pcat_sfGFP11_TnL | 5184 |
| pSL3498 | pUC57_TnR(WT)_sfGFP11_TnL | 5185 |
| pSL4187 | pCOLA_T7_sfGFP1-10_TnR(WT)_sfGFP11 | 5186 |
| pSL4188 | pCOLA_T7_sfGFP1-10_TnR(ORF1a)_sfGFP11 | 5187 |
| pSL4189 | pCOLA_T7_sfGFP1-10_TnR(ORF1b)_sfGFP11 | 5188 |
| pSL4190 | pCOLA_T7_sfGFP1-10_TnR(ORF1c)_sfGFP11 | 5189 |
| pSL4191 | pCOLA_T7_sfGFP1-10_TnR(ORF2a)_sfGFP11 | 5190 |
| pSL4192 | pCOLA_T7_sfGFP1-10_TnR(ORF3a)_sfGFP11 | 5191 |
| pSL4193 | pCOLA_T7_sfGFP1-10_TnR(ORF3b)_sfGFP11 | 5192 |
| pSL4194 | pCOLA_T7_sfGFP1-10_TnR(ORF3c)_sfGFP11 | 5193 |
| pSL4195 | pCOLA_T7_sfGFP1-10_TnR(ORF3d)_sfGFP11 | 5194 |
| pSL3494 | pCOLA_T7_sfGFP | 5195 |
| pSL1021 | pEffector_crRNA-nt | 5196 |
| pSL4283 | pEffector_crRNA-505 (msrB) | 5197 |
| pSL4450 | pSIM6_pSC101_Donor_TnR(WT)_GGS | 5198 |
| | linker_sfGFP_T7Term_TnL | |
| pSL4451 | pSIM6_pSC101_Donor_TnR(ORF2a)_GGS | 5199 |
| | linker_sfGFP_T7Term_TnL | |
| pSL4052 | pACYC_T7_IHFa_IHFb | 5200 |
| pSL2383 | pSPIN_Tn7007_atypical | 5201 |
| pSL2372 | pSPIN_Tn7011_atypical | 5202 |
| pSL2376 | pSPIN_Tn7014_atypical | 5203 |
| pSL2386 | pSPIN_Tn7016_atypical | 5204 |
| pSL2370 | pSPIN_Tn7000_atypical | 5205 |
| pSL1213 | pCDFL_Vch_PT7_CRISPR(Target4)_QCascade_TnsABC_Tn7R_{—} | 5206 |
| | CmR_Tn7L | |
| pSL1131 | pCDFL_Vch_PJ23101_CRISPR(Target4)_QCascade_TnsABC_{—} | 5207 |
| | Tn7R_CmR_Tn7L | |
| pSL1796 | pCDFL_Vch_PJ23119_CRISPR(Target4)_QCascade_TnsABC | 5208 |
| pSL5047 | pUC57_Tn6677_TnR_cargo_TnR (800 bp- | 5209 |
| | miniTn_symmetric_ends_R-R) | |
| pSL5048 | pUC57_Tn6677_TnL_cargo_TnL (800 bp- | 5210 |
| | miniTn_symmetric_ends_L-L) | |
| pSL4306 | pCDF_Tn7_J23119_TnsABCD_TnL_genomic-PBSs_TnR | 5211 |
| pSL1233 | pSC101_PAM_MSC1_T7_MSC2 | 5212 |
| pSL2684 | pSIM6_pSC101_GamBetaExo | 5213 |
| pSL4218 | | 5214 |
| pSL4373 | | 5215 |
| pSL4374 | | 5216 |
| pSL4050 | pACYC_bCO_scIHF2 | 5217 |
| pSL4134 | hCO dcIHFA-NLS | 5218 |
| pSL4135 | hCO NLS-dcIHFA | 5219 |
| pSL4136 | hCO dcIHFB-NLS | 5220 |
| pSL4137 | hCO NLS-dcIHFB | 5221 |
| pSL4146 | hCO NLS-scIHF2 | 5222 |
| pSL4147 | hCO scIHF2 | 5223 |
| pSL4170 | TnsA-NLS-GSGSGG-IHF-XTEN-GS-TnsB | 5224 |
| pSL4171 | TnsA-NLS-GSGSGG-XTEN-IHF-XTEN-GS-TnsB | 5225 |
| pSL4172 | TnsA-NLS-(GGS)6-IHF-(XTEN)3-TnsB | 5226 |
| pSL4173 | TnsA-NLS-(XTEN)3-IHF-(GGS)6-TnsB | 5227 |
| pSL4178 | IHF-dCas9 | 5228 |

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

1. A system for RNA-guided nucleic acid modification, comprising:
a) an engineered Clustered Regularly Interspaced Short Palindromic Repeats (CRISPR)-associated transposon (CAST) system or one or more nucleic acids encoding the engineered CAST system, wherein the CAST system comprises at least one or all of:
i) at least one Cas protein;
ii) at least one transposon-associated protein; and
iii) at least one guide RNA (gRNA) complementary to at least a portion of a target nucleic acid sequence; and
b) a donor nucleic acid comprising a cargo nucleic acid sequence flanked by at least one or both of: an engineered transposon right end sequence or an engineered transposon left end sequence; and/or
c) at least one integration co-factor protein, or a nucleic acid encoding thereof.

2. The system of claim 1, wherein the engineered transposon right end sequence and/or the engineered left end sequence encodes an amino acid linker sequence.

3. The system of claim 1, wherein the engineered transposon right end sequence and/or the engineered left end sequence is fully or partially AT rich.

4. The system of claim 1, wherein the engineered transposon right end sequence and/or the engineered left end sequence comprises at least two TnsB binding sites (TBSs).

5. The system of claim 4, wherein each TBS comprises a sequence individually selected from: CAMCCATAWRDTGATAWYKH (SEO ID NO: 11), or CMMCBRWAWNNTGAHWWYWN (SEO ID NO: 12),
wherein each M is individually A or C; each W is independently A or T; each R is independently A or G; each D is independently A, G or T; each Y is independently T or C; each K is G or T; B is G, T, or C; and each H is independently A, C or T.

6. The system of claim 1, wherein the engineered transposon right end sequence and/or the engineered left end sequence comprises a 5 to 8 bp terminal end sequence.

7. The system of claim 1, wherein the engineered transposon right end sequence is at least about 75 basepairs (bp).

8. The system of claim 1, wherein the engineered transposon right end sequence comprises a sequence of:
SEQ ID NO: 1, or a variant sequence having one or more additions, substitutions, or deletions thereof,
any of SEQ ID NOs: 2-8;
any of SEQ ID NOs: 18-844;
SEQ ID NOs: 9, or a variant sequence having one or more additions, substitutions, or deletions thereof,
any of SEQ ID NOs: 845-2690;
any of SEQ ID NOs: 2691-2702; or
any of SEQ ID NOs: 2703-3119.

9. The system of claim 1, wherein the engineered transposon left end sequence is at least about 115 basepairs (bp).

10. The system of claim 1, wherein the engineered transposon left end sequence further comprises an Integration Host Factor (IHF) binding site (IBS), wherein the IBS comprises a sequence of WATCARNNNNTTR, wherein W is A or T, R is A or G, and N is any nucleotide.

11. The system of claim 1, wherein the engineered transposon left end sequence comprises a sequence of:
SEQ ID NO: 10, or a variant sequence having one or more substitutions thereof.
any of SEQ ID NOs: 3120-4665;
any of SEQ ID NOs: 4666-4673; or
any of SEQ ID NOs: 4674-5135.

12. The system of claim 1, wherein the cargo nucleic acid sequence encodes a peptide tag or a polypeptide.

13. The system of claim 1, wherein the at least one integration co-factor protein comprises Integration Host Factor (IHF), Factor for Inversion Stimulation (Fis), or a combination thereof.

14. The system of claim 1, wherein the engineered transposon right end sequence and/or the engineered transposon left end sequence is derived from Vibrio cholerae Tn6677 or Pseudoalteromonas Tn7016.

15. A method for DNA integration or labeling a gene product, comprising contacting a target nucleic acid sequence with the system of claim 1.
